(19) United States
(12) Patent Application Publication
(10) Pub. No.: US 2003/0212256 A1
(43) Pub. Date: Nov. 13, 2003

(54) Proteins and nucleic acids encoding same

(76) Inventors: Shlomit R. Edinger, New Haven, CT (US); Valerie Gerlach, Branford, CT (US); John R. MacDougall, Hamden, CT (US); Uriel M. Malyankar, Branford, CT (US); Glennda Smithson, Guildford, CT (US); Isabelle Millet, Milford, CT (US); John A. Peyman, New Haven, CT (US); David J. Stone, Guilford, CT (US); Erik Gunther, Branford, CT (US); Karen Ellerman, Branford, CT (US); Richard A. Shimkets, West Haven, CT (US); Muralidhara Padigaru, Branford, CT (US); Xiaojia Guo, Branford, CT (US); Meera Patturajan, Branford, CT (US); Raymond J. Taupier JR., East Haven, CT (US); Catherine E. Burgess, Wethersfield, CT (US); Bryan D. Zerhusen, Branford, CT (US); Ramesh Kekuda, Stamford, CT (US); Kimberly A. Spytek, New Haven, CT (US); Esha A. Gangolli, Madison, CT (US); Elma R. Fernandes, Branford, CT (US); Linda Gorman, East Haven, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 09/981,151

(22) Filed: Oct. 16, 2001

Related U.S. Application Data
(60) Provisional application No. 60/241,040, filed on Oct. 17, 2000.
(60) Provisional application No. 60/241,058, filed on Oct. 17, 2000.
(60) Provisional application No. 60/241,063, filed on Oct. 17, 2000.
(60) Provisional application No. 60/241,243, filed on Oct. 17, 2000.
(60) Provisional application No. 60/242,152, filed on Oct. 20, 2000.
(60) Provisional application No. 60/242,482, filed on Oct. 23, 2000.
(60) Provisional application No. 60/242,611, filed on Oct. 23, 2000.
(60) Provisional application No. 60/242,612, filed on Oct. 23, 2000.
(60) Provisional application No. 60/242,880, filed on Oct. 24, 2000.
(60) Provisional application No. 60/242,881, filed on Oct. 24, 2000.
(60) Provisional application No. 60/259,028, filed on Dec. 29, 2000.
(60) Provisional application No. 60/269,813, filed on Feb. 20, 2001.
(60) Provisional application No. 60/286,324, filed on Apr. 25, 2001.
(60) Provisional application No. 60/294,108, filed on May 29, 2001.
(60) Provisional application No. 60/303,968, filed on Jul. 9, 2001.

(51) Int. Cl. C07K 14/435; C12P 21/02; C12N 5/06; C07H 21/04; C07K 16/40

(52) U.S. Cl. 530/350; 530/388.1; 435/69.1; 435/325; 435/320.1; 536/23.2

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from U.S. S. No. 60/241,040, filed Oct. 17, 2000; U.S. S. No. 60/241,058, filed Oct. 17, 2000; U.S. S. No. 60/241,063, filed Oct. 17, 2000; U.S. S. No. 60/241,243 filed Oct. 17, 2000; U.S. S. No. 60/242,152, filed Oct. 20, 2000; U.S. S. No. 60/242,482, filed Oct. 23, 2000; U.S. S. No. 60/242,611, filed Oct. 23, 2000; U.S. S. No. 60/242,612, filed Oct. 23, 2000; U.S. S. No. 60/242,880, filed Oct. 24, 2000; U.S. S. No. 60/242,881, filed Oct. 24, 2000; U.S. S. No. 60/259,028, filed Dec. 29, 2000; U.S. S. No. 60/269,813, filed Feb. 20, 2001; U.S. S. No. 60/286,324, filed Apr. 25, 2001; U.S. S. No. 60/294,108, filed May 29, 2001; and U.S. S. No. 60/303,968, filed Jul. 9, 2001 each of which is incorporated by reference in its entirety.

### FIELD OF THE INVENTION

The invention generally relates to nucleic acids and polypeptides encoded thereby.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, NOV8, NOV9, and NOV10 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS:1, 3, 5, 7, 9, 11, 13, 15, 17, 19, 21, 23, 25, and 28. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS:2, 4, 6, 8, 10, 12, 14, 16, 18, 20, 22, 24, 27, and 29. The nucleic acid can be, for example, a genomic DNA fragment or a cDNA molecule that includes the nucleic acid sequence of any of SEQ ID NOS:1, 3, 5, 7, 9, 11, 13, 15, 17, 19, 21, 23, 25, and 28.

Also included in the invention is an oligonucleotide, e.g., an oligonucleotide which includes at least 6 contiguous nucleotides of a NOVX nucleic acid (e.g., SEQ ID NOS: 1, 3, 5, 7, 9, 11, 13, 15, 17, 19, 21, 23, 25, and 28) or a complement of said oligonucleotide.

Also included in the invention are substantially purified NOVX polypeptides (SEQ ID NOS:2, 4, 6, 8, 10, 12, 14, 16, 18, 20, 22, 24, 27, and 29). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., adrenoleukodystrophy, congenital adrenal hyperplasia, leukodystrophies, breast cancer, Small-cell cancer of lung, squamous cell carcinomas, Colorectal cancer, Malignant melanoma, cutaneous, Neuroblastoma, Prostate cancer-brain cancer susceptibility, Alzheimer's disease, epilepsy, Huntington's disease, anxiety, ataxia-telangiectasia, behavioral disorders, multiple sclerosis, muscular dystrophy, myasthenia gravis, neurodegeneration, neuroprotection, Parkinson's disease, pain, stroke, Stroke, Aneurysm, Embolism, autoimmune disease, allergies, addiction, asthma, ARDS, allergy, endometriosis, endocrine dysfunctions, graft versus host disease, graft versus host disease (GVHD), idiopathic thrombocytopenic purpura, immunodeficiencies, IgA nephropathy, lymphaedema, systemic lupus erythematosus, scleroderma, transplantation, psoriasis, crohn's disease, HIV infection, Muscle-eye-brain disease, Neuropathy, paraneoplastic sensory, Charcot-Marie-Tooth neuropathy-2A, SCID due to LCK deficiency, bone marrow transplantation, Kostmann neutropenia, immunodeficiency, thrombocytopenia, eczema, lymphoid malignancy, impaired monocyte motility, Lymphaedema, atherosclerosis, aortic stenosis, atrial septal defect (ASD), atrioventricular (A-V) canal defect, ductus arteriosus, cerebral palsy, cirrhosis, cardiomyopathy, congenital heart defects, hypertension, hemophilia, hypercoagulation, pulmonary stenosis, subaortic stenosis, ventricular septal defect (VSD), valve diseases, bacterial and viral infections, cerebral vascular disease, osteoarthritis, rheumatoid arthritis, Heart block, nonprogressive, Heart block, progressive, 2, Ventricular fibrillation, idiopathic, entricular tachycardia, idiopathic, Thrombocytopenia, congenital amegakaryocytic, Bypass surgery, Bleeding disorders, Adrenoleukodystrophy, Congenital Adrenal Hyperplasia, hypercoagulation, diabetes, obesity, metabolic disorders such as familial amyloidotic polyneuropathy, hyperkinetic diseases, Galactose epimerase deficiency, Glucose transport defect, blood-brain barrier, diverticular disease, emphysema, glomerulonephritis, hypercalceimia, interstitial nephritis, inflammatory bowel disease, Lesch-Nyhan syndrome, polycystic kidney disease, pancreatitis, renal artery stenosis, renal tubular acidosis, tuberous sclerosis, Von Hippel-Lindau (VHL) syndrome, ulcers, neonatal apnea, eagle's syndrome, renal fibrogenesis, Meckel syndrome, skin disorders, connective tissue disorders such as type VIIC Ehlers-Danros syndrome, Primary bile acid malabsorption (PBAM, an idiopathic intestinal disorder), congenital diarrhea, steatorrhea, refractory infantile diarrhea, interruption of the enterohepatic circulation of bile acids, reduced plasma cholesterol levels, Hirschsprung's disease, Cirrhosis, growth failure, Aicardi-Goutieres syndrome 1, Brugada syndrome, Deafness, autosomal recessive 6, Ichthyosiforme erythroderma, congenital, nonbullous, Long QT syndrome-3, Night blindness, congenital stationary, Pituitary ACTH-secreting adenoma, Elliptocytosis-1, Fucosidosis, Hypophosphatasia (adult, childhood, infantile), Porphyria cutanea tarda, Porphyria, hepatoerythropoietic, Schwartz-Jampel syndrome, Myopathy due to succinate dehydrogenase deficiency, Bartter syndrome, type 3, Corneal dystrophy, crystalline, Schnyder, Hyperprolinemia, type II, erythrokeratodermia variabilis, palmoplantar keratoderma, diseases and disorders involving intercellular metabolic and electrical communication, diseases and disorders involving coordination, proliferation and differentiation, diseases and disorders involving maintainance of tissue homeostasis, growth control, development, and synchronized response of cells to stimuli, Wiskott-Aldrich syndrome, cytoskeletal abnormalities, trauma, tissue regeneration (in vitro and in vivo), respiratory disease, gastro-intestinal diseases, muscle, bone, joint and skeletal disorders, hematopoietic disorders, urinary system disorders, Tissue and organ transplantation, Fibromuscular dysplasia, Hyperparathyroidism, Hypoparathyroidism, Hyperthyroidism and Hypothyroidism, SIDS, Xerostomia, Tonsilitis, Osteoporosis, Ankylosing spondylitis, Scoliosis, Tendinitis, Dental disease and infection, growth and reproductive disorders, hypogonadism, fertility, and/or other pathologies and disorders of the like.

The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their encoded polypeptides. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID | | |
|---|---|---|---|---|
| | | NO | | |
| NOVX | | (nucleic | SEQ ID NO | |
| Assignment | Internal Identification | acid) | (polypeptide) | Homology |
| 1a | SC138213196_A | 1 | 2 | Zinc Metalloprotease-like |
| 1b | 137043926_EXT_1 | 3 | 4 | zinc metalloprotease-1-like |
| 1c | CG52952-03 | 5 | 6 | ADAM-TS 7-like |
| 1d | CG52952-04 | 7 | 8 | ADAM-TS 7-like |
| 2 | SC_78316254_A | 9 | 10 | Alpha-2-macroglobulin |
| | | | | precursor-like |
| 3 | GMAC079237_A | 11 | 12 | Ileal Sodium/Bile Acid |
| | | | | Cotransporter-like |
| 4 | AL161453_A | 13 | 14 | Prohibitin-like |
| 5 | dj1182a14_da1 | 15 | 16 | Macrophage Stimulating |
| | | | | Protein Precursor-like |
| 6 | GM382a20_A | 17 | 18 | Fatty Acid-Binding Protein- |
| | | | | like |
| 7 | sggc_draft_dj895c5_{—} | 19 | 20 | Gap junction beta-5 |
| | 20000819 | | | protein-like |
| 8 | 56072181_da1 | 21 | 22 | Metallothionein-like |
| 9 | 2855519_0_19_da1 | 23 | 24 | CIP4-like |
| 10a | 129297354_EXT | 25 | 27 | hepsin/plasma transmembrane |
| | | | | serine protease-like |
| 10b | CG106783-02 | 28 | 29 | Spinesin-like |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

NOV1 is homologous to a Zinc Metalloprotease/ADAM-TS 7-like family of proteins.

Thus, the NOV1 nucleic acids, polypeptides, antibodies and related compounds according to the invention will be useful in therapeutic and diagnostic applications implicated in, for example; adrenoleukodystrophy, Alzheimer's disease, autoimmune disease, allergies, addiction, anxiety, ataxia-telangiectasia, asthma, ARDS, atherosclerosis, behavioral disorders, aortic stenosis, atrial septal defect (ASD), atrioventricular (A-V) canal defect, ductus arteriosus, allergy, cerebral palsy, congenital adrenal hyperplasia, cirrhosis, cardiomyopathy, congenital heart defects, diabetes, diverticular disease, epilepsy, emphysema, endometriosis, endocrine dysfunctions, graft versus host disease, glomerulonephritis, graft versus host disease (GVHD), growth and reproductive disorders, hemophilia, hypercoagulation, hypercalceimia, Huntington's disease, hypertension, hypogonadism, fertility, idiopathic thrombocytopenic purpura, immunodeficiencies, interstitial nephritis, IgA nephropathy, lymphaedema, inflammatory bowel disease, Lesch-Nyhan syndrome, leukodystrophies, multiple sclerosis, muscular dystrophy, myasthenia gravis, neurodegeneration, neuroprotection, obesity, Parkinson's disease, pain, polycystic kidney disease, pulmonary stenosis, pancreatitis, renal artery stenosis, renal tubular acidosis, stroke, systemic lupus erythematosus, scleroderma, subaortic stenosis, transplantation, tuberous sclerosis, Von Hippel-Lindau (VHL) syndrome, ventricular septal defect (VSD), valve diseases, Von Hippel-Lindau (VHL) syndrome, ulcers, bacterial and viral infections, neonatal apnea, eagle's syndrome, atherosclerosis, metabolic disorders such as familial amyloidotic polyneuropathy, hyperkinetic diseases, muscular dystrophy, cerebral vascular disease, hypertension, cardiovascular diseseases, renal fibrogenesis, inflammatory bowel disease, Meckel syndrome, colorectal cancer, papillomavirus infection and cervical carcinoma, liver malignancies, skin disorders, connective tissue disorders such as type VIIC Ehlers-Danros syndrome, osteoarthritis, rheumatoid arthritis, or other pathologie or conditions.

NOV2 is homologous to the Alpha-2-macroglobulin precursor-like family of proteins. Thus NOV2 nucleic acids, polypeptides, antibodies and related compounds according to the invention will be useful in therapeutic and diagnostic applications implicated in, for example; asthma, allergy and psoriasis, Alzheimer disease, Emphysema, pulmonary disease, immune disorders and Cancer and/or other pathologies and disorders.

NOV3 is homologous to a family of Ileal Sodium/Bile Acid Cotransporter-like proteins. Thus, the NOV3 nucleic acids and polypeptides, antibodies and related compounds according to the invention will be useful in therapeutic and diagnostic applications implicated in, for example: Primary bile acid malabsorption (PBAM, an idiopathic intestinal disorder), congenital diarrhea, steatorrhea, refractory infantile diarrhea, interruption of the enterohepatic circulation of bile acids, reduced plasma cholesterol levels, crohn's disease, Inflammatory bowel disease, Diverticular disease, Hirschsprung's disease, Cirrhosis, Transplantation, Hypercalceimia, Ulcers, growth failure and/or other pathologies.

NOV4 is homologous to the Prohibitin-like family of proteins. Thus, NOV4 nucleic acids, polypeptides, antibodies and related compounds according to the invention will be useful in therapeutic and diagnostic applications implicated in, for example: breast cancer (In a sporadic breast cancer, Sato et al. (1992) found a missense mutation from valine (GTC) to alanine (GCC) at codon 88 of the PHB gene), and/or other pathologies.

NOV5 is homologous to the Macrophage Stimulating Protein Precursor-like family of proteins. Thus NOV5 nucleic acids, polypeptides, antibodies and related compounds according to the invention will be useful in therapeutic and diagnostic applications implicated in, for example: Aicardi-Goutieres syndrome 1, Brugada syndrome, Deafness, autosomal recessive 6, Heart block, nonprogressive, Heart block, progressive, 2, Ichthyosiforme erythroderma, congenital, nonbullous, Long QT syndrome-3, Night blindness, congenital stationary, Pituitary ACTH-secreting adenoma, Small-cell cancer of lung, Ventricular fibrillation, idiopathic, entricular tachycardia, idiopathic, HIV infection, susceptibility/resistance to, Von Hippel-Lindau (VHL) syndrome, Cirrhosis, Transplantation, and/or other pathologies/disorders.

NOV6 is homologous to the Fatty Acid-Binding Protein-like family of proteins. Thus NOV6 nucleic acids, polypeptides, antibodies and related compounds according to the invention will be useful in therapeutic and diagnostic applications implicated in, for example: Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Transplantation, Endometriosis, Inflammatory bowel disease, Diverticular disease, Hirschsprung's disease, Crohn's Disease, Hemophilia, hypercoagulation, Idiopathic thrombocytopenic purpura, immunodeficiencies, Osteoporosis, Hypercalceimia, Arthritis, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Asthma, allergy, ARDS, Lesch-Nyhan syndrome, Multiple sclerosis, Leukodystrophies, Behavioral disorders, Addiction, Anxiety, Pain, Neuroprotection Fertility, psoriasis, cancer including but not limited to basal and squamous cell carcinomas, obesity, diabetis, and/or other pathologies and disorders involving fatty acid transport of skin, oral mucosa as well as other organs, and/or other pathologies/disorders.

NOV7 is homologous to members of the Gap junction beta-5 protein-like family of proteins. Thus, the NOV7 nucleic acids, polypeptides, antibodies and related compounds according to the invention will be useful in therapeutic and diagnostic applications implicated in, for example; Deafness, autosomal dominant 2, Elliptocytosis-1, Fucosidosis, Hypophosphatasia (adult, childhood, infantile), Muscle-eye-brain disease, Neuropathy, paraneoplastic sensory, Porphyria cutanea tarda, Porphyria, hepatoerythropoietic, Schwartz-Jampel syndrome, Thrombocytopenia, congenital amegakaryocytic, Charcot-Marie-Tooth neuropathy-2A, Galactose epimerase deficiency, Glucose transport defect, blood-brain barrier, Kostmann neutropenia, Muscular dystrophy, congenital, with early spine rigidity, Myopathy due to succinate dehydrogenase deficiency, SCID due to LCK deficiency, Colorectal cancer, resistance to, Bartter syndrome, type 3, Breast cancer, ductal, Corneal dystrophy, crystalline, Schnyder, Hyperprolinemia, type II, Inflammatory bowel disease 7, Malignant melanoma, cutaneous, Neuroblastoma, Prostate cancer-brain cancer susceptibility, erythrokeratodermia variabilis, palmoplantar keratoderma, diseases and disorders involving intercellular metabolic and electrical communication, diseases and disorders involving coordination, proliferation and differentiation, diseases and disorders involving maintainance of tissue homeostasis, growth control, development, and synchronized response of cells to stimuli, diseases and disorders involving the the immune system, diseases and disorders involving regulation of bone cell differentiation, and/or other pathologies/disorders.

NOV8 is homologous to the Metallothionein-like family of proteins. Thus, NOV8 nucleic acids and polypeptides, antibodies and related compounds according to the invention will be useful in therapeutic and diagnostic applications implicated in, for example; Gitelman syndrome, Menkes disease, Wilson's disease, acrodermatitis enteropathica, myelomonocytic leukemia, eosinophil disorders, hepatic disorders such as hepatic copper toxicity, and/or other pathologies/disorders.

NOV9 is homologous to the CDC-42 interacting protein 4-like family of proteins. Thus, NOV9 nucleic acids and polypeptides, antibodies and related compounds according to the invention will be useful in therapeutic and diagnostic applications implicated in, for example; Wiskott-Aldrich syndrome, immunodeficiency, thrombocytopenia, eczema, lymphoid malignancy cytoskeletal abnormalities, impaired monocyte motility, Muscular dystrophy, Lesch-Nyhan syndrome, Myasthenia gravis, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Ataxia-telangiectasia, Leukodystrophies, Behavioral disorders, Addiction, Anxiety, Pain, Neuroprotection, Fertility, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Diabetes, Pancreatitis, Obesity, Systemic lupus erythematosus, Autoimmune disease, Asthma, Emphysema, Scleroderma, allergy, ARDS, Cirrhosis, Transplantation, Diabetes, Autoimmune disease, Renal artery stenosis, Interstitial nephritis, Glomerulonephritis, Polycystic kidney disease, Renal tubular acidosis, IgA nephropathy, Hypercalceimia, and/or other pathologies/disorders.

NOV10 is homologous to the hepsin/plasma transmembrane serine protease/spinesin-like family of proteins. Thus, NOV10 nucleic acids and polypeptides, antibodies and related compounds according to the invention will be useful in therapeutic and diagnostic applications implicated in, for example; cancer, trauma, tissue regeneration (in vitro and in vivo), viral/bacterial/parasitic infections, immunological disease, respiratory disease, gastro-intestinal diseases, reproductive health, neurological and neurodegenerative diseases, bone marrow transplantation, metabolic and endocrine diseases, allergy and inflammation, nephrological disorders, cardiovascular diseases, muscle, bone, joint and skeletal disorders, hematopoietic disorders, urinary system disorders, Tissue and organ transplantation, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Scleroderma, Obesity, Hypertension, Fibromuscular dysplasia, Stroke, Aneurysm, Myocardial infarction, Embolism, Bypass surgery, Anemia, Bleeding disorders, Adrenoleukodystrophy, Congenital Adrenal Hyperplasia, Diabetes, Von Hippel-Lindau (VHL) syndrome, Pancreatitis, Hyperparathyroidism, Hypoparathyroidism, Hyperthyroidism and Hypothyroidism, SIDS, Endometriosis, infertility, Xerostomia, Hypercalceimia, Ulcers, Cirrhosis, Inflammatory bowel disease, Diverticular disease, Hirschsprung's disease, Crohn's Disease, Appendicitis, Hemophilia, hypercoagulation, autoimmume disease, allergies, immunodeficiencies, transplantation, Graft vesus host disease (GVHD), Ataxia-telangiectasia, Autoimmume disease, Hemophilia, Hypercoagulation, Idiopathic thrombocytopenic purpura, Immunodeficiencies, Lymphedema, Allergies, Hemophilia, hypercoagulation, idiopathic thrombocytopenic purpura, Lymphaedema, Tonsilitis, Osteoporosis, Hypercalceimia, Arthritis, Ankylosing spondylitis, Scoliosis, Tendinitis, Muscular dystrophy, Lesch-Nyhan syndrome, Myasthenia gravis, Dental disease and infection, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Behavioral disorders, Addiction, Anxiety, Pain, Neuroprotection, Endocrine dysfunctions, Growth and reproductive disorders, Myasthenia gravis, Leukodystrophies, Pain, Neuroprotection, Systemic lupus erythematosus, Autoimmune disease, Emphysema, Scleroderma, ARDS, Pharyngitis, Laryngitis, Asthma, Hearing loss, Tinnitus, Psoriasis, Actinic keratosis, Tuberous sclerosis, Acne, Hair growth, allopecia, pigmentation disorders, endocrine disorders, cystitis, incontinence, Autoimmune disease, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome, Vesicoureteral reflux, and/or other pathologies/disorders.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

NOV1 includes three novel zinc metalloprotease/ADAM-TS 7-like proteins disclosed below. The disclosed sequences have been named NOV1a, NOV1b, NOV1c, and NOV1d.

NOV1a

A disclosed NOV1a nucleic acid of 2997 nucleotides (also referred to as SC138213196_A) encoding a novel Zinc Metalloprotease-like protein is shown in Table 1A. An open reading frame was identified beginning with an ATG initiation codon at nucleotides 10-12 and ending with a TAA codon at nucleotides 2968-2970. A putative untranslated region upstream from the initiation codon and downstream from the termination codon is underlined in Table 1A. The start and stop codons are in bold letters.

TABLE 1A
NOV1a nucleotide sequence.

(SEQ ID NO:1)

CGCTCCTGGATGAAGCCCCGCGCGCGCGGATGGCGGGGCTTGGCGGCGCTGTGGATGCTGTTGGCGCAGGTG
GCCGAGCAGGTGAGTCCCGGGCGCTCCCACCAGCGCGGAAACCGCGGGTCCGGACAGCTGGAGGCGAGTCCC
CCGCGGCTCCTCTCCCGCGGACCCCGCCGTCTCACCGCGATGTCGCCGCTGTTTTCCGCAGGCACCTGCGTG
CGCCATGGGACCCGCAGCGGCAGCGCCTGGGAGCCCGAGCGTCCCGCGTCCTCCTCCACCCGCGGAGCGGCC
GGGCTGGATGGAAAAGGGCGGGACATGGATGAAGCTGGAAACCATCGTTCTCAGCAAACTAACACAGGAACA
GAAAACCAAACACTGCATGTTCTCACTCAATATGACCTGGTCTCTGCCTACGAGGTTGACCACAGGGGCGAT
TACGTGTCCCATGAAATCATGCACCATCAGCGGCGGAGAAGAGCAGTGGCCGTGTCCGAGGTTGAGTCTCTT
CACCTTCGGCTGAAAGGCCCCAGGCACGACTTCCACATGGATCTGAGGACTTCCAGCAGCCTAGTGGCTCCT
GGCTTTATTGTGCAGACGTTGGGAAAGACAGGCACTAAGTCTGTGCAGACTTTACCGCCAGAGGACTTCTGT
TTCTATCAAGGCTCTTTGCGATCACACAGAAACTCGCCATCGCATGGAGGGAAGTTCTGTGAGGGCTCCACT
CGCACTCTGAAGCTCTGCAACAGTCAGAAATGTCCCCGGGACAGTGTTGACTTCCGTGCTGCTCAGTGTGCC
GAGCACAACAGCAGACGATTCAGAGGGCGGCACTACAAGTGGAAGCCTTACACTCAAGTAGAAGNNGACTTA
TGCAAACTCTACTGTATCGCAGAAGGATTTGATTTCTTCTTTTCTTTGTCAAATAAAGTCAAAGATGGGACT
CCATGCTCGGAGGATAGCCGTAATGTTTGTATAGATGGGATATGTGAGCTCAGTGTGGTGTCCACATCTGCG
CACATGCCCCAGCCTCCCAAGGAAGACCTCTTCATCTTGCCAGATGAGTATAAGTCTTGCTTACGGCATAAG
CGCTCTCTTCTGAGGTCCCATAGAAATGAAGAACTGAACGTGGAGACCTTGGTGGTGGTCGACAAAAAGATG
ATGCAAAACCATGGCCATGAAAATATCACCACCTACGTGCTCACGATACTCAACATGGTATCTGCTTTATTC
AAAGATGGAACAATAGGAGGAAACATCAACATTGCAATTGTAGGTCTGATTCTTCTAGAAGATGAACAGCCA
GGACTGGTGATAAGTCACCACGCAGACCACACCTTAAGTAGCTTCTGCCAGTGGCAGTCTGGATTGATGGGG
AAAGATGGGACTCGTCATGACCACGCCATCTTACTGACTGGTCTGGATATATGTTCCTGGAAGAATGAGCCC
TGTGACACTTTGGGATTTGCACCCATAAGTGGAATGTGTAGTAAATATCGCAGCTGCACGATTAATGAAGAT
ACAGGTCTTGGACTGGCCTTCACCATTGCCCATGAGTCTGGACACAACTTTGGCATGATTCATGATGGAGAA
GGGAACATGTGTAAAAAGTCCGAGGGCAACATCATGTCCCCTACATTGGCAGGACGCAATGGAGTCTTCTCC
TGGTCACCCTGCAGCCGCCAGTATCTACACAAATTTCTAAGCACCGCTCAAGCTATCTGCCTTGCTGATCAG
CCAAAGCCTGTGAAGGAATACAAGTATCCTGAGAAATTGCCAGGAGAATTATATGATGCAAACACACAGTGC
AAGTGGCAGTTCGGAGAGAAAGCCAAGCTCTGCATGCTGGACTTTAAAAAGGACATCTGTAAAGCCCTGTGG
TGCCATCGTATTGGAAGGAAATGTGAGACTAAATTTATGCCAGCAGCAGAAGGCACAATTTGTGGGCATGAC
ATGTGGTGCCGGGGAGGACAGTGTGTGAAATATGGTGATGAAGGCCCCAAGCCCACCCATGGCCACTGGTCG
GACTGGTCTTCTTGGTCCCCATGCTCCAGGACCTGCGGAGGGGGAGTATCTCATAGGAGTCGCCTCTGCACC
AACCCCAATCCATCGCATGGAGGGAAGTTCTGTGAGGGCTCCACTCGCACTCTGAAGCTCTGCAACAGTCAG
AAATGTCCCCGGGACAGTGTTGACTTCCGTGCTGCTCAGTGTGCCGAGCACAACAGCAGACGATTCAGAGGG
CGGCACTACAAGTGGAAGCCTCAGGACTTATGCAAACTCTACTGTATCGCAGAAGGATTTGATTTCTTCTTT
TCTTTGTCAAATAAAGTCAAAGATGGGACTCCATGCTCGGAGGATAGCCGTAATGTTTGTATAGATGGGATA
TGTGAGNTTGGATGTGACAATGTCCTTGGATCTGATGCTGTTGAAGACGTCTGTGGGGTGTGTAACGGGAAT
AACTCAGCCTGCACGATTCACAGGGGTCTCTACACCAAGCACCACCACACCAACCATTATCACATGGTCACC
ATTCCTTCTGGAGCCCGGAGTATCCGCATCTATGAAATGAACGTCTCTACCTCCTACATTTCTGTGCGCAAT
GCCCTCAGAAGGTACTACCTGAATGGGCACTGGACCGTGGACTGGCCCGGCCGGTACAAATTTTCGGGCACT
ACTTTCGACTACAGACGGTCCTATAATGAGCCCGAGAACTTAATCGCTACTGGACCAACCAACGAGACACTG
ATTGTGGAGCTGCTGTTTCAGGGAAGGAACCCGGGTGTTGCCTGGGAATACTCCATGCCTCGCTTGGGGACC
GAGAAGCAGCCCCCTGCCCAGCCCAGCTACACTTGGGCCATCGTGCGCTCTGAGTGCTCCGTGTCCTGCGGA
GGGGGTAGGTGCCTTCCAGTGCTGCTCCTGGAGGCAGCATGTCAGCCTTCAGCCACTGCGTACATTGCACTG
GCCTTTCTTGAATCCTAATGAGCAGCCCGGNGCTTCTCCCTGCCA

In a search of public sequence databases, the NOV1a nucleic acid sequence, located on chromsome 5 has 250 of 375 bases (66%) identical to a zinc metalloprotease ADAMTS6 (ADAMTS6) mRNA from Homo sapiens (GENBANK-ID: AF140674). Public nucleotide databases include all GenBank databases and the GeneSeq patent database.

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the NOV1 BLAST analysis, e.g., thioredoxin mRNA from Ovis aries, matched the Query NOV1 sequence purely by chance is 9.4e⁻²⁵. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNNNNNN”) or the letter “X” in protein sequences (e.g., “XXXXXXXXX”). Low-complexity regions can result in high scores that reflect compositional bias rather than significant position-by-position alignment. (Wootton and Federhen, Methods Enzymol 266:554-571, 1996).

The disclosed NOV1a polypeptide (SEQ ID NO:2) encoded by SEQ ID NO:1 has 986 amino acid residues and is presented in Table 1B using the one-letter amino acid code. Signal P, Psort and/or Hydropathy results predict that NOV1a has a signal peptide and is likely to be localized in extracellularly with a certainty of 0.5469. In other embodiments, NOV1a may also be localized to the lysosome (lumen) with acertainty of 0.1900, the microbody (peroxisome) with a certainty of 0.1297 or in the endoplasmic reticulum (membrane) with a certainty of 0.1000. The most likely cleavage site for a NOV1a peptide is between amino acids 26 and 27, at: VAE-QV.

TABLE 1B
Encoded NOV1a protein sequence.

(SEQ ID NO:2)

MKPRARGWRGLAALWMLLAQVAEQVSPGRSHQRGNRGSGQLEASPPRLLSRGPRRLTAMSPLFSAGTCVRHG
TRSGSAWEPERPASSSTRGAAGLDGKGRDMDEAGNHRSQQTNTGTENQTLHVLTQYDLVSAYEVDHRGDYVS
HEIMHHQRRRRAVAVSEVESLHLRLKGPRHDFHMDLRTSSSLVAPGFIVQTLGKTGTKSVQTLPPEDFCFYQ
GSLRSHRNSPSHGGKFCEGSTRTLKLCNSQKCPRDSVDFRAAQCAEHNSRRFRGRHYKWKPYTQVEXDLCKL
YCIAEGFDFFFSLSNKVKDGTPCSEDSRNVCIDGICELSVVSTSAHMPQPPKEDLFILPDEYKSCLRHKRSL
LRSHRNEELNVETLVVVDKKMMQNHGHENITTYVLTILNMVSALFKDGTIGGNINIAIVGLILLEDEQPGLV
ISHHADHTLSSFCQWQSGLMGKDGTRHDHAILLTGLDICSWKNEPCDTLGFAPISGMCSKYRSCTINEDTGL
GLAFTIAHESGHNFGMIHDGEGNMCKKSEGNIMSPTLAGRNGVFSWSPCSRQYLHKFLSTAQAICLADQPKP
VKEYKYPEKLPGELYDANTQCKWQFGEKAKLCMLDFKKDICKALWCHRIGRKCETKFMPAAEGTICGHDMWC
RGGQCVKYGDEGPKPTHGHWSDWSSWSPCSRTCGGGVSHRSRLCTNPNPSHGGKFCEGSTRTLKLCNSQKCP
RDSVDFRAAQCAEHNSRRFRGRHYKWKPQDLCKLYCIAEGFDFFFSLSNKVKDGTPCSEDSRNVCIDGICEX
GCDNVLGSDAVEDVCGVCNGNNSACTIHRGLYTKHHHTNHYHMVTIPSGARSIRIYEMNVSTSYISVRNALR
RYYLNGHWTVDWPGRYKFSGTTFDYRRSYNEPENLIATGPTNETLIVELLFQGRNPGVAWEYSMPRLGTEKQ
PPAQPSYTWAIVRSECSVSCGGGRCLPVLLLEAACQPSATAYIALAFLES

A search of sequence databases reveals that the NOV1a amino acid sequence has 257 of 579 amino acid residues (44%) identical to, and 356 of 579 amino acid residues (61%) similar to, the 997 amino acid residue Zinc Metalloprotease Adamts7 protein from Homo sapiens (Human) (Q9UKP4) (E=6.1ee⁻¹⁴⁹). Public amino acid databases include the GenBank databases, SwissProt, PDB and PIR.

NOV1a is expressed in the lung.

NOV1b

A disclosed NOV1b nucleic acid of 2433 nucleotides (also referred to as 137043926_EXT_{—}1) encoding a novel zinc metalloprotease-1-like protein is shown in Table 1 C. An open reading frame was identified beginning with an ATG initiation codon at nucleotides 31-33 and ending with a TAA codon at nucleotides 2404-2406. A putative untranslated regions upstream from the initiation codon and downstream of the termination codon are underlined in Table 1C. The start and stop codons are in bold letters.

TABLE 1C
NOV1b nucleotide sequence.

(SEQ ID NO:3)

GTGGCCCCTAGCCCCTCGGAGCGCTCCTGGATGAAGCCCCGCGCGCGCGGATGGCGGGGCTTGGCGGCGCTG
TGGATGCTGCTGGCGCAGGTGGCCGAGCAGGTGAGTCCCGGGCGCTCCCACCAGCGCGGAAACCGCGGGTCC
GGACAGCTGGAGGCGAGTCCCCCGCGGCTCCTCTCCCGCGGACCCCGCCGTCTCACCGCGATGTCGCCGCTG
TTTTCCGCAGGCACCTGCGTGCGCCATGGGACCCGCAGCGGCAGCGCCTGGGAGCCCGAGCGTCCCGCGTCC
TCCTCCACCCGCGGAGCGGCCGGGCTGGATGGAAAAGGGCGGGACATGGATGAAGCTGGAAACCATCGTTCT
CAGCAAACTAACACAGGAACAGAAAACCAAACACTGCATGTTCTCACTCGTGAATATGACCTGGTCTCTGCC
TACGAGGTTGACCACAGGGGCGATTACGTGTCCCATGAAATCATGCACCATCAGCGGCGGAGAAGAGCAGTG
GCCGTGTCCGAGGTTGAGTCTCTTCACCTTCGGCTGAAAGGCCCCAGGCACGACTTCCACATGGATCTGAGG
ACTTCCAGCAGCCTAGTGGCTCCTGGCTTTATTGTGCAGACGTTGGGAAAGACAGGCACTAAGTCTGTGCAG
ACTTTACCGCCAGAGGACTTCTGTTTCTATCAAGGCTCTTTGCGATCACACAGAAACTCCCCATCGCATGGA
GGGAAGTTCTGTGAGGGCTCCACTCGCACTCTGAAGCTCTGCAACAGTCAGAAATGTCCCCGGGACAGTGTT
GACTTCCGTGCTGCTCAGTGTGCCGAGCACAACAGCAGACGATTCAGAGGGCGGCACTACAAGTGGAAGCCT
TACACTCAAGTAGAACAGGACTTATGCAAACTCTACTGTATCGCAGAAGGATTTGATTTCTTCTTTTCTTTG
TCAAATAAAGTCAAAGATGGGACTCCATGCTCGGAGGATAGCCGTAATGTTTGTATAGATGGGATATGTGAG
ATGCCCCAGCCTCCCAAGGAAGACCTCTTCATCTTGCCAGATGAGTATAAGTCTTGCTTACGGCATAAGCGC
TCTCTTCTGAGGTCCCATAGAAATGAAGAACTGAACGTGGAGACCTTGGTGGTGGTCGACAAAAAGATGATG
CAAAACCATGGCCATGAAAATATCACCACCTACGTGCTCACGATACTCAACATGGTATCTGCTTTATTCAAA
GATGGAACAATAGGAGGAAACATCAACATTGCAATTGTAGGTCTGATTCTTCTAGAAGATGAACAGGACATC
TGTAAAGCCCTGTGGTGCCATCGTATTGGAAGGAAATGTGAGACTAAATTTATGCCAGCAGCAGAAGGCACA
ATTTGTGGGCATGACATGTGGTGCCGGGGAGGACAGTGTGTGAAATATGGTGATGAAGGCCCCAAGCCCACC
CATGGCCACTGGTCGGACTGGTCTTCTTGGTCCCCATGCTCCAGGACCTGCGGAGGGGGAGTATCTCATAGG
AGTCGCCTCTGCACCAACCCCAGGCCATCGCATGGAGGGAAGTTCTGTGAGGGCTCCACTCGCACTCTGAAG
CTCTGCAACAGTCAGAAATGTCCCCGGGACAGTGTTGACTTCCGTGCTGCTCAGTGTGCCGAGCACAACAGC
AGACGATTCAGAGGGCGGCACTACAAGTGGAAGCCTCAGGACTTATGCAAACTCTACTGTATCGCAGAAGGA
TTTGATTTCTTCTTTTCTTTGTCAAATAAAGTCAAAGATGGGACTCCATGCTCGGAGGATAGCCGTAATGTT
TGTATAGATGGGATATGTGAGGGATGTGACAATGTCCTTGGATCTGATGCTGTTGAAGACGTCTGTGGGGTG
TGTAACGGGAATAACTCAGCCTGCACGATTCACAGGGGTCTCTACACCAAGCACCACCACACCAACTATTAT
CACATGGTCACCATTCCTTCTGGAGCCCGGAGTATCCGCATCTATGAAATGAACGTCTCTACCTCCTACATT
TCTGTGCGCAATGCCCTCAGAAGGTACTACCTGAATGGGCACTGGACCGTGGACTGGCCCGGCCGGTACAAA
TTTTCGGGCACTACTTTCGACTACAGACGGTCCTATAATGAGCCCGAGAACTTAATCGCTACTGGACCAACC
AACGAGACACTGATTGTGGAGCTGCTGTTTCAGGGAAGGAACCCGGGTGTTGCCTGGGAATACTCCATGCCT
CGCTTGGGGACCGAGAAGCAGCCCCCTGCCCAGCCCAGCTACACTTGGGCCATCGTGCGCTCTGAGTGCTCC
GTGTCCTGCGGAGGGGGTAGGTGCCTTCCAGTGCTGCTCCTGGAGGCAGCATGTCAGCCTTCAGCCACTGCG
TACATTGCACTGGCCTTTCTTGAATCCTAATGAGCAGCCCGGGGCTTCTCCCTGCCA

In a search of public sequence databases, the NOV1b nucleic acid sequence, located on chromsome 5 has 101 of 126 bases (80%) identical to agb:GENBANK-ID:HSA400877|acc:AJ400877.1 mRNA from Homo sapiens (Homo sapiens ASCL3 gene, CEGP1 gene, C11orf14 gene, C11orf15 gene, C11orf16 gene and C11orf17 gene) (E=2.3e⁻⁷. Public nucleotide databases include all GenBank databases and the GeneSeq patent database.

The disclosed NOV1b polypeptide (SEQ ID NO:4) encoded by SEQ ID NO:3 has 791 amino acid residues and is presented in Table ID using the one-letter amino acid code. Signal P, Psort and/or Hydropathy results predict that NOV1b has a signal peptide and is likely to be localized in extracellularlye with a certainty of 0.5469. In other embodiments, NOV1b may also be localized to the lysosome (lumen) with acertainty of 0.1900, the microbody (peroxisome) with a certainty of 0.1144 or in the endoplasmic reticulum (membrane) with a certainty of 0.1000. The most likely cleavage site for a NOV1b peptide is between amino acids 23 and 24, at: VAE-QV.

TABLE 1D
Encoded NOV1b protein sequence.

(SEQ ID NO:4)

MKPRARGWRGLAALWMLLAQVAEQVSPGRSHQRGNRGSGQLEASPPRLLSRGPRRLTAMSPLFSAGTCVRHG
TRSGSAWEPERPASSSTRGAAGLDGKGRDMDEAGNHRSQQTNTGTENQTLHVLTREYDLVSAYEVDHRGDYV
SHEIMHHQRRRRAVAVSEVESLHLRLKGPRHDFHMDLRTSSSLVAPGFIVQTLGKTGTKSVQTLPPEDFCFY
QGSLRSHRNSPSHGGKFCEGSTRTLKLCNSQKCPRDSVDFRAAQCAEHNSRRFRGRHYKWKPYTQVEQDLCK
LYCIAEGFDFFFSLSNKVKDGTPCSEDSRNVCIDGICEMPQPPKEDLFILPDEYKSCLRHKRSLLRSHRNEE
LNVETLVVVDKKMMQNHGHENITTYVLTILNMVSALFKDGTIGGNINIAIVGLILLEDEQDICKALWCHRIG
RKCETKFMPAAEGTICGHDMWCRGGQCVKYGDEGPKPTHGHWSDWSSWSPCSRTCGGGVSHRSRLCTNPRPS
HGGKFCEGSTRTLKLCNSQKCPRDSVDFRAAQCAEHNSRRFRGRHYKWKPQDLCKLYCIAEGFDFFFSLSNK
VKDGTPCSEDSRNVCIDGICEGCDNVLGSDAVEDVCGVCNGNNSACTIHRGLYTKHHHTNYYHMVTIPSGAR
SIRIYEMNVSTSYISVRNALRRYYLNGHWTVDWPGRYKFSGTTFDYRRSYNEPENLIATGPTNETLIVELLF
QGRNPGVAWEYSMPRLGTEKQPPAQPSYTWAIVRSECSVSCGGGRCLPVLLLEAACQPSATAYIALAFLES

A search of sequence databases reveals that the NOV1b amino acid sequence has 152 of 357 amino acid residues(42%) identical to, and 216 of357 amino acid residues (60%) similar to, the 860 amino acid residue ptnr:SWISSNEW-ACC:Q9UKP5 protein from Homo sapiens (Human) (ADAM-TS 6 precursor (EC 3.4.24.-) (A Disintegrin And Metalloproteinase With Thrombospondin Motifs 6) (ADAMTS-6) (ADAM-TS6) (E=4.8e⁻¹⁰¹). Public amino acid databases include the GenBank databases, SwissProt, PDB and PIR.

NOV1b is expressed in at least the following tissues: brain, liver, spleen, uterus, colon, tonsil, lung, germ cells. This information was derived by determining the tissue sources of the sequences that were included in the invention including but not limited to SeqCalling sources, Public EST sources, Genomic Clone sources, Literature sources, and/or RACE sources.

NOV1c

In the present invention, the target sequence identified previously, NOV 1 a, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated NOV1c. This differs from the previously identified sequence NOV1a in having a different N-terminus

A disclosed NOV1c nucleic acid of 2902 nucleotides (also referred to as CG52952-03) encoding a novel ADAM-TS 7-like protein is shown in Table 1E. An open reading frame was identified beginning with an ATG initiation codon at nucleotides 182-184 and ending with a TAA codon at nucleotides 2750-2752. A putative untranslated region upstream from the initiation codon and downstream from the termination codon is underlined in Table IE. The start and stop codons are in bold letters.

TABLE 1E
NOV1c nucleotide sequence.

(SEQ ID NO:5)

TTGGCGGCGCTGTGGATGCTGCTGGCGCAGGTGGCCGAGCAGGTGAGTCCCGGGCGCTCCCACCAGCGCGGA
AACCGCGGGTCCGGACAGCTGGAGGCGAGTCCCCCGCGGCTCCTCTCCCGCGGACCCCGCCGTCTCACCGCG
ATGTCGCCGCTGTTTTCCGCAGGCACCTGCGTGCGCCATGGGACCCGCAGCGGCAGCGCCTGGGAGCCCGAG
CGTCCCGCGTCCTCCTCCAGACCTGGTCTCTGCCTACGAGGTTGACCACAGGGGCGATTACGTGTCCCATGA
AATCATGCACCATCAGCGGCGGAGAAGAGCAGTGGCCGTGTCCGAGGTTGAGCCAGCCTTTCTCCAGGTATG
CAGAGCCAGAGAGCTCAGACTGTGTGTGGAGGCCTTTCCCATTGCTAATTCTCAGCCGGGGTTTTTGAACCT
TTCCAATGTTCGCTCTCACTGGAGGGAACAGCATGCTTCCAAGAGAATAATAACAAATGCAATGCTTGGAGA
ATCGGCCCTGGCTTCAACCAGAAAGTCTAATTGTGTTTTCTTTCTTTCCTTTTATTTTTTCCAGTCAGGCAT
GATACGAACAGAAGAGGCAGATTACTTCCTAAGGCCACTTCCTTCACACCTCTCATGGAAACTCGGCAGAGC
TGCCCAAGGCAGCTCGCCATCCCACGTACTGTACAAGAGAGAGGTCCTGGTGACCTCAAGGACATGGGAGCT
GGCACATCAACCCCTGCACAGCAGCGACCTTCGCCTGGGACTGCCACAAAAGCAGCATTTCTGTGGAAGACG
CAAGAAATACATGCCCCAGCCTCCCAAGGAAGACCTCTTCATCTTGCCAGATGAGTATAAGTCTTGCTTACG
GCATAAGCGCTCTCTTCTGAGGTCCCATAGAAATGAAGAACTGAACGTGGAGACCTTGGTGGTGGTCGACAA
AAAGATGATGCAAAACCATGGCCATGAAAATATCACCACCTACGTGCTCACGATACTCAACATGGTATCTGC
TTTATTCAAAGATGGAACAATAGGAGGAAACATCAACATTGCAATTGTAGGTCTGATTCTTCTAGAAGATGA
ACAGCCAGGACTGGTGATAAGTCACCACGCAGACCACACCTTAAGTAGTTTCTGCCAGTGGCAGTCTGGATT
GATGGGGAAAGATGGGACTCGTCATGACCACGCCATCTTACTGACTGGTCTGGATATATGTTCCTGGAAGAA
TGAGCCCTGTGACACTTTGGGATTTGCACCCATAAGTGGAATGTGTAGTAAATATCGCAGCTGCACGATTAA
TGAAGATACAGGTCTTGGACTGGCCTTCACCATTGCCCATGAGTCTGGACACAACTTTGGCATGATTCATGA
TGGAGAAGGGAACATGTGCAAAAAGTCCGAGGGCAACATCATGTCCCCTACATTGGCAGGACGCAATGGAGT
CTTCTCCTGGTCACCCTGCAGCCGCCAGTATCTACACAAATTTCTAAGCACCGCTCAAGCTATCTGCCTTGC
TGATCAGCCAAAGCCTGTGAAGGAATACAAGTATCCTGAGAAATTGCCAGGAGAATTATATGGTGCAAACAC
ACAGTGCAAGTGGCAGTTCGGAGAGAAAGCCAAGCTCTGCATGCTGGACTTTAAAAAGGACATCTGTAAAGC
CCTGTGGTGCCATCGTATTGGAAGGAAATGTGAGACTAAATTTATGCCAGCAGCAGAAGGCACAATTTGTGG
GCATGAACATGGTGCCGGAGGACAGTGTGTGAAATATGGTGATGAAGGCCCCAAGCCCACCCATGGCCACTG
GTCGGACTGGTCTTCTTGGTCCCCATGCTCCAGGACCTGCGGAGGGGGAGTATCTCATAGGAGTCGCTCTCA
AAATACACATTCCAGGCCATCGCATGGAGGGAAGTTCTGTGAGGGCTCCACTCGCACTCTGAAGCTCTGCAA
CAGTCAGAAATGTCCCCGGGACAGTGTTGACTTCCGTGCTGCTCAGTGTGCCGAGCACAACAGCAGACGATT
CAGAGGGCGGCACTACAAGTGGAAGCCTGATCAGGACTTATGCAAACTCTACTGTATCGCAGAAGGATTTGA
TTTCTTCTTTTCTTTGTCAAATAAAGTCAAAGATGGGACTCCATGCTCGGAGGATAGCCGTAATGTTTGTAT
AGATGGGATATGTGAGAGAGTTGGATGTGACAATGTCCTTGGATCTGATGCTGTTGAAGACGTCTGTGGGGT
GTGTAACGGGAATAACTCAGCCTGCACGATTCACAGGGGTCTCTACCTAGAGTATTATCACATGGTCACCAT
TCCTTCTGGAGCCCGGAGTATCCGCATCTATGAAATGAACGTCTCTACCTCCTACATTTCTGTGCGCAATGC
CCTCAGAAGGTACTACCTGAATGGGCACTGGACCGTGGACTGGCCCGGCCGGTACAAATTTTCGGGCACTAC
TTTCGACTACAGACGGTCCTATAATGAGCCCGAGAACTTAATCGCTACTGGACCAACCAACGAGACACTGAT
TGTGGAGCTGCTGTTTCAGGGAAGGAACCCGGGTGTTGCCTGGGAATACTCCATGCCTCGCTTGGGGACCGA
GAAGCAGCCCCCTGCCCAGCCCAGCTACACTTGGGCCATCGTGCGCTCTGAGTGCTCCGTGTCCTGCGGAGG
GGGTAGGTGCCTTCCAGTGCTGCTCCTGGAGGCAGCATGTCAGCCTTTAGCCACTGCGTACATTGCACTGGC
CTTTCTTGAATCCTAATGAGCAGCCCGGGGCTTCTCCCTGCCAGTAGCAGTGACATTCCCAAGGTGGGGAGT
GGTGGTCCTGAGTGTCACTTGTCGGCCCGAGCTGCCTTCTCCAGTCTATCTGCTTCAGTGTGTGACTCTGAG
GAAGTCAGTAGATGCATTGCTT

In a search of public sequence databases, the NOV1c nucleic acid sequence, located on chromsome 5 has 646 of 1089 bases (59%) identical to a gb:GENBANK-ID:AF140675|acc:AF140675.1 mRNA from Homo sapiens (Homo sapiens zinc metalloprotease ADAMTS7 (ADAMTS7) mRNA, complete cds) (E=1.0e ²³). Public nucleotide databases include all GenBank databases and the GeneSeq patent database.

The disclosed NOV1c polypeptide (SEQ ID NO:6) encoded by SEQ ID NO:5 has 856 amino acid residues and is presented in Table 1F using the one-letter amino acid code. Signal P, Psort and/or Hydropathy results predict that NOV1c has no signal peptide and is likely to be localized in the nucleus with a certainty of 0.7000. In other embodiments, NOV1c may also be localized to the microbody (peroxisome) with a certainty of 0.3813, the mitochondrial matrix space with a certainty of 0.1000, or in the lysosome (lumen) with a certainty of 0.1000.

TABLE 1F
Encoded NOV1c protein sequence.

(SEQ ID NO:6)

MGPAAAAPGSPSVPRPPPDLVSAYEVDHRGDYVSHEIMHHQRRRRAVAVSEVEPAFLQVCRARELRLCVEAF
PIANSQPGFLNLSNVRSHWREQHASKRIITNAMLGESALASTRKSNCVFFLSFYFFQSGMIRTEEADYFLRP
LPSHLSWKLGRAAQGSSPSHVLYKREVLVTSRTWELAHQPLHSSDLRLGLPQKQHFCGRRKKYMPQPPKEDL
FILPDEYKSCLRHKRSLLRSHRNEELNVETLVVVDKKMMQNHGHENITTYVLTILNMVSALFKDGTIGGNIN
IAIVGLILLEDEQPGLVISHHADHTLSSFCQWQSGLMGKDGTRHDHAILLTGLDICSWKNEPCDTLGFAPIS
GMCSKYRSCTINEDTGLGLAFTIAHESGHNFGMIHDGEGNMCKKSEGNIMSPTLAGRNGVFSWSPCSRQYLH
KFLSTAQAICLADQPKPVKEYKYPEKLPGELYGANTQCKWQFGEKAKLCMLDFKKDICKALWCHRIGRKCET
KFMPAAEGTICGHEHGAGGQCVKYGDEGPKPTHGHWSDWSSWSPCSRTCGGGVSHRSRSQNTHSRPSHGGKF
CEGSTRTLKLCNSQKCPRDSVDFRAAQCAEHNSRRFRGRHYKWKPDQDLCKLYCIAEGFDFFFSLSNKVKDG
TPCSEDSRNVCIDGICERVGCDNVLGSDAVEDVCGVCNGNNSACTIHRGLYLEYYHMVTIPSGARSIRIYEM
NVSTSYISVRNALRRYYLNGHWTVDWPGRYKFSGTTFDYRRSYNEPENLIATGPTNETLIVELLFQGRNPGV
AWEYSMPRLGTEKQPPAQPSYTWAIVRSECSVSCGGGRCLPVLLLEAACQPLATAYIALAFLES

A search of public sequence databases reveals that the NOV1c amino acid sequence has 256 of 579 amino acid residues (44%) identical to, and 352 of 579 amino acid residues (60%) similar to, the 997 amino acid residue ptnr:SWISSNEW-ACC:Q9UKP4 protein from Homo sapiens (Human) (ADAM-TS 7 precursor (EC 3.4.24.-) (A Disintegrin And Metalloproteinase With Thrombospondin Motifs 7) (ADAMTS-7) (ADAM-TS7) (E=1.9e⁻144). Public amino acid databases include the GenBank databases, SwissProt, PDB and PIR.

NOV1c is expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. Expression information was derived from the tissue sources of the sequences that were included in the derivation of the sequence of NOV1c.

NOV1d

A disclosed NOV1d nucleic acid of 2895 nucleotides (also referred to as CG52952-04) encoding a novel ADAM-TS 7-like protein is shown in Table IG. An open reading frame was identified beginning with an ATG initiation codon at nucleotides 10-12 and ending with a 10 TAA codon at nucleotides 2866-2868. A putative untranslated region upstream from the initiation codon and downstream from the termination codon is underlined in Table 1G. The start and stop codons are in bold letters.

TABLE 1G
NOV1d nucleotide sequence.

(SEQ ID NO:7)

CGCTCCTGGATGAAGCCCCGCGCGCGCGGATGGCGGGGCTTGGCGGCGCTGTGGATGCTGTTGGCGCAGGTG
GCCGAGCAGGTGAGTCCCGGGCGCTCCCACCAGCGCGGAAACCGCGGGTCCGGACAGCTGGAGGCGAGTCCC
CCGCGGCTCCTCTCCCGCGGACCCCGCCGTCTCACCGCGATGTCGCCGCTGTTTTCCGCAGGCACCTGCGTG
CGCCATGGGACCCGCAGCGGCAGCGCCTGGGAGCCCGAGCGTCCCGCGTCCTCCTCCACCCGCGGAGCGGCC
GGGCTGGATGGAAAAGGGCGGGACATGGATGAAGCTGGAAACCATCGTTCTCAGCAAACTAACACAGGAACA
GAAAACCAAACACTGCATGTTCTCACTCAATATGACCTGGTCTCTGCCTACGAGGTTGACCACAGGGGCGAT
TACGTGTCCCATGAAATCATGCACCATCAGCGGCGGAGAAGAGCAGTGGCCGTGTCCGAGGTTGAGTCTCTT
CACCTTCGGCTGAAAGGCCCCAGGCACGACTTCCACATGGATCTGAGGACTTCCAGCAGCCTAGTGGCTCCT
GGCTTTATTGTGCAGACGTTGGGAAAGACAGGCACTAAGTCTGTGCAGACTTTACCGCCAGAGGACTTCTGT
TTCTATCAAGGCTCTTTGCGATCACACAGAAACTCGCCATCGCATGGAGGGAAGTTCTGTGAGGGCTCCACT
CGCACTCTGAAGCTCTGCAACAGTCAGAAATGTCCCCGGGACAGTGTTGACTTCCGTGCTGCTCAGTGTGCC
GAGCACAACAGCAGACGATTCAGAGGGCGGCACTACAAGTGGAAGCCTTACACTCAAGTAGAAGCCGACTTA
TGCAAACTCTACTGTATCGCAGAAGGATTTGATTTCTTCTTTTCTTTGTCAAATAAAGTCAAAGATGGGACT
CCATGCTCGGAGGATAGCCGTAATGTTTGTATAGATGGGATATGTGAGCTCAGTGTGGTGTCCACATCTGCG
CACATGCCCCAGCCTCCCAAGGAAGACCTCTTCATCTTGCCAGATGAGTATAAGTCTTGCTTACGGCATAAG
CGCTCTCTTCTGAGGTCCCATAGAAATGAAGAACTGAACGTGGAGACCTTGGTGGTGGTCGACAAAAAGATG
ATGCAAAACCATGGCCATGAAAATATCACCACCTACGTGCTCACGATACTCAACATGGTATCTGCTTTATTC
AAAGATGGATTGATGGGGAAAGATGGGACTCGTCATGACCACGCCATCTTACTGACTGGTCTGGATATATGT
TCCTGGAAGAATGAGCCCTGTGACACTTTGGGATTTGCACCCATAAGTGGAATGTGTAGTAAATATCGCAGC
TGCACGATTAATGAAGATACAGGTCTTGGACTGGCCTTCACCATTGCCCATGAGTCTGGACACAACTTTGGC
ATGATTCATGATGGAGAAGGGAACATGTGTAAAAAGTCCGAGGGCAACATCATGTCCCCTACATTGGCAGGA
CGCAATGGAGTCTTCTCCTGGTCACCCTGCAGCCGCCAGTATCTACACAAATTTCTAAGCACCGCTCAAGCT
ATCTGCCTTGCTGATCAGCCAAAGCCTGTGAAGGAATACAAGTATCCTGAGAAATTGCCAGGAGAATTATAT
GATGCAAACACACAGTGCAAGTGGCAGTTCGGAGAGAAAGCCAAGCTCTGCATGCTGGACTTTAAAAAGGAC
ATCTGTAAAGCCCTGTGGTGCCATCGTATTGGAAGGAAATGTGAGACTAAATTTATGCCAGCAGCAGAAGGC
ACAATTTGTGGGCATGACATGTGGTGCCGGGGAGGACAGTGTGTGAAATATGGTGATGAAGGCCCCAAGCCC
ACCCATGGCCACTGGTCGGACTGGTCTTCTTGGTCCCCATGCTCCAGGACCTGCGGAGGGGGAGTATCTCAT
AGGAGTCGCCTCTGCACCAACCCCAAGCCATCGCATGGAGGGAAGTTCTGTGAGGGCTCCACTCGCACTCTG
AAGCTCTGCAACAGTCAGAAATGTCCCCGGGACAGTGTTGACTTCCGTGCTGCTCAGTGTGCCGAGCACAAC
AGCAGACGATTCAGAGGGCGGCACTACAAGTGGAAGCCTTACACTCAAGTAGAAGATCAGGACTTATGCAAA
CTCTACTGTATCGCAGAAGGATTTGATTTCTTCTTTTCTTTGTCAAATAAAGTCAAAGATGGGACTCCATGC
TCGGAGGATAGCCGTAATCTTTGTATAGATGGGATATGTGAGAGAGTTGGATGTGACAATGTCCTTGGATCT
GATGCTGTTGAAGACGTCTGTGGGGTGTGTAACGGGAATAACTCAGCCTGCACGATTCACAGGGGTCTCTAC
ACCAAGCACCACCACACCAACCAGTATTATCACATGGTCACCATTCCTTCTGGAGCCCGGAGTATCCGCATC
TATGAAATGAACGTCTCTACCTCCTACATTTCTGTGCGCAATGCCCTCAGAAGGTACTACCTGAATGGGCAC
TGGACCGTGGACTGGCCCGGCCGGTACAAATTTTCGGGCACTACTTTCGACTACAGACGGTCCTATAATGAG
CCCGAGAACTTAATCGCTACTGGACCAACCAACGAGACACTGATTGTGGAGCTGCTGTTTCAGGGAAGGAAC
CCGGGTGTTGCCTGGGAATACTCCATGCCTCGCTTGGGGACCGAGAAGCAGCCCCCTGCCCAGCCCAGCTAC
ACTTGGGCCATCGTGCGCTCTGAGTGCTCCGTGTCCTGCGGAGGGGGTAGGTGCCTTCCAGTGCTGCTCCTG
GAGGCAGCATGTCAGCCTTCAGCCACTGCGTACATTGCACTGGCCTTTCTTGAATCCTAATGAGCAGCCCGG
GGCTTCTCCCTGCCA

In a search of public sequence databases, the NOV1d nucleic acid sequence, located on chromsome 5 has 380 of 614 bases (61%) identical to a gb:GENBANK-ID:AF140675|acc:AF140675.1 mRNA from Homo sapiens (Homo sapiens zinc metalloprotease ADAMTS7 (ADAMTS7) mRNA, complete cds) (E=7.0e6⁻¹⁶). Public nucleotide databases include all GenBank databases and the GeneSeq patent database.

The disclosed NOV1d polypeptide (SEQ ID NO:8) encoded by SEQ ID NO:7 has 952 amino acid residues and is presented in Table 1H using the one-letter amino acid code. Signal P, Psort and/or Hydropathy results predict that NOV1d has a signal peptide and is likely to be localized extracellualrly with a certainty of 0.3700. In other embodiments, NOV1d may also be localized to the lysosome (lumen) with a certainty of 0.1900, the microbody (peroxisome) with a certainty of 0.1270, or in the endoplasmic reticulum (membrane) with a certainty of 0.1000. The most likely cleavage site for a NOV1a peptide is between amino acids 23 and 24, at: VAE-QV.

TABLE 1H
Encoded NOV1d protein sequence.

(SEQ ID NO:8)

MKPRARGWRGLAALWMLLAQVAEQVSPGRSHQRGNRGSGQLEASPPRLLSRGPRRLTAMSPLFSAGTCVRHG
TRSGSAWEPERPASSSTRGAAGLDGKGRDMDEAGNHRSQQTNTGTENQTLHVLTQYDLVSAYEVDHRGDYVS
HEIMHHQRRRRAVAVSEVESLHLRLKGPRHDFHMDLRTSSSLVAPGFIVQTLGKTGTKSVQTLPPEDFCFYQ
GSLRSHRNSPSHGGKFCEGSTRTLKLCNSQKCPRDSVDFRAAQCAEHNSRRFRGRHYKWKPYTQVEADLCKL
YCIAEGFDFFFSLSNKVKDGTPCSEDSRNVCIDGICELSVVSTSAHMPQPPKEDLFILPDEYKSCLRHKRSL
LRSHRNEELNVETLVVVDKKMMQNHGHENITTYVLTILNMVSALFKDGLMGKDGTRHDHAILLTGLDICSWK
NEPCDTLGFAPISGMCSKYRSCTINEDTGLGLAFTIAHESGHNFGMIHDGEGNMCKKSEGNIMSPTLAGRNG
VFSWSPCSRQYLHKFLSTAQAICLADQPKPVKEYKYPEKLPGELYDANTQCKWQFGEKAKLCMLDFKKDICK
ALWCHRIGRKCETKFMPAAEGTICGHDMWCRGGQCVKYGDEGPKPTHGHWSDWSSWSPCSRTCGGGVSHRSR
LCTNPKPSHGGKFCEGSTRTLKLCNSQKCPRDSVDFRAAQCAEHNSRRFRGRHYKWKPYTQVEDQDLCKLYC
IAEGFDFFFSLSNKVKDGTPCSEDSRNVCIDGICERVGCDNVLGSDAVEDVCGVCNGNNSACTIHRGLYTKH
HHTNQYYHMVTIPSGARSIRIYEMNVSTSYISVRNALRRYYLNGHWTVDWPGRYKFSGTTFDYRRSYNEPEN
LIATGPTNETLIVELLFQGRNPGVAWEYSMPRLGTEKQPPAQPSYTWAIVRSECSVSCGGGRCLPVLLLEAA
CQPSATAYIALAFLES

A search of public sequence databases reveals that the NOV1d amino acid sequence has 207 of 483 amino acid residues (42%) identical to, and 287 of 483 amino acid residues (59%) similar to, the 997 amino acid residue ptnr:SWISSNEW-ACC:Q9UKP4 protein from Homo sapiens (Human) (ADAM-TS 7 precursor (EC 3.4.24.-) (A Disintegrin And Metalloproteinase With Thrombospondin Motifs 7) (ADAMTS-7) (ADAM-TS7)) (E=7.0e⁻¹⁶). Public amino acid databases include the GenBank databases, SwissProt, PDB and PIR.

NOV 1d is expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. Expression information was derived from the tissue sources of the sequences that were included in the derivation of the sequence of NOV1 d and the expression pattern of (GENBANK-ID: gb:GENBANK-ID:AF140675|acc:AF140675.1) a closely related Homo sapiens zinc metalloprotease ADAMTS7 (ADAMTS7) mRNA, complete cds homolog in species Homo sapiens.

The proteins encoded by the NOV1a, 1b, 1c, and 1d nucleotides are very closely homologous as is shown in the alignment in Table 1I. TaqMan data for NOV1 can be found below in Example 2. SNP data for NOV1a can be found below in Example 3.

[image]

[image]

[image]

Homologies to any of the above NOV1 proteins will be shared by the other two NOV1 proteins insofar as they are homologous to each other as shown above. Any reference to NOV1 is assumed to refer to all three of the NOV 1 proteins in general, unless otherwise noted.

The disclosed NOV1 a polypeptide has homology to the amino acid sequences shown in the BLASTP data listed in Table 1J.

TABLE 1J
BLAST results for NOV1a

| Gene Index/ | | | Identity | Positives | |
|---|---|---|---|---|---|
| Identifier | Protein/Organism | Length (aa) | (%) | (%) | Expect |
| gi\|13569928\|ref\|NP_{—} | a disintegrin- | 1593 | 269/616 | 371/616 | e−135 |
| 112217.1\| | like and | | (43%) | (59%) | |
| | metalloprotease | | | | |
| | with | | | | |
| | thrombospondin | | | | |
| | type 1 motif, 12 | | | | |
| | [Homo sapiens] | | | | |
| gi\|11493589\|gb\|AAG3 | zinc | 1077 | 253/624 | 359/624 | e−128 |
| 5563.1\|AF163762_1 | metalloendopeptid | | (40%) | (56%) | |
| (AF163762) | ase [Homo | | | | |
| | sapiens] | | | | |
| gi\|10645199\|ref\|NP_{—} | a disintegrin and | 997 | 247/571 | 344/571 | e−127 |
| 055087.1\| | metalloprotease | | (43%) | (59%) | |
| | with | | | | |
| | thrombospondin | | | | |
| | motifs-7 | | | | |
| | preproprotein; a | | | | |
| | disintegrin-like | | | | |
| | and | | | | |
| | metalloprotease | | | | |
| | (reprolysin type) | | | | |
| | with | | | | |
| | thrombospondin | | | | |
| | type 1 motif, 7 | | | | |
| | [Homo sapiens] | | | | |
| gi\|15309931\|ref\|XP_{—} | a disintegrin- | 854 | 245/604 | 348/604 | e−124 |
| 054419.1\| | like and | | (40%) | (57%) | |
| | metalloprotease | | | | |
| | domain with | | | | |
| | thrombospondin | | | | |
| | type I repeats 10 | | | | |
| | [Homo sapiens] | | | | |
| gi\|7656869\|ref\|NP_{—} | a disintegrin and | 860 | 244/630 | 345/630 | e−115 |
| 055088.1\| | metalloprotease | | (38%) | (54%) | |
| | with | | | | |
| | thrombospondin | | | | |
| | motifs-6 | | | | |
| | preproprotein; a | | | | |
| | disintegrin-like | | | | |
| | and | | | | |
| | metalloprotease | | | | |
| | (reprolysin type) | | | | |
| | with | | | | |
| | thrombospondin | | | | |
| | type 1 motif, 6 | | | | |
| | [Homo sapiens] | | | | |

The homology between these and other sequences is shown graphically in the ClustalW analysis shown in Table 1K. In the ClustalW alignment of the NOV1 proteins, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

[image]

[image]

[image]

[image]

[image]

[image]

The presence of identifiable domains in NOV1, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results for NOV1 as disclosed in Tables 1L-10, were collected from the Conserved Domain Database (CDD) with Reverse Position Specific BLAST analyses. This BLAST analysis software samples domains found in the Smart and Pfam collections. For Table 1 E and all successive DOMAIN sequence alignments, fully conserved single residues are indicated by black shading or by the sign (|) and “strong” semi-conserved residues are indicated by grey shading or by the sign (+). The “strong” group of conserved amino acid residues may be any one of the following groups of amino acids: STA, NEQK, NHQK, NDEQ, QHRK, MILV, MILF, HY, FYW.

Tables 1L-10 lists the domain description from DOMAIN analysis results against NOV1. This indicates that the NOV1 sequence has properties similar to those of other proteins known to contain this domain.

TABLE 1L
Domain Analysis of NOV1

gnl|Pfam|pfam01421, Reprolysin, Reprolysin (M12B) family zinc
metalloprotease. The members of this family are enzymes that cleave
peptides. These proteases require zinc for catalysis. Members of this
family are also known as adamalysins. Most members of this family are
snake venom endopeptidases, but there are also some mammalian
proteins, and fertilin. Fertilin and closely related proteins appear
to not have some active site residues and may not be active enzymes.
(SEQ ID NO:76)
CD-Length = 199 residues, 95.0% aligned
Score = 122 bits (307) , Expect = 7e-29

| | | | |
|---|---|---|---|
| Query: | 378 | DKKMMQNHG--HENITTYVLTILNMVSALFKDGTIGGNINIAIVGLILLEDEQPGLVISH | 435 |
| | | \| \| +\| \| \| \|+\|+\|+ +++ \|\| + +\|\|\| + \| + + | |
| Sbjct: | 10 | DHGMFTKYGSDLNKIRQRVHQIVNLVNEIYRPL----NIRVVLVGLEIWSDGDK-ITVQG | 64 |
| Query: | 436 | HADHTLSSFCQWQSGLMGKDGTRHDHAILLTGLDICSWKNEPCDTLGFAPISGMCSKYRS | 495 |
| | | \|+ \|\| \| +\|+ + \| \|\|+\| \|\|\|\|+\| +\|+\| \| + \|\|\|\| \|\| | |
| Sbjct: | 65 | DANDTLHRFLEWRETDLLKR-KSHDNAQLLTGIDF------DGNTIGAAYVGGMCSPKRS | 117 |
| Query: | 496 | CTINED---TGLGLAFTIAHESGHNFGMIHDGEGN-MCKKSEGNIMSPTLAGRNGVFSWS | 551 |
| | | + +\| \| +\| \|+\|\|\| \|\|\| \|\| \|\| \|+ \| \|\|+\| + \| +\| | |
| Sbjct: | 118 | VGVVQDHSPIVLLVAVTMAHELGHNLGMTHDDINKCTCEGGGGCIMNPVASSSPGKK-FS | 176 |
| Query: | 552 | PCSRQYLHKFLSTAQAICLADQ 573 | |
| | | \|\| +\|\|+ + \|\| ++ | |
| Sbjct: | 177 | NCSMDDYQQFLTKGKPQCLLNK 198 | |

TABLE 1M
Domain Analysis of NOV1

gnl|Smart|smart00209, TSP1, Thrormbospondin type 1 repeats; Type 1
repeats in thrombospondin-1 bind and activate TGF-beta.
(SEQ ID NO:77)
CD-Length = 51 residues, 100.0% aligned
Score = 63.5 bits (153), Expect = 5e-11

| | | | |
|---|---|---|---|
| Query: | 668 | WSDWSSWSPCSRTCGGGVSHRSRLCTNPNPSHGGKFCEGSTRTLKLCNSQKCP | 720 |
| | | \| +\|\| \|\|\|\|\| \|\|\|\|\|\| \|+\| \| \| \|\| \| \| + \|\| \| \|\| | |
| Sbjct: | 1 | WGEWSEWSPCSVTCGGGVQTRTRCCNPPPN--GGGPCTGPDTETRACNEQPCP | 51 |

TABLE 1N
Domain Analysis of NOV1

gnl|pfam|pfam00090, tsp_1, Thrombospondin type 1 domain. (SEQ ID
NO:78)
CD-Length = 48 residues, 100.0% aligned
Score = 54.7 bits (130), Expect = 2e-08

| | | | |
|---|---|---|---|
| Query: | 669 | SDWSSWSPCSRTCGGGVSHRSRLCTNPNPSHGGKFCEGSTRTLKLCNSQKC | 719 |
| | | \| \|\| \|\|\|\|\| \|\|\| \|+ \| \| \| +\| \|\|\| \| \| + + \| \| | |
| Sbjct: | 1 | SPWSEWSPCSVTCGKGIRTRQRTCNSP---AGGKPCTGDAQETEACMMDPC | 48 |

TABLE 1O
Domain Analysis of NOV1

gnl|Pfam|pfam01562, Pep_M12B_propep, Reprolysin family propeptide.
This region is the propeptide for members of peptidase family M12B.
The propeptide contains a sequence motif similar to the “cysteine
switch” of the matrixins. This motif is found at the C terminus of the
alignment but is not well aligned, repeats. (SEQ ID NO:79)
CD-Length = 117 residues, only 39.3% aligned
Score = 38.5 bits (88), Expect = 0.002

| | | | |
|---|---|---|---|
| Query: | 180 | LRTSSSLVAPGFIVQTLGKTGTKSVQTLPPEDFCFYQGSLRSHRNS | 225 |
| | | \| + \|\|+\|\| \| \| \| \|\| + +\| \|+\|\|\| + + \|\| | |
| Sbjct: | 2 | LEKNRSLLAPDFTVTTYDDDGTLVTEHPLIQDHCYYQGYVEGYPNS | 47 |

Thrombospondin-1 (THBS 1) associates with the extracellular matrix and inhibits angiogenesis in vivo. In vitro, THBS1 blocks capillary-like tube formation and endothelial cell proliferation. The antiangiogenic activity is mediated by a region that contains 3 type 1 (properdin or thrombospondin) repeats. By searching an EST database for sequences containing the antiangiogenic motif of THBS 1, Vazquez et al. (1999) identified heart and lung cDNAs encoding ADAMTS1 and ADAMTS8, which they called METH1 and METH2, respectively. Sequence analysis predicted that the 890-amino acid ADAMTS8 protein shares 52% amino acid identity with ADAMTS1. ADAMTS8 is a secreted protein that has an N-terminal signal peptide, a zinc metalloprotease domain containing a zinc-binding site, and a cysteine-rich region containing 2 putative disintegrin loops. The C terminus of ADAMTS8 has 2 heparin-binding thrombospondin repeats with 6 cys and 3 trp residues. Southern blot analysis showed that ADAMTS8 is a single-copy gene distinct from that encoding ADAMTS1. Northern blot analysis detected highest expression of a 3.7 -kb ADAMTS8 transcript in adult and fetal lung, with lower expression in brain, placenta, heart, and stomach, as well as fetal brain and kidney. Expression was also detected in a colon carcinoma cell line. SDS-PAGE analysis demonstrated that ADAMTS8 is expressed as a 98 -kD protein, a 79 -kD protein after cleavage at the subtilisin site, or as a 64 -kD protein, which is most abundant, generated by an additional processing event. Functional analysis determined that ADAMTS8 disrupts angiogenesis in vivo and in vitro more efficiently than THBS1 or endostatin but somewhat less efficiently than ADAMTS 1.

By interspecific backcross analysis, Georgiadis et al. (1999) mapped the mouse Adamts8 gene to chromosome 9 in a region showing homology of synteny with human 11q23-qter. They mapped the human ADAMTS8 gene to 11q25 by PCR analysis of a radiation hybrid mapping panel. The authors noted that a number of disorders have been mapped in the vicinity of the ADAMTS8 gene in mice and humans, most notably, given the expression and functional analyses, lung neoplasms.

The novel protein described here contains thrombospondin type I domains and Reprolysin domain. It is homologous to ADAM genes. Thrombospondin type I domain [IPR000884; (TSP1)] was found in the thrombospondin protein where it is repeated 3 times. Now a number of proteins involved in the complement pathway (properdin, C6, C7, C8A, C8B, C9) as well as extracellular matrix protein like mindin, F-spondin, SCO-spondin and even the circumsporozoite surface protein 2 and TRAP proteins of Plasmodium contain one or more instance of this repeat. It has been involved in cell-cell interraction, inhibition of angiogenesis, apoptosis. The intron-exon organisation of the properdin gene confirms the hypothesis that the repeat might have evolved by a process involving exon shuffling. A study of properdin structure provides some information about the structure of the thrombospondin type I repeat.

Reprolysin family propeptide [IPR002870; (Pep_M12B_propep)] domain is contained in the propeptide for members of peptidase family Ml 2B. The propeptide contains a sequence motif similar to the ‘cysteine switch’ of the matrixins. This motif is found at the C terminus of the alignment but is not well aligned.

Through a subtractive hybridization approach to identify genes specifically expressed in the caput epididymidis, the mouse homologue of a member of the ADAM (a disintegrin and metalloprotease) family of proteins was identified. This rapidly growing gene family encodes cell surface proteins that possess putative adhesion and protease domains. Northern blot analyses demonstrated that the mouse ADAM gene, termed ADAM7, is expressed in the caput region of the epididymis and in the anterior pituitary gonadotropes with no detectable expression in the twenty-six other tissues examined. Furthermore, in situ hybridization experiments revealed that the ADAM7 messenger RNA (mRNA) exhibited an apical localization within the proximal caput epididymal epithelium that may correlate with an unusual sparsely granulated endoplasmic reticulum uniquely present in the proximal region of the epididymidis and to which no known function has been ascribed. Hormonal, surgical, and genetic strategies demonstrated that ADAM7 gene expression requires, in a region-dependent manner, androgens as well as testicular factors for expression. Interestingly, the apical localization of ADAM7 mRNA is dependent upon an intact testis, because in situ hybridization analyses of the proximal caput epididymidis from a testosterone maintained castrate mouse did not show the apical localization of ADAM7 mRNA. Finally, chromosomal mapping demonstrated that the ADAM7 gene maps to the central region of mouse Chromosome 14, approximately 4-5 cM distal from the fertilin beta locus, which encodes another reproductive-specific ADAM protein (1).

Because of the presence of the domain and the homology to the , we anticipate that the novel sequence described here will have useful properties and functions similar to these genes.

The disclosed NOV1 nucleic acid of the invention encoding a ADAM-TS 7-like protein includes the nucleic acid whose sequence is provided in Table 1A, IC, IE, and IG or a fragment thereof. The invention also includes a mutant or variant nucleic acid any of whose bases may be changed from the corresponding base shown in Table 1A, 1C, 1E and 1G while still encoding a protein that maintains its ADAM-TS 7-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 33% percent of the bases may be so changed.

The disclosed NOV1 protein of the invention includes the ADAM-TS 7-like protein whose sequence is provided in Table 1 B, 1D, 1F, or 1H. The invention also includes a mutant or variant protein any of whose residues may be changed from the corresponding residue shown in Table 1B, 1D, 1F, or 1H while still encoding a protein that maintains its ADAM-TS 7-like activities and physiological functions, or a functional fragment thereof. In the mutant or variant protein, up to about 62% percent of the residues may be so changed.

The invention further encompasses antibodies and antibody fragments, such as F_{ab} or (F_{ab})₂, that bind immunospecifically to any of the proteins of the invention.

The above defined information for this invention suggests that this ADAM-TS 7-like protein (NOV1) may function as a member of a “ADAM-TS 7 family”. Therefore, the NOV1 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV1 nucleic acids and proteins of the invention are useful in potential therapeutic applications implicated in cancer including but not limited to various pathologies and disorders as indicated below. For example, a cDNA encoding the ADAM-TS 7-like protein (NOV1) may be useful in gene therapy, and the ADAM-TS 7-like protein (NOV1) may be useful when administered to a subject in need thereof. By way of nonlimiting example, the compositions of the present invention will have efficacy for treatment of patients suffering from adrenoleukodystrophy, Alzheimer's disease, autoimmune disease, allergies, addiction, anxiety, ataxia-telangiectasia, asthma, ARDS, atherosclerosis, behavioral disorders, aortic stenosis, atrial septal defect (ASD), atrioventricular (A-V) canal defect, ductus arteriosus, allergy, cerebral palsy, congenital adrenal hyperplasia, cirrhosis, cardiomyopathy, congenital heart defects, diabetes, diverticular disease, epilepsy, emphysema, endometriosis, endocrine dysfunctions, graft versus host disease, glomerulonephritis, graft versus host disease (GVHD), growth and reproductive disorders, hemophilia, hypercoagulation, hypercalceimia, Huntington's disease, hypertension, hypogonadism, fertility, idiopathic thrombocytopenic purpura, immunodeficiencies, interstitial nephritis, IgA nephropathy, lymphaedema, inflammatory bowel disease, Lesch-Nyhan syndrome, leukodystrophies, multiple sclerosis, muscular dystrophy, myasthenia gravis, neurodegeneration, neuroprotection, obesity, Parkinson's disease, pain, polycystic kidney disease, pulmonary stenosis, pancreatitis, renal artery stenosis, renal tubular acidosis, stroke, systemic lupus erythematosus, scleroderma, subaortic stenosis, transplantation, tuberous sclerosis, Von Hippel-Lindau (VHL) syndrome, ventricular septal defect (VSD), valve diseases, Von Hippel-Lindau (VHL) syndrome, ulcers, bacterial and viral infections, neonatal apnea, eagle's syndrome, atherosclerosis, metabolic disorders such as familial amyloidotic polyneuropathy, hyperkinetic diseases, muscular dystrophy, cerebral vascular disease, hypertension, cardiovascular diseseases, renal fibrogenesis, inflammatory bowel disease, Meckel syndrome, colorectal cancer, papillomavirus infection and cervical carcinoma, liver malignancies, skin disorders, connective tissue disorders such as type VIIC Ehlers-Danros syndrome, osteoarthritis, rheumatoid arthritis, or other pathologie or conditions. The NOV1 nucleic acid encoding the ADAM-TS 7-like protein of the invention, or fragments thereof, may further be useful in diagnostic applications, wherein the presence or amount of the nucleic acid or the protein are to be assessed.

NOV1 nucleic acids and polypeptides are further useful in the generation of antibodies that bind immuno-specifically to the novel NOV1 substances for use in therapeutic or diagnostic methods. These antibodies may be generated according to methods known in the art, using prediction from hydrophobicity charts, as described in the “Anti-NOVX Antibodies” section below. The disclosed NOV1a and b proteins have multiple hydrophilic regions, each of which can be used as an immunogen. In one embodiment, a contemplated NOV1a and b epitope is from about amino acids 20 to 70. In another embodiment, a NOV1a and b epitope is from about amino acids 80 to 180. In additional embodiments, NOV1a and b epitopes are from about amino acids 200 to 280, from about amino acids 300 to 360, from about amino acid 400 to 530, from about amino acid 540 to 550, from about amino acid 580 to 610, from about amino acid 630 to 680, and from about amino acids 710 to 750. Also, the disclosed NOV1c protein has multiple hydrophilic regions, each of which can be used as an immunogen. In one embodiment, a contemplated NOV1c epitope is from about amino acids 1 to 50. In another embodiment, a NOV1c epitope is from about amino acids 80 to 100. In additional embodiments, NOV1c epitopes are from about amino acids 110 to 260, from about amino acids 280 to 320, from about amino acid 350 to 400, from about amino acid 420 to 470, from about amino acid 480 to 620, from about amino acid 700 to 750, and from about amino acids 770 to 810. Also, the disclosed NOV1d protein has multiple hydrophilic regions, each of which can be used as an immunogen. In one embodiment, a contemplated NOV1d epitope is from about amino acids 20 to 180. In another embodiment, a NOV1d epitope is from about amino acids 190 to 280. In additional embodiments, NOV1d epitopes sre from about amino acids 300 to 360, from about amino acids 400 to 530, from about amino acid 540 to 550, from about amino acid 580 to 610, from about amino acid 630 to 680, and from about amino acids 710 to 750. These novel proteins can be used in assay systems for functional analysis of various human disorders, which will help in understanding of pathology of the disease and development of new drug targets for various disorders.

NOV2

A disclosed NOV2 nucleic acid of 4488 nucleotides (also referred to as SC_{—}78316254_A) encoding a novel alpha-2-macroglobulin precursor-like protein is shown in Table 2A. An open reading frame was identified beginning with an ATG initiation codon at nucleotides 1-3 and ending with a TGA codon at nucleotides 4477-4479. A putative untranslated region upstream from the initiation codon and downstream from the termination codon is underlined in Table 2A, and the start and stop codons are in bold letters.

TABLE 2A
NOV2 nucleotide sequence.

(SEQ ID NO:9)

ATGTGGGCTCAGCTCCTTCTAGGAATGTTGGCCCTATCACCAGCCATTGCAGAAGAACTTCCAAACTACCTG
GTGACATTACCAGCCCGGCTAAATTTCCCCTCCGTTCAGAAGGTTTGTTTGGACCTGAGCCCTGGGTACAGT
GATGTTAAATTCACGGTTACTCTGGAGACCAAGGACAAGACCCAGAAGTTGCTAGAATACTCTGGACTGAAG
AAGAGGCACTTACATTGTATCTCCTTTCTTGTACCACCTCCTGCTGGTGGCACAGAAGAAGTGGCCACAATC
CGGGTGTCGGGAGTTGGAAATAACATCAGCTTTGAGGAGAAGAAAAAGGTTCTAATTCAGAGGCAGGGGAAC
GGCACCTTTGTACAGACTGACAAACCTCTCTACACCCCAGGGCAGCAAGTGTATTTCCGCATTGTCACCATG
GATAGCAACTTCGTTCCAGTGAATGACAAGTACTCCATGGTGGAACTACAGGATCCAAATAGCAACAGGATT
GCACAGTGGCTGGAAGTGGTACCTGAGCAAGGCATTGTAGACCTGTCCTTCCAACTGGCACCAGAGGCAATG
CTGGGCACCTACACTGTGGCAGTGGCTGAGGGCAAGACCTTTGGTACTTTCAGTGTGGAGGAATATGTGCTT
TCTCCATTTCTCCTTTTACTCTCTTCAGTGCTGCCGAAGTTTAAGGTGGAAGTGGTGGAACCCAAGGAGTTA
TCAACGGTGCAGGAATCTTTCTTAGTAAAAATTTGTTGTAGGTACACCTATGGAAAGCCCATGCTAGGGGCA
GTGCAGGTATCTGTGTGTCAGAAGGCAAATACTTACTGGTATCGAGAGGTGGAACGGGAACAGCTTCCTGAC
AAATGCAGGAACCTCTCTGGACAGACTGACAAAACAGGATGTTTCTCAGCACCTGTGGACATGGCCACCTTT
GACCTCATTGGATATGCGTACAGCCATCAAATCAATATTGTGGCTACTGTTGTGGAGGAAGGGACAGGTGTG
GAGGCCAATGCCACTCAGAATATCTACATTTCTCCACAAATGGGATCAATGACCTTTGAAGACACCAGCAAT
TTTTACCATCCAAATTTCCCCTTCAGTGGGAAGATGCTGCTCAAGTTTCCGCAAGGCGGTGTGCTCCCTTGC
AAGAACCATCTAGTGTTTCTGGTGATTTATGGCACAAATGGAACCTTCAACCAGACCCTGGTTACTGATAAC
AATGGCCTAGCTCCCTTTACCTTGGAGACATCCGGTTGGAATGGGACAGACGTTTCTCTGGAGGGAAAGTTT
CAAATGGAAGACTTAGTATATAATCCGGAACAAGTGCCACGTTACTACCAAAATGCCTACCTGCACCTGCGA
CCCTTCTACAGCACAACCCGCAGCTTCCTTGGCATCCACCGGCTAAACGGCCCCTTGAAATGTGGCCAGCCC
CAGGAAGTGCTGGTGGATTATTACATCGACCCGGCCGATGCAAGCCCTGACCAAGAGATCAGCTTCTCCTAC
TATTTAATAGGGAAAGGAAGTTTGGTGATGGAGGGGCAGAAACACCTGAACTCTAAGAAGAAAGGACTGAAA
GCCTCCTTCTCTCTCTCACTGACCTTCACTTCGAGACTGGCCCCTGATCCTTCCCTGGTGATCTATGCCATT
TTTCCCAGTGGAGGTGTTGTAGCTGACAAAATTCAGTTCTCAGTCGAGATGTGCTTTGACAATCAGCAGCTT
CCAGGAGCAGAAGTGGAGCTGCAGCTGCAGGCAGCTCCCGGATCCCTGTGTGCGCTCCGGGCGGTGGATGAG
AGTGTCTTACTGCTTAGGCCAGACAGAGAGCTGAGCAACCGCTCTGTCTATGGGATGTTTCCATTCTGGTAT
GGTCACTACCCCTATCAAGTGGCTGAGTATGATCAGTGTCCAGTGTCTGGCCCATGGGACTTTCCTCAGCCC
CTCATTGACCCAATGCCCCAAGGGCATTCGAGCCAGCGTTCCATTATCTGGAGGCCCTCGTTCTCTGAAGGC
ACGGACCTTTTCAGCTTTTTCCGGGACGTGGGCCTGAAAATACTGTCCAATGCCAAAATCAAGAAGCCAGTA
GATTGCAGTCACAGATCTCCAGAATACAGCACTGCTATGGGTGGCGGTGGTCATCCAGAGGCTTTTGAGTCA
TCAACTCCTTTACATCAAGCAGAGGATTCTCAGGTCCGCCAGTACTTCCCAGAGACCTGGCTCTGGGATCTG
TTTCCTATTGGTAACTCGGGGAAGGAGGCGGTCCACGTCACAGTTCCTGACGCCATCACCGAGTGGAAGGCG
ATGAGTTTCTGCACTTCCCAGTCAAGAGGCTTCGGGCTTTCACCCACTGTTGGACTAACTGCTTTCAAGCCG
TTCTTTGTTGACCTGACTCTCCCTTACTCAGTAGTCCGTGGGGAATCCTTTCGTCTTACTGCCACCATCTTC
AATTACCTAAAGGATTGCATCAGGGTTCAGACTGACCTGGCTAAATCGCATGAGTACCAGCTAGAATCATGG
GCAGATTCTCAGACCTCCAGTTGTCTCTGTGCTGATGACGCAAAAACCCACCACTGGAACATCACAGCTGTC
AAATTGGGTCACATTAACTTTACTATTAGTACAAAGATTCTGGACAGCAATGAACCATGTGGGGGCCAGAAG
GGGTTTGTTCCCCAAAAGGGCCGAAGTGACACGCTCATCAAGCCAGTTCTCGTCAAACCTGAGGGAGTCCTG
GTGGAGAAGACACACAGCTCATTGCTGTGCCCAAAAGGAGGAAAGGTGGCATCTGAATCTGTCTCCCTGGAG
CTCCCAGTGGACATTGTTCCTGACTCGACCAAGGCTTATGTTACGGTTCTGGGAGACATTATGGGCACAGCC
CTGCAGAACCTGGATGGTCTGGTGCAGATGCCCAGTGGCTGTGGCGAGCAGAACATGGTCTTGTTTGCTCCC
ATCATCTATGTCTTGCAGTACCTGGAGAAGGCAGGGCTGCTGACGGAGGAGATCAGGTCTCGGGCAGTGGGT
TTCCTGGAAATAGGGTACCAGAAGGAGCTGATGTACAAACACAGCAATGGCTCATACAGTGCCTTTGGGGAG
CGAGATGGAAATGGAAACACATGGCTGACAGCGTTTGTCACAAAATGCTTTGGCCAAGCTCAGAAATTCATC
TTCATTGATCCCAAGAACATCCAGGATGCTCTCAAGTGGATGGCAGGAAACCAGCTCCCCAGTGGCTGCTAT
GCCAACGTGGGAAATCTCCTTCACACAGCTATGAAGGGTGGTGTTGATGATGAGGTCTCCTTGACTGCGTAT
GTCACAGCTGCATTGCTGGAGATGGGAAAGGATGTAGATGACCCAATGGTGAGTCAGGGTCTACGGTGTCTC
AAGAATTCGGCCACCTCCACGACCAACCTCTACACACAGGCCCTGTTGGCTTACATTTTCTCCCTGGCTGGG
GAAATGGACATCAGAAACATTCTCCTTAAACAGTTAGATCAACAGGCTATCATCTCAGGAGAATCCATTTAC
TGGAGCCAGAAACCTACTCCATCATCGAACGCCAGCCCTTGGTCTGAGCCTGCGGCTGTAGATGTGGAACTC
ACAGCATATGCATTGTTGGCCCAGCTTACCAAGCCCAGCCTGACTCAAAAGGAGATAGCGAAGGCCACTAGC
ATAGTGGCTTGGTTGGCCAAGCAACACAATGCATATGGGGGCTTCTCTTCTACTCAGGATACTGTAGTTGCT
CTCCAAGCTCTTGCCAAATATGCCACTACCGCCTACATGCCATCTGAGGAGATCAACCTGGTTGTAAAATCC
ACTGAGAATTTCCAGCGCACATTCAACATACAGTCAGTTAACAGATTGGTATTTCAGCAGGATACCCTGCCC
AATGTCCCTGGAATGTACACGTTGGAGGCCTCAGGCCAGGGCTGTGTCTATGTGCAGACGGTGTTGAGATAC
AATATTCTCCCTCCCACAAATATGAAGACCTTTAGTCTTAGTGTGGAAATAGGAAAAGCTAGATGTGAGCAA
CCGACTTCACCTCGATCCTTGACTCTCACTATTCACACCAGTTATGTGGGGAGCCGTAGCTCTTCCAATATG
GCTATTGTGGAAGTGAAGATGCTATCTGGGTTCAGTCCCATGGAGGGCACCAATCAGTTACTTCTCCAGCAA
CCCCTGGTGAAGAAGGTTGAATTTGGAACTGACACACTTAACATTTACTTGGATGAGCTCATTAAGAACACT
CAGACTTACACCTTCACCATCAGCCAAAGTGTGCTGGTCACCAACTTGAAACCAGCAACCATCAAGGTCTAT
GACTACTACCTACCAGGTTCTTTTAAATTATCTCAGTACACAATTGTGTGGTCCATGAACAATGACAGCATA
GTGGACTCTGTGGCACGGCACCCAGAACCACCCCCTTTCAAGACAGAAGCATTTATTCCTTCACTTCCTGGG
AGTGTTAACAACTGATAGCTACCA

The disclosed NOV2 nucleic acid sequence, localized to chromsome 12, has 840 of 1324 bases (63%) identical to a Rattus norvegicus Alpha-2-Macroglobulin Precursor mRNA (GENBANK-ID: RATA2M) (E=1.3e-⁻¹⁹⁹).

A NOV2 polypeptide (SEQ ID NO:10) encoded by SEQ ID NO:9 has 1492 amino acid residues and is presented using the one-letter code in Table 2B. Signal P, Psort and/or Hydropathy results predict that NOV2 does contain a signal peptide and is likely to be localized extracellularly with a certainty of 0.3703. In other embodiments, NOV2 may also be localized to the lysosome (lumen) with a certainty of 0.1900, the microbody (peroxisome) with a certainty of 0.1585, or the endoplasmic reticulum (membrane) with a certainty of 0.1000. The most likely cleavage site for a NOV2 peptide is between amino acids 17 and 18, at: AIA-EE.

TABLE 2B
Encoded NOV2 protein sequence.

(SEQ ID NO:10)

MWAQLLLGMLALSPAIAEELPNYLVTLPARLNFPSVQKVCLDLSPGYSDVKFTVTLETKDKTQKLLEYSGLK
KRHLHCISFLVPPPAGGTEEVATIRVSGVGNNISFEEKKKVLIQRQGNGTFVQTDKPLYTPGQQVYFRIVTM
DSNFVPVNDKYSMVELQDPNSNRIAQWLEVVPEQGIVDLSFQLAPEAMLGTYTVAVAEGKTFGTFSVEEYVL
SPFLLLLSSVLPKFKVEVVEPKELSTVQESFLVKICCRYTYGKPMLGAVQVSVCQKANTYWYREVEREQLPD
KCRNLSGQTDKTGCFSAPVDMATFDLIGYAYSHQINIVATVVEEGTGVEANATQNIYISPQMGSMTFEDTSN
FYHPNFPFSGKMLLKFPQGGVLPCKNHLVFLVIYGTNGTFNQTLVTDNNGLAPFTLETSGWNGTDVSLEGKF
QMEDLVYNPEQVPRYYQNAYLHLRPFYSTTRSFLGIHRLNGPLKCGQPQEVLVDYYIDPADASPDQEISFSY
YLIGKGSLVMEGQKHLNSKKKGLKASFSLSLTFTSRLAPDPSLVIYAIFPSGGVVADKIQFSVEMCFDNQQL
PGAEVELQLQAAPGSLCALRAVDESVLLLRPDRELSNRSVYGMFPFWYGHYPTQVAEYDQCPVSGPWDFPQP
LIDPMPQGHSSQRSIIWRPSFSEGTDLFSFFRDVGLKILSNAKIKKPVDCSHRSPEYSTAMGGGGHPEAFES
STPLHQAEDSQVRQYFPETWLWDLFPIGNSGKEAVHVTVPDAITEWKAMSFCTSQSRGFGLSPTVGLTAFKP
FFVDLTLPYSVVRGESFRLTATIFNYLKDCIRVQTDLAKSHEYQLESWADSQTSSCLCADDAKTHHWNITAV
KLGHINFTISTKILDSNEPCGGQKGFVPQKGRSDTLIKPVLVKPEGVLVEKTHSSLLCPKGGKVASESVSLE
LPVDIVPDSTKAYVTVLGDIMGTALQNLDGLVQMPSGCGEQNMVLFAPIIYVLQYLEKAGLLTEEIRSRAVG
FLEIGYQKELMYKHSNGSYSAFGERDGNGNTWLTAFVTKCFGQAQKFIFIDPKNIQDALKWMAGNQLPSGCY
ANVGNLLHTAMKGGVDDEVSLTAYVTAALLEMGKDVDDPMVSQGLRCLKNSATSTTNLYTQALLAYIFSLAG
EMDIRNILLKQLDQQAIISGESIYWSQKPTPSSNASPWSEPAAVDVELTAYALLAQLTKPSLTQKEIAKATS
IVAWLAKQHNAYGGFSSTQDTVVALQALAKYATTAYMPSEEINLVVKSTENFQRTFNIQSVNRLVFQQDTLP
NVPGMYTLEASGQGCVYVQTVLRYNILPPTNMKTFSLSVEIGKARCEQPTSPRSLTLTIHTSYVGSRSSSNM
AIVEVKMLSGFSPMEGTNQLLLQQPLVKKVEFGTDTLNIYLDELIKNTQTYTFTISQSVLVTNLKPATIKVY
DYYLPGSFKLSQYTIVWSMNNDSIVDSVARHPEPPPFKTEAFIPSLPGSVNN

The disclosed NOV2 amino acid sequence has 595 of 1450 amino acid residues (41%) identical to, and 873 of 1450 residues (60%) positive with, the 1474 amino acid residue Alpha-2-Macroglobulin Precursor protein from Homo sapiens (ptnr:SPTREMBL-ACC:P01023) (E=2.0e⁻²⁷⁹).

NOV2 is expressed in at least the following tissues: Hematopoietic tissues, blood plasma, fetal lung, and the coronary artery.

NOV2 also has homology to the amino acid sequences shown in the BLASTP data listed in Table 2C.

TABLE 2C
BLAST results for NOV2

| Gene Index/ | Protein/ | Length | | | |
|---|---|---|---|---|---|
| Identifier | Organism | (aa) | Identity (%) | Positives (%) | Expect |
| gi\|14765710\|ref\|XP_{—} | alpha 2 | 1474 | 593/1486 | 870/1486 | 0.0 |
| 006925.4\| | macroglobulin | | (39%) | (57%) | |
| | precursor | | | | |
| | [Homo | | | | |
| | sapiens] | | | | |
| gi\|4557225\|ref\|NP_{—} | alpha 2 | 1474 | 591/1486 | 869/1486 | 0.0 |
| 000005.1\| | macroglobulin | | (39%) | (57%) | |
| | precursor | | | | |
| | [Homo | | | | |
| | sapiens] | | | | |
| gi\|224053\|prf\|\| | macroglobulin | 1450 | 585/1471 | 861/1471 | 0.0 |
| 1009174A | alpha2 | | (39%) | (57%) | |
| | [Homo | | | | |
| | sapiens] | | | | |
| gi\|6978425\|ref\|NP_{—} | alpha-2- | 1472 | 578/1483 | 867/1483 | 0.0 |
| 036620.1\| | macroglobulin | | (38%) | (57%) | |
| | [Rattus | | | | |
| | norvegicus] | | | | |
| gi\|2144118\|pir\|\| | alpha- | 1476 | 570/1495 | 858/1495 | 0.0 |
| JC5143 | macroglobulin | | (38%) | (57%) | |
| | precursor - | | | | |
| | guinea pig | | | | |

The homology of these sequences is shown graphically in the ClustalW analysis shown in Table 2D.

[image]

[image]

[image]

[image]

Tables 2E-F list the domain description form DOMAIN analysis results against NOV2. This indicates that the NOV2 sequence has properties similar to those of other proteins known to contain this domain.

TABLE 2E
Domain Analysis of NOV2

gnl|Pfam|pfam00207, A2M, Alpha-2-macroglobulin family. This family
includes the C-terminal region of the alpha-2-macroglobulin family.
(SEQ ID NO:80)
CD-Length = 751 residues, 98.5% aligned
Score = 563 bits (1451), Expect = 2e-161

| | | | |
|---|---|---|---|
| Query: | 728 | EDSQVRQYFPETWLWDLFPIGNSGKEAVHVTVPDAITEWKAMSFCTSQSRGFGLSPTVGL | 787 |
| | | +\| +\| \|\|\|\|+\|\|\|++ + \| \|++\|+\|\|+\|\| \|+ ++ \| ++\| ++ \| \| | |
| Sbjct: | 4 | DDITIRSYFPESWLWEVEEVDRSPVLTVNITLPDSITTWEILAVSLSNTKGLCVADPVEL | 63 |
| Query: | 788 | TAFKPFFVDLTLPYSVVRGESFRLTATIFNYL-KDCIRVQTDLAKSHEYQLESWADSQTS | 846 |
| | | \| \|+ \|\|++\| \|\|\|\|\|\|\|\|\| \| \| ++\|\|\| \|+\| \|\|\| \| | |
| Sbjct: | 64 | TVFQDFFLELRLPYSVVRGEQVELRAVLYNYLPSQDIKV--------VVQLEVEPLCQAG | 115 |
| Query: | 847 | SCLCADDAKTHHWNITAVKLGHINFTISTKILDSNEPCGGQKGFVPQKGRSDTLIKPVLV | 906 |
| | | \| \| ++ \| ++\| + \| \| \|\|+ \| ++\| + \| | |
| Sbjct: | 116 | FCSLATQRTRSSQSVRPKSLSSVSFPVVVVPLASGLSLVEVVASVPEFFVKDAVVKTLKV | 175 |
| Query: | 907 | KPEGVLVEKTHSSLLCP---KGGKVASESVSLELPVDIVPD-STKAYVTVLGDIMGTALQ | 962 |
| | | +\|\|\| \|+\| \|\|\|\| \| \|\| + \| \| \|\|\| +\| ++\| \|\| + \|+\| | |
| Sbjct: | 176 | EPEGARKEETVSSLLLPPEHLGGGLEVSEVPALKLPDDVPDTEAEAVISVQGDPVAQAIQ | 235 |
| Query: | 963 | N------LDGLVQMPSGCGEQNMVLFAPIIYVLQYLEKAGLLTE---EIRSRAVGFLEIG | 1013 |
| | | \| \|+ \|+++\|\|\|\|\|\|\|\|\|+ \|\| +\|\|\| \|\|++ + + + +\|+ + \| | |
| Sbjct: | 236 | NTLSGEGLNNLLRLPSGCGEQNMIYMAPTVYVLHYLDETWQWEKPGTKKKQKAIDLINKG | 295 |
| Query: | 1014 | YQKELMYKHSNGSYSAFGERDGNGNTWLTAFVTKCFGQAQKFIFIDPKNIQDALKW-MAG | 1072 |
| | | \|\|++\| \|+ ++\|\|\|+\|\| \| +\|\|\|\|\|\|\| \| \| \|\|+ ++\|\|\| ++\| \|+\|\| + | |
| Sbjct: | 296 | YQRQLNYRKADGSYAAFLHRA--SSTWLTAFVLKVFSQARNYVFIDEEHICGAVKWLILN | 353 |
| Query: | 1073 | NQLPSGCYANVGNLLHTAMKGGVDD----EVSLTAYVTAALLEMGKDVDDPMVSQGLRCL | 1128 |
| | | \| \| + \| ++\| \|\|\|\|\| \| \|\|+\|\|\|++\| \|\|\|\| \|+\|+ \| \| | |
| Sbjct: | 354 | QQKDDGVFRESGPVIHNEMKGGVGDDAEVEVTLTAFITIALLEAKLVCISPVVANALSIL | 413 |
| Query: | 1129 | KNSATSTTN------LYTQALLAYIFSLAGEMDIRNILLKQLDQQAIISGESIYWS--QK | 1180 |
| | | \| \| \| +\|\| \|\| \|\| +\|\|\| + +\|\| \| ++ + +\| \|\| | |
| Sbjct: | 414 | KASDYLLENYANGQRVYTLALTAYALALAGVLHKLKEILKSLKEELYKALVKGHWERPQK | 473 |
| Query: | 1181 | PTPSSNASPWSEPAAVDVELTAYALLAQLTKPSLTQKEIAKATSIVAWLAKQHNAYGGFS | 1240 |
| | | \| + +\| \| \|\|+\|+\|\|\|\|\| \|\| \| ++ \| +\| \|\| +\| \|\|\| | |
| Sbjct: | 474 | PKDAPGHPYSPQPQAAAVEMTSYALLALLT--LLPFPKVEMAPKVVKWLTEQQYYGGGFG | 531 |
| Query: | 1241 | STQDTVVALQALAKYATTAYMPSE-EINLVVKSTEN-FQRTFNIQSVNRLVFQQDTLP-N | 1297 |
| | | \|\|\|\|\|\|+\|\|\|\|\|+\|\| \| +++ ++\| \|+ \| \| + \| + + \|\| \| | |
| Sbjct: | 532 | STQDTVMALQALSKYGIATPTHKEKNLSVTIQSPSGSFKSHFQILNNNAFLLRPVELPLN | 591 |
| Query: | 1298 | VPGMYTLEASGQGCVYVQTVLRYNILPPTNMKTFSLSVEIGKARCEQPTSPR-SLTLTIH | 1356 |
| | | \| + +\|\|\| + + \| \|\| +\| \| \| \| +\| \| +\| + \| \|+\| | |
| Sbjct: | 592 | EGFTVTAKVTGQGTLTLVTTYRYKVLDKKNTFCFDLKIETVPDTCVEPKGAKNSDYLSIC | 651 |
| Query: | 1357 | TSYVGSRSSSNMAIVEVKMLSGFSPMEGT--NQLLLQQPLVKKVEFGTDTLNIYLDELIK | 1414 |
| | | \| \| \|\|\|\| \| \|\|\| ++ \|\|+\|\| \|++ \| \| \| + + +\|\|\|++ | |
| Sbjct: | 652 | TRYAGSRSDSGMAIADISMLTGFIPLKPDLKKLENGVDRYVSKYEIDGNHVLLYLDKVSH | 711 |
| Query: | 1415 | -NTQTYTFTISQSVLVTNLKPATIKVYDYYLP 1445 | |
| | | \|+ \| \| \| \| \|+\|\|++\|\|\|\|\|\| \| | |
| Sbjct: | 712 | SETECVGFKIHQDFEVGLLQPASVKVYDYYEP 743 | |

TABLE 2F
Domain Analysis of NOV2

gnl|Pfam|pfam01835, A2M_N, Alpha-2-macroglobulin family N-terminal
region. This family includes the N-terminal region of the alpha-2-
macroglobulin family. (SEQ ID NO:81)
CD-Length = 620 residues, 98.4% aligned
Score = 236 bits (603) , Expect = 5e-63

| | | | |
|---|---|---|---|
| Query: | 5 | LLLGMLALSPAIAEEL--PNYLVTLPARLNFPSVQKVCLDLSPGYSDVKFTVTLETKDKT | 62 |
| | | \|\| +\| \| \| \| \|+\| +\|+ \| + +\|\|\|+ \| \| \|\|+\| + | |
| Sbjct: | 2 | LLWLLLLLLLFFDSSLQKPRYMVIVPSILRTETPEKVCVQLHDLNETVTVTVSLHSFPGK | 61 |
| Query: | 63 | QKLLEYSGLK---KRHLHCISFLVPPPA---GGTEEVATIRVSGVGNNISFEEKKKVLIQ | 116 |
| | | + \| + \| \|\|+\|\| \|\| \| \| + + \| \| +\|+\|\| \|\|+ | |
| Sbjct: | 62 | RNLSSLFTVLLSSKDLFHCVSFTVPQPGLFKSSKGEESFVVVQVKGPTHTFKEKVTVLVS | 121 |
| Query: | 117 | RQGNGTFVQTDKPLYTPGQQVYFRIVTMDSNFVPVNDKYSMVELQDPNSNRIAQWLEVVP | 176 |
| | | + \|+\|\|\|\|\|+\|\|\|\|\| \| +\|+ ++\| \| \|+\|+ +\| ++\|\| \|\|+ \|\| | |
| Sbjct: | 122 | SRRGLVFIQTDKPIYTPGQTVRYRVFSVDENLRPLNELI-LVYIEDPEGNRVDQWEVNKL | 180 |
| Query: | 177 | EQGIVDLSFQLAPEAMLGTYTVAV---AEGKTFGT--FSVEEYVLSPFLLLLSSVLPKFK | 231 |
| | | \| \|\| \|\|\| + \| + \|\|+ + + ++ \| \| \|+\|\| + | |
| Sbjct: | 181 | EGGIFQLSFPIPSEPIQGTWKIVARYESGPESNYTHYFEVKEY---------VLPSFEVS | 231 |
| Query: | 232 | VEVVEPKELSTVQESFLVKICCRYTYGKPMLGAVQVSVCQKANTYWYREVEREQLPDKCR | 291 |
| | | + +\| + \| \| \|\| \|\|\|\|\|\|\|+ \| \| \| \| +++\| | |
| Sbjct: | 232 | ITPPKPFIYYDNFKEFEVTICARYTYGKPVPGVAYVRFGVK------DEDGKKELLAGLE | 285 |
| Query: | 292 | NLSGQTDKTG--CFSAPVDMATFDLIGYAY-SHQINIVATVVEEGTGVEANA-TQNIYIS | 347 |
| | | + \| \| \| \| \| + \| + + \|+\| \| \| \| \| | |
| Sbjct: | 286 | ERAKLLDGNGEICLSQEVLLKELQLKNEDLEGKSLYVAVAVIESEGGDMEEAELGGIKIV | 345 |
| Query: | 348 | PQMGSMTFEDTSNFYHPNFPFSGKMLLKFPQGGVLPCKNHLVFLVIYGTNGTFNQTLVTD | 407 |
| | | + \| \| + + \| \|\| \|+\|+ \| \| \| \| \| + + ++ \|\| | |
| Sbjct: | 346 | RSPYKLKFVKTPSHFKPGIPFFLKVLVVDPDGS--PAPNVPVK--VSAQDASYYSNGTTD | 401 |
| Query: | 408 | NNGLAPFTLETSGWNGTDVSLEGKFQMEDLVYNPEQVPRYYQNAYLHLRPFYSTTRSFLG | 467 |
| | | +\|\|\| \|++ \|\| + +\|+ + ++\| + \|\| + \| + \| | |
| Sbjct: | 402 | EDGLAQFSINTS--GISSLSITVRTNHKELPEEVQAHAEAQATAYSTVSL--SKSYIHLS | 457 |
| Query: | 468 | IHRLNGPLKCGQPQEVLVDYYIDPADASPDQEISFSYYLIGKGSLVMEGQKHLNSKKKGL | 527 |
| | | \| \| \| \|\| ++ + + + \| \| ++ \|\| +\| \|++ ++ | |
| Sbjct: | 458 | IER---TLPCGPGVGEQANFILRGKSLGELKILHFYYLIMSKGKIVKTGRE----PREPG | 510 |
| Query: | 528 | KASFSLSLTFTSRLAPDPSLVIYAIFPSGGVVADKIQFSVEMCFDN-----------QQL | 576 |
| | | + \|\|\|\|+ \| \|\|\| \|\| \| \| \| \| \|\|\|\| + \|\| \| \| +\| | |
| Sbjct: | 511 | QGLFSLSIPVTPDLAPSFRLVAYYILPQGEVVADSVWIDVEDCCANKLDLSFSPSKDYRL | 570 |
| Query: | 577 | PGAEVELQLQAAPGSLCALRAVDESVLLLRPDRELSNRSVY 617 | |
| | | \| +\|+\|+++\| \| \|\| \|\|\|\|\|\|++\| \|\|+\| +\|\| \|\| | |
| Sbjct: | 571 | PAQQVKLRVEADPQSLVALRAVDQAVYLLKPKAKLSMSKVY 611 | |

The proteinase-binding alpha-macroglobulins (A2M) [1] are large glycoproteins found in the plasma of vertebrates, in the hemolymph of some invertebrates and in reptilian and avian egg white. A2M-like proteins are able to inhibit all four classes of proteinases by a ‘trapping’ mechanism. They have a peptide stretch, called the ‘bait region’, which contains specific cleavage sites for different proteinases. When a proteinase cleaves the bait region, a conformational change is induced in the protein, thus trapping the proteinase. The entrapped enzyme remains active against low molecular weight substrates, whilst its activity toward larger substrates is greatly reduced, due to steric hindrance. Following cleavage in the bait region, a thiol ester bond, formed between the side chains of a cysteine and a glutamine, is cleaved and mediates the covalent binding of the A2M-like protein to the proteinase.

Alpha2-Macroglobulin (A2M) is a proteinase inhibitor found in association with senile plaques (SP) in Alzheimer's disease (AD). A2M has been implicated biochemically in binding and degradation of the amyloid beta (Abeta) protein which accumulates in SP. We studied the relationship between Alzheimer's disease and a common A2M polymorphism, Val1000 (GTC)/Ile1000 (ATC), which occurs near the thiolester active site of the molecule. In an initial exploratory data set (90 controls and 171 Alzheimer's disease) we noted an increased frequency of the G/G genotype from 0.07 to 0.12. We therefore tested the hypothesis that the G/G genotype is over-represented in Alzheimer's disease in an additional independent data set: a group of 359 controls and 566 Alzheimer's disease patients. In the hypothesis testing cohort, the G/G genotype increased from 0.07 in controls to 0.12 in Alzheimer's disease (P<0.05, Fisher's exact test). The odds ratio for Alzheimer's disease associated with the G/G genotype was 1.77 (1.16-2.70, P<0.01) and in combination with APOE4 was 9.68 (95% CI 3.91-24.0, P<0.001). The presence of the G allele was associated with an increase in Abeta burden in a small series. The A2M receptor, A2M-r/LRP, is a multifunctional receptor whose ligands include apolipoprotein E and the amyloid precursor protein. These four proteins have each been genetically linked to Alzheimer's disease, suggesting that they may participate in a common disease pathway.

Six alpha 2-macroglobulin cDNA clones were isolated from two liver cDNA libraries produced from rats undergoing acute inflammation. The coding sequence for rat alpha 2-macroglobulin including its 27-residue signal peptide and the 3′- and part of the 5′ nontranslated regions were determined. The mature protein consisting of 1445 amino acids is coded for by a 4790+/−40 nucleotide messenger RNA. It contains a typical internal thiol ester region and 25 cysteine residues which are conserved between rat and human alpha 2-macroglobulin. Although the amino acid sequences of rat and human alpha 2-macroglobulin share 73% identity, two small divergent areas of 17 and 38 residues were found, corresponding to 29 and 11% identity, respectively. These areas are located in the bait region and, therefore, may confer specific proteinase recognition capabilities on rat alpha 2-macroglobulin. Following an inflammatory stimulation, rat alpha 2-macroglobulin mRNA levels increased 214-fold over control values and reached a maximum at 18 h. By 24 h the levels had decreased to less than 30% of the maximum value. Transcription rates from the alpha 2-macroglobulin gene as measured in nuclear run-on experiments showed a less than 3-fold increase in nuclei from acutely inflamed rats as compared to controls. These results suggest that the accummulation of alpha 2M mRNA is due to the combined effects of increased transcription rates and post-transcriptional processing.

The alpha 2-macroglobulin (alpha 2M) receptor complex as purified by affinity chromatography contains three polypeptides: a 515 -kDa heavy chain, an 85 -kDa light chain, and a 39-kDa associated protein. Previous studies have established that the 515/85-kDa components are derived from a 600 -kDa precursor whose complete sequence has been determined by cDNA cloning (Herz, J., Hamann, U., Rogne, S., Myklebost, O., Gassepohl, H., and Stanley, K. (1988) EMBO J. 7,4119-4127). We have now determined the primary structure of the human 39 -kDa polypeptide, termed alpha 2M receptor-associated protein, by cDNA cloning. The deduced amino acid sequence contains a putative signal sequence that precedes the 323-residue mature protein. Comparative sequence analysis revealed that alpha 2M receptor-associated protein has 73% identity with a rat protein reported to be a pathogenic domain of Heymann nephritis antigen gp 330 and 77% identity to a mouse heparin-binding protein termed HBP-44. The high overall identity suggests that these molecules are interspecies homologues and indicates that the pathogenic domain, previously thought to be a portion of gp 330, is in fact a distinct protein. Further, the 120-residue carboxyl-terminal region of alpha 2M receptor-associated protein has 26% identity with a region of apolipoprotein E containing the low density lipoprotein receptor binding domain. Pulse-chase experiments revealed that the newly formed alpha 2M receptor-associated protein remains cell-associated, while surface labeling experiments followed by immunoprecipitation suggest that this protein is present on the cell surface forming a complex with the alpha 2M receptor heavy and light chains.

Alpha-2-macroglobulin is a serum pan-protease inhibitor. A possible correlation to this gene is the following reference on Rhinovirus infections causing exacerbations of eosinophilic airway disease. The acute effects of allergen-challenge on nasal interleukin-8 (IL-8), eosinophil cationic protein (ECP), and alpha2-macroglobulin were examined in atopic subjects with common cold symptoms. Twenty-three patients with seasonal allergic rhinitis were inoculated with human rhinovirus 16 outside the pollen season. Diluent and allergen challenges, followed by nasal lavages, were carried out about 3 months before and 4 days after virus inoculation. Seventeen patients developed significant common cold symptoms with increased nasal lavage fluid levels of alpha2-macroglobulin, IL-8, and ECP at baseline (p<0.001-0.05 versus before inoculation), and were further increased by allergen challenge (p<0.001-0.05); IL-8 and ECP levels were correlated. See Eur Respir J 1999 Jan;13(1):41-7. Allergen challenge-induced acute exudation of IL-8, ECP and alpha2-macroglobulin in human rhinovirus-induced common colds. Greiff L, Andersson M, Svensson C, Linden M, Myint S, Persson CG Dept of Otorhinolaryngology, Head and Neck Surgery, University Hospital, Lund, Sweden.

In the CNS area Alpha-2-macroglobulin has been implicated in Alzheimer disease (AD) based on its ability to mediate the clearance and degradation of A-beta, the major component of amyloid beta deposits. Blacker et al. (1998) analyzed a deletion in the A2M gene at the 5-prime splice site of ‘exon II’ of the bait region (exon 18) and found that inheritance of the deletion, designated A2M-2, conferred increased risk for AD.

The disclosed NOV2 nucleic acid of the invention encoding a Alpha-2-macroglobulin precursor-like protein includes the nucleic acid whose sequence is provided in Table 2A or a fragment thereof. The invention also includes a mutant or variant nucleic acid any of whose bases may be changed from the corresponding base shown in Table 2A while still encoding a protein that maintains its Alpha-2-macroglobulin precursor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 40% percent of the bases may be so changed.

The disclosed NOV2 protein of the invention includes the Alpha-2-macroglobulin precursor-like protein whose sequence is provided in Table 2B. The invention also includes a mutant or variant protein any of whose residues may be changed from the corresponding residue shown in Table 2B while still encoding a protein that maintains its Alpha-2-macroglobulin precursor-like activities and physiological functions, or a functional fragment thereof. In the mutant or variant protein, up to about 62% percent of the residues may be so changed.

The NOV2 nucleic acids and proteins of the invention are useful in potential therapeutic applications implicated in asthma, allergy and psoriasis, Alzheimer disease, Emphysema, pulmonary disease, immune disorders and Cancer and/or other pathologies and disorders. The NOV2 nucleic acid encoding Alpha-2-macroglobulin precursor-like protein, and the Alpha-2-macroglobulin precursor-like protein of the invention, or fragments thereof, may further be useful in diagnostic applications, wherein the presence or amount of the nucleic acid or the protein are to be assessed.

NOV2 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV2 protein has multiple hydrophilic regions, each of which can be used as an immunogen. In one embodiment, a contemplated NOV2 epitope is from about amino acids 80 to 100. In another embodiment, a NOV2 epitope is from about amino acids 110 to 200. In additional embodiments, NOV2 epitopes are from about amino acids 290 to 340, from about 380 to 400, from about amino acids 410 to 580, from about amino acids 620 to 730, from about amino acids 810 to 900, from about amino acids 990 to 1100, from about amino acids 1190 to 1210, from about amino acids 1240 to 1320, and from about amino acids 1400 to 1450. These novel proteins can be used in assay systems for functional analysis of various human disorders, which are useful in understanding of pathology of the disease and development of new drug targets for various disorders.

NOV3

A disclosed NOV3 nucleic acid of 987 nucleotides (also referred to as GMAC079237_A) encoding a novel Ileal Sodium/Bile Acid Cotransporter-like protein is shown in Table 3A. An open reading frame was identified beginning with a ATG initiation codon at nucleotides 1-3 and ending with a TGA codon at nucleotides 979-981. The start and stop codons are in bold letters, and the 3′ untranslated region is underlined.

TABLE 3A
NOV3 Nucleotide Sequence

(SEQ ID NO:11)

ATGAGAGCCAATTGTTCCAGCAGCTCAGCCTGCCCTGCCAACAGTTCAGAGGAGGAGCTGCCAGTGGGACT
GGAGGTGCATGGAAACCTGGAGCTCGTTTTCACAGTGGTGTCCACTATCATGATGGGGCTGCTCATGTTCT
CTTTGGGATGTTCCGTGGAGATCCGGAAGCTGTGGTCGCACATCAGGAGACCCTGGGGCATTGCTGTGGGA
CTGCTCTGCCAGTTTGGGCTCATGCCTTTTACAGCTTATCTCCTGGCCATTAGCTTTTCTCTGAAGCCAGT
CCAAGCTATTGCTGTTCTCATCATGGGCTGCTGCCGGGGGGCACCATCTCTAACATTTTCACCTTCTGGGT
TGATGGAGATATGGATCTCAGGTGCCCTGGGAATGATGCCACTCTGCATTTATCTCTACACCTGGTCCTGG
AGTCTTCAGCAGAATCTCACCATTCCTTATCAGAACATAGGTCTGTCTTTAGGAATTACCCTTGTGTGCCT
GACCATTCCTGTGGCCTTTGGTGTCTATGTGAATTACAGATGGCCAAAACAATCCAAAATCATTCTCAAGG
CCGTTGTTGGTGGGGTCCTCCTTCTGGTGGTCGCAGTTGCTGGTGTGGTCCTGGCGAAAGGATCTTGGAAT
TCAGACATCACCCTTCTGACCATCAGTTTCATCTTTCCTTTGATTGGCCATGTCACGGGTTTTCTGCTGGC
ACTTTTTACCCACCAGTCTTGGCAAAGGACCTTGCCTATCTTTTTAGGTTTAGCTTTCAAGACACCCTGTG
ATACCCTACTCGCAATGACTTCGTGTCCTGAATGTTCCAGGCTCATCTATGCCTTCATTCCTCTGCTATAT
GGACTCTTCCAGCTGATAGATGGATTTCTTATTGTTGAAGAGAGAACAGAAGATACAGACTGCGATGGTTC
ACCTTTACCTGAGTATTTTACTGAGGTAACAATAATACCTAAACAACCTAGGATATGACAGCTT

The disclosed NOV3 nucleic acid sequence maps to the q33 region of chromosome 13 and has 257 of 382 bases (67%) identical to a O.cuniculus ileal sodium-dependent bile acid transporter mRNA from O. cuniculus (GENBANK-ID: Z54357) (E=4.9e ⁻³⁴).

A disclosed NOV3 protein (SEQ ID NO:12) encoded by SEQ ID NO:11 has 326 amino acid residues, and is presented using the one-letter code in Table 3B. Signal P, Psort and/or Hydropathy results predict that NOV3 does not have a signal peptide, and is likely to be localized to the plasma membrane with a certainty of 0.6000. In other embodiments NOV3 is also likely to be localized to Golgi body with a certainty of 0.4000, to the mitochondrial inner membrane with a certainty of 0.3815, or to the endoplasmic reticulum (membrane) with a certainty of 0.3000.

TABLE 3B
ENCODED NOV3 PROTEIN SEQUENCE.

(SEQ ID NO:12)

MRANCSSSSACPANSSEEELPVGLEVHGNLELVFTVVSTIMMGLLMFSLGCSVEIRKLWSHIRRPWGIAVG
LLCQFGLMPFTAYLLAISFSLKPVQAIAVLIMGCCRGAPSLTFSPSGLMEIWISGALGMMPLCIYLYTWSW
SLQQNLTIPYQNIGLSLGITLVCLTIPVAFGVYVNYRWPKQSKIILKAVVGGVLLLVVAVAGVVLAKGSWN
SDITLLTISFIFPLIGHVTGFLLALFTHQSWQRTLPIFLGLAFKTPCDTLLAMTSCPECSRLIYAFIPLLY
GLFQLIDGFLIVEERTEDTDCDGSPLPEYFTEVTIIPKQPRI

The disclosed NOV3 amino acid has 129 of 331 amino acid residues (38%) identical to, and 198 of 331 amino acid residues (59%) similar to, the 348 amino acid residue ileal sodium/bile acid cotransporter protein from Cricetulus griseus (Chinese hamster) (Q60414) (E=1.6e⁻⁵⁶).

The NOV3 sequence is predicted to be expressed in ileum because of the expression pattern of (GENBANK-ID: NTCI_HUMAN) a closely related ILEAL SODIUM/BILE ACID COTRANSPORTER homolog in species Homo sapien.

TaqMan data for NOV3 can be found below in Example 2. NOV3 also has homology to the amino acid sequences shown in the BLASTP data listed in Table 3E.

TABLE 3E
BLAST results for NOV3

| Gene Index/ | Protein/ | Length | Identity | Positives | |
|---|---|---|---|---|---|
| Identifier | Organism | (aa) | (%) | (%) | Expect |
| gi\|12858115\|dbj\| | putative | 373 | 187/310 | 226/310 | 6e−87 |
| BAB31203.1\| (AK018423) | [Mus | | (60%) | (72%) | |
| | musculus] | | | | |
| gi\|3024224\|sp\|Q28727\| | ILEAL | 347 | 116/279 | 173/279 | 1e−52 |
| NTCI_RABIT | SODIUM/BILE | | (41%) | (61%) | |
| | ACID | | | | |
| | COTRANSPORTER | | | | |
| | (ILEAL | | | | |
| | NA(+)/BILE | | | | |
| | ACID | | | | |
| | COTRANSPORTER) | | | | |
| | (NA+ | | | | |
| | DEPENDENT | | | | |
| | ILEAL BILE | | | | |
| | ACID | | | | |
| | TRANSPORTER) | | | | |
| | (ILEAL | | | | |
| | SODIUM- | | | | |
| | DEPENDENT | | | | |
| | BILE ACID | | | | |
| | TRANSPORTER) | | | | |
| | (ISBT) | | | | |
| | (SODIUM/TAUR | | | | |
| | OCHOLATE | | | | |
| | COTRANSPORTING | | | | |
| | POLYPEPTIDE, | | | | |
| | ILEAL) | | | | |
| gi\|8394281\|ref\|NP_{—} | solute | 348 | 130/344 | 195/344 | 2e−52 |
| 058918.1\| | carrier | | (37%) | (55%) | |
| | family 10, | | | | |
| | member 2 | | | | |
| | [Rattus | | | | |
| | norvegicus] | | | | |
| gi\|6755530\|ref\|NP_{—} | solute | 348 | 125/313 | 191/313 | 4e−52 |
| 035518.1\| | carrier | | (39%) | (60%) | |
| | family 10, | | | | |
| | member 2 | | | | |
| | [Mus | | | | |
| | musculus] | | | | |
| gi\|6755530\|ref\|NP_{—} | solute | 348 | 125/313 | 191/313 | 4e−52 |
| 035518.1\| | carrier | | (39%) | (60%) | |
| | family 10, | | | | |
| | member 2 | | | | |
| | [Mus | | | | |
| | musculus] | | | | |
| gi\|2842631\|sp\|Q60414\| | ILEAL | 348 | 121/306 | 185/306 | 4e−52 |
| NTCI_CRIGR | SODIUM/BILE | | (39%) | (59%) | |
| | ACID | | | | |
| | COTRANSPORTER | | | | |
| | (ILEAL | | | | |
| | NA(+)/BILE | | | | |
| | ACID | | | | |
| | COTRANSPORTER) | | | | |
| | (NA+ | | | | |
| | DEPENDENT | | | | |
| | ILEAL BILE | | | | |
| | ACID | | | | |
| | TRANSPORTER) | | | | |
| | (ILEAL | | | | |
| | SODIUM- | | | | |
| | DEPENDENT | | | | |
| | BILE ACID | | | | |
| | TRANSPORTER) | | | | |
| | (ISBT) | | | | |
| | (SODIUM/TAUR | | | | |
| | OCHOLATE | | | | |
| | COTRANSPORTING | | | | |
| | POLYPEPTIDE, | | | | |
| | ILEAL) | | | | |

The homology of these sequences is shown graphically in the ClustalW analysis shown in Table 3F.

[image]

[image]

[image]

Table 3G list the domain description from DOMAIN analysis results against NOV3. This indicates that the NOV3 sequence has properties similar to those of other proteins known to contain this domain.

TABLE 3G
DOMAIN ANALYSIS OF NOV3

gnl|Pfam|pfam01758, SBF, Sodium Bile acid symporter family. This
family consists of Na+/bile acid co-transporters. These transmembrane
proteins function in the liver in the uptake of bile acids from portal
blood plasma a process mediated by the co-transport of Na+. Also in
the family is ARC3 from S. cerevisiae this is a putative transmembrane
protein involved in resistance to arsenic compounds. (SEQ ID NO:82)
CD-Length = 186 residues, 80.1% aligned
Score = 75.9 bits (185), Expect = 3e−15

| | | | |
|---|---|---|---|
| Query: | 39 | TIMMGLLMFSLGCSVEIRKLWSHIRRPWGIAVGLLCQFGLMPFTAYLLAIS-FSLKPVQA | 97 |
| | | + + \|+\|\|\|+\| \| \| +\|\|\| + +\|\|\| \|+ +\|\| ++\|\| \| \| \| | |
| Sbjct: | 1 | ALGLFLMMFSMGLKVRFEDLKEALRRPKALILGLLLQWIIMPLLMFILAWLLLRLPPELA | 60 |
| Query: | 98 | IAVLIMGCCRG---APSLTFSPSGLMEIWIS-------GALGMMPLCIYLYTWSWSLQQN | 147 |
| | | ++++\|\| \| + \|+ \| +\|+ + \| + \|\| +\| | |
| Sbjct: | 61 | TGLILVGCAPGGAMSNVWTYLAKGDVELSVVMVALSTLLAPLVTPLLSFLLAGLL----- | 115 |
| Query: | 148 | LTIPYQNIGLSLGITLVCLTIPVAFGVYVNYRWP 181 | |
| | | ++ + + \|\| + \|\|+ \|+ \| \| | |
| Sbjct: | 116 | VHVDAVSPWSLIKSVLVYVIIPLIAGMLTRYFLP 149 | |

Bile acids are synthesized from cholesterol in the liver and secreted into the small intestine, where they facilitate absorption of fat-soluble vitamins and cholesterol. Wong et al. (1996) noted that, rather than being excreted, the majority of bile acids are reabsorbed from the intestine and returned to the liver via the portal circulation. In the liver, bile acids are quantitatively extracted and resecreted into bile, thereby eliminating the need for substantial de novo hepatic bile acid synthesis. The ileum is the major site of active uptake of bile acids from the intestine, where the sodium-gradient-driven transporter has been identified in the ileal enterocyte. Mutations in the transporter or other ileal genes that participate in the transepithelial transport of bile acids are predicted to affect bile acid and cholesterol metabolism significantly. For example, Heubi et al. (1982) described an apparent familial defect in active ileal bile acid transport.

Using homologous sequences from hamster and rat, Wong et al. (1995) cloned a cDNA encoding an ileal sodium/bile acid cotransporter gene (designated ISBT by them). They also isolated a genomic clone for human ISBT. The gene encodes a 348-amino acid polypeptide with 7 predicted transmembrane domains and a predicted molecular mass of 38 kD. The native human protein has a relative molecular mass of 40 kD on SDS gel electrophoresis due to N-linked glycosylation. Wong et al. (1995) demonstrated a dysfunctional mutation (P290S) in the ileal sodium-bile acid cotransporter gene in the course of cloning the human cDNA.

Wong et al. (1996) mapped the SLC10A2 gene to chromosome 13 by study of a human/rodent cell hybrid mapping panel and refined the localization to 13q33 by fluorescence in situ hybridization. The ileal sodium-bile acid cotransporter gene is clearly distinct from the hepatic sodium-bile acid cotransporter gene (SLC10A1; 182396) which maps to chromosome 14. Lammert et al. (1998) mapped the Slc10a2 gene to mouse chromosome 8 in a region homologous to chromosome 13q33.

Primary bile acid malabsorption (PBAM) is an idiopathic intestinal disorder associated with congenital diarrhea, steatorrhea, interruption of the enterohepatic circulation of bile acids, and reduced plasma cholesterol levels. Oelkers et al. (1997) screened the SLC10A2 gene for PBAM-associated mutations using SSCP analysis. Four polymorphisms were identified and sequenced in a family with congenital PBAM. One allele encoded an A171S missense mutation and a mutated donor splice site for exon 3 (601295.0002). The other allele encoded 2 missense mutations at conserved amino acid positions, L243P and T262M (601295.0001). In transfected COS cells, the L243P, T262M, and double mutant (L243P/T262M) did not affect transporter protein expression or trafficking to the plasma membrane; however, transport of taurocholate and other bile acids was abolished. In contrast, the A171S mutation had no effect on taurocholate uptake. The dysfunctional mutations were not detected in 104 unaffected control subjects, whereas the A171 S was present in 28% of that population. The findings of Oelkers et al. (1997) established that SLC10A2 mutations can cause PBAM and underscored the ileal Na(+)/bile acid cotransporter's role in intestinal reclamation of bile acids. Autosomal recessive inheritance had been suggested by earlier studies of PBAM patients and was supported by the findings that the proband's son, who inherited an SLC10A2 allele encoding the L243P and T262M missense mutations, was free of clinical symptoms. The authors stated this was the second reported defect of a Na(+)/solute cotransporter, the first having been SLC5A1 (182380), the cotransporter defective in glucose/galactose malabsorption.

As reviewed by Small (1997), the enterohepatic circulation (EHC) is an in vivo ecologic system for the conservation of bile salts, allowing them to be used over and over for the absorption of fat. The EHC confines the bile salt pool to the liver, bile ducts, gallbladder, small intestine, and portal vein. There is a virtual absence of bile salts in the systemic circulation. A minimum of 6 (known or postulated) molecules (3 in the ileal mucosal cells and 3 analogous molecules in the hepatocytes) constitutes the active players in the EHC (Dawson and Oelkers, 1995). The polar ileal enterocytes and the hepatocytes each have 3 unique molecules: a receptor that binds bile salts on one surface and translocates them into the cell, a cellular bile salt binding protein that moves them across the cell, and an exit molecule that moves bile salts out of the other side of the cell. In the intestine, ileal sodium/bile salt transporter (ISBT) is present on the brush boarders of the ileum but not the jejunum. It binds bile salts in the gut lumen and transports them across the brush boarder membrane and hands them to the ileal lipid-binding protein (ILBP; 600422) which binds bile acid in the cytoplasm of the cell. ILBP allows the bile salts to move through the cytoplasm to the basolateral membrane of the ileal intestinal epithelial cell, where a sodium-independent organic ion exchange system secretes bile salts into portal capillaries. Bile salts in portal capillaries bind to albumin and flow to the liver. There they are recognized by a transporter with high homology to ISBT, the sodium taurocholate cotransporting polypeptide (SLC10A1). Small et al. (1972) suggested that a genetic defect in the predicted bile acid receptor in the ileum would lead to diarrhea and/or steatorrhea and suggested that bile acid turnover and fecal bile acid excretion be studied in patients with unexplained diarrhea. Hess Thaysen and Pedersen (1976) described several patients who had diarrhea and excessive bile acid loss, without other ileal pathology. Heubi et al. (1979, 1982) reported a case study of a boy who presented 48 hours after birth with severe diarrhea, steatorrhea, and malabsorption. Intestinal absorption of bile acid was nearly absent and resulted in a small pool size, a low interluminal bile acid concentration, and severe malabsorption of water and fat. Ileal biopsies had no active bile acid transport. Parenteral nutrition was necessary to sustain the child. At the other extreme, a child with marked bile acid malabsorption but with almost normal development, nearly normal fat absorption, and a moderately well-maintained bile acid pool, was described by Jonas et al. (1986). This patient had a 15-fold increase in bile acid synthesis that was adequate to maintain pool size, interluminal bile acids, and fat absorption. Thus, the clinical phenotype apparently can vary from severe diarrhea, fat malabsorption, and malnutrition, to modest diarrhea without significant fat malabsorption. Small (1997) suggested that the bile acid malabsorption and the variable severity could represent mutations in any of the 3 main players in the ileal transport.

The disclosed NOV3 nucleic acid of the invention encoding a Ileal Sodium/Bile Acid Cotransporter-like protein includes the nucleic acid whose sequence is provided in Table 3A or a fragment thereof. The invention also includes a mutant or variant nucleic acid any of whose bases may be changed from the corresponding base shown in Table 3A while still encoding a protein that maintains its Ileal Sodium/Bile Acid Cotransporter-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 33% percent of the bases may be so changed.

The disclosed NOV3 protein of the invention includes the Ileal Sodium/Bile Acid Cotransporter-like protein whose sequence is provided in Table 3B or 3D. The invention also includes a mutant or variant protein any of whose residues may be changed from the corresponding residue shown in Table 3B or 3D while still encoding a protein that maintains its Ileal Sodium/Bile Acid Cotransporter-like activities and physiological functions, or a functional fragment thereof. In the mutant or variant protein, up to about 61% percent of the residues may be so changed.

The protein similarity information, expression pattern, and map location for the Ileal Sodium/Bile Acid Cotransporter-like protein and nucleic acid (NOV3) disclosed herein suggest that NOV3 may have important structural and/or physiological functions characteristic of the citron kinase-like family. Therefore, the NOV3 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV3 nucleic acids and proteins of the invention are useful in potential diagnostic and therapeutic applications implicated in various diseases and disorders described below. For example, the compositions of the present invention will have efficacy for treatment of patients suffering from Primary bile acid malabsorption (PBAM, an idiopathic intestinal disorder), congenital diarrhea, steatorrhea, refractory infantile diarrhea, interruption of the enterohepatic circulation of bile acids, reduced plasma cholesterol levels, crohn's disease, Inflammatory bowel disease, Diverticular disease, Hirschsprung's disease, Cirrhosis, Transplantation, Hypercalceimia, Ulcers, growth failure and/or other pathologies. The NOV3 nucleic acid, or fragments thereof, may further be useful in diagnostic applications, wherein the presence or amount of the nucleic acid or the protein are to be assessed.

NOV3 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV3 protein have multiple hydrophilic regions, each of which can be used as an immunogen. In one embodiment, contemplated NOV3 epitope is from about amino acids 5 to 30. In another embodiment, a NOV3 epitope is from about amino acids 55 to 60. In additional embodiments, NOV3 epitopes are from about amino acids 140 to 150, from about amino acids 180 to 190, and from about amino acids 280 to 330. This novel protein also has value in development of powerful assay system for functional analysis of various human disorders, which will help in understanding of pathology of the disease and development of new drug targets for various disorders.

NOV4

A disclosed NOV4 nucleic acid of 850 nucleotides (designated CuraGen Acc. No. AL161453 A) encoding a novel Prohibitin-like protein is shown in Table 4A. An open reading frame was identified beginning with an ATG initiation codon at nucleotides 6-8 and ending with a TGA codon at nucleotides 822-824. A putative untranslated regions upstream of the initiation codon and downstream from the termination codon are underlined in Table 4A, and the start and stop codons are in bold letters.

TABLE 4A
NOV4 NUCLEOTIDE SEQUENCE

(SEQ ID NO:13)

AAAACATGGCTGCCAAAATGTTTGAGTTCATCGGCAAGTTTGGCCTGGCCTTAGTTGTTGCAG
GAGGCGTGGTGAACTCTGCCTTATATAGTGTGGATGCTGGGCACAGAGCTGTCGTCTTTGACC
GATTCCGTGGAGTGCAGGACATTGTGGTAGGCAAAGGGACTCACTGTCTCATCCCATGGTTAC
AGAAATCAATAATCTTTGACTGCCGTTCTCAGCCACGTAATGTGCCAGTCATCACCGGTAGCA
AAGATTTACAGAATGTCAACCTCACACTGCGCATCATCTTCCGGCCCGTAGCTAGCCAGCTTC
CTCACATCTTCACCAGCAGCGGAGAGGACCATGATGAGCGTGTGCCGCCGTCCATCACGAACAAGA
TCCTCAAGTCAGTGGTGGCTCGCTTTGAAGCTGGAGAACTAATCACCCAGAGAGAGCAGATCTCCAG
GCAGGTGAGCGATGACCTTACGGAGCCAGCAGCCACCTTTGGGCTCATTCTGGACGACGTGTC
CTTGACATATCTGACCTTCGGGAAGGAGTTCATAGAAGCGGTGGAAGCCAAACAGATAGCTCA
GCAGGAAGCAGAGAGGGCCAGATTTGTGGTGGAAAAGGCTGAGCAGCAGAAAAAGGCGGCCAT
CATTTCTGCTGAGGGCGACTCCAAGGTGGCCGAGCTGATCACCAACTCACTGGCCACAGCAGG
GGACGCCCTGATCGAGCTGCGCAAGCTGGAAGCTGTGGAGGACATCACCTACCAGCTTTTACG
CTCTCGGAACATCACCTACCTGCGGGCAGGGCAGTCCATGCCCCTGCAGCTGCGCTGGTGAGG
GCCCACCCTGCCTGCACCTCCGAG

The nucleic acid sequence of NOV4, localized on chromosome 9, has 782 of 849 bases (92%) identical to a Prohibitin mRNA from Homo sapiens (GENBANK-ID: S85655) (E=7.2e-⁻¹⁵⁸).

A NOV4 polypeptide (SEQ ID NO: 14) encoded by SEQ ID NO: 13 is 272 amino acid residues and is presented using the one letter code in Table 4B. Signal P, Psort and/or Hydropathy results predict that NOV4 has a signal peptide and is likely to be localized at the endoplasmic reticulum (membrane) with a certainty of 0.5500. In other embodiments, NOV4 may also be localized to the lysosome (lumen) with a certainty of 0.2631, the endoplasmic reticulum (lumen) with a certainty of 0.1000, or extracellularly with a certainty of 0.1000. The most likely cleavage site for a NOV4 peptide is between amino acids 25 and 26, at: VNS-AL.

TABLE 4B
NOV4 PROTEIN SEQUENCE

(SEQ ID NO:14)

MAAKMFEFIGKFGLALVVAGGVVNSALYSVDAGHRAVVFDRFRGVQDIVV
GKGTHCLIPWLQKSIIFDCRSQPRNVPVITGSKDLQNVNLTLRIIFRPVA
SQLPHIFTSSGEDHDERVPPSITNKILKSVVARFEAGELITQREQISRQV
SDDLTEPAATFGLILDDVSLTYLTFGKEFIEAVEAKQIAQQEAERARFVV
EKAEQQKKAAIISAEGDSKVAELITNSLATAGDALIELRKLEAVEDITYQ
LLRSRNITYLRAGQSMPLQLRW

The full amino acid sequence of the protein of the invention was found to have 236 of 270 amino acid residues (87%) identical to, and 251 of 270 amino acid residues (92%) similar to, the 272 amino acid residue Prohibitin, protein from Homo sapiens ACC: P35232) (E=3.0e⁻¹¹⁷).

The disclosed NOV4 protein is widely expressed in a variety of tissues.

TaqMan data for NOV4 can be found below in Example 2. NOV4 also has homology to the amino acid sequences shown in the BLASTP data listed in Table 4C.

TABLE 4C
BLAST results for NOV4

| Gene Index/ | | Length | Identity | | |
|---|---|---|---|---|---|
| Identifier | Protein/Organism | (aa) | (%) | Positives (%) | Expect |
| gi\|4505773\|ref\|NP_{—} | prohibitin [Homo | 272 | 236/270 | 251/270 | e−123 |
| 002625.1\| | sapiens] | | (87%) | (92%) | |
| gi\|92643\|pir\|\|A39682 | prohibitin - rat | 272 | 235/270 | 251/270 | e−123 |
| | | | (87%) | (92%) | |
| gi\|7298546\|gb\|AAF53 | 1(2)37Cc gene | 276 | 178/270 | 220/270 | 8e−96 |
| 765.1\| (AE003661) | product | | (65%) | (80%) | |
| | [Drosophila | | | | |
| | melanogaster] | | | | |
| gi\|2055454\|gb\|AAB53 | prohibitin-like | 274 | 153/264 | 209/264 | 9e−80 |
| 231.1\| (U97204) | molecule TC-PRO-1 | | (57%) | (78%) | |
| | [Toxocara canis] | | | | |
| gi\|13491275\|gb\|AAK2 | Hypothetical | 275 | 154/270 | 210/270 | 6e−79 |
| 7865.1\| (AC087079) | protein Y37E3.9 | | (57%) | (77%) | |
| | [Caenorhabditis | | | | |
| | elegans] | | | | |

The homology of these sequences is shown graphically in the ClustalW analysis shown in Table 4D.

[image]

[image]

Tables 4E-F list the domain description from DOMAIN analysis results against NOV4. This indicates that the NOV4 sequence has properties similar to those of other proteins known to contain this domain.

TABLE 4E
DOMAIN ANALYSIS OF NOV4

gnl|Pfam|pfam01145, Band_7, SPFH domain/Band 7 family. This family
also includes proteins with high blast scores to known Band 7 protein:
Hf1C from E. coli Hf1K from E. coli, and Prohibitin family members (SEQ ID NO:83)
CD-Length = 191 residues, 91.6% aligned
Score = 157 bits (397), Expect = 7e-40

| | | | |
|---|---|---|---|
| Query: | 28 | YSVDAGHRAVVFDRFRGVQDIVVGKGTHCLIPWLQKSIIFDCRSQPRNVPVITGSKDLQN | 87 |
| | | \| \| \|+\| \| \|+ \|\|\| \| \| +\|\|++ \| \|+ +\|\| | |
| Sbjct: | 17 | KIVKEYERGVIFRLGRYVRQ-VVGPGLHFIIPFIDTVKKVDLRTVVYDVPSQEIITKDNV | 75 |
| Query: | 88 | VNLTLRIIFRPVASQLPHIFTSSGEDHDERVPPSITNKILKSVVARFEAGELITQREQIS | 147 |
| | | \| + +++ \| \| ++ \|\| \| + \|++\|+ +\| \|++\|+\|\|+\|+ | |
| Sbjct: | 76 | VVIVDAVVYYRVVDPLKAVYEVED---AERALPQLAQTTLRNVIGQFTLDEILTERERIN | 132 |
| Query: | 148 | RQVSDDLTEPAATFGLILDDVSLTYLTFGKEFIEAVEAKQIAQQEAERARFVVEKAEQQK | 207 |
| | | \|+ + \| \| +\|+ ++ \| + + +\| \|+ \|+ \|++\|\| \|\|+ + + \|\|+ | |
| Sbjct: | 133 | SQLREILDEATDPWGIKVERVEIKDIRLPEEVQRAMAAQMEAEREA-RAKILEAEGEQEA | 191 |

TABLE 4F
DOMAIN ANALYSIS OF NOV4

gnl|Smart|smart00244, PHB, prohibitin homologues; prohibitin
homologues (SEQ ID NO:84)
CD-Length = 160 residues, 98.8% aligned
Score = 97.4 bits (241), Expect = 9e−22

| | | | |
|---|---|---|---|
| Query: | 28 | YSVDAGHRAVVFDRFRGVQDIVVGKGTHCLIPWLQKSIIFDCRSQPRNVPVITG-SKDLQ | 86 |
| | | + \| \| +\| \| \|+\| \| \| +\|\|++ \| \|+\| +\|\| +\|\| | |
| Sbjct: | 3 | FYVIGEGERGVVERLGRVLK-VLGPGLHFVIPFIDDVKRVDLRAQTDDVPPQEVITKDNV | 61 |
| Query: | 87 | NVNLTLRIIFRPVASQLPHIFTSSGEDHDERVPPSITNKILKSVVARFEAGELIT-QREQ | 145 |
| | | \|++ + +\| \| \| ++ \| \| \| + \|+\|\|+ + \|\|+\| +\|\|+ | |
| Sbjct: | 62 | TVSVDAVVYYR-VLDPLKAVYGV--LDADYRALRQLAQTTLRSVIGKRTLDELLTDEREK | 118 |
| Query: | 146 | ISRQVSDDLTEPAATFGLILDDVSLTYLTFGKEFIEAVEAKQ 187 | |
| | | \|\| + ++\| \| \| +\|+ ++\|\| + + +\| \|\|+\|\|+\| | |
| Sbjct: | 119 | ISENIREELNEAAEPWGIEVEDVEIKDIRLPEEIKEAMEAQQ 160 | |

Genes that negatively regulate proliferation inside the cell are of considerable interest because of the implications in processes such as development and cancer. Prohibitin, a novel cytoplasmic anti-proliferative protein widely expressed in a variety of tissues, inhibits DNA synthesis. Studies have suggested that Prohibitin may be a suppressor gene and is associated with tumor development and/or progression of at least some breast cancers. Sequence comparisons suggest that the Prohibitin gene is an analogue of Cc, a Drosophila gene that is vital for normal development.

Prohibitin is a 30 -kD intracellular, antiproliferative protein. White et al. (1991) mapped the gene to chromosome 17 by analysis of human-mouse somatic cell hybrid cell lines using a genomic fragment of human Prohibitin DNA isolated from a library using the rat Prohibitin cDNA clone. By a study of cell lines containing portions of human chromosome 17, they determined that the PHB gene was located in the 17q11.2-q23 region. By in situ hybridization, they localized the gene to 17q21. Sato et al. (1992) isolated the human homolog of the rat Prohibitin gene and mapped it to 17qI2-q21 by in situ hybridization. Sato et al. (1993) showed that the human Prohibitin gene family consists of 1 functional PHB gene on 17q21 and 4 processed pseudogenes, each on a different chromosome: PHBP1 on 6q25, PHBP2 on 11p11.2, PHBP3 on 1p31.3, and PHBP4 on 2q2l.

By DNA sequence analysis of 2 exons in the PHB gene in 23 sporadic breast cancers that showed loss of heterozygosity on 17q or developed in patients 35 years old or younger, they identified 4 cases of somatic mutation: 2 of these were missense mutations, 1 showed a 2-bp deletion resulting in truncation of the gene product due to frameshift, and the fourth had a C-to-T transition in an intron adjacent to an intron-exon boundary. Sato et al. (1993) found no mutations in the PHB gene in other forms of tumors, namely, those of ovary, liver, and lung.

The disclosed NOV4 nucleic acid of the invention encoding a Prohibitin-like protein includes the nucleic acid whose sequence is provided in Table 4A or a fragment thereof. The invention also includes a mutant or variant nucleic acid any of whose bases may be changed from the corresponding base shown in Table 4A while still encoding a protein that maintains its Prohibitin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 10% percent of the bases may be so changed.

The disclosed NOV4 protein of the invention includes the Prohibitin-like protein whose sequence is provided in Table 4B. The invention also includes a mutant or variant protein any of whose residues may be changed from the corresponding residue shown in Table 4B while still encoding a protein that maintains its Prohibitin-like activities and physiological functions, or a functional fragment thereof. In the mutant or variant protein, up to about 43% percent of the residues may be so changed.

The protein similarity information, expression pattern, and map location for the Prohibitin-like protein and nucleic acid (NOV4) disclosed herein suggest that this NOV4 protein may have important structural and/or physiological functions characteristic of the Prohibitin family. Therefore, the NOV4 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV4 nucleic acids and proteins of the invention are useful in potential diagnostic and therapeutic applications implicated in various diseases and disorders described below. For example, the compositions of the present invention will have efficacy for treatment of patients suffering from breast cancer (In a sporadic breast cancer, Sato et al. (1992) found a missense mutation from valine (GTC) to alanine (GCC) at codon 88 of the PHB gene), and/or other pathologies. The NOV4 nucleic acids, or fragments thereof, may further be useful in diagnostic applications, wherein the presence or amount of the nucleic acid or the protein are to be assessed.

NOV4 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example, the disclosed NOV4 protein has multiple hydrophilic regions, each of which can be used as an immunogen. In one embodiment, a contemplated NOV4 epitope is from about amino acids 40 to 45. In another embodiment, a NOV4 epitope is from about amino acids 60 to 75. In additional embodiments, NOV4 epitopes are from about amino acids 100 to 130, from about amino acids 140 to 160,, from about amino acids 180 to 220, and from about amino acids 240 to 260. These novel proteins can be used in assay systems for functional analysis of various human disorders, which will help in understanding of pathology of the disease and development of new drug targets for various disorders.

NOV5

A disclosed NOV5 nucleic acid of 2011 nucleotides (also referred to as dj1182a14_da1) encoding a novel Macrophage Stimulating Protein Precursor-like protein is shown in Table 5A. An open reading frame was identified beginning with an ATG initiation codon at nucleotides 1-3 and ending with a TAG codon at nucleotides 1999-2001. A putative untranslated region downstream from the termination codon is underlined in Table 5A, and the start and stop codons are in bold letters.

TABLE 5A
NOV5 NUCLEOTIDE SEQUENCE

(SEQ ID NO:15)

ATGGGGTGGCTCCCACTCCTGCTGCTTCTGACTCAATGCTTAGGGGTCCC
TGGTCAGCGCTCGCCATTGAATGACTTCCAAGTGCTCCGGGGCACAGAGC
TACAGCACCTGCTACATGCGGTGGTGCCCGGGCCTTGGCAGGAGGATGTG
GCAGATGCTGAAGAGTGTGCTGGTCGCTGTGGGCCCTTAATGGACTGCCG
GGCCTTCCACTACAACGTGAGCAGCCATGGTTGCCAACTGCTGCCATGGA
CTCAACACTCGCCCCACACGAGGCTGCGGCGTTCTGGGCGCTGTGACCTC
TTCCAGAAGAAAGACTACGTACGGACCTGCATCATGAACAATGGGGTTGG
GTACCGGGGCACCATGGCCACGACCGTGGGTGGCCTGCCCTGCCAGGCTT
GGAGCCACAAGTTCCCAAATGATCACAAGTACACGCCCACTCTCCGGAAT
GGCCTGGAAGAGAACTTCTGCCGTAACCCTGATGGCGACCCCGGAGGTCC
CTGGTGCTACACAACAGACCCTGCTGTGCGCTTCCAGAGCTGCGGCATCA
AATCCTGCCGGGAGGCCGCGTGTGTCTGGTGCAATGGCGAGGAATACCGC
GGCGCGGTAGACCGCACGGAGTCAGGGCGCGAGTGCCAGCGCTGGGATCT
TCAGCACCCGCACCAGCACCCCTTCGAGCCGGGCAAGTTCCTCGACCAAG
GTCTGGACGACAACTATTGCCGGAATCCTGACGGCTCCGAGCGGCCATGG
TGCTACACTACGGATCCGCAGATCGAGCGAGAGTTCTGTGACCTCCCCCG
CTGCGGTTCCGAGGCACAGCCCCGCCAAGAGGCCACAACTGTCAGCTGCT
TCCGCGGGAAGGGTGAGGGCTACCGGGGCACAGCCAATACCACCACCGCG
GGCGTACCTTGCCAGCGTTGGGACGCGCAAATCCCGCATCAGCACCGATT
TACGCCAGAAAAATACGCGTGCAAGGACCTTCGGGAGAACTTCTGCCGGA
ACCCCGACGGCTCAGAGGCGCCCTGGTGCTTCACACTGCGGCCCGGCATG
CGCGCGGCCTTTTGCTACCAGATCCGGCGTTGTACAGACGACGTGCGGCC
CCAGACTGCTACCACGGCGCAGGGGAGCAGTACCGCGGCACGGTCAGCAA
GACCCGCAAGGGTGTCCAGTGCCAGCGCTGGTCCGCTGAGACGCCGCACA
AGCCGCAGTTCACGTTTACCTCCGAACCGCATGCACAACTGGAGGAGAAC
TTCTGCCGGAACCCAGATGGGGATAGCCATGGGCCCTGGTGCTACACGAT
GGACCCAAGGACCCCATTCGACTACTGTGCCCTGCGACGCTGCGCTGATG
ACCAGCCGCCATCAATCCTGGACCCCCCAGACCAGGTGCAGTTTGAGAAG
TGTGGCAAGAGGGTGGATCGGCTGGATCAGCGGCGTTCCAAGCTGCGCGT
GGTTGGGGGCCATCCGGGCAACTCACCCTGGACAGTCAGCTTGCGGAATC
GGTATGCTGCCTCTCACGGGCTATGAGGTATGGTTGGGCACCCTGTTCCA
GAACCCACAGCATGGAGAGCCAAGCCTACAGCGGGTCCCAGTAGCCAAGA
TGGTGTGTGGGCCCTCAGGCTCCCAGCTTGTCCTGCTCAAGCTGGAGAGA
TCTGTGACCCTGAACCAGCGTGTGGCCCTGATCTGCCTGCCCCCTGAATG
GTATGTGGTGCCTCCAGGGACCAAGTGTGAGATTGCAGGCTGGGGTGAGA
CCAAAGGTACGGGTAATGACACAGTCCTAAATGTGGCCTTGCTGAATGTC
ATCTCCAACCAGGAGTGTAACATCAAGCACCGAGGACGTGGTGACTACGG
GGGCCCACTTGCCTGCTTTACCCACAACTGCTGGGTCCTGGAAGGAATTA
TAATCCCCAACCGAGTATGCGCAAGGTCCTGCTGGCCAGCTGTCTTCACG
CGTGTCTCTGTGTTTGTGGACTGGATTCACAAGGTCATGAGACTGGGTTA
GGCCCAGCCTT

The NOV5 nucleic acid was identified on the q21 region of chromosome 3 and has 1508 of 1524 bases (98%) identical to a gb:GENBANK-ID:HUMMST1A|acc:L11924 mRNA from Homo sapiens (Homo sapiens macrophage-stimulating protein (MST1) mRNA, complete cds (E=0.0).

A disclosed NOV5 polypeptide (SEQ ID NO: 16) encoded by SEQ ID NO: 15 is 666 amino acid residues and is presented using the one-letter code in Table 5B. Signal P, Psort and/or Hydropathy results predict that NOV5 has a signal peptide and is likely to be localized in the lysosome (lumen) with a certainty of 0.5493. In other embodiments, NOV5 may also be localized to extracellularly with acertainty of 0.3700, the microbody (peroxisome) with a certainty of 0.1588, or the endoplasmic reticulum (membrane) with a certainty of 0.1000. The most likely cleavage site for a NOV5 peptide is between amino acids 18 and 19, at: VPG-QR.

TABLE 5B
ENCODED NOV5 PROTEIN SEQUENCE

(SEQ ID NO:16)

MGWLPLLLLLTQCLGVPGQRSPLNDFQVLRGTELQHLLHAVVPGPWQEDVADAEECAGRCGPLMDCRAFHYN
VSSHGCQLLPWTQHSPHTRLRRSGRCDLFQKKDYVRTCIMNNGVGYRGTMATTVGGLPCQAWSHKFPNDHKY
TPTLRNGLEENFCRNPDGDPGGPWCYTTDPAVRFQSCGIKSCREAACVWCNGEEYRGAVDRTESGRECQRWD
LQHPHQHPFEPGKFLDQGLDDNYCRNPDGSERPWCYTTDPQIEREFCDLPRCGSEAQPRQEATTVSCFRGKG
EGYRGTANTTTAGVPCQRWDAQIPHQHRFTPEKYACKDLRENFCRNPDGSEAPWCFTLRPGMRAAFCYQIRR
CTDDVRPQTATTAQGSSTAARSARPARVSSASAGPLRRRTSRSSRLPPNRMHNWRRTSAGTQMGIAMGPGAT
RWTQGPHSTTVPCDAALMTSRHQSWTPQTRCSLRSVARGWIGWISGVPSCAWLGAIRATHPGQSACGIGMLP
LTGYEVWLGTLFQNPQHGEPSLQRVPVAKMVCGPSGSQLVLLKLERSVTLNQRVALICLPPEWYVVPPGTKC
EIAGWGETKGTGNDTVLNVALLNVISNQECNIKHRGRGDYGGPLACFTHNCWVLEGIIIPNRVCARSCWPAV
FTRVSVFVDWIHKVMRLG

The disclosed NOV5 amino acid sequence has 368 of 368 amino acid residues (100%) identical to, and 368 of 368 amino acid residues (100%) similar to, the 711 amino acid residue ptnr:SPTREMBL-ACC:Q14870 protein from Homo sapiens (Human) (Macrophage-Stimulating Protein Precursor (E=9.9e⁻³¹⁰).

NOV5 is expressed in at least HepG2 (liver) In addition, the sequence is predicted to be expressed in the following tissues because of the expression pattern of (GENBANK-ID: HUMMST1A|acc:L11924) a closely related {Homo sapiens macrophage-stimulating protein (MST1) mRNA, complete cds homolog in species Homo sapiens: HepG2, and liver.

TaqMan data for NOV5 can be found below in Example 2. NOV5 also has homology to the amino acid sequences shown in the BLASTP data listed in Table 5C.

TABLE 5C
BLAST results for NOV5

| Gene Index/ | | Length | Identity | Positives | |
|---|---|---|---|---|---|
| Identifier | Protein/Organism | (aa) | (%) | (%) | Expect |
| gi\|15294659\|ref\|XP_{—} | macrophage | 711 | 561/720 | 577/720 | 0.0 |
| 054070.1\| | stimulating 1 | | (77%) | (79%) | |
| | (hepatocyte | | | | |
| | growth factor- | | | | |
| | like) [Homo | | | | |
| | sapiens] | | | | |
| gi\|10337615\|ref\|NP_{—} | macrophage | 711 | 560/720 | 576/720 | 0.0 |
| 066278.1\| | stimulating 1 | | (77%) | (79%) | |
| | (hepatocyte | | | | |
| | growth factor- | | | | |
| | like) [Homo | | | | |
| | sapiens] | | | | |
| gi\|123114\|sp\|P26927\| | HEPATOCYTE GROWTH | 711 | 560/720 | 576/720 | 0.0 |
| HGFL_HUMAN | FACTOR-LIKE | | (77%) | (79%) | |
| | PROTEIN PRECURSOR | | | | |
| | (MACROPHAGE | | | | |
| | STIMULATORY | | | | |
| | PROTEIN) (MSP) | | | | |
| | (MACROPHAGE | | | | |
| | STIMULATING | | | | |
| | PROTEIN) | | | | |
| gi\|15299258\|ref\|XP_{—} | hypothetical | 529 | 440/532 | 456/532 | 0.0 |
| 017811.2\| | protein XP_017811 | | (82%) | (85%) | |
| | [Homo sapiens] | | | | |
| gi\|90615\|pir\|\|A40332 | macrophage- | 716 | 449/725 | 507/725 | 0.0 |
| | stimulating | | (61%) | (69%) | |
| | protein 1 | | | | |
| | precursor - mouse | | | | |

The homology of these sequences is shown graphically in the ClustalW analysis shown in Table 5D.

[image]

[image]

[image]

Tables 5E-N list the domain description from DOMAIN analysis results against NOV5. This indicates that the NOV5 sequence has properties similar to those of other proteins known to contain this domain.

TABLE 5E

Domain Analysis of NOV5

gnl|Pfam|pfam00051, kringle, Kringle domain. Kringle domains have been
found in plasminogen, hepatocyte growth factors, prothrombin, and
apolipoprotein A. Structure is disulfide-rich, nearly all-beta. (SEQ ID NO:85)
CD-Length = 79 residues, 100.0% aligned
Score = 117 bits (292), Expect = 3e−27

| | | | |
|---|---|---|---|
| Query: | 191 | CVWCNGEEYRGAVDRTESGRECQRWDLQHPHQHPF-EPGKFLDQGLDDNYCRNPDGSERP | 249 |
| | | \| \|\|\| \|\|\| \|\|\|\| \|\|\|\|\| \| \|\|+\| \| ++ +\|\| +\|\|\|\|\|\|\|\| \|\|\| | |
| Sbjct: | 1 | CYHGNGENYRGTASTTESGAPCQRWDSQTPHRHSKYTPERYPAKGLGENYCRNPDGDERP | 60 |
| Query: | 250 | WCYTTDPQIEREFCDLPRC 268 | |
| | | \|\|\|\|\|\|\|++ \|+\|\|+\|\|\| | |
| Sbjct: | 61 | WCYTTDPRVRWEYCDIPRC 79 | |

TABLE 5F

Domain Analysis of NOV5

gnl|Pfam|pfam00051, kringle, Kringle domain. Kringle domains have been
found in plasminogen, hepatocyte growth factors, prothrombin, and
apolipoprotein A. Structure is disulfide-rich, nearly all-beta. (SEQ ID NO:85)
CD-Length = 79 residues, 100.0% aligned
Score = 112 bits (279), Expect = 9e−26

| | | | |
|---|---|---|---|
| Query: | 283 | CFRGKGEGYRGTANTTTAGVPCQRWDAQIPHQHRF-TPEKYACKDLRENFCRNPDGSEAP | 341 |
| | | \|+ \| \|\| \|\|\|\|\|+\|\| +\| \|\|\|\|\|\|+\| \|\|+\| \|\|\|+\| \| \| \|\|+\|\|\|\|\|\| \| \| | |
| Sbjct: | 1 | CYHGNGENYRGTASTTESGAPCQRWDSQTPHRHSKYTPERYPAKGLGENYCRNPDGDERP | 60 |
| Query: | 342 | WCFTLRPGMRAAFCYQIRRC 361 | |
| | | \|\|+\| \| +\| +\| \| \|\| | |
| Sbjct: | 61 | WCYTTDPRVRWEYC-DIPRC 79 | |

TABLE 5G

Domain Analysis of NOV5

gnl|Pfam|pfam00051, kringle, Kringle domain. Kringle domains have been
found in plasminogen, hepatocyte growth factors, prothrombin, and
apolipoprotein A. Structure is disulfide-rich, nearly all-beta. (SEQ ID NO:85)
CD-Length = 79 residues, 100.0% aligned
Score = 104 bits (259), Expect = 2e−23

| | | | |
|---|---|---|---|
| Query: | 110 | CIMNNGVGYRGTMATTVGGLPCQAWSHKFPNDH-KYTPT--LRNGLEENFCRNPDGDPGG | 166 |
| | | \| \|\| \|\|\|\| +\|\| \| \|\|\| \| 10 + \|+ \| \|\|\|\| \|\| \|\|+\|\|\|\|\|\|\| | |
| Sbjct: | 1 | CYHGNGENYRGTASTTESGAPCQRWDSQTPHRHSKYTPERYPAKGLGENYCRNPDGDE-R | 59 |
| Query: | 167 | PWCYTTDPAVRFQSCGIKSC 186 | |
| | | \|\|\|\|\|\|\|\| \|\|++ \| \| \| | |
| Sbjct: | 60 | PWCYTTDPRVRWEYCDIPRC 79 | |

TABLE 5H

Domain Analysis of NOV5

gnl|Smart|smart00130, KR, Kringle domain; Named after a Danish pastry.
Found in several serine proteases and in ROR-like receptors. Can occur
in up to 38 copies (in apolipoprotein(a)). Plasminogen-like kringles
possess affinity for free lysine and lysine-containing peptides.
(SEQ ID NO:86)
CD-Length = 83 residues, 97.6% aligned
Score = 111 bits (278), Expect = 1e−25

| | | | |
|---|---|---|---|
| Query: | 191 | CVWCNGEEYRGAVDRTESGRECQRWDLQHPHQHPFEPGKFLDQGLDDNYCRNPDG-SERP | 249 |
| | | \| \|\|\| \|\|\| \|+\|\|+ \|\|\|\|\| \| \|\| \| \| \| +\| + \|\|+ \|\|\|\|\|\|\|\| \|\| \| | |
| Sbjct: | 3 | CYAGNGESYRGTASTTKSGKPCQRWDSQTPHLHRFTPERFPELGLEHNYCRNPDGDSEGP | 62 |
| Query: | 250 | WCYTTDPQIEREFCDLPRCGS 270 | |
| | | \|\|\|\|\|\|\| + \|+\|\|+\|+\| \| | |
| Sbjct: | 63 | WCYTTDPNVRWEYCDIPQCES 83 | |

TABLE 5I
Domain Analysis of NOV5

gnl|Smart|smart00130, KR, Kringle domain; Named after a Danish pastry.
Found in several serine proteases and in ROR-like receptors. Can occur
in up to 38 copies (in apolipoprotein(a)). Plasminogen-like kringles
possess affinity for free lysine and lysine-containing peptides.
(SEQ ID NO:86)
CD-Length =83 residues, 97.6% aligned
Score = 106 bits (265), Expect = 4e−24

| | | | |
|---|---|---|---|
| Query: | 108 | RTCIMNNGVGYRGTMATTVGGLPCQAWSHKFPNDHKYTPTLRN--GLEENFCRNPDGDPG | 165 |
| | | \| \| \|\| \|\|\|\| +\|\| \| \|\|\| \| + \|+ \|++\|\| \|\|\| \|+\|\|\|\|\|\|\| | |
| Sbjct: | 1 | RDCYAGNGESYRGTASTTKSGKPCQRWDSQTPHLHRFTPERFPELGLEHNYCRNPDGDSE | 60 |
| Query: | 166 | GPWCYTTDPAVRFQSCGIKSC 186 | |
| | | \|\|\|\|\|\|\|\|\| \|\|++ \| \| \| | |
| Sbjct: | 61 | GPWCYTTDPNVRWEYCDIPQC 81 | |

TABLE 5J

Domain Analysis of NOV5

gnl|Smart|smart00130, KR, Kringle domain; Named after a Danish pastry.
Found in several serine proteases and in ROR-like receptors. Can occur
in up to 38 copies (in apolipoprotein(a)). Plasminogen-like kringles
possess affinity for free lysine and lysine-containing peptides.
(SEQ ID NO:86)
CD-Length = 83 residues, 97.5% aligned
Score = 104 bits (260), Expect = 1e−23

| | | | |
|---|---|---|---|
| Query: | 283 | CFRGKGEGYRGTANTTTAGVPCQRWDAQIPHQHRFTPEKYACKDLRENFCRNPDG-SEAP | 341 |
| | | \|+ \| \|\| \|\|\|\|\|+\|\| +\| \|\|\|\|\|\|+\| \|\| \|\|\|\|\|\|++ \| \|+\|\|\|\|\|\| \|\| \| | |
| Sbjct: | 3 | CYAGNGESYRGTASTTKSGKPCQRWDSQTPHLHRFTPERFPELGLEHNYCRNPDGDSEGP | 62 |
| Query: | 342 | WCFTLRPGMRAAFCYQIRRCTD 363 | |
| | | \|\|+\| \| +\| +\| \| +\| | |
| Sbjct: | 63 | WCYTTDPNVRWEYCD-IPQCES 83 | |

TABLE 5K

Domain Analysis of NOV5

gnl|Smart|smart00020, Tryp_SPc, Trypsin-like serine protease; Many of
these are synthesised as inactive precursor zymogens that are cleaved
during limited proteolysis to generate their active forms. A few,
however, are active as single chain molecules, and others are inactive
due to substitutions of the catalytic triad residues. (SEQ ID NO:87)
CD-Length = 230 residues, 79.1% aligned
Score = 110 bits (274), Expect = 3e−25

| | | | |
|---|---|---|---|
| Query: | 504 | PLTGYEVWLGTLFQNPQHGEPSLQRVPVAKMVCGPSGSQ------LVLLKLERSVTLNQR | 557 |
| | | + \| \|\|+ + \| \| \|+\|++ \|+ + + \|\|\|\| \|\|\|+ | |
| Sbjct: | 49 | APSSIRVRLGSHDLSSGEET---QTVKVSKVIVHPNYNPSTYDNDIALLKLSEPVTLSDT | 105 |
| Query: | 558 | VALICLPPEWYVVPPGTKCEIAGWGETKGTG--NDTVLNVALLNVISNQECNIKHRGR-- | 613 |
| | | \| \|\|\|\| \| \|\| \|\| \| ++\|\|\| \| + \| + ++\|\| \| + \| | |
| Sbjct: | 106 | VRPICLPSSGYNVPAGTTCTVSGWGRTSESSGSLPDTLQEVNVPIVSNATCRRAYSGGPA | 165 |
| Query: | 614 | -------------------GDYGGPLACFTHNCWVLEGIIIPNRV-CARSCWPAVFTRVS | 653 |
| | | \|\| \|\|\|\| \| \|\|\| \|\|+ \|\|\| \| \|+\|\|\|\| | |
| Sbjct: | 166 | ITDNMLCAGGLEGGKDACQGDSGGPLVC-NDPRWVLVGIVSWGSYGCARPNKPGVYTRVS | 224 |
| Query: | 654 | VFVDWI 659 | |
| | | ++\|\|\| | |
| Sbjct: | 225 | SYLDWI 230 | |

TABLE 5L

Domain Analysis of NOV5

gnl|Pfam|pfam00089, trypsin, Trypsin. Proteins recognized include all
proteins in families S1, S2A, S2B, S2C, and S5 in the classification
of peptidases. Also included are proteins that are clearly members,
but that lack peptidase activity, such as haptoglobin and protein Z
(PRTZ^{*}). (SEQ ID NO:88)
CD-Length =217 residues, 77.9% aligned
Score = 92.0 bits (227), Expect = 9e—20

| | | | |
|---|---|---|---|
| Query: | 508 | YEVWLGTLFQNPQHGEPSLQRVPVAKMV----CGPSGSQLVLLKLERSVTLNQRVALICL | 563 |
| | | \| \|\| \| \| + \|+ \| \|++ \| + + \|\|\|\|+ \|\|\| \| \|\|\| | |
| Sbjct: | 49 | VRVVLGE--HNLGTTEGTEQKFDVKKIIVHPNYNPDTNDIALLKLKSPVTLGDTVRPICL | 106 |
| Query: | 564 | PPEWYVVPPGTKCEIAGWGETKGTGNDTVLNVALLNVISNQECNIKHRG----------- | 612 |
| | | \| +\| \|\| \| ++\|\|\| \|\| \| \| ++ ++\| + \| + \| | |
| Sbjct: | 107 | PSASSDLPVGTTCSVSGWGRTKNLGTSDTLQEVVVPIVSRETCRSAYGGTVTDTMICAGA | 166 |
| Query: | 613 | -------RGDYGGPLACFTHNCWVLEGIIIPNRVCARSCWPAVFTRVSVFVDWI 659 | |
| | | +\|\| \|\|\|\| \| \| \|\|+ \|\| +\| \|+\|\|\|\| ++\|\|\| | |
| Sbjct: | 167 | LGGKDACQGDSGGPLVCSDG---ELVGIVSWGYGCAVGNYPGVYTRVSRYLDWI 217 | |

TABLE 5M

Domain Analysis of NOV5

gnl|Smart|smart00473, PAN_AP, divergent subfamily of APPLE domains;
Apple-like domains present in Plasminogen, C. elegans hypothetical
ORFs and the extracellular portion of plant receptor-like protein
kinases. Predicted to possess protein- and/or carbohydrate-binding
functions. (SEQ ID NO:89)
CD-Length = 79 residues, 94.9% aligned
Score = 52.0 bits (123), Expect = 1e−07

| | | | |
|---|---|---|---|
| Query: | 25 | DFQVLRGTELQHLLHAVVPGPWQEDVADAEECAGRC-GPLMDCRAFHYNVSSHGCQLLPW | 83 |
| | | \| \| \|+\| \| \|\| \|\|\|\| +\| \|\|+\| \|\| + \|\| \| | |
| Sbjct: | 5 | CFVRLPNTKL------PDFSPIVISVASLEECAQKCLNSNCSCRSFTYNNDTKGCLLWSE | 58 |
| Query: | 84 | TQHSPHTRLRRSGRCDLFQKK 104 | |
| | | + +\| \|\| \| ++\| | |
| Sbjct: | 59 | SSLGDARQLLPSGGVDYYEKI 79 | |

TABLE 5N
Domain Analysis of NOV5

gnl|Pfam|pfama0024, PAN, PAN domain. The PAN domain contains a
conserved core of three disulphide bridges. In some members of the
family there is an additional fourth disulphide bridge the links the N
and C termini of the domain. The domain is found in diverse proteins,
in some they mediate protein-protein interactions, in others they
mediate protein-carbohydrate interactions. (SEQ ID NO:159)
CD-Length=78 residues, 76.9% aligned
Score=50.1 bits (118), Expect=4e-07

| | | | |
|---|---|---|---|
| Query: | 45 | PWQEDVADAEECAGRCGPLMD-CRAFEYNVSSEGCQLLPWTQESPHTRLRRSGRCDLFQK | 103 |
| | | +\|\|\|\|\|\|\|\|+\|\|\|\|\|\|+\|\|\|+\|++\| | |
| Sbjct: | 17 | ISVVNVPSLEECAALCLEEPRVCRSFTYNNKSKQCLLKSESSGSLPRLKARPSQKVDYYEK | |

Macrophage-stimulating protein (MSP) is an 80 -kD serum protein with homology to hepatocyte growth factor (HGF)(Sakamoto O, et.al.; J Clin Invest 1997 Feb 15;99(4):701-9). Its receptor, RON tyrosine kinase, is a new member of the HGF receptor family. The MSP-RON signaling pathway has been implicated in the functional regulation of mononuclear phagocytes. However, the function of this pathway in other types of cells has not been elucidated. Here we show that in contrast to the HGF receptor, which was expressed at the basolateral surface, RON was localized at the apical surface of ciliated epithelia in the airways and oviduct. In addition, MSP was found in the bronchoalveolar space at biologically significant concentrations. MSP bound to RON on normal human bronchial epithelial cells with a high affinity (Kd=0.5 nM) and induced autophosphorylation of RON. Activation of RON by MSP led to a significant increase in ciliary beat frequency of human nasal cilia. These findings indicate that the ciliated epithelium of the mucociliary transport apparatus is a novel target of MSP. Ciliary motility is critical for mucociliary transport. Our findings suggest that the MSP-RON signaling pathway is a novel regulatory system of mucociliary function and might be involved in the host defense and fertilization.

Macrophage stimulating protein (MSP; 142408), also known as hepatocyte growth factor-like protein (HGFL), is structurally related to hepatocyte growth factor. Gaudino et al. (EMBO J. 13: 3524-3532, 1994) showed that the RON gene is expressed at the cell surface of several epithelial cell types in addition to granulocytes and monocytes. The RON mRNA is translated into a glycosylated precursor that is cleaved into a 185 -kD heterodimer of 35 -kD (alpha) and 150 -kD (beta) subunits joined by the predicted disulfide linkage. Gaudino et al. (1994) further demonstrated that the beta chain undergoes tyrosine phosphorylation upon stimulation by MSP. By isotopic in situ hybridization, Ronsin et al. (Oncogene 8: 1195-1202, 1993) mapped the RON gene to 3p21, with the most probable location being 3p21.3. The gene encoding MSP is also located on 3p21, a region of frequent deletion or mutation in small cell lung and renal carcinoma.

The genes encoding hepatocyte growth factor (HGF; 142409) and its MET receptor are located on 7q. The location of ligand and receptor on the same chromosome, and the structural similarities between MSP and HGF suggested that the ligand for RON might be MSP. Wang et al. (Science 266: 117-119, 1994) showed that this is indeed the case. Their experiments established that the RON gene product is a specific cell surface receptor for MSP. A human hepatoma (HepG2) cell line library was screened with an oligonucleotide probe for macrophage stimulating protein (MSP) to clone an MSP cDNA(Yoshimura T, et.al.; J Biol Chem 1993 Jul 25;268(21):15461-8). Deduced sequences of isolated clones were compared with peptide fragment sequences of MSP. MSP9 cDNA encoded most of the known sequence of MSP except for a small segment of the 5′ end of the open reading frame. Consequently, a hybrid 2300-base pair cDNA that encoded the complete MSP amino acid sequence was constructed from 2 clones. Culture fluid from COS-7 cells transfected with this full-length MSP cDNA had MSP biological activity, and the expressed MSP was detected by immunoprecipitation with antibody against native MSP. The deduced amino acid sequence of MSP includes 4 kringle domains, which have been found in hepatocyte growth factor and several proteins of the blood coagulation system. Among them, MSP has the highest sequence similarity to hepatocyte growth factor (45% identity). The MSP cDNA hybridized strongly to mRNA from liver, and to a lesser extent to mRNA from kidney and pancreas, suggesting that a cell type in the liver is the source of MSP. Several cloned and sequenced MSP cDNAs had insertions or deletions, suggesting that alternatively spliced MSP mRNAs may occur. This was reflected in Northern blots probed with an MSP cDNA, which showed more than one mRNA species. Furthermore, although the gene coding for MSP is on chromosome 3, the sequence of one of the cDNAs was identical with a unique sequence in chromosome 1, indicating that there may be a family of MSP genes, located on chromosomes 3 and 1.

Although the hepatocyte growth factor-like protein (HLP) shares a 50% homology with the hepatocyte growth factor, the biological function of HLP has remained unknown (Shimamoto A, et.al.; FEBS Lett 1993 Oct 25;333(1-2):61-6). Addition of conditioned medium of COS-7 cells transfected with the expression plasmid for HLP cDNA to cultures of resident peritoneal macrophages induced specific activation of macrophages, and the factor which stimulates macrophages was purified from the conditioned medium. The purified protein showed M(r) of 85 kDa on SDS-PAGE, and this M(r) is in agreement with that of macrophage-stimulating protein (MSP) previously purified from human serum, as well as with the predicted M(r) of HLP. Amino acid composition of the purified protein coincided with the compositions of human HLP and MSP. Together with the finding that the partial amino acid sequences of MSP are highly homologous to that of HLP, we conclude that the biological function of HLP is to activate macrophages and that HLP and MSP are identical molecules.

The disclosed NOV5 nucleic acid of the invention encoding a Macrophage Stimulating Protein Precursor-like protein includes the nucleic acid whose sequence is provided in Table 5A or a fragment thereof. The invention also includes a mutant or variant nucleic acid any of whose bases may be changed from the corresponding base shown in Table 5A while still encoding a protein that maintains its Macrophage Stimulating Protein Precursor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 10% percent of the bases may be so changed.

The disclosed NOV5 protein of the invention includes the Macrophage Stimulating Protein Precursor-like protein whose sequence is provided in Table 5B. The invention also includes a mutant or variant protein any of whose residues may be changed from the corresponding residue shown in Table 5B while still encoding a protein that maintains its Macrophage Stimulating Protein Precursor-like activities and physiological functions, or a functional fragment thereof. In the mutant or variant protein, up to about 39% percent of the residues may be so changed.

The NOV5 nucleic acids and proteins of the invention are useful in potential therapeutic applications implicated in Aicardi-Goutieres syndrome 1; Brugada syndrome; Deafness, autosomal recessive 6; Heart block, nonprogressive; Heart block, progressive, 2; Ichthyosiforme erythroderma, congenital, nonbullous; Long QT syndrome-3; Night blindness; congenital stationary; Pituitary ACTH-secreting adenoma; Small-cell cancer of lung; Ventricular fibrillation, idiopathic; entricular tachycardia, idiopathic; HIV infection, susceptibility/resistance to; Von Hippel-Lindau (VHL) syndrome; Cirrhosis; Transplantation as well as other diseases, disorders and conditions. The NOV5 nucleic acid, or fragments thereof, may further be useful in diagnostic applications, wherein the presence or amount of the nucleic acid or the protein are to be assessed.

NOV5 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV5 protein have multiple hydrophilic regions, each of which can be used as an immunogen. In one embodiment, contemplated NOV5 epitope is from about amino acids 20 to 80. In other embodiments, NOV5 epitope is from about amino acids 90 to 120, from about amino acids 140 to 180, from about amino acids 190 to 340, from about amino acids 350 to 460, from about amino acids 500 to 530, from about amino acids 570 to 590, and from about amino acids 600 to 620, or from about amino acids 420 to 460. This novel protein also has value in development of powerful assay system for functional analysis of various human disorders, which will help in understanding of pathology of the disease and development of new drug targets for various disorders.

NOV6

A disclosed NOV6 nucleic acid of 634 nucleotides (also referred to as GM382a20_A) encoding a novel Fatty Acid-Binding Protein-like protein is shown in Table 6A. An open reading frame was identified beginning with an ATG initiation codon at nucleotides 58-60 and ending with a TAA codon at nucleotides 460-462. A putative untranslated region upstream from the initiation codon and downstream from the termination codon is underlined in Table 6A, and the start and stop codons are in bold letters.

TABLE 6A
NOV6 Nucleotide Sequence

(SEQ ID NO:17)

CAAGCTGCCCACGCCGACGGCAACCCTGCTCTGCATGCCCGCCCGCCCGTGCCCACCATGGCCACAGTTC
AGCAGCTGGGAGGAAGATGGCGCCTGGTGGACAGCAAACGCTTTGATGAATACATGAAGGAGGGAGGAGT
GGGAACTGCTTTGCGAAAAATGGACGCAATGGCCAAGCCAGATTGTATCATCACTTGTGATGGCAAAAAC
CTCACCATAAAAACCGAGAGCACTTTGAAAACACAGTTTTCTTGTACCCTGGGAGAGAAGTTTGAAGAAA
CCACAGCTGATGGCAGAAAAACTCAGACTGTGTGCAGCTTTGCAGATGGTGCATTGGTTCAGCATCAGGA
GTGGGATGGGAAGGAAAACACAATAACAAGAAAACTGAAAGATGGGAAATTAGTGGTGTACTGTGTCATG
AACAATGTCGCCTGTACTCGGATCTATGAAAAAGTAGAATAAAAATTCCATCATCACTTTGGACAGGAGT
TAACTAATAGAATGATCAAGCTCAGTTCAATGAGCAAATCTCCATAGTGTTTTTTTTCATTACTGTGTTC
AATTATCTTTATCACAAACGTTTCACATGCAGCTATTTCAAAGTGTCTTGGATTAATTAGGATCATCCCT
TTGG

The disclosed NOV6 nucleic acid sequence, located on chromosome 15, has 537 of 589 bases (91%) identical to a Fatty Acid-Binding Protein mRNA from Homo sapiens (GENBANK-ID: HUMFABPHA) (E=3.7e⁻¹⁰²).

A disclosed NOV6 polypeptide (SEQ ID NO:18) encoded by SEQ ID NO:17 is 134 amino acid residues and is presented using the one-letter amino acid code in Table 6B. Signal P, Psort and/or Hydropathy results predict that NOV6 contains no signal peptide and is likely to be localized in the cytoplasm with a certainty of 0.4500. In other embodiments, NOV6 is also likely to be localized to the mitochondrial matrix space with a certainty of 0.3600, or to the lysosome (lumen) with a certainty of 0.1000.

TABLE 6B
Encoded NOV6 protein sequence.

(SEQ ID NO:18)

MATVQQLGGRWRLVDSKRFDEYMKEGGVGTALRKMDAMAKPDCIITCDGKNLTIKTESTLKTQFSCTLGEKFEET
TADGRKTQTVCSFADGALVQHQEWDGKENTITRKLKDGKLWYCVMNNVACTRIYEKVE

The disclosed NOV6 amino acid sequence has 124 of 135 amino acid residues (91%) identical to, and 126 of 135 amino acid residues (93%) similar to, the 135 amino acid residue Fatty Acid-Binding protein from Homo sapiens (Q01469) (E=2.1e⁻⁶¹).

NOV6 is expressed in Sensory System.Skin; Nervous System.Brain; Male Reproductive System.Testis; Respiratory System.Lung, Larynx; Female Reproductive System; .Placenta; Whole Organism; Cardiovascular System.Heart; Endocrine System.Parathyroid Gland; Hematopoietic and Lymphatic System, Hematopoietic Tissues, Liver, Tonsils; Gastro-intestinal/Digestive System.Large Intestine, Colon, Stomach, Oesophagus; Urinary System.Kidney. In addition, the sequence is predicted to be expressed in the following tissues because of the expression pattern of (GENBANK-ID: ACC:Q05816) a closely related Fatty Acid-Binding Protein homolog in species Mus musculus}: Sensory System.Skin; Nervous System.Brain; Male Reproductive System.Testis; Respiratory System.Lung, Larynx; Female Reproductive System; Placenta; Whole Organism; Cardiovascular System.Heart; Endocrine System.Parathyroid Gland; Hematopoietic and Lymphatic System, Hematopoietic Tissues, Liver, Tonsils; Gastro-intestinal/Digestive System.Large Intestine, Colon, Stomach, Oesophagus; Urinary System.Kidney.

TaqMan data for NOV6 can be found below in Example 2. NOV6 also has homology to the amino acid sequences shown in the BLASTP data listed in Table 6C.

TABLE 6C
BLAST results for NOV6

| Gene Index/ | | Length | Identity | Positives | |
|---|---|---|---|---|---|
| Identifier | Protein/Organism | (aa) | (%) | (%) | Expect |
| gi\|13651468\|ref\|XP_{—} | similar to | 135 | 122/135 | 126/135 | 1e−58 |
| 016351.1\| | GASTRIN/CHOLECYST | | (90%) | (92%) | |
| | OKININ TYPE B | | | | |
| | RECEPTOR (CCK-B | | | | |
| | RECEPTOR) (CCK- | | | | |
| | BR) (H. sapiens) | | | | |
| | [Homo sapiens] | | | | |
| gi\|4557581\|ref\|NP_{—} | fatty acid | 135 | 124/135 | 126/135 | 1e−58 |
| 001435.1\| | binding protein 5 | | (91%) | (92%) | |
| | (psoriasis- | | | | |
| | associated); E- | | | | |
| | FABP [Homo | | | | |
| | sapiens] | | | | |
| gi\|13651882\|ref\|XP_{—} | fatty acid | 135 | 119/135 | 124/135 | 6e−57 |
| 011655.5\| | binding protein 5 | | (88%) | (91%) | |
| | (psoriasis- | | | | |
| | associated) [Homo | | | | |
| | sapiens] | | | | |
| gi\|13651563\|ref\|XP_{—} | similar to | 135 | 118/135 | 125/135 | 5e−56 |
| 015760.1\| | GASTRIN/CHOLECYST | | (87%) | (92%) | |
| | OKININ TYPE B | | | | |
| | RECEPTOR (CCK-B | | | | |
| | RECEPTOR) (CCK- | | | | |
| | BR) (H. sapiens) | | | | |
| | [Homo sapiens] | | | | |
| gi\|6648071\|sp\|P55052\| | FATTY ACID- | 135 | 117/135 | 124/135 | 1e−55 |
| FABE_BOVIN | BINDING PROTEIN, | | (86%) | (91%) | |
| | EPIDERMAL (E- | | | | |
| | FABP) | | | | |

The homology of these sequences is shown graphically in the ClustalW analysis shown in Table 6D.

[image]

[image]

Table 6E lists the domain description from DOMAIN analysis results against NOV6. This indicates that the NOV6 sequence has properties similar to those of other proteins known to contain this domain.

TABLE 6E
Domain Analysis of NOV6

gnl|Pfam|pfam00061, lipocalin, Lipocalin/cytosolic fatty-acid
binding protein family. Lipocalins are transporters for small
hydrophobic molecules, such as lipids, steroid hormones, bilins, and
retinoids. Alignment subsumes both the lipocalin and fatty acid
binding protein signatures from PROSITE. This is supported on
structural and functional grounds. Structure is an eight-stranded beta
barrel. (SEQ ID NO:90)
CD-Length=145 residues, 76.6% aligned
Score=53.1 bits (126), Expect=1e-08

| | | | |
|---|---|---|---|
| Query: | 9 | GRWRLVDSKRFDEYMK-EGGVGTALRKNDAMAK-PDCIITCDGKNLTIKTESTLKTQFSC | 66 |
| \|+\|\|\|\|\|+\|\|\|\|\|\|\|+\|\|\|\|\|+\|++ | | | |
| Sbjct: | 4 | GRWYLVASANFDPELKEELGVLEATRKEITPLKEGNLEIVFDGDKNGICEETFGKLEKTK | 63 |
| Query: | 67 | TLGEKFEETTADGRKTQTVCSFADCALVQRQEWDGKENIITRXLRDGKLVV 117 | |
| \|\|+\|+\|\|\|++\|\|\|+\|\|\|+\|+\|+ | | | |
| Sbjct: | 64 | KLGVEFDYYTGDNRFVVLDTDYDNYLLVCVQRGDGNETSRTAELYGRTPEL 114 | |

Fatty acid metabolism in mammalian cells depends on a flux of fatty acids, between the plasma membrane and mitochondria or peroxisomes for beta-oxidation, and between other cellular organelles for lipid synthesis. The fatty acid-binding protein (FABP) family consists of small, cytosolic proteins believed to be involved in the uptake, transport, and solubilization of their hydrophobic ligands. Members of this family have highly conserved sequences and tertiary structures. Fatty acid-binding proteins were first isolated in the intestine (FABP2; OMIM-134640) and later found in liver (FABPl; OMIM-134650), striated muscle (FABP3; OMIM-134651), adipocytes (FABP4; OMIM-600434) and epidermal tissues (E-FABP; GDB ID:136450).

Epidermal fatty acid binding protein (E-FABP) was cloned by as a novel keratinocyte protein by Madsen et al (1992, PMID: 1512466) from skin of psoriasis patients. Later using quantitative Western blot analysis, Kingma et al. (1998, PMID: 9521644) have shown that in addition to the skin, bovine E-FABP is expressed in retina, testis, and lens. Since E-FABP was originally identified from the skin of psoriasis patients, it is also known as psoriasis-associated fatty acid-binding protein (PA-FABP). PA-FABP is a cytoplasmic protein, and is expressed in keratinocytes. It is highly up-regulated in psoriatic skin. It shares similarity to other members of the fatty acid-binding proteins and belongs to the fabp/p2/crbp/crabp family of transporter. PA-FABP is believed to have a high specificity for fatty acids, with highest affinity for c18 chain length. Decreasing the chain length or introducing double bonds reduces the affinity. PA-FABP may be involved in keratinocyte differentiation.

Immunohistochemical localization of the expression of E-FABP in psoriasis, basal and squamous cell carcinomas has been carried out in order to obtain indirect information, at the cellular level, on the transport of the fatty acidss. (Masouye et al, 1996, PMID: 8726632). E-FABP was localized in the upper stratum spinosum and stratum granulosum in normal and non-lesional psoriatic skin. In contrast, lesional psoriatic epidermis strongly expressed E-FABP in all suprabasal layers, like nonkeratinized oral mucosa. The basal layer did not express E-FABP reactivity in any of these samples. Accordingly, basal cell carcinomas were E-FABP negative whereas only well-differentiated cells of squamous cell carcinomas expressed E-FABP. This suggests that E-FABP expression is related to the commitment of keratinocyte differentiation and that the putative role of E-FABP should not be restricted to the formation of the skin lipid barrier. Since the pattern of E-FABP expression mimics cellular FA transport, our results suggest that lesional psoriatic skin and oral mucosa have a higher metabolism/transport for FAs than normal and non-lesional psoriatic epidermis.

The disclosed NOV6 nucleic acid of the invention encoding a Fatty acid binding protein-like protein includes the nucleic acid whose sequence is provided in Table 6A or a fragment thereof. The invention also includes a mutant or variant nucleic acid any of whose bases may be changed from the corresponding base shown in Table 6A while still encoding a protein that maintains its Fatty acid binding protein-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 10% percent of the bases may be so changed.

The disclosed NOV6 protein of the invention includes the Fatty acid binding protein-like protein whose sequence is provided in Table 6B. The invention also includes a mutant or variant protein any of whose residues may be changed from the corresponding residue shown in Table 6B while still encoding a protein that maintains its Fatty acid binding protein-like activities and physiological functions, or a functional fragment thereof. In the mutant or variant protein, up to about 14% percent of the residues may be so changed.

The above defined information for this invention suggests that these Fatty acid binding protein-like proteins (NOV6) may function as a member of a “Fatty acid binding proteinfamily”. Therefore, the NOV6 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of NOV6 are useful in Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderrna, Transplantation, Endometriosis, Inflammatory bowel disease, Diverticular disease, Hirschsprung's disease, Crohn's Disease, Hemophilia, hypercoagulation, Idiopathic thrombocytopenic purpura, immunodeficiencies, Osteoporosis, Hypercalceimia, Arthritis, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Asthma, allergy, ARDS, Lesch-Nyhan syndrome, Multiple sclerosis, Leukodystrophies, Behavioral disorders, Addiction, Anxiety, Pain, Neuroprotection Fertility, psoriasis, cancer including but not limited to basal and squamous cell carcinomas, obesity, diabetis, and/or other pathologies and disorders involving fatty acid transport of skin, oral mucosa as well as other organs. The novel NOV6 nucleic acid encoding NOV6 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

NOV6 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV6 protein have multiple hydrophilic regions, each of which can be used as an immunogen. In one embodiment, contemplated NOV6 epitope is from about amino acids 10 to 35. In other embodiments, NOV6 epitope is from about amino acids 40 to 45, from about amino acids 50 to 85, or from about amino acids 90 to 110. This novel protein also has value in development of powerful assay system for functional analysis of various human disorders, which will help in understanding of pathology of the disease and development of new drug targets for various disorders.

NOV7

A disclosed NOV7 nucleic acid of 822 nucleotides (also referred to sggc_draft_dj895c5_{—}20000819) encoding a novel Gap junction beta-5 protein-like protein is shown in Table 7A. An open reading frame was identified beginning with an ATG initiation codon at nucleotides 1-3 and ending with a TAA codon at nucleotides 800-802. In Table 7A, the 5′ and 3′ untranslated regions are underlined and the start and stop codons are in bold letters.

TABLE 7A
NOV7 Nucleotide Sequence

(SEQ ID NO:19)

CATGAACTGGGCATTTCTGCAGGGCCTGCTGAGTGGCGTGAACAAGTACTCCACAGTGCTGAGCCGCATCT
GGCTGTCTGTGGTGTTCATCTTTCGTGTGCTGGTGTACGTGGTGGCAGCGGAGGAGGTGTGGGACGATGAG
CAGAAGGACTTTGTCTGCAACACCAAGCAGCCCGGCTGCCCCAACGTCTGCTATGACGAGTTCTTCCCCGT
GTCCCACGTGCGCCTCTGGGCCCTACAGCTCATCCTGGTCACGTGCCCCTCACTGCTCGTGGTCATGCACG
TGGCCTACCGCGAGGAACGCGAGCGCAAGCACCACCTGAAACACGGGCCCAATGCCCCGTCCCTGTACGAC
AACCTGAGCAAGAAGCGGGGCGGACTGTGGTGGACGTACTTGCTGAGCCTCATCTTCAAGGCCGCCGTGGA
TGCTGGCTTCCTCTATATCTTCCACCGCCTCTACAAGGATTATGACATGCCCCGCGTGGTGGCCTGCTCCG
TGGAGCCTTGCCCCCACACTGTGGACTGTTACATCTCCCGGCCCACGGAGAAGAAGGTCTTCACCTACTTC
ATGGTGACCACAGCTGCCATCTGCATCCTGCTCAACCTCAGTGAAGTCTTCTACCTGGTGGGCAAGAGGTG
CATGGAGATCTTCGGCCCCAGGCACCGGCGGCCTCGGTGCCGCCAATGCCTACCCCATACGTGCCCACCAT
ATGTCCTCTCCCAGGGAGGGCACCCTGAGGATGGGAACTCTGTCCTAATGAAGGCTGGGTCGGCCCCAGTG
GATGCAGGTGGGTATCCATAACCTGCGAGATCAGCAGATAA

The disclosed NOV7 nucleic acid sequence, localized to the p34.3-36.11 region of chromosome 1, has 682 of 807 bases (84%) identical to a gb:GENBANK-ID:RNCON303|acc:X76168 mRNA from Rattus norvegicus (R.norvegicus mRNA for connexin 30.3) (E=3.7e−125).

A disclosed NOV7 polypeptide (SEQ ID NO:20) encoded by SEQ ID NO:19 is 266 amino acid residues and is presented using the one-letter amino acid code in Table 7B. Signal P, Psort and/or Hydropathy results predict that NOV7 has a signal peptide and is likely to be localized in the plasma membrane with a certainty of 0.6000. In other embodiments, NOV7 is also likely to be localized to the Golgi body with a certainty of 0.4000, to the endoplasmic reticulum (membrane) with a certainty of 0.3000, or the nucleus with a certainty of 0.2400. The most likely cleavage site for a NOV7 peptide is between amino acids 40 and 41, at: VAA-EE.

TABLE 7B
Encoded NOV7 protein sequence.

(SEQ ID NO:20)

MNWAFLQGLLSGVNKYSTVLSRIWLSVVFIFRVLVYVVAAEEVWDDEQKDFVCNTKQPGCPNVCYDEFFPV
SHVRLWALQLILVTCPSLLVVMHVAYREERERKHHLKHGPNAPSLYDNLSKKRGGLWWTYLLSLIFKAAVD
AGFLYIFHRLYKDYDMPRVVACSVEPCPHTVDCYISRPTEKKVFTYFMVTTAAICILLNLSEVFYLVGKRC
MEIFGPRHRRPRCRECLPDTCPPYVLSQGGHPEDGNSVLMKAGSAPVDAGGYP

The disclosed NOV7 amino acid sequence has 230 of 266 amino acid residues (86%) identical to, and 244 of 266 amino acid residues (91%) similar to, the 266 amino acid residue .g) 15 ptnr:SWISSPROT-ACC:Q02738 protein from Mus musculus (Mouse) (Gap Junction Beta-5 Protein (Connexin 30.3) (CX30.3) (4.4e−129).

NOV7 is expressed in at least kidney tissue. The sequence is predicted to be expressed in the following tissues because of the expression pattern of (GENBANK-ID:

RNCON₃₀₃|acc:X76168) a closely related {R.norvegicus mRNA for connexin 30.3 homolog 20 in species Rattus norvegicus:kidney and thymus.

SNP data for NOV7 can be found below in Example 3. NOV7 also has homology to the amino acid sequence shown in the BLASTP data listed in Table 7C.

TABLE 7C
BLAST results for NOV7

| Gene Index/ | | Length | Identity | Positives | |
|---|---|---|---|---|---|
| Identifier | Protein/Organism | (aa) | (%) | (%) | Expect |
| gi\|12229761\|sp\|Q9NTQ9\| | GAP JUNCTION | 266 | 266/266 | 266/266 | e−155 |
| CXB4_HUMAN | BETA-4 PROTEIN | | (100%) | (100%) | |
| | (CONNEXIN 30.3) | | | | |
| | (CX30.3) | | | | |
| gi\|544118\|sp\|P36380\| | GAP JUNCTION | 265 | 230/266 | 245/266 | e−132 |
| CXB5_RAT | BETA-5 PROTEIN | | (86%) | (91%) | |
| | (CONNEXIN 30.3) | | | | |
| | (CX30.3) | | | | |
| gi\|6680015\|ref\|NP_{—} | gap junction | 266 | 230/266 | 244/266 | e−131 |
| 032153.1\| | membrane channel | | (86%) | (91%) | |
| | protein beta 4; | | | | |
| | connexin 30.3 | | | | |
| | [Mus musculus] | | | | |
| gi\|10835079\|ref\|NP_{—} | gap junction | 273 | 153/226 | 177/226 | 3e−88 |
| 005259.1\| | protein, beta 5 | | (67%) | (77%) | |
| | (connexin 31.1) | | | | |
| | [Homo sapiens] | | | | |
| gi\|4009522\|gb\|AAC95472.1\| | connexin 31.1 | 273 | 152/226 | 176/226 | 1e−87 |
| (AF099731) | [Homo sapiens] | | (67%) | (77%) | |

The homology of these sequences is shown graphically in the ClustalW analysis shown in Table 7D.

[image]

[image]

Table 7E-F lists the domain description from DOMAIN analysis results against NOV7. This indicates that the NOV7 sequence has properties similar to those of other proteins known to contain this domain.

TABLE 7E
Domain Analysis of NOV7

gnl|Pfam|pfam00029, connexin, Connexin. (SEQ ID NO:91)
CD-Length=218 residues, 100.0% aligned
Score=318 bits (814), Expect=3e-88

| | | | |
|---|---|---|---|
| Query: | 1 | MNWAFLQGLLSGVNKYSTVLSRIWLSVVFIFRVLVYVVAAEEVWDDEQKDFVCNTKQPGC | 60 |
| | | \|+\|+\|\|\|\|\|\|\|\|+\|\|++\|\|\|\|\|+\|\|++\|\|\|\|\|+\|\|\|\|+\|\|\|\|\|\|\|\|\|\|\|\|\|\|\|\|\|+\|\|\|\| | |
| Sbjct: | 1 | MDWSFLGRLLEGVNKHSTAIGKIWLSVLFIFRILVLGVAAESVWGDEQSDFVCNTQQPGC | 60 |
| Query: | 61 | PNVCYDEFFPVSEVRLWALQLILVTCPSLLVVMHVAYREERERKHHLKHGPNAPSLYDNL | 120 |
| | | \|\|\|\|\|+\|\|\|+\|\|\|\|\|\|\|\|\|\|\|+\|\|\|\|\|\|\|\|\|++\|\| | |
| Sbjct: | 61 | ENVCYDQFFPISEVRLWVLQLIFVSTPSLLYLGHVAYRVRREEKLREKEEEESKGLYSEE | 120 |
| Query: | 121 | SKK-----------RGGLWWTYLLSLIFKAAVDAGFLYIFHRLYKDYDMPRVVACSVEPC | 169 |
| | | +\|\|\|\|\|\|\|\|\|\|+\|+\|\|\|++\|\|\|\|+\|\|+\|+\|\|\|\| | |
| Sbjct: | 121 | AKKRCGSEDGKVRIRGGLWWTYVFSIIFKSIFEVGFLYGQYLLY-GFTMSPLVVCSRAPC | 179 |
| Query: | 170 | PHTVDCYISRPTEKKVFTYFMVTTAAICILLNLSEVFYL 208 | |
| \|\|\|\|\|\|++\|\|\|\|\|\|+\|\|\|++\|\|\|+\|\|\|\|+\|+\|\|\| | | | |
| Sbjct: | 180 | PHTVDCFVSRPTEKTIFIVFMLVVSAICLLLNLAELFYL 218 | |

TABLE 7F
Domain Analysis of NOV7

gnl|Smart|smart00037, cNX, Connexin homologues; Connexin channels
participate in the regulation of signaling between developing and
differentiated cell types. (SEQ ID NO:160)
CD-Length=34 residues, 100.0% aligned
Score=79.0 bits (193), Expect=3e-16

| | | | |
|---|---|---|---|
| Query: | 42 | EVWDDEQKDFVCNTKQPGCPNVCYDEFFPVSHVR | 75 |
| | | \|\|\|\|\|\|\|\|\|\|+\|\|\|\|\|\|\|\|\|\|+\|\|\|\| | |
| Sbjct: | 1 | SVWGDEQSDFTCNTQQPGCENVCYDQFFPISHVR | 34 |

Gap junctions are conduits that allow the direct cell-to-cell passage of small cytoplasmic molecules, including ions, metabolic intermediates, and second messengers, and thereby mediate intercellular metabolic and electrical communication. Gap junction channels consist of connexin protein subunits, which are encoded by a multigene family. Richard et al. (Nature Genet. 20: 366-369,1998) identified 2 expressed sequence tags (ESTs) from the human EST database by their similarity to mouse Gjb3 (see 603324) and Gjb5. By radiation hybrid mapping, they placed them in proximity to a sequence tagged site (STS) that is linked to GJA4 (121012) at Ip35.1. Richard et al. (1998) determined the cDNA sequences of the genes from which both ESTs were derived. Sequence similarity to rodent connexin genes established them as human homologs of Gjb3 and Gjb5, encoding Cx32 and Cx31.1, respectively. Richard et al. (1998) excluded GJB5 as a candidate gene for erythrokeratodermia variabilis (133200) by sequence analysis. Gap junctions are intercellular channels which connect adjacent cells and allow direct exchange of molecules of low molecular weight between them (Alves L A et.al.; Braz J Med Biol Res 2000 Apr;33(4):457-65). Such a communication has been described as fundamental in many systems due to its importance in coordination, proliferation and differentiation. Recently, it has been shown that gap junctional intercellular communication (GJIC) can be modulated by several extracellular soluble factors such as classical hormones, neurotransmitters, interleukins, growth factors and some paracrine substances. Herein, we discuss some aspects of the general modulation of GJIC by extracellular messenger molecules and more particularly the regulation of such communication in the thymus gland. Additionally, we discuss recent data concerning the study of different neuropeptides and hormones in the modulation of GJIC in thymic epithelial cells. We also suggest that the thymus may be viewed as a model to study the modulation of gap junction communication by different extracellular messengers involved in non-classical circuits, since this organ is under bidirectional neuroimmunoendocrine control. The intercellular signaling system mediated by connexin channels is crucial for maintaining tissue homeostasis, growth control, development, and synchronized response of cells to stimuli (Richard G; Exp Dermatol 2000 Apr;9(2):77-96). This review summarizes the structure, assembly, and properties of the components of the complex and diverse connexin system, and their biological functions in skin. The importance of gap junctional intercellular communication for normal development and differentiation of human epidermis as well as the hearing function of the inner ear is illustrated by the examples of erythrokeratodermia variabilis and palmoplantar keratoderma associated with hearing loss. These autosomal dominant inherited disorders are caused by germline mutations in the connexin genes GJB3 (encoding connexin-31) and GJB2 (encoding connexin-26), respectively. Recent functional studies of individual connexin mutations suggest that they may exert a dominant inhibitory effect on normal connexin channel function and perturb gap junctional intercellular communication, resulting in phenotypic manifestation in patients with these disorders. Gap junction channels are sites of cytoplasmic communication between contacting cells. In vertebrates, they consist of protein subunits denoted connexins (Cxs) which are encoded by a gene family (Saez J C et.al.; Braz J Med Biol Res 2000 Apr;33(4):447-55). According to their Cx composition, gap junction channels show different gating and permeability properties that define which ions and small molecules permeate them. Differences in Cx primary sequences suggest that channels composed of different Cxs are regulated differentially by intracellular pathways under specific physiological conditions. Functional roles of gap junction channels could be defined by the relative importance of permeant substances, resulting in coordination of electrical and/or metabolic cellular responses. Cells of the native and specific immune systems establish transient homo- and heterocellular contacts at various steps of the immune response. Morphological and functional studies reported during the last three decades have revealed that many intercellular contacts between cells in the immune response present gap junctions or “gap junction-like” structures. Partial characterization of the molecular composition of some of these plasma membrane structures and regulatory mechanisms that control them have been published recently. Studies designed to elucidate their physiological roles suggest that they might permit coordination of cellular events which favor the effective and timely response of the immune system. Antitumor suicide gene therapy is one of the emerging strategies against cancer (Mesnil M et. al.; Cancer Res 2000 Aug 1 ;60(15):3989-99) . It consists of the introduction into cancer cells of a gene capable of converting a nontoxic prodrug into a cytotoxic drug. Because this therapeutic gene cannot be easily introduced into the whole cell population of a tumor, the successful eradication of tumors depends on a phenomenon called the “bystander effect,” by which the introduced gene can affect even cells in which it is not itself present. From a therapeutic point of view, it may be crucial to enhance this phenomenon through various means to achieve tumor eradication. One such suicide gene, the thymidine kinase gene from the herpes simplex virus, in combination with the prodrug ganciclovir, has been extensively and successfully used in some animal models exhibiting a strong bystander effect. Among the mechanisms involved in this phenomenon, gap junctional intercellular communication (GJIC) is directly involved in the transfer of the toxic metabolites of ganciclovir, which pass directly from herpes simplex virus thymidine kinase-expressing cells to surrounding cells that do not express it. Because GJIC appears to be a mediator of the bystander effect both in vitro and in vivo, here we review possible molecular strategies for enhancing the extent of tumor cell death by increasing the intratumoral GJIC capacity. Synapses are classically defined as close connections between two nerve cells or between a neuronal cell and a muscle or gland cell across which a chemical signal (i.e., a neurotransmitter) and/or an electrical signal (i.e., current-carrying ions) can pass (Rozental R et.al.; Brain Res Brain Res Rev 2000 Apr;32(1):11-5). The definition of synapse was developed by Charles Sherrington and by Ramon y Cajal at the beginning of this century and refined by John Eccles and Bernard Katz 50 years later; in this collection of papers, the definition of synapses is discussed further in the chapter by Mike Bennett. who provided the first functional demonstration of electrical transmission via gap junction channels between vertebrate neurons. As is evidenced by the range of topics covered in this issue, research dealing with gap junctions in the nervous system has expanded enormously in the past decade, major findings being that specific cell types in the brain expresses specific types of connexins and that expression patterns coincide with tissue compartmentalization and function and that these compartments change during development. Connexins, the protein molecules forming gap junction channels, are reduced in number or redistributed from intercalated disks to lateral cell borders in a variety of cardiac diseases (Jongsma H J et.al.; Circ Res 2000 Jun 23;86(12):1193-7). This “gap junction remodeling” is considered to be arrhythmogenic. Using a simple model of human ventricular myocardium, we found that quantitative remodeling data extracted from the literature gave rise to only small to moderate changes in conduction velocity and the anisotropy ratio. Especially for longitudinal conduction, cytoplasmic resistivity (and thus cellular geometry) is much more important than commonly realized. None of the remodeling data gave rise to slow conduction on the order of a few centimeters per second. Physical signals, in particular mechanical loading, are clearly important regulators of bone turnover (Donahue H J; Bone 2000 May;26(5):417-22). Indeed, the structural success of the skeleton is due in large part to the bone's capacity to recognize some aspect of its functional environment as a stimulus for achievement and retention of a structurally adequate morphology. However, while the skeleton's ability to respond to its mechanical environment is widely accepted, identification of a reasonable mechanism through which a mechanical “load” could be transformed to a signal relevant to the bone cell population has been elusive. In addition, the downstream response of bone cells to load-induced signals is unclear. In this work, we review evidence suggesting that gap junctional intercellular communication (GJIC) contributes to mechanotransduction in bone and, in so doing, contributes to the regulation of bone cell differentiation by biophysical signals. In this context, mechanotransduction is defined as transduction of a load-induced biophysical signal, such as fluid flow, substrate deformation, or electrokinetic effects, to a cell and ultimately throughout a cellular network. Thus, mechanotransduction would include interactions of extracellular signals with cellular membranes, generation of intracellular second messengers, and the propagation of these messengers, or signals they induce, through a cellular network. We propose that gap junctions contribute largely to the propagation of intracellular signals.

The disclosed NOV7 nucleic acid of the invention encoding a Gap Junction Beta-5 Protein-like protein includes the nucleic acid whose sequence is provided in Table 7A or a fragment thereof. The invention also includes a mutant or variant nucleic acid any of whose bases may be changed from the corresponding base shown in Table 7A while still encoding a protein that maintains its Gap Junction Beta-5 Protein-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 16% percent of the bases may be so changed.

The disclosed NOV7 protein of the invention includes the Gap Junction Beta-5 Protein-like protein whose sequence is provided in Table 7B. The invention also includes a mutant or variant protein any of whose residues may be changed from the corresponding residue shown in Table 7B while still encoding a protein that maintains its Gap Junction Beta-5 Protein-like activities and physiological functions, or a functional fragment thereof. In the mutant or variant protein, up to about 33% percent of the residues may be so changed.

The protein similarity information, expression pattern, and map location for the Gap Junction Beta-5 Protein-like protein and nucleic acid (NOV7) disclosed herein suggest that NOV7 may have important structural and/or physiological functions characteristic of the Gap Junction Beta-5 Protein-like family. Therefore, the NOV7 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV7 nucleic acids and proteins of the invention are useful in potential diagnostic and therapeutic applications implicated in various diseases and disorders described below and/or other pathologies. For example, the compositions of the present invention will have efficacy for treatment of patients suffering from Deafness, autosomal dominant 2; Elliptocytosis-1; Fucosidosis; Hypophosphatasia (adult, childhood, infantile); Muscle-eye-brain disease; Neuropathy, paraneoplastic sensory; Porphyria cutanea tarda; Porphyria, hepatoerythropoietic; Schwartz-Jampel syndrome; Thrombocytopenia, congenital amegakaryocytic; Charcot-Marie-Tooth neuropathy-2A; Galactose epimerase deficiency; Glucose transport defect, blood-brain barrier; Kostmann neutropenia; Muscular dystrophy, congenital, with early spine rigidity; Myopathy due to succinate dehydrogenase deficiency; SCID due to LCK deficiency; Colorectal cancer, resistance to; Bartter syndrome, type 3; Breast cancer, ductal; Corneal dystrophy, crystalline, Schnyder; Hyperprolinemia, type II; Inflammatory bowel disease 7; Malignant melanoma, cutaneous; Neuroblastoma; Prostate cancer-brain cancer susceptibility; erythrokeratodermia variabilis; palmoplantar keratoderma; diseases and disorders involving intercellular metabolic and electrical communication; diseases and disorders involving coordination, proliferation and differentiation; diseases and disorders involving maintainance of tissue homeostasis, growth control, development, and synchronized response of cells to stimuli; diseases and disorders involving the the immune system; diseases and disorders involving regulation of bone cell differentiation, and/or other pathologies/disorders. The NOV7 nucleic acid, or fragments thereof, may further be useful in diagnostic applications, wherein the presence or amount of the nucleic acid or the protein are to be assessed.

NOV7 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV7 protein have multiple hydrophilic regions, each of which can be used as an immunogen. In one embodiment, contemplated NOV7 epitope is from about amino acids 40 to 70. In other embodiments, NOV7 epitope is from about amino acids 90 to 140, from about amino acids 170 to 180, or from about amino acids 220 to 255. This novel protein also has value in development of powerful assay system for functional analysis of various human disorders, which will help in understanding of pathology of the disease and development of new drug targets for various disorders.

NOV8

A disclosed NOV8 nucleic acid of 546 nucleotides (also referred to as 56072181 dal) encoding a novel MT-like protein is shown in Table 8A. An open reading frame was identified beginning with an ATG initiation codon at nucleotides 214-216 and ending with a TGA codon at nucleotides 397-399. A putative untranslated region upstream from the initiation codon and downstream from the termination codon is underlined in Table 8A. The start and stop codons are in bold letters.

TABLE 8A
NOV8 nucleotide sequence

(SEQ ID NO:21)

CAATCTGACATGTAACTGTAAAATCACTGGGCCCGAGGAGTGACTGTAATTAAGGGCTTACGTTGAAAATGT
ATAAACAGAGCTTGATTCTTAAGTTTCGAAAATTCTTCACAGTGATGGTGTGAACTAAATCCACAGCCACAT
AATAATCAAAACAGAAAAGCAAAAGAAAGCCACTTCAGGAAATACCACGGTCACACCTCAGTTGGCTTCATG
CTACAGATTATAGAAAATATGTTGCTGCCCGGGCCACCAATCTGTTGGTTCACATTACTACGTGAGCAATGT
AAGTGTTTGCAAGAAGCCATCCACTATCTAAATATCAGATATAGATGCTCCAAAGCAGCTACGTCAGTGATG
AGAACAGAGAAAATACGTAGCAACATTTCATTAAGTTGAATTCTAATACTTAAAAGGCTCCTTTTAGTACTG
ACATTCTGGATTTTAAAAGTTATGTTGACCGCATGTTCTCACTCACAAGTGGGAGTTGAACAATGAGAACAC
ACGGACACGGGGAAGGGAACATCACACACCAGGGCCTGTCAG.

The NOV8 nucleic acid sequence is located on the q13 region of chromsome 16.

The disclosed NOV8 polypeptide (SEQ ID NO:22) encoded by SEQ ID NO:21 has 61 amino acid residues and is presented in Table 8B using the one-letter amino acid code. Signal P, Psort and/or Hydropathy results predict that NOV8 has no signal peptide and is likely to be localized in the cytoplasm with a certainty of 0.6500. In other embodiments, NOV8 may also be localized to the mitochondrial matrix space with a certainty of 0.3100, or the lysosome (lumen) with a certainty of 0.1000.

TABLE 8B
Encoded NOV8 protein sequence

| | |
|---|---|
| MLQIIENMLLPGPPICWFTLLREQCKCLQEAIHYLNIRYRCSKAATSVMRTEKIRSNISLS. | (SEQ ID NO:22) |

A search of sequence databases reveals that the NOV8 amino acid sequence has 16 of 41 amino acid residues (39%) identical to, and 25 of 41 amino acid residues (60%) similar to, the 48 amino acid residue Metallothionein protein from Rhizomucor racemosus (Mucor circinelloides f. lusitanicus) (SPTREMBL-ACC: Q9Y762) (E=0.049).

SNP data for NOV8 can be found below in Example 3.

Masters et al. (Proc. Nat. Acad. Sci. 91: 584-588, 1994) described metallothioneins (MTs) as a family of low molecular weight, heavy metal-binding proteins characterized by a high cysteine content and lack of aromatic amino acids. MTs bind 7 to 12 heavy metal atoms per molecule of protein. They are ubiquitous in the animal and plant kingdoms and are found in prokaryotes. In mammals, the cysteine residues are absolutely conserved and serve to coordinate heavy metal atoms such as zinc, cadmium, and copper via mercaptide linkages. In human liver, MTs occur in 2 major forms, MT-I and MT-II (156360). In HeLa cells, MT synthesis is induced by either ionized zinc or ionized cadmium and by glucocorticoid hormones. In man, metallothioneins are encoded by at least 10 to 12 genes separated into 2 groups designated MT-I and MT-II. Masters et al. (1994) noted that, unlike MT-I and MT-II, which are expressed in most organs, MT-III (139255) expression appears to be restricted to the brain, and MT-IV is only expressed in certain stratified squamous epithelia.

MTs have been postulated to detoxify metals; to play a role in zinc and copper homeostasis during development; to regulate synthesis, assembly, or activity of zinc metalloproteins; and to protect against reactive oxygen species. MTs may also protect against copper toxicity in the Menkes disease (309400) and murine ‘Mottled’ phenotypes (X-linked diseases resulting in copper deficiency) as well as in Wilson disease (277900); see also the Animal Models section.

Karin and Richards (Nature 299: 797-802, 1982) described the molecular cloning and sequence analysis of human metallothionein transcripts. Karin et al. (1984) characterized DNA sequences that are involved in the induction of MT gene expression by cadmium and glucocorticoids.

Mapping

Karin et al. (Proc. Nat. Acad. Sci. 81: 5494-5498, 1984) used several different hybridization probes derived from cloned and functional human MT1 and MT2 genes to map the genes in somatic cell hybridization studies. They concluded that most of the human genes are clustered on chromosome 16. Analysis of RNA from somatic cell hybrids indicated that all hybrids that contain human chromosome 16 express both MT1 and MT2 mRNA and that expression is regulated by both heavy metal ions and glucocorticoid hormones.

In the mouse, the metallothionein genes are on chromosome 8, which has other homology to human chromosome 16; by somatic cell hybridization, Cox and Palmiter (Hum. Genet. 64: 61-64, 1983) assigned the MT-1 structural gene to mouse chromosome 8, which also carries glutathione reductase in the mouse. (By chance the human 8 also carries glutathione reductase.)

Schmidt et al. (Science 224: 1104-1106, 1984) concluded that MT1 is located between PGP (172280) and DIA4 (125860) and is probably on the long arm 16cen-16q21 because APRT (102600), a 16q marker, and MT1 are both on mouse chromosome 8, whereas HB alpha (141800), a 16p marker, is on mouse chromosome 11. They stated that analysis of the involvement of the MT genes in Wilson disease (277900) and in acrodermatitis enteropathica (201100) would be of great interest.

By gel transfer hybridization analysis of the DNA from human-rodent cell hybrids, Schmidt et al. (J. Biol. Chem. 260: 7731-7737, 1985) showed that chromosome 16 contains a cluster of metallothionein sequences, including 2 functional metallothionein I genes (156351 and 156352) and a functional metallothionein II gene. The remaining sequences, including a processed pseudogene, are dispersed to at least 4 other autosomes. The absence of metallothionein sequences from the X chromosome indicates that the Menkes disease mutation affects metallothionein expression by a ‘trans-acting’ mechanism. The processed pseudogene is on chromosome 4 and shows allelic variation (Karin and Richards, Nucleic Acids Res. 10: 3165-3173, 1982). Two MT genes are on chromosome 1 but not close together: one is on the distal two-thirds of the short arm and the second probably on the long arm. One metallothionein gene is on chromosome 20 and another is on chromosome 18.

By in situ hybridization, Le Beau et al. (Nature 313: 709-711, 1985) assigned the metallothionein gene cluster to 16q22. This band is a breakpoint in 2 specific rearrangements, inv(16)(p13q22) and t(16;16)(pl3;q22), found in a subgroup of patients with acute myelomonocytic leukemia. Hybridization of an MT probe to malignant cells from patients with one or the other of these rearrangements showed that the breakpoint at 16q22 splits the MT gene cluster. The findings were interpreted as indicating that the MT genes or their regulatory regions may function as an ‘activating’ sequence for an as yet unidentified cellular gene located at 16p13. Band 16p22 carries 2 fragile sites: the rare FRA16B and the common FRA16C. Simmers et al. (Science 236: 92-94, 1987) showed that the specific leukemic break that is situated in the metallothionein gene cluster lies proximal to both fragile sites; therefore, neither of these fragile sites could have played a role in the breakage.

Using high-resolution in situ hybridization, Sutherland et al. (Cytogenet. Cell Genet. 51: 1087, 1989, Genomics 6: 144-148, 1990) corrected the mapping of the human metallothionein gene complex to 16q13. They found, furthermore, that the complex is not disrupted by the rearrangement breakpoint on 16q in the patients with myelomonocytic leukemia with abnormal eosinophils, as had previously been reported. They showed that the order is cen—MT—FRA16B—D16S4—inversion breakpoint—HB—qter.

Foster et al. (J. Biol. Chem. 263: 11528-11535, 1988) indicated that 4 functional MT1 genes had been identified and mapped to 16q: MT1A, MT1B (156349), MT1E (156351), and MMT1F (156352). They also characterized a fifth MT gene, MT1G (156353). West et al. (Genomics 8: 513-518, 1990) mapped the cluster of MT genes in an 82.1-kb region of 16q13. Of the 14 tightly linked genes, 6 had not previously been described. The mapped genes included the single MT2 gene, MT2A, and at least 2 pseudogenes, MT1C and MT1D. The genes were flanked by the single MT2A gene at one end and a gene labeled MT1X (156359) at the other. The order of genes, beginning at the MT2A end, was 1L—1E—1K—1J—1A—1D—1C—1B—1F—1G—1H—1I. This was also the 5-prime to 3-prime direction of transcription for all the genes except MT1G, which had a tail-to-tail, head-to-head orientation to MT1F and MT1H, respectively.

Animal Model

To test the proposed detoxification and homeostasis functions of mammalian MTs in vivo, Masters et al. (1994) inactivated both alleles of the Mt1 and Mt2 genes in embryonic stem cells and generated mice homozygous for these mutant alleles. These mice were viable and reproduced normally when reared under normal laboratory conditions. They were, however, more susceptible to hepatic poisoning by cadmium. This suggested to Masters et al. (1994) that these widely expressed MTs are not essential for development but do protect against cadmium toxicity.

Human Menkes disease (309400) and the murine ‘Mottled’ phenotype are X-linked diseases that result from copper deficiency due to mutations in ATP7A, a copper-effluxing ATPase (300011). Male mice with the Mottled-Brindled allele accumulate copper in the intestine, fail to export copper to peripheral organs, and die a few weeks after birth. Much of the intestinal copper is bound by metallothionein. To determine the function of MT in the presence of Atp7a deficiency, Kelly and Palmiter (Nature Genet. 13: 219-222, 1996) crossed Mottled-Brindled females with males that bear a targeted disruption of the Mtl and Mt2 genes. On the metallothionein-deficient background most Mottled males as well as heterozygous Mottled females died before embryonic day 11. The authors explained the lethality in females by preferential inactivation of the paternal X chromosome in extra embryonic tissues and resultant copper toxicity in the absence of MT.

In support of this hypothesis, Kelly and Palmiter (1996) found that cell lines derived from metallothionein deficient, Mottled embryos were very sensitive to copper toxicity. They concluded that MT is essential to protect against copper toxicity in embryonic placenta, providing a second line of defense when copper effluxers are defective. They also stated that MT probably protects against hepatic copper toxicity in Wilson disease and the LEC rat model in which a similar copper effluxer, ATP7B (277900), is defective, because MT accumulates to high levels in the liver in those diseases.

Beattie et al. (Proc. Nat. Acad. Sci. 95: 358-363, 1998) noted that mice with targeted disruption of the metallothionein-I and metallothionein-II genes were more sensitive to toxic metal and oxidative stress. In addition they were larger than most strains of mice, becoming significantly heavier at age 5 to 6 weeks. At age 14 weeks, the body weight and food intake of MT-null mice was 16 and 30% higher, respectively, compared with control mice. Most 22- to 39-week-old male MT-null mice were obese. Seven-week-old MT-null also had significantly higher levels of plasma leptin (601694) and elevated expression of OB (164160), lipoprotein lipase (238600), and CCAAT enhancer binding protein alpha (189965) genes as compared with age-matched control mice. Abnormal accretion of body fat and adipocyte maturation was initiated at 5 to 7 weeks of age, possibly coincident with sexual maturation. Beattie et al. (1998) concluded that a link between MT and the regulation of energy balance is implied by these observations. They noted the possibility that obesity and the associated biochemical changes in the MT-null mice may be caused by factors other than lack of MT. For example, disruption of MT genes by homologous recombination with DNA containing various modifications may have affected other genes around this locus or may have had downstream effects on gene expression.

The disclosed NOV8 nucleic acid of the invention encoding a MT-like protein includes the nucleic acid whose sequence is provided in Table 8A, or a fragment thereof. The invention also includes a mutant or variant nucleic acid any of whose bases may be changed from the corresponding base shown in Table 8A while still encoding a protein that maintains its MT-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 10% percent of the bases may be so changed.

The disclosed NOV8 protein of the invention includes the MT-like protein whose sequence is provided in Table 8B. The invention also includes a mutant or variant protein any of whose residues may be changed from the corresponding residue shown in Table 2 while still encoding a protein that maintains its MT-like activities and physiological functions, or a functional fragment thereof. In the mutant or variant protein, up to about 61% percent of the residues may be so changed.

The invention further encompasses antibodies and antibody fragments, such as F_{ab} or (F_{ab})₂, that bind immunospecifically to any of the proteins of the invention.

The above defined information for this invention suggests that this MT-like protein (NOV8) may function as a member of a “MT family”. Therefore, the NOV8 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV8 nucleic acids and proteins of the invention are useful in potential therapeutic applications implicated in cancer including but not limited to Inflamation, Autoimmune disorders, Aging and Cancer. For example, a cDNA encoding the MT-like protein (NOV8) may be useful in gene therapy, and the MT-like protein (NOV8) may be useful when administered to a subject in need thereof. By way of nonlimiting example, the compositions of the present invention will have efficacy for treatment of patients suffering from Gitelman syndrome, Menkes disease, Wilson's disease, acrodermatitis enteropathica, myelomonocytic leukemia, eosinophil disorders, hepatic disorders such as hepatic copper toxicity and other such conditions. The NOV8 nucleic acid encoding MT-like protein, and the MT-like protein of the invention, or fragments thereof, may further be useful in diagnostic applications, wherein the presence or amount of the nucleic acid or the protein are to be assessed.

NOV8 nucleic acids and polypeptides are further useful in the generation of antibodies that bind immuno-specifically to the novel NOV8 substances for use in therapeutic or diagnostic methods. These antibodies may be generated according to methods known in the art, using prediction from hydrophobicity charts, as described in the “Anti-NOVX Antibodies” section below. The disclosed NOV8 protein has multiple hydrophilic regions, each of which can be used as an immunogen. In one embodiment, a contemplated NOV8 epitope is from about amino acids 25 to 40. In another embodiment, a NOV8 epitope is from about amino acids 45 to 55. These novel proteins can be used in assay systems for functional analysis of various human disorders, which will help in understanding of pathology of the disease and development of new drug targets for various disorders.

NOV9

A disclosed NOV9 nucleic acid of 2309 nucleotides (also referred to as 2855519_{—}0_{—}19_da1) encoding a novel CIP4-like protein is shown in Table 9A. An open reading frame was identified beginning with an ATG initiation codon at nucleotides 78-80 and ending with a TGA codon at nucleotides 1719-1721. A putative untranslated region upstream from the initiation codon and downstream from the termination codon is underlined in Table 9A. The start and stop codons are in bold letters.

TABLE 9A
NOV9 nucleotide sequence

(SEQ ID NO:23)

GAGTGAGAGGTCGGACAGACTGTGGAGCCGACAGACTGAAGGACAGCGGCACCGCCAGACGGCCAGAAAGTT
CCGCCATGAGCTGGGGCACGGAGCTGTGGGATCAGTTCGACAGCTTAGACAAGCATACACAATGGGGAATTG
ACTTCTTGGAAAGATATGCCAAATTTGTTAAAGAGAGGATAGAAATTGAACAGAACTATGCGAAACAATTGA
GAAATCTGGTTAAGAAGTACTGCCCCAAACGTTCATCCAAAGATGAAGAGCCACGGTTTACCTCGTGTGTAG
CCTTTTTTAATATCCTTAATGAGTTAAATGACTATGCAGGACAGCGAGAAGTTGTAGCAGAAGAAATGGCGC
ACAGAGTGTATGGTGAATTAATGAGATATGCTCATGATCTGAAAACTGAAAGAAAAATGCATCTGCAAGAAG
GACGAAAAGCTCAACAATATCTTGACATGTGCTGGAAACAGATGGGTAATAGTAAAAAGAAGTTTGAAAGAG
AATGTAGAGAGGCAGAAAAGGCACAACAGAGTTATGAAAGATTGGATAATGATACTAATGCAACCAAGGCAG
ATGTTGAAAATGCCAAACAGCAGTTGAATCTGCGTACGCATATGGCCGATGAAAATAAAAATGCATATGCTG
CACAATTACAAAACTTTAATGGAGAACAACATAAACATTTTTATGTAGTGATTCCTCAGATTTACAAGCAAC
TACAAGAAATGGACGAACGAAGGACTATTAAACTCAGTGAGTGTTACAGAGGATTTGCTGACTCAGAACGCA
AAGTTATTCCCATCATTTCAAAATGTTTGGAAGGAATGATTCTTGCAGCAAAATCAGTTGATGAAAGAAGAG
ACTCTCAAATGGTGGTAGACTCCTTCAAATCTGGTTTTGAACCTCCAGGAGACTTTCCATTTGAAGATTACA
GTCAACATATATATAGAACCATTTCTGATGGGACTATCAGTGCATCCAAACAGGAGAGTGGGAAGATGGATG
CCAAAACCCCAGTAGGAAAGGCCAAGGGCAAATTGTGGCTCTTTGGAAAGAAGCCAAAGGGCCCAGCACTAG
AAGATTTCAGTCATCTGCCACCAGAACAGAGACGTAAAAAACTACAGCAGCGCATTGATGAACTTAACAGAG
AACTACAGAAAGAATCAGACCAAAAAGATGCACTCAACAAAATGAAAGATGTATATGAGAAGGATCCACAAA
TGGGGGATCCAGGGAGTTTGCAGCCTAAATTAGCAGAGACCATGAATAACATTGACCGCCTACGAATGGAAA
TCCATAAGAATGAGGCTTGGCTCTCTGAAGTCGAAGGCAAAACAGGTGGGAGAGGAGACAGAAGACATAGCA
GTGACATAAATCATCTTGTAACACAGGGACGAGAAAGTCCTGAGGGAAGTTACACTGATGATGCAAACCAGG
AAGTCCGTGGGCCACCCCAGCAGCATGGTCACCACAATGAGTTTGATGATGAATTTGAGGATGATGATCCCT
TGCCTGCTATTGGACACTGCAAAGCTATCTACCCTTTTGATGGACATAATGAAGGTACTCTAGCAATGAAAG
AAGGTGAAGTTCTCTACATTATAGAGGAGGACAAAGGTGACGGATGGACAAGAGCTCGGAGACAGAACGGTG
AAGAAGGCTACGTTCCCACGTCATACATAGATGTAACTCTAGAGAAAAACAGTAAAGGTTCCTGAAGAGGGT
TTCTGAGGAAATGGGCAAGATGTTGAAGGAGGTTACATGCAGCTGCTTTTGGGGGAGGGTATTAGAGTTGTC
AGGCTCAAAGAGAGTGAGAGAAGCAAGTTGCATGAGTGCATGCAGACATGATTTTTTTTTTACTAACTTCAT
TAGCATTTCCATACATTGTTTTTAAAAATCATAATACCAACCCTTAAGTTCCTAGTTCACAGTTATTCCCAC
AAAAGAAAAAGCCAACAATAGTGTACCATTTTTCTATTTTATTTTATTGCTGTCTAATCAATAAAGAATGCA
GAGCTGTCAAAAAATGTGTCTTACATTTAGCTGTCCCAACAGGATTGTCTTCCCTCCCAGCTCTGGTTTTAA
TTGGCTTTTAGACCCACTATCTGTCAGATCCTTGCCATCTGTCAGTGTCTGCCTGCGCCACCTCCGTGCTTG
CCTAACATCCTGTTGCATGTCTAGCGTGATTGAGCNAGATTTTCAGGCATGTCTTTAGAATCCCCTGGTNCT
GTCAAAGCCTGGTTTGGTTTACATTGGTNGTGCAATCNCTTTGTCAACATCTCCAGCACTATNGTTCCNTCT
TAGGT.

The disclosed NOV9 nucleic acid sequence, localized to the p21.2-22.2 region of chromosome 1, has 916 of 1460 bases (62%) identical to a 2001 bp cdc42-interacting protein 4 (CIP4 mRNA from Homo sapiens (GENBANK-ID: HSCIP4|acc:AJ000414) (E=1.3e ⁻⁹⁷).

The disclosed NOV9 polypeptide (SEQ ID NO:24) encoded by SEQ ID NO:23 has 547 amino acid residues and is presented in Table 9B using the one-letter amino acid code. Signal P, Psort and/or Hydropathy results predict that NOV9 has no signal peptide and is likely to be localized in the nucleus with a certainty of 0.7000. In other embodiments, NOV9 may also be localized to the microbosy (peroxisome) with a certainty of 0.3000, the mitochondrial matrix space with a certainty of 0.1000, or the lysosome (lumen) with a certainty of 0.1000.

TABLE 9B
Encoded NOV9 protein sequence

| | |
|---|---|
| MSWGTELWDQFDSLDKHTQWGIDFLERYAKFVKERIEIEQNYAKQLRNLVKKYCPKRSSKDEEP | (SEQ ID NO:24) |
| RFTSCVAFFNILNELNDYAGQREVVAEEMAHRVYGELMRYAHDLKTERKMHLQEGRKAQQYLDM | |
| CWKQMGNSKKKFERECREAEKAQQSYERLDNDTNATKADVENAKQQLNLRTHMADENKNAYAAQ | |
| LQNFNGEQHKHFYVVIPQIYKQLQEMDERRTIKLSECYRGFADSERKVIPIISKCLEGMILAAK | |
| SVDERRDSQMVVDSFKSGFEPPGDFPFEDYSQHIYRTISDGTISASKQESGKMDAKTPVGKAKG | |
| KLWLFGKKPKGPALEDFSHLPPEQRRKKLQQRIDELNRELQKESDQKDALNKMKDVYEKDPQMG | |
| DPGSLQPKLAETMNNIDRLRMEIHKNEAWLSEVEGKTGGRGDRRHSSDINHLVTQGRESPEGSY | |
| TDDANQEVRGPPQQHGHHNEFDDEFEDDDPLPAIGHCKAIYPFDGHNEGTLAMKEGEVLYIIEE | |
| DKGDGWTRARRQNGEEGYVPTSYIDVTLEKNSKGS. | |

A search of sequence databases reveals that the NOV9 amino acid sequence has 303 of 544 amino acid residues (55%) identical to, and 403 of 544 amino acid residues (74%) similar to, the 545 amino acid residue CDC42-Interacting Protein 4 protein from Homo sapiens (SPTREMBL-ACC:015184) (E=8.0e⁻¹⁶⁰).

TaqMan data for NOV9 can be found below in Example 2. The disclosed NOV9 polypeptide has homology to the amino acid sequences shown in the BLASTP data listed in Table 9C.

TABLE 9C
BLAST results for NOV9

| Gene Index/ | | Length | Identity | | |
|---|---|---|---|---|---|
| Identifier | Protein/Organism | (aa) | (%) | Positives (%) | Expect |
| gi\|13591536\|emb\| | dJ1033H22.1 | 434 | 373/430 | 375/430 | 0.0 |
| CAC36351.1\| (AL109613) | (KIAA0554 | | (86%) | (86%) | |
| | protein) [Homo | | | | |
| | sapiens] | | | | |
| gi\|8923249\|ref\|NP_{—} | hypothetical | 330 | 328/330 | 329/330 | e−175 |
| 060207.1\| | protein FLJ20275 | | (99%) | (99%) | |
| | [Homo sapiens] | | | | |
| gi\|10435680\|dbj\| | unnamed protein | 592 | 319/595 | 432/595 | e−160 |
| BAB14638.1\| (AK023681) | product [Homo | | (53%) | (71%) | |
| | sapiens] | | | | |
| gi\|13936547\|gb\| | formin-binding | 679 | 307/624 | 422/624 | e−148 |
| AAK49824.1\|AF265550_1 | protein 17 [Homo | | (49%) | (67%) | |
| (AF265550) | sapiens] | | | | |
| gi\|3043632\|dbj\| | KIAA0554 protein | 674 | 307/624 | 422/624 | e−148 |
| BAA25480.1\| (AB011126) | [Homo sapiens] | | (49%) | (67%) | |

The homology between these and other sequences is shown graphically in the ClustalW analysis shown in Table 9D. In the ClustalW alignment of the NOV9 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

[image]

Tables 9E-H lists the domain description from DOMAIN analysis results against NOV9. This indicates that the NOV9 sequence has properties similar to those of other proteins known to contain this domain.

TABLE 9E
Domain Analysis of NOV9

gnl|Smart|smart00326, SH3, Src homology 3 domains; Src homology
3 (SH3) domains bind to target proteins through sequences containing
proline and hydrophobic amino acids. Pro-containing polypeptides may
bind to SH3 domains in 2 different binding orientations. (SEQ ID
NO:92)
CD-Length = 59 residues, 88.1% aligned
Score = 64.7 bits (156), Expect = 1e−11

| | | | |
|---|---|---|---|
| Query: | 484 | HCKAIYPFDGHNEGTLAMKEGEVLYIIEEDKGDGWTRARRQNGEEGYVPTSYI | 536 |
| | | +\|+\| + + \|+ \|+\|+++ ++\|+ \|\|\| + \| \|+\|\| \|++\|+ | |
| Sbjct: | 4 | QVRALYDYTAQDPDELSFKKGDIITVLEKS-DDGWWKGRLGTGKEGLFPSNYV | 55 |

TABLE 9F
Domain Analysis of NOV9

gnl|Pfam|pfam00018, SH3, SH3 domain. SH3 (Src homology 3) do-
mains are often indicative of a protein involved in signal trans-
duction related to cytoskeletal organization. First described in the
Src cytoplasmic tyrosine kinase. The structure is a partly opened
beta barrel. (SEQ ID NO:93)
CD-Length = 57 residues, 91.2% aligned
Score = 63.3 bits (153), Expect = 3e−11

| | | | |
|---|---|---|---|
| Query: | 486 | KAIYPFDGHNEGTLAMKEGEVLYIIEEDKGDGWTRARRQNGEEGYVPTSYID | 537 |
| | | A+Y + L+ K+G+++ ++E+ GW + R + +EG +P++Y++ | |
| Sbjct: | 4 | VALYDYQARESDELSFKKGDIIIVLEKSDDGGWWKGRLKGTKEGLIPSNYVE | 55 |

TABLE 9G
Domain Analysis of NOV9

gnl|Smart|smart00055, FCH, Fes/CIP4 homology domain; Alignment
extended from original report. Highly alpha-helical. Also known as the
RAEYL motif or the S. pombe Cdc15 N-terminal domain. (SEQ ID NO:94)
CD-Length = 91 residues, 97.8% aligned
Score = 58.2 bits (139), Expect = 1e−09

| | | | |
|---|---|---|---|
| Query: | 1 | MSWGTELWDQFDSLDKHTQWGIDFLERYAKFVKERIEIEQNYAKQLRNLVKKYCPKRSSK | 60 |
| | | \| + +\|\| \| \|++\| + \|+ \|\| \|\|++\|\| +\|\|+ \|\|\|+\|+ \| \|\| + | |
| Sbjct: | 1 | MGFWSELDDGFEALLSRLKNGLRLLEDLKKFMRERAKIEEEYAKKLQKLSKK--LRAVRD | 58 |
| Query: | 61 | DEEPRFTSCVAFFNILNELNDYAGQREVVAE 91 | |
| | | \| + \|+ +\|+\| + \| \| ++\| | |
| Sbjct: | 59 | TESELGSLRKAWEVLLSETDALAKQHLQLSE 89 | |

TABLE 9H
Domain Analysis of NOV9

gnl|Pfam|pfam0061, ECH, Fes/CIP4 homology domain. Alignment extended
from. Highly alpha-helical. (SEQ ID NO:95)
CD-Length = 94 residues, 97.9% aligned
Score = 40.0 bits (92), Expect = 3e−04

| | | | |
|---|---|---|---|
| Query: | 1 | MSWGTELWDQ-FDSLDKHTQWGIDFLERYAKFVKERIEIEQNYAKQLRNLVKKYCPKRSS | 59 |
| | | \| +\|+\|\| + +\| + \|\| \|\|+ \|\| +\|\|+ \|\| +\|++\| + ++ | |
| Sbjct: | 1 | MGFGSELCPEGHKALLSRQDNELRLLEEMKKFMAERAKIEKEYAGKLQHLSAQVGKGPAT | 60 |
| Query: | 60 | KDEEPRFTSCVAFFNILNELNDYAGQREVVAE 91 | |
| | | + \| +\| ++ \|\|+\| + ++\| | |
| Sbjct: | 61 | AEGEDELSSLKSWAVILSETEQQSKIHLQISE 92 | |

The thyroid hormone receptors (TRs) are hormone-dependent transcription factors that regulate expression of a variety of specific target genes. They must specifically interact with a number of proteins as they progress from their initial translation and nuclear translocation to heterodimerization with retinoid X receptors (RXRs), functional interactions with other transcription factors and the basic transcriptional apparatus, and eventually, degradation. To help elucidate the mechanisms that under lie the transcriptional effects and other potential functions of TRs, Lee et al. (Molec. Endocr. 9: 243-254, 1995) used the yeast interaction trap, a version of the yeast 2-hybrid system, to identify proteins that specifically interact with the ligand-binding domain of rat TR-beta (THRB; 190160). They isolated HeLa cell cDNAs encoding several different TR-interacting proteins (TRIPs), including TRIP10. TRIP10 interacted with rat Thrb only in the presence of thyroid hormone. It also showed a ligand-dependent interaction with RXR-alpha (RXRA; 180245), but did not interact with the glucocorticoid receptor (NR3C1; 138040) under any condition. TRIP10 contains a Src homology-3 (SH3) domain and shows sequence similarity to a Xenopus fyn homolog (see 137025) and chicken src (see 190090). Northern blot analysis detected a 2.6 -kb TRIP10 transcript in several tissues, with highest expression in skeletal muscle.

Using the yeast 2-hybrid system to identify proteins that bind to a constitutively active mutant of CDC42 (116952), Aspenstrom (1997) isolated a human B-cell cDNA encoding CDC42-interacting protein-4 (CIP4), which is identical to TRIP10. The predicted 545-amino acid CIP4 protein contains an N-terminal domain that bears resemblance to the nonkinase domain of the FER (176942) and Fes/Fps family of tyrosine kinases, and a C-terminal SH3 domain. In addition, CIP4 shares sequence similarity with a number of proteins that have roles in regulating the actin cytoskeleton. Aspenstrom (Curr. Biol. 7: 479-487, 1997) demonstrated that CIP4 can bind to activated CDC42 in vitro and in vivo. Overexpression of CIP4 in fibroblasts reduced the amount of stress fibers in these cells. Recombinant CIP4 protein accumulated at the cell periphery, particularly in areas that exhibited membrane ruffling. Coexpression of activated CDC42 and CIP4 led to clustering of CIP4 to a large number of foci at the dorsal side of the cells. Northern blot analysis showed a major 2.2 -kb CIP4 transcript that was abundant in skeletal muscle, heart, and placenta, present at lower levels in pancreas, lung, liver, and kidney, and barely detectable in brain. Minor transcripts of 3.5 and 5 kb were also detected.

Aspenstrom (1997) concluded that CIP4 is a downstream target of activated GTP-bound CDC42, and is similar in sequence to proteins involved in signaling and cytoskeletal control. The author suggested that CIP4 may act as a link between CDC42 signaling and regulation of the actin cytoskeleton.

The Wiskott-Aldrich syndrome is an inherited X-linked immunodeficiency characterized by thrombocytopenia, eczema, and a tendency toward lymphoid malignancy (Tian L, et.al.; J Biol Chem 2000 Mar 17;275(11):7854-61). Lymphocytes from affected individuals have cytoskeletal abnormalities, and monocytes show impaired motility. The Wiskott-Aldrich syndrome protein (WASP) is a multi-domain protein involved in cytoskeletal organization. In a two-hybrid screen, we identified the protein Cdc42-interacting protein 4 (CIP4) as a WASP interactor. CIP4, like WASP, is a Cdc42 effector protein involved in cytoskeletal organization. We found that the WASP-CIP4 interaction is mediated by the binding of the Src homology 3 domain of CIP4 to the proline-rich segment of WASP. Cdc42 was not required for this interaction. Co-expression of CIP4 and green fluorescent protein-WASP in COS-7 cells led to the association of WASP with microtubules. In vitro experiments showed that CIP4 binds to microtubules via its NH(2) terminus. The region of CIP4 responsible for binding to active Cdc42 was localized to amino acids 383417, and the mutation 1398S abrogated binding. Deletion of the Cdc42-binding domain of CIP4 did not affect the colocalization of WASP with microtubules in vivo. We conclude that CIP4 can mediate the association of WASP with microtubules. This may facilitate transport of WASP to sites of substrate adhesion in hematopoietic cells.

The disclosed NOV9 nucleic acid of the invention encoding a CIP4-like protein includes the nucleic acid whose sequence is provided in Table 9A, or a fragment thereof. The invention also includes a mutant or variant nucleic acid any of whose bases may be changed from the corresponding base shown in Table 9A while still encoding a protein that maintains its CIP4-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 38% percent of the bases may be so changed.

The disclosed NOV9 protein of the invention includes the CIP4-like protein whose sequence is provided in Table 9B. The invention also includes a mutant or variant protein any of whose residues may be changed from the corresponding residue shown in Table 2 while still encoding a protein that maintains its CIP4-like activities and physiological functions, or a functional fragment thereof. In the mutant or variant protein, up to about 51% percent of the residues may be so changed.

The invention further encompasses antibodies and antibody fragments, such as F_{ab} or (F_{ab})₂ that bind immunospecifically to any of the proteins of the invention.

The above defined information for this invention suggests that this CIP4-like protein (NOV9) may function as a member of a “MT family”. Therefore, the NOV9 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV9 nucleic acids and proteins of the invention are useful in potential therapeutic applications implicated in cancer including but not limited to Inflamation, Autoimmune disorders, Aging and Cancer. For example, a cDNA encoding the CIP4-like protein (NOV9) may be useful in gene therapy, and the CIP4-like protein (NOV9) may be useful when administered to a subject in need thereof. By way of nonlimiting example, the compositions of the present invention will have efficacy for treatment of patients suffering from Wiskott-Aldrich syndrome, immunodeficiency, thrombocytopenia, eczema, lymphoid malignancy cytoskeletal abnormalities, impaired monocyte motility, Muscular dystrophy, Lesch-Nyhan syndrome, Myasthenia gravis, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Ataxia-telangiectasia, Leukodystrophies, Behavioral disorders, Addiction, Anxiety, Pain, Neuroprotection, Fertility, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Diabetes, Pancreatitis, Obesity, Systemic lupus erythematosus, Autoimmune disease, Asthma, Emphysema, Scleroderma, allergy, ARDS, Cirrhosis, Transplantation, Diabetes, Autoimmune disease, Renal artery stenosis, Interstitial nephritis, Glomerulonephritis, Polycystic kidney disease, Renal tubular acidosis, IgA nephropathy, Hypercalceimia, or other pathologies or conditions. The NOV9 nucleic acid encoding CIP4-like protein, and the CIP4-like protein of the invention, or fragments thereof, may further be useful in diagnostic applications, wherein the presence or amount of the nucleic acid or the protein are to be assessed.

NOV9 nucleic acids and polypeptides are further useful in the generation of antibodies that bind immuno-specifically to the novel NOV9 substances for use in therapeutic or diagnostic methods. These antibodies may be generated according to methods known in the art, using prediction from hydrophobicity charts, as described in the “Anti-NOVX Antibodies” section below. The disclosed NOV9 protein has multiple hydrophilic regions, each of which can be used as an immunogen. In one embodiment, a contemplated NOV9 epitope is from about amino acids 25 to 40. In another embodiment, a NOV9 epitope is from about amino acids 45 to 55. These novel proteins can be used in assay systems for functional analysis of various human disorders, which will help in understanding of pathology of the disease and development of new drug targets for various disorders.

NOV10

NOV10 includes three novel hepsin/plasma transmembrane serine protease-like proteins disclosed below. The disclosed sequences have been named NOV10a and NOV10b.

NOV10a

A disclosed NOV10a nucleic acid of 1787 nucleotides (also referred to as 129297354_EXT) encoding a novel hepsin/plasma transmembrane serine protease-like protein is shown in Table 10A. An open reading frame was identified beginning with an ATG initiation codon at nucleotides 54-56 and ending with a TAA codon at nucleotides 1470-1472. A putative untranslated region upstream from the initiation codon and downstream from the termination codon is underlined in Table 10A. The start and stop codons are in bold letters.

TABLE 10A
NOV10a nucleotide sequence

(SEQ ID NO:25)

GCGGAACATTGCCTAGTAGACCCTGAGGCTTTACAACAGTGCCACTGACCCCTATGAGCCTGATGCTGGATG
ACCAACCCCCTATGGAGGCCCAGTATGCAGAGGAGGGCCCAGGACCTGGGATCTTCAGAGCAGAGCCTGGAG
ACCAGCAGCATCCCATTTCTCAGGCGGTGTGCTGGCGTTCCATGCGACGTGGCTGTGCAGTGCTGGGAGCCC
TGGGGCTGCTGGCCGGTGCAGGTGTTGGCTCATGGCTCCTAGTGCTGTATCTGTGTCCTGCTGCCTCTCAGC
CCATTTCCGGGACCTTGCAGGATGAGGAGATAACTTTGAGCTGCTCAGAGGCCAGCGCTGAGGAAGCTCTGC
TCCCTGCACTTCCCAAAACAGTATCTTTCAGAATAAACAGCGAAGACTTCTTGCTGGAAGCGCAAGTGAGGG
ATCAGCCACGCTGGCTCCTGGTCTGCCATGAGGGCTGGAGCCCCGCCCTGGGGCTGCAGATCTGCTGGAGCC
TTGGGCATCTCAGACTCACTCACCACAAGGGAGTAAACCTCACTGACATCAAACTCAACAGTTCCCAGGAGT
TTGCTCAGCTCTCTCCTAGACTGGGAGGCTTCCTGGAGGAGGCGTGGCAGCCCAGTAGGACTACTGAGGCTG
TTAGGAACAACTGCACTTCTGGTCAAGTTGTTTCCCTCAGATGCTCTGAGTGTGGAGCGAGGCCCCTGGCTT
CCCGGATAGTTGGTGGGCAGTCTGTGGCTCCTGGGCGCTGGCCGTGGCAGGCCAGCGTGGCCCTGGGCTTCC
GGCACACGTGTGGGGGCTCTGTGCTAGCGCCACGCTGGGTGGTGACTGCTGCACATTGTATGCACAGTTTCA
GGCTGGCCCGCCTGTCCAGCTGGCGGGTTCATGCGGGGCTGGTCAGCCACAGTGCCGTCAGGCCCCACCAAG
GGGCTCTGGTGGAGAGGATTATCCCACACCCCCTCTACAGTGCCCAGAATCATGACTACGACGTCGCCCTCC
TGAGGCTCCAGACCGCTCTCAACTTCTCAGACACTGTGGGCGCTGTGTGCCTGCCGGCCAAGGAACAGCATT
TTCCGAAGGGCTCGCGGTGCTGGGTGTCTGGCTGGGGCCACACCCACCCTAGCCATACTTACAGCTCGGATA
TGCTCCAGGACACGGTGGTGCCCCTGCTCAGCACTCAGCTCTGCAACAGCTCTTGCGTGTACAGCGGAGCCC
TCACCCCCCGCATGCTTTGCGCTGGCTACCTGGACGGAAGGGCTGATGCATGCCAGGGAGATAGCGGGGGCC
CCCTAGTGTGCCCAGATGGGGACACATGGCGCCTAGTGGGGGTGGTCAGCTGGGGGCGTGGCTGCGCAGAGC
CCAATCACCCAGGTGTCTACGCCAAGGTAGCTGAGTTTCTGGACTGGATCCATGACACTGCTCAGGTGAGTG
TGGGGGCAGGAGTAGGGCAGGGAGATTTCTAAAGGACCTGCCCTCGAATGCAAGGAACCTTACCCCTTAGGC
CCGGGCCCTGCTGGGGACTGGGGAGGGTGCTAGGACATATTCCCCAGAGTGAGTGGAGGAAGAAGTGAAGCT
TAAACATGGAATCCATTGGATTTCTATCAGTTTAAGGATGAACTGGGTAAGAGTATGCCTGAGTTTGTATCC
CAGATCTACCATTTCCTGTGTCGACCTTTGGCAAATTTCTAACTTTGTTAAACCTTAATTTCCTGATAATAA
CCATGATGGCTACTTATATGCTATTGTTATATGCTATTAAATAAGACCCGTACAATGCC.

The reverse complement of NOV10a is shown in Table 10B.

TABLE 10B
NOV10a reverse complement sequence.

(SEQ ID NO:26)

GGCATTGTACGGGTCTTATTTAATAGCATATAACAATAGCATATAAGTAGCCATCATGGTTATTATCAGGAA
ATTAAGGTTTAACAAAGTTAGAAATTTGCCAAAGGTCGACACAGGAAATGGTAGATCTGGGATACAAACTCA
GGCATACTCTTACCCAGTTCATCCTTAAACTGATAGAAATCCAATGGATTCCATGTTTAAGCTTCACTTCTT
CCTCCACTCACTCTGGGGAATATGTCCTAGCACCCTCCCCAGTCCCCAGCAGGGCCCGGGCCTAAGGGGTAA
GGTTCCTTGCATTCGAGGGCAGGTCCTTTAGAAATCTCCCTGCCCTACTCCTGCCCCCACACTCACCTGAGC
AGTGTCATGGATCCAGTCCAGAAACTCAGCTACCTTGGCGTAGACACCTGGGTGATTGGGCTCTGCGCAGCC
ACGCCCCCAGCTGACCACCCCCACTAGGCGCCATGTGTCCCCATCTGGGCACACTAGGGGGCCCCCGCTATC
TCCCTGGCATGCATCAGCCCTTCCGTCCAGGTAGCCAGCGCAAAGCATGCGGGGGGTGAGGGCTCCGCTGTA
CACGCAAGAGCTGTTGCAGAGCTGAGTGCTGAGCAGGGGCACCACCGTGTCCTGGAGCATATCCGAGCTGTA
AGTATGGCTAGGGTGGGTGTGGCCCCAGCCAGACACCCAGCACCGCGAGCCCTTCGGAAAATGCTGTTCCTT
GGCCGGCAGGCACACAGCGCCCACAGTGTCTGAGAAGTTGAGAGCGGTCTGGAGCCTCAGGAGGGCGACGTC
GTAGTCATGATTCTGGGCACTGTAGAGGGGGTGTGGGATAATCCTCTCCACCAGAGCCCCTTGGTGGGGCCT
GACGGCACTGTGGCTGACCAGCCCCGCATGAACCCGCCAGCTGGACAGGCGGGCCAGCCTGAAACTGTGCAT
ACAATGTGCAGCAGTCACCACCCAGCGTGGCGCTAGCACAGAGCCCCCACACGTGTGCCGGAAGCCCAGGGC
CACGCTGGCCTGCCACGGCCAGCGCCCAGGAGCCACAGACTGCCCACCAACTATCCGGGAAGCCAGGGGCCT
CGCTCCACACTCAGAGCATCTGAGGGAAACAACTTGACCAGAAGTGCAGTTGTTCCTAACAGCCTCAGTAGT
CCTACTGGGCTGCCACGCCTCCTCCAGGAAGCCTCCCAGTCTAGGAGAGAGCTGAGCAAACTCCTGGGAACT
GTTGAGTTTGATGTCAGTGAGGTTTACTCCCTTGTGGTGAGTGAGTCTGAGATGCCCAAGGCTCCAGCAGAT
CTGCAGCCCCAGGGCGGGGCTCCAGCCCTCATGGCAGACCAGGAGCCAGCGTGGCTAGTCCCTCACTTGCGC
TTCCAGCAAGAAGTCTTCGCTGTTTATTCTGAAAGATACTGTTTTGGGAAGTGCAGGGAGCAGAGCTTCCTC
AGCGCTGGCCTCTGAGCAGCTCAAAGTTATCTCCTCATCCTGCAAGGTCCCGGAAATGGGCTGAGAGGCAGC
AGGACACAGATACAGCACTAGGAGCCATGAGCCAACACCTGCACCGGCCAGCAGCCCCAGGGCTCCCAGCAC
TGCACAGCCACGTCGCATGGAACGCCAGCACACCGCCTGAGAAATGGGATGCTGCTGGTCTCCAGGCTCTGC
TCTGAAGATCCCAGGTCCTGGGCCCTCCTCTGCATACTGGGCCTCCATAGGGGGTTGGTCATCCAGCATCAG
GCTCATAGGGGTCAGTGGCACTGTTGTAAAGCCTCAGGGTCTACTAGGCAATGTTCCGC

In a search of public sequence databases, the NOV10a nucleic acid sequence has 424 of 699 bases (60%) identical to a gb:GENBANK-ID:AF243500|acc:AF243500 transmembrane protein X mRNA, complete cds, 1735 bp. mRNA from mouse/ Mus musculus (E=4.9e⁻¹⁹). Public nucleotide databases include all GenBank databases and the GeneSeq patent database.

The disclosed NOV10a polypeptide (SEQ ID NO:27) encoded by SEQ ID NO:25 has 472 amino acid residues and is presented in Table 10B using the one-letter amino acid code. Signal P. Psort and/or Hydropathy results predict that NOV 10a has a signal peptide and is likely to be localized in the plasma membrane with a certainty of 0.7900. In other embodiments, NOV10a may also be localized to the microbody (peroxisome) with acertainty of 0.5425, the Golgi body with a certainty of 0.3000, or in the endoplasmic reticulum (membrane) with a certainty of 0.2000. The most likely cleavage site for a NOV10a peptide is between amino acids 13 and 14, at: MEA-QY.

TABLE 10B
Encoded NOV10a protein sequence.

(SEQ ID NO:27)

MSLMLDDQPPMEAQYAEEGPGPGIFRAEPGDQQHPISQAVCWRSMRRGCAVLGALGLLAGAGVGSWLLVLYL
CPAASQPISGTLQDEEITLSCSEASAEEALLPALPKTVSFRINSEDFLLEAQVRDQPRWLLVCHEGWSPALG
LQICWSLGHLRLTHHKGVNLTDIKLNSSQEFAQLSPRLGGFLEEAWQPSRTTEAVRNNCTSGQVVSLRCSEC
GARPLASRIVGGQSVAPGRWPWQASVALGFRHTCGGSVLAPRWVVTAAHCMHSFRLARLSSWRVHAGLVSHS
AVRPHQGALVERIIPHPLYSAQNHDYDVALLRLQTALNFSDTVGAVCLPAKEQHFPKGSRCWVSGWGHTHPS
HTYSSDMLQDTVVPLLSTQLCNSSCVYSGALTPRMLCAGYLDGRADACQGDSGGPLVCPDGDTWRLVGVVSW
GRGCAEPNHPGVYAKVAEFLDWIHDTAQVSVGAGVGQGDF

A search of sequence databases reveals that the NOV10a amino acid sequence has 130 of 346 amino acid residues (37%) identical to, and 190 of 346 amino acid residues (54%) similar to, the 417 amino acid residue Serine Protease Hepsin (EC 3.4.21.-) (Transmembrane Protease, Serine 1) protein from Homo sapiens (ptnr:SWISSPROT-ACC:PO₅₉₈₁ (E=3.5e⁻⁵⁶). Public amino acid databases include the GenBank databases, SwissProt, PDB and PIR.

NOV10a is expressed in at least the adrenal gland.

NOV10b

A disclosed NOV10b nucleic acid of 2148 nucleotides (also referred to as CG106783-02) encoding a novel Spinesin-like protein is shown in Table 10C. An open reading frame was identified beginning with an ATG initiation codon at nucleotides 156-158 and ending with a TGA codon at nucleotides 1410-1412. A putative untranslated region upstream from the initiation codon is underlined in Table 10C. The start and stop codons are in bold letters.

TABLE 10C
NOV10b nucleotide sequence.

(SEQ ID NO:28)

ACGCGGGATACAGGGAGGGGCCATGTGCGAACCAGGGAGACCTCATCTTCCAACCAAGCTTGCTGGGCTTGC
ATTTAATCAATGCATGGCCAGAGAACAGGAGCGGAACATTGCCTAGTAGACCCTGAGGCTTTACAACAGTGC
TACTGACCCCTATGAGCCTGATGCTGGATGACCAACCCCCTATGGAGGCCCAGTATGCAGAGGAGGGCCCAG
GACCTGGGATCTTCAGAGCAGAGCCTGGAGACCAGCAGCATCCCATTTCTCAGGCGGTGTGCTGGCGTTCCA
TGCGACGTGGCTGTGCAGTGCTGGGAGCCCTGGGGCTGCTGGCCGGTGCAGGTGTTGGCTCATGGCTCCTAG
TGCTGTATCTGTGTCCTGCTGCCTCTCAGCCCATTTCCGGGACCTTGCAGGATGAGGAGATAACTTTGAGCT
GCTCAGAGGCCAGCGCTGAGGAAGCTCTGCTCCCTGCACTCCCCAAAACAGTATCTTTCAGAATAAACAGCG
AAGACTTCTTGCTGGAAGCGCAAGTGAGGGATCAGCCACGCTGGCTCCTGGTCTGCCATGAGGGCTGGAGCC
CCGCCCTGGGGCTGCAGATCTGCTGGAGCCTTGGGCATCTCAGACTCACTCACCACAAGGGAGTAAACCTCA
CTGACATCAAACTCAACAGTTCCCAGGAGTTTGCTCAGCTCTCTCCTAGACTGGGAGGCTTCCTGGAGGAGG
CGTGGCAGCCCAGGAACAACTGCACTTCTGGTCAAGTTGTTTCCCTCAGATGCTCTGAGTGTGGAGCGAGGC
CCCTGGCTTCCCGGATAGTTGGTGGGCAGTCTGTGGCTCCTGGGCGCTGGCCGTGGCAGGCCAGCGTGGCCC
TGGGCTTCCGGCACACGTGTGGGGGCTCTGTGCTAGCGCCACGCTGGGTGGTGACTGCTGCACATTGTATGC
ACAGTGCCCAGAATCATGACTACGACGTCGCCCTCCTGAGGCTCCAGACCGCTCTCAACTTCTCAGACACTG
TGGGCGCTGTGTGCCTGCCGGCCAAGGAACAGCATTTTCCGAAGGGCTCGCGGTGCTGGGTGTCCGGCTGGT
GCCACACCCACCCTAGCCATACTTACAGCTCGGATATGCTCCAGGACACGGTGGTGCCCCTGCTCAGCACTC
AGCTCTGCAACAGCTCTTGCGTGTACAGCGGAGCCCTCACCCCCCGCATGCTTTGCGCTGGCTACCTGGACG
GAAGGGCTGATGCATGCCAGGGAGATAGCGGGGGCCCCCTAGTGTGCCCAGATGGGGACACATGGCGCCTAG
TGGGGGTGGTCAGCTGGGGGCGTGGCTGCGCAGAGCCCAATCACCCAGGTGTCTACGCCAAGGTAGCTGAGT
TTCTGGACTGGATCCATGACACTGCTCAGGACTCCCTCCTCTGAGTCCTGCTGTTTCCTCCAGTCTCACTGC
ACACCACTGCCTCATGCTTCCTGGGGCCTCCAGCAGCTCCACTAATGGAGGAGAGGCAGTAGCCTCCGACAC
AGAACGCATGGACCTCCTACTACTGTGTGTGAGGAACAGTCACTACCCACTGGCCAGCCACCCAGCCAACAG
GTCTCTCCTCTTGGGCCCTGATTTCAGAGTCCTCTTTCTCACTAGAGACTCAATGACAGAAGAGAGGCTGGG
ACTTGGTTGGGCATGCTGTGGTTGCTGAGGGATGAGGGGGAGGAGAGAGGTAGGAGCTGGAGATGAAGAGAC
TGCTAGAAGCAGCAGGAAGCCTGCCCTTCTGCCCTCTCCCCTCCCTGCCCCTGTGTGAGTCTTTTAGGGAGG
GTGACTGGGAGGTGCCCCCCGTCCCACCTTTTTCCTGTGCTCTAGGTGGGCTAAGTGCCTCCCTAGAGGACT
CCATGGCTGAGAGGCTCCTGGGCAGATGGGGTCAAGGCTGGGCCAGTCCCAGATGAAGCCTATGGGAGTCAG
GACCCTCTCCACTCTCCCTCTCCACTCCCCTTCCTGTTCTCACCTGGCTGTGGCTGGCCCTGTGTGGGGTGG
GTACACTGGAAAACAAGAAGGTTGGAGTTGGTCTAGGACATTGGTTTTAAATGACAGTTCTGTGAACTGGTC
CAAGGAGGTTCTGTTATTAAAGTGATATATGGTCTTGAAAAAAAAAAAAAAAAAAAAAAA

In a search of public sequence databases, the NOV10b nucleic acid sequence, located on chromosome 7 has 1343 of 1446 bases (92%) identical to a gb:GENBANK-ID:ABo28140|acc:AB028140.1 mRNA from Homo sapiens (Homo sapiens mRNA for spinesin, complete cds) (E=3.2e⁻²⁶⁸). Public nucleotide databases include all GenBank databases and the GeneSeq patent database.

The disclosed NOV10b polypeptide (SEQ ID NO:29) encoded by SEQ ID NO:28 has 418 amino acid residues and is presented in Table 10D using the one-letter amino acid code. Signal P, Psort and/or Hydropathy results predict that NOV10a has a signal peptide and is likely to be localized in the plasma membrane with a certainty of 0.7900. In other embodiments, NOV10a may also be localized to the Golgi body with acertainty of 0.3000, the microbody (peroxisome) with a certainty of 0.2036 or in the endoplasmic reticulum (membrane) with a certainty of 0.2000. The most likely cleavage site for a NOV 10a peptide is between amino acids 13 and 14, at: MEA-QY.

TABLE 10D
Encoded NOV10a protein sequence.

(SEQ ID NO:29)

MSLMLDDQPPMEAQYAEEGPGPGIFRAEPGDQQHPISQAVCWRSMRRGCAVLGALGLLAGAGVGSWLLVLYL
CPAASQPISGTLQDEEITLSCSEASAEEALLPALPKTVSFRINSEDFLLEAQVRDQPRWLLVCHEGWSPALG
LQICWSLGHLRLTHHKGVNLTDIKLNSSQEFAQLSPRLGGFLEEAWQPRNNCTSGQVVLSRCSECGARPLAS
RIVGGQSVAPGRWPWQASVALGFRHTCGGSVLAPRWVVTAAHCMHSAQNHDYDVALLRLQTALNFSDTVGAV
CLPAKEQHFPKGSRCWVSGWCHTHPSHTYSSDMLQDTVVPLLSTQLCNSSCVYSGALTPRMLCAGYLDGRAD
ACQGDSGGPLVCPDGDTWRLVGVVSWGRGCAEPNHPGVYAKVAEFLDWIHDTAQDSLL

A search of sequence databases reveals that the NOV 10b amino acid sequence has 262 of 262 amino acid residues (100%) identical to, and 262 of 262 amino acid residues (100%) similar to, the 457 amino acid residue ptnr:SWISSNEW-ACC:Q9H3S3 protein from Homo sapiens (Human) (Transmembrane protease, serine 5 (EC 3.4.21.-) (Spinesin) (E=8.7e⁻²³³). Public amino acid databases include the GenBank databases, SwissProt, PDB and PIR.

NOV 10b is expressed in at least the following tissues: Colon, Brain, Placenta, Testis, Adrenal Gland/Suprarenal gland, Retina. Expression information was derived from the tissue sources of the sequences that were included in the derivation of the sequence of NOV10b. The sequence is predicted to be expressed in the following tissues because of the expression pattern of (GENBANK-ID: gb:GENBANK-ID:AB028140|acc:AB028140.1) a closely related Homo sapiens mRNA for spinesin, complete cds homolog in species Homo sapiens: brain.

TaqMan data for NOV10a can be found below in Example 2. The proteins encoded by the NOV10a and 10b nucleotides are very closely homologous as is shown in the alignment in Table 10E.

[image]

[image]

Homologies to any of the above NOV10 proteins will be shared by the other NOV 10 protein insofar as they are homologous to each other as shown above. Any reference to NOV10 is assumed to refer to both of the NOV10 proteins in general, unless otherwise noted.

The disclosed NOV10a polypeptide has homology to the amino acid sequences shown in the BLASTP data listed in Table 10F.

TABLE 10F
BLAST results for NOV10a

| Gene Index/ | | Length | Identity | Positives | |
|---|---|---|---|---|---|
| Identifier | Protein/Organism | (aa) | (%) | (%) | Expect |
| gi\|13540535\|ref\|NP_{—} | transmembrane | 457 | 452/463 | 453/463 | 0.0 |
| 110397.1\| | protease, serine | | (97%) | (97%) | |
| | 5; spinesin [Homo | | | | |
| | sapiens] | | | | |
| gi\|13878822\|sp\|Q9ER04\| | TRANSMEMBRANE | 455 | 360/461 | 394/461 | 0.0 |
| TMS5_MOUSE | PROTEASE, SERINE 5 | | (78%) | (85%) | |
| | (SPINESIN) | | | | |
| gi\|13507652\|ref\|NP_{—} | transmembrane | 445 | 354/451 | 388/451 | 0.0 |
| 109634.1\| | protease, serine 5 | | (78%) | (85%) | |
| | (spinesin) [Mus | | | | |
| | musculus] | | | | |
| gi\|14770563\|ref\|XP_{—} | transmembrane | 398 | 354/362 | 355/362 | 0.0 |
| 041427.1\| | protease, serine 5 | | (97%) | (97%) | |
| | [Homo sapiens] | | | | |
| gi\|12248777\|dbj\| | type 2 spinesin | 311 | 260/317 | 281/317 | e−146 |
| BAB20276.1\| (AB016229) | [Mus musculus] | | (82%) | (88%) | |

The homology between these and other sequences is shown graphically in the ClustalW analysis shown in Table 10G. In the ClustalW alignment of the NOV10 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Tables 1H-1I lists the domain description from DOMAIN analysis results against NOV10. This indicates that the NOV10 sequence has properties similar to those of other proteins known to contain this domain.

TABLE 10H
Domain Analysis of NOV10

gnl|Smart|smart00020, Tryp_SPc, Trypsin-like serine protease;
Many of these are synthesised as inactive precursor zymogens that are cleaved
during limited proteolysis to generate their active forms. A few,
however, are active as single chain molecules, and others are inactive
due to substitutions of the catalytic triad residues. (SEQ ID NO:96)
CD-Length = 230 residues, 100.0% aligned
Score = 266 bits (681), Expect = 2e−72

| | | | |
|---|---|---|---|
| Query: | 224 | RIVGGQSVAPGRWPWQASVAL-GFRHTCGGSVLAPRWVVTAAHCMHSFRLARLSSWRVHA | 282 |
| | | \|\|\|\|\| \| +\|\|\| \|+ \| \|\| \|\|\|\|+++\|\|\|\|+\|\|\|\|\|++ \|\| \|\| | |
| Sbjct: | 1 | RIVGGSEANIGSFPWQVSLQYRGGRHFCGGSLISPRWVLTAAHCVYGSAP---SSIRVRL | 57 |
| Query: | 283 | GLVSHSAVRPHQGALVERIIPHPLYSAQNHDYDVALLRLQTALNFSDTVGAVCLPAKEQH | 342 |
| | | \| \|+ \| \| ++\| \|\| \|+ +\| \|+\|\|\|+\| + \|\|\|\| +\|\|\|+ + | |
| Sbjct: | 58 | GSHDLSSGEETQTVKVSKVIVHPNYNPSTYDNDIALLKLSEPVTLSDTVRPICLPSSGYN | 117 |
| Query: | 343 | FPKGSRCWVSGWGHTHPSHTYSSDMLQDTVVPLLSTQLCNSSCVYSGALTPRMLCAGYLD | 402 |
| | | \| \|+ \| \|\|\|\|\| \| \| \| \|\|+ \|\|++\| \| + \|+\| \|\|\|\|\| \|+ | |
| Sbjct: | 118 | VPAGTTCTVSGWGRTSESSGSLPDTLQEVNVPIVSNATCRRAYSGGPAITDNMLCAGGLE | 177 |
| Query: | 403 | GRADACQGDSGGPLVCPDGDTWRLVGVVSWGR-GCAEPNHPGVYAKVAEFLDWI 455 | |
| | | \| \|\|\|\|\|\|\|\|\|\|\|\|\| + \| \|\|\|+\|\|\|\| \|\|\| \|\| \|\|\|\| +\|+ +\|\|\|\| | |
| Sbjct: | 178 | GGKDACQGDSGGPLVC-NDPRWVLVGIVSWGSYGCARPNKPGVYTRVSSYLDWI 230 | |

TABLE 10I
Domain Analysis of NOV10

gnl|Pfam|pfam00089, trypsin, Trypsin. Proteins recognized include all
proteins in families S1, S2A, S2B, S2C, and S5 in the classification
of peptidases. Also included are proteins that are clearly members,
but that lack peptidase activity, such as haptoglobin and protein Z
(PRTZ^{*}). (SEQ ID NO:97)
CD-Length = 217 residues, 100.0% aligned
core = 211 bits (538), Expect = 6e−56

| | | | |
|---|---|---|---|
| Query: | 225 | IVGGQSVAPGRWPWQASVALGFRHTCGGSVLAPRWVVTAAHCMHSFRLARLSSWRVHAGL | 284 |
| | | \|\|\|\|+ \| +\|\|\| \|+ + \| \|\|\|\|+++ \|\|+\|\|\|\|\|+ \|\| \|\| \| | |
| Sbjct: | 1 | IVGGREAQAGSFPWQVSLQVSSGHFCGGSLISENWVLTAAHCVSG-----ASSVRVVLGE | 55 |
| Query: | 285 | VSHSAVRPH-QGALVERIIPHPLYSAQNHDYDVALLRLQTALNFSDTVGAVCLPAKEQHF | 343 |
| | | + \| \|++\|\| \|\| \|+ \|+\|\|\|+\|++ + \|\|\| +\|\|\|+ | |
| Sbjct: | 561 | HNLGTTEGTEQKFDVKKIIVHPNYNPDT--NDIALLKLKSPVTLGDTVRPICLPSASSDL | 113 |
| Query: | 344 | PKGSRCWVSGWGHTHPSHTYSSDMLQDTVVPLLSTQLCNSSCVYSGALTPRMLCAGYLDG | 403 |
| | | \| \|+ \| \|\|\|\|\| \| + +\|\| \|\|+ \|\|\|++\| + \| \|+ \| \| +\| \|+\|\|\| \| \| | |
| Sbjct: | 114 | PVGTTCSVSGWGRTK--NLGTSDTLQEVVVPIVSRETCRSA--YGGTVTDTMICAGALGG | 169 |
| Query: | 404 | RADACQGDSGGPLVCPDGDTWRLVGVVSWGRGCAEPNHPGVYAKVAEFLDWI 455 | |
| | | + \|\|\|\|\|\|\|\|\|\|\|\|\| \|\| \|\|\|+\|\|\|\| \|\|\| \|+\|\|\|\| +\|+ +\|\|\|\| | |
| Sbjct: | 170 | K-DACQGDSGGPLVCSDG---ELVGIVSWGYGCAVGNYPGVYTRVSRYLDWI 217 | |

Failure to thrive, nutritional edema, and hypoproteinemia with normal sweat electrolytes were features of 2 affected male infants reported by Townes (1965) and Townes et al. (1967). A protein hydrolysate diet was beneficial. A male sib of the first patient reported by Townes (1965) had died, apparently of the same condition. Morris and Fisher (1967) reported an affected female who also had imperforate anus. The clinical picture in enterokinase deficiency (226200) is closely similar; however, the defect is not in the synthesis of trypsinogen but in the synthesis of the enterokinase which activates proteolytic enzymes produced by the pancreas. Oral pancreatin represents a therapeutically successful form of enzyme replacement (Townes, 1972). Trypsin (EC 3.4.21.4), like elastase (130120), is a member of the pancreatic family of serine proteases. MacDonald et al. (1982) reported nucleotide sequences of cDNAs representing 2 pancreatic rat trypsinogens. Using a rat cDNA probe, Honey et al. (1984, 1984) found that a 3.8-kb DNA fragment containing human trypsin-1 gene sequences cosegregated with chromosome 7, and assigned the gene further to 7q22-7qter by study of hybrids with a deletion of this segment. The trypsin gene is on mouse chromosome 6 (Honey et al., 1984). Carboxypeptidase A (114850) and trypsin are a syntenic pair conserved in mouse and man. Emi et al. (1986) isolated cDNA clones for 2 major human trypsinogen isozymes from a pancreatic cDNA library. The deduced amino acid sequences had 89% homology and the same number of amino acids (247), including a 15-amino acid signal peptide and an 8-amino acid activation peptide. Southern blot analysis of human genomic DNA with the cloned cDNA as a probe showed that the human trypsinogen genes constitute a family of more than 10, some of which may be pseudogenes or may be expressed in other stages of development.

Rowen et al. (1996) found that there are 8 trypsinogen genes embedded in the beta T-cell receptor locus or cluster of genes (TCRB; 186930) mapping to 7q35. In the 685-kb DNA segment that they sequenced they found 5 tandemly arrayed 10-kb locus-specific repeats (homology units) at the 3-prime end of the locus. These repeats exhibited 90 to 91% overall nucleotide similarity, and embedded within each is a trypsinogen gene. Alignment of pancreatic trypsinogen cDNAs with the germline sequences showed that these trypsinogen genes contain 5 exons that span approximately 3.6 kb. Further analyses revealed 2 trypsinogen pseudogenes and 1 relic trypsinogen gene at the 5-prime end of the sequence, all in inverted transcriptional orientation. They denoted 8 trypsinogen genes T1 through T8 from 5-prime to 3-prime.

Rowen et al. (1996) found that only 2 of 3 pancreatically expressed trypsinogen cDNAs correspond to trypsinogen genes in the TCRB locus; T4 was denoted trypsinogen 1 and T8 was denoted trypsinogen 2 (601564). The third pancreatic cDNA, identified independently as trypsinogen 3 (Tani et al., 1990) and 4 (Wiegand et al., 1993), is distinct from the third apparently functional trypsinogen gene (T6) in the TCRB locus but related to the other pancreatic trypsinogens. Rowen et al. (1996) stated that the T6 gene is deleted in a common insertion-deletion polymorphism; if it is functional, its function is apparently not essential. Some of the trypsinogen genes are expressed in nonpancreatic tissues where their function is unknown. Rowen et al. (1996) noted that the intercalation of the trypsinogen genes in the TCRB locus is conserved in mouse and chicken, suggesting shared functional or regulatory constraints, as has been postulated for genes in the major histocompatibility complex (such as class I, II, and III genes) that share similar long-term organizational relationships.

Rowen et al. (1996) mapped the gene corresponding to the third pancreatic trypsinogen cDNA by fluorescence in situ hybridization. They used a cosmid clone containing 3 trypsinogen genes. Strong hybridization to chromosome 7 and weaker hybridization to chromosome 9 were observed. They isolated and partially sequenced 4 cosmid clones from the chromosome 9 region. They found that the region represents a duplication and translocation of a DNA segment from the 3-prime end of the TCRB locus that includes at least 7 V(beta) elements and a functional trypsinogen gene denoted T9. The assignment of the PRSS 1 gene to 7q35 is established by the demonstration of its sequence within the sequence of the ‘locus’ for the T-cell receptor beta-chain (Rowen et al., 1996). Since hereditary pancreatitis (167800) has been mapped rather precisely to 7q35 and since a defect in the trypsinogen gene has been identified in hereditary pancreatitis, the assignment of the trypsinogen gene can be refined from 7q32-qter to 7q35.

Whitcomb et al. (1996) stated that the high degree of DNA sequence homology (more than 91%) present among this cluster of 5 trypsinogen genes identified by Rowen et al. (1996) demanded that highly specific sequence analysis strategies be developed for mutational screening in families with hereditary pancreatitis. This was necessary to ensure that each sequencing run contained only the 2 alleles corresponding to a single gene, thereby permitting detection of heterozygotes in this autosomal dominant disorder, and not a dozen or more alleles from multiple related trypsinogen-like genes, which would make detection of heterozygotes nearly impossible. In a family with hereditary pancreatitis, Whitcomb et al. (1996) found that affected individuals had a single G-to-A transition mutation in the third exon of cationic trypsinogen (276000.0001). This mutation was predicted to result in an arg105-to-his substitution in the trypsin gene (residue number 117 in the more common chymotrypsin number system). Subsequently, the same mutation was found in a total of 5 different hereditary pancreatitis kindreds (4 from the U.S. and 1 from Italy) containing a total of 20 affected individuals and 6 obligate carriers. The mutation was found in none of the obligate unaffected members (individuals who married into the family). Subsequent haplotyping revealed that all 4 of the American families displayed the same high risk haplotype over a 4-cM region encompassing 7 STR markers, confirming the likelihood that these kindreds shared a common ancestor, although no link could be found through 8 generations. A fifth family from Italy displayed a unique haplotype indicating that the same mutation had occurred on at least 2 occasions. The G-to-A mutation at codon 117 created a novel enzyme recognition site for AflIII which provided a facile means to screen for the mutation. As with the obligate unaffected members of the pancreatitis kindreds, none of 140 controls possessed the G-to-A mutation as assayed by the lack of AflIII digestion of the amplified exonic DNA.

Ferec et al. (1999) studied 14 families with hereditary pancreatitis and found mutations in the PRSS1 gene in 8 families. In 4 of these families, the mutation (R117H; 276000.0001) had been described by Whitcomb et al. (1996). Three mutations were described in 4 other families (276000.0002, 276000.0003, 276000.0005).

Sahin-Toth et al. (1999) studied the roles of the 2 most frequent PRSS1 mutations in hereditary pancreatitis, R117H and N211 (276000.0002). They stated that the R117H mutation is believed to cause pancreatitis by eliminating an essential autolytic cleavage site in trypsin, thereby rendering the protease resistant to inactivation through autolysis. Sahin-Toth et al. (1999) demonstrated that the R117H mutation also significantly inhibited autocatalytic trypsinogen breakdown under Ca(2+)-free conditions and stabilized the zymogen form of rat trypsin. Taken together with findings demonstrating that the N21I mutation stabilized rat trypsinogen against

autoactivation and consequent autocatalytic degradation, the observations suggested a unifying molecular pathomechanism for hereditary pancreatitis in which zymogen stabilization plays a central role.

Sahin-Toth and Toth (2000) demonstrated that the R 117H and N21I mutations significantly enhance autoactivation of human cationic trypsinogen in vitro, in a manner that correlates with the severity of clinical symptoms in hereditary pancreatitis. In addition, the R117H mutation inhibited autocatalytic inactivation of trypsin, while the N21I mutation had no such effect. Thus, increased trypsinogen activation in the pancreas is presumably the common initiating step in both forms of hereditary pancreatitis, whereas trypsin stabilization may also contribute to hereditary pancreatitis associated with the R117H mutation.

The disclosed NOV10 nucleic acid of the invention encoding a spinesin-like protein includes the nucleic acid whose sequence is provided in Table 10A and 10D or a fragment thereof. The invention also includes a mutant or variant nucleic acid any of whose bases may be changed from the corresponding base shown in Table 10A and 10D while still encoding a protein that maintains its spinesin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 40% percent of the bases may be so changed.

The disclosed NOV10 protein of the invention includes the spinesin-like protein whose sequence is provided in Table 10B or 10E. The invention also includes a mutant or variant protein any of whose residues may be changed from the corresponding residue shown in Table 10B or 10E while still encoding a protein that maintains its spinesin-like activities and physiological functions, or a functional fragment thereof. In the mutant or variant protein, up to about 22% percent of the residues may be so changed.

The invention further encompasses antibodies and antibody fragments, such as F_{ab} or (F_{ab})₂, that bind immunospecifically to any of the proteins of the invention.

The above defined information for this invention suggests that this spinesin-like protein (NOV10) may function as a member of a “spinesin family”. Therefore, the NOV10 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV10 nucleic acids and proteins of the invention are useful in potential therapeutic applications implicated in cancer including but not limited to various pathologies and disorders as indicated below. For example, a cDNA encoding the spinesin-like protein (NOV10) may be useful in gene therapy, and the spinesin-like protein (NOV10) may be useful when administered to a subject in need thereof. By way of nonlimiting example, the compositions of the present invention will have efficacy for treatment of patients suffering from cancer, trauma, tissue regeneration (in vitro and in vivo), viral/bacterial/parasitic infections, immunological disease, respiratory disease, gastro-intestinal diseases, reproductive health, neurological and neurodegenerative diseases, bone marrow transplantation, metabolic and endocrine diseases, allergy and inflammation, nephrological disorders, cardiovascular diseases, muscle, bone, joint and skeletal disorders, hematopoietic disorders, urinary system disorders, Tissue and organ transplantation, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Scleroderma, Obesity, Hypertension, Fibromuscular dysplasia, Stroke, Aneurysm, Myocardial infarction, Embolism, Bypass surgery, Anemia, Bleeding disorders, Adrenoleukodystrophy, Congenital Adrenal Hyperplasia, Diabetes, Von Hippel-Lindau (VHL) syndrome, Pancreatitis, Hyperparathyroidism, Hypoparathyroidism, Hyperthyroidism and Hypothyroidism, SIDS, Endometriosis, infertility, Xerostomia, Hypercalceimia, Ulcers, Cirrhosis, Inflammatory bowel disease, Diverticular disease, Hirschsprung's disease, Crohn's Disease, Appendicitis, Hemophilia, hypercoagulation, autoimmume disease, allergies, immunodeficiencies, transplantation, Graft vesus host disease (GVHD), Ataxia-telangiectasia, Autoimmume disease, Hemophilia, Hypercoagulation, Idiopathic thrombocytopenic purpura, Immunodeficiencies, Lymphedema, Allergies, Hemophilia, hypercoagulation, idiopathic thrombocytopenic purpura, Lymphaedema, Tonsilitis, Osteoporosis, Hypercalceimia, Arthritis, Ankylosing spondylitis, Scoliosis, Tendinitis, Muscular dystrophy, Lesch-Nyhan syndrome, Myasthenia gravis, Dental disease and infection, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Behavioral disorders, Addiction, Anxiety, Pain, Neuroprotection, Endocrine dysfunctions, Growth and reproductive disorders, Myasthenia gravis, Leukodystrophies, Pain, Neuroprotection, Systemic lupus erythematosus, Autoimmune disease, Emphysema, Scleroderma, ARDS, Pharyngitis, Laryngitis, Asthma, Hearing loss, Tinnitus, Psoriasis, Actinic keratosis, Tuberous sclerosis, Acne, Hair growth, allopecia, pigmentation disorders, endocrine disorders, cystitis, incontinence, Autoimmune disease, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome, Vesicoureteral reflux, and other pathologies and conditions. The NOV10 nucleic acid encoding the spinesin-like protein of the invention, or fragments thereof, may further be useful in diagnostic applications, wherein the presence or amount of the nucleic acid or the protein are to be assessed.

NOV10 nucleic acids and polypeptides are further useful in the generation of antibodies that bind immuno-specifically to the novel NOV10 substances for use in therapeutic or diagnostic methods. These antibodies may be generated according to methods known in the art, using prediction from hydrophobicity charts, as described in the “Anti-NOVX Antibodies” section below. The disclosed NOV 10 protein has multiple hydrophilic regions, each of which can be used as an immunogen. In one embodiment, a contemplated NOV10 epitope is from about amino acids 5 to 50. In another embodiment, a NOV10 epitope is from about amino acids 60 to 70. In additional embodiments, NOV10 epitope is from about amino acids 100 to 130, from about amino acids 140 to 210, from about amino acid 270 to 320, from about amino acid 340 to 360, from about amino acid 390 to 410, and from about amino acids 430 to 450. These novel proteins can be used in assay systems for functional analysis of various human disorders, which will help in understanding of pathology of the disease and development of new drug targets for various disorders.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS:1, 3, 5, 7, 9, 11, 13, 15, 17, 19, 21, 23, 25, and 28, or a complement of this aforementioned nucleotide sequence, can be isolated using standard molecular biology techniques and the sequence information provided herein. Using all or a portion of the nucleic acid sequence of SEQ ID NOS: 1, 3, 5, 7, 9, 11, 13, 15, 17, 19, 21, 23, 25, and 28 as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS:1, 3, 5, 7, 9, 11, 13, 15, 17, 19, 21, 23, 25, and 28, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:1, 3, 5, 7, 9, 11, 13, 15, 17, 19, 21, 23, 25, and 28, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NOS:1, 3, 5, 7, 9, 11, 13, 15, 17, 19, 21, 23, 25, or 28 is one that is sufficiently complementary to the nucleotide sequence shown SEQ ID NOS: 1, 3, 5, 7, 9, 11, 13, 15, 17, 19, 21, 23, 25, or 28 that it can hydrogen bond with little or no mismatches to the nucleotide sequence shown SEQ ID NOS:1, 3, 5, 7, 9, 11, 13, 15, 17, 19, 21, 23, 25, and 28, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS:1, 3, 5, 7, 9, 11, 13, 15, 17, 19, 21, 23, 25, and 28, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS: 1, 3, 5, 7, 9, 11, 13, 15, 17, 19, 21, 23, 25, or 28; or an anti-sense strand nucleotide sequence of SEQ ID NOS:1, 3, 5, 7, 9, 11, 13, 15, 17, 19, 21, 23, 25, or 28; or of a naturally occurring mutant of SEQ ID NOS:1, 3, 5, 7, 9, 11, 13, 15, 17, 19, 21, 23, 25, and 28.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS:1, 3, 5, 7, 9, 11, 13, 15, 17, 19, 21, 23, 25, or 28, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS:1, 3, 5, 7, 9, 11, 13, 15, 17, 19, 21, 23, 25, and 28 due to degeneracy of the genetic code and thus encode the same NOVX proteins as that encoded by the nucleotide sequences shown in SEQ ID NOS:1, 3, 5, 7, 9, 11, 13, 15, 17, 19, 21, 23, 25, and 28. In another embodiment, an isolated nucleic acid molecule of the invention has a nucleotide sequence encoding a protein having an amino acid sequence shown in SEQ ID NOS:2, 4, 6, 8, 10, 12, 14, 16, 18, 20, 22, 24, 27, or 29.

In addition to the human NOVX nucleotide sequences shown in SEQ ID NOS: 1, 3, 5, 7, 9, 11, 13, 15, 17, 19, 21, 23, 25, and 28, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS:1, 3, 5, 7, 9, 11, 13, 15, 17, 19, 21, 23, 25, and 28 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:1, 3, 5, 7, 9, 11, 13, 15, 17, 19, 21, 23, 25, and 28. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6× SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS:1, 3, 5, 7, 9, 11, 13, 15, 17, 19, 21, 23, 25, and 28, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:1, 3, 5, 7, 9, 11, 13, 15, 17, 19, 21, 23, 25, and 28, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NOS:1, 3, 5, 7, 9, 11, 13, 15, 17, 19, 21, 23, 25, and 28, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS:1, 3, 5, 7, 9, 11, 13, 15, 17, 19, 21, 23, 25, and 28, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS:2, 4, 6, 8, 10, 12, 14, 16, 18, 20, 22, 24, 27, or 29. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS:1, 3, 5, 7, 9, 11, 13, 15, 17, 19, 21, 23, 25, and 28 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS:2, 4, 6, 8, 10, 12, 14, 16, 18, 20, 22, 24, 27, and 29. Preferably, the protein encoded by the nucleic acid molecule is at least about 60% homologous to SEQ ID NOS:2, 4, 6, 8, 10, 12, 14, 16, 18, 20, 22, 24, 27, and 29; more preferably at least about 70% homologous SEQ ID NOS:2, 4, 6, 8, 10, 12, 14, 16, 18, 20, 22, 24, 27, or 29; still more preferably at least about 80% homologous to SEQ ID NOS:2, 4, 6, 8, 10, 12,14, 16, 18, 20, 22, 24, 27, or 29; even more preferably at least about 90% homologous to SEQ ID NOS:2, 4, 6, 8, 10, 12, 14, 16, 18, 20, 22, 24, 27, or 29; and most preferably at least about 95% homologous to SEQ ID NOS:2, 4, 6, 8, 10, 12, 14, 16, 18, 20, 22, 24, 27, or 29.

An isolated nucleic acid molecule encoding an NOVX protein homologous to the protein of SEQ ID NOS:2, 4, 6, 8, 10, 12, 14, 16, 18, 20, 22, 24, 27, or 29 can be created by introducing one or more nucleotide substitutions, additions or deletions into the nucleotide sequence of SEQ ID NOS:1, 3, 5, 7, 9, 11, 13, 15, 17, 19, 21, 23, 25, and 28, such that one or more amino acid substitutions, additions or deletions are introduced into the encoded protein.

Mutations can be introduced into SEQ ID NOS:1, 3, 5, 7, 9, 11, 13, 15, 17, 19, 21, 23, 25, and 28 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS:1, 3, 5, 7, 9, 11, 13, 15, 17, 19, 21, 23, 25, and 28, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SOND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS: 1, 3, 5, 7, 9, 11, 13, 15, 17, 19, 21, 23, 25, and 28, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS:2, 4, 6, 8, 10, 12, 14, 16, 18, 20, 22, 24, 27, or 29, or antisense nucleic acids complementary to an NOVX nucleic acid sequence of SEQ ID NOS:1, 3, 5, 7, 9, 11, 13, 15, 17, 19, 21, 23, 25, and 28, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong po II or pol III promoter are preferred.

In yet another embodiment, the antisense nucleic acid molecule of the invention is an α-anomeric nucleic acid molecule. An x-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS: 1, 3, 5, 7, 9, 11, 13, 15, 17, 19, 21, 23, 25, and 28). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS:2, 4, 6, 8, 10, 12, 14, 16, 18, 20, 22, 24, 27, or 29. The invention also includes a mutant or variant protein any of whose residues may be changed from the corresponding residues shown in SEQ ID NOS:2, 4, 6, 8, 10, 12, 14, 16, 18, 20, 22, 24, 27, or 29 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS:2, 4, 6, 8, 10, 12, 14, 16, 18, 20, 22, 24, 27, or 29) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS:2, 4, 6, 8, 10, 12, 14, 16, 18, 20, 22, 24, 27, or 29. In other embodiments, the NOVX protein is substantially homologous to SEQ ID NOS:2, 4, 6, 8, 10, 12, 14, 16, 18, 20, 22, 24, 27, or 29, and retains the functional activity of the protein of SEQ ID NOS:2, 4, 6, 8, 10, 12, 14, 16, 18, 20, 22, 24, 27, or 29, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS:2, 4, 6, 8, 10, 12, 14, 16, 18, 20, 22, 24, 27, or 29, and retains the functional activity of the NOVX proteins of SEQ ID NOS:2, 4, 6, 8, 10, 12, 14, 16, 18, 20, 22, 24, 27, or 29.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS: 1, 3, 5, 7, 9, 11, 13, 15, 17, 19, 21, 23, 25, and 28.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS:2, 4, 6, 8, 10, 12, 14, 16, 18, 20, 22, 24, 27, or 29, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termin, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an Fab expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG1, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of Fab expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an Fab fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol comProhibiting agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγR11 (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (Mx-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, P-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I , ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET lid (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS:1, 3, 5, 7, 9, 11, 13, 15, 17, 19, 21, 23, 25, and 28 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS:1, 3, 5, 7, 9, 11, 13, 15, 17, 19, 21, 23, 25, and 28), but more preferably, is a non-human homologue of a human NOVX gene. For example, a mouse homologue of human NOVX gene of SEQ ID NOS:1, 3, 5, 7, 9, 11, 13, 15, 17, 19, 21, 23, 25, and 28 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl—N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS:1, 3, 5, 7, 9, 11, 13, 15, 17, 19, 21, 23, 25, and 28, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS: 1, 3, 5, 7, 9, 11, 13, 15, 17, 19, 21, 23, 25, and 28 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS:1, 3, 5, 7, 9, 11, 13, 15, 17, 19, 21, 23, 25, and 28, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qp Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313. 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6:1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-termninus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof, (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example 1

### Identification of NOVX Clones

The novel NOVX target sequences identified in the present invention were subjected to the exon linking process to confirm the sequence. PCR primers were designed by starting at the most upstream sequence available, for the forward primer, and at the most downstream sequence available for the reverse primer. Table 11 A shows the sequences of the PCR primers used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. Table 17B shows a list of these bacterial clones. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

TABLE 11A

PCR Primers for Exon Linking

| | | | | |
|---|---|---|---|---|
| | | SEQ | | SEQ |
| NOVX | | ID | | ID |
| Clone | Primer 1 (5′-3′) | NO | Primer 2 (5′-3′) | NO |

| | | | | |
|---|---|---|---|---|
| NOV5 | CTCCCACTCCTGCTGCTTCTGACT | 98 | AAGGCTGGGCCTAACCCAGTCTCAT | 99 |
| NOV7 | CATGAACTGGGCATTTCTGCAGG | 100 | TTATCTGCTGATCTCGCAGGTTATGGA | 101 |
| NOV8 | CTGACAGGCCCTGGTGTGTGAT | 102 | TCACACATGTTTCATGTGGGAGTTAGA | 103 |
| NOV9 | GAGTGAGAGGTCGGACAGACTGTG | 104 | ACTCATGCAACTTGCTTCTCTCACTCT | 105 |
| NOV10b | CCTATGAGCCTGATGCTGGATGAC | 106 | AGGACTCAGAGGAGGGAGTCCTGAG | 107 |

Physical clone: Exons were predicted by homology and the intron/exon boundaries were determined using standard genetic rules. Exons were further selected and refined by means of similarity determination using multiple BLAST (for example, tBlastN, BlastX, and 5 BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

TABLE 11B
Physical Clones for PCR products

| NOVX Clone | Bacterial Clone |
|---|---|
| NOV1a | Physical clone: AC010269 |
| NOV1b | Physical clone: 137043926, 138213196, AC010269.5 |
| NOV1d | Physical clone: 168392429, 138213193 |
| NOV2 | Physical clone: 78316254, 123164361 |
| NOV3 | Physical clone: GMAC079237.2 |
| NOV4 | Physical clone: AP002795.2, AL161453 |
| NOV9 | Bacterial clone: sggc_draft_{—} |
| | ba327p22_20000819.698237.A7 |
| NOV10a | Physical clone: 129297354, AC068190.2, |
| | AC036188.2, AC002436.1 |
| NOV10b | Physical clone: 206164528, 206184919, |
| | 165210772, 206764887, 162395636 |

### Example 2

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on a Perkin-Elmer Biosystems ABI PRISM® 7700 Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), Panel CNSD.01 (containing samples from normal and diseased brains) and CNS_neurodegeneration_panel (containing samples from normal and diseased brains).

RNA integrity from all samples is controlled for quality by visual assessment of agarose gel electropherograms using 28S and 18S ribosomal RNA staining intensity ratio as a guide (2:1 to 2.5:1 28s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1×TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

GENERAL_SCREENING_PANEL_V1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDR1). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDR1 or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

PANEL 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDR1) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 Fg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 FM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 tg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD 14 and CD19 cells using CD8, CD56, CD14 and CD19 Miltenyi beads and positive selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDR1. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmningen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 □g/ml) were used to direct to Th1, while IL-4 (5 ng/ml) and anti-IFN gamma (1 □g/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 □g/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD 106 and an airway epithelial tumor line NC1-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NC1-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

### Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) pateins, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Broddmann Area 21), parietal cortex (Broddmann area 7), and occipital cortex (Brodmann area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

NOV1a and NOV1d

Expression of gene NOV1a and variant NOV1a was assessed using the primer-probe sets Ag4164, Ag1313b, Ag2197, and Ag708 described in Tables 12, 13, 14, and 15. Please note that Ag4164 contains a single mismatch in the probe relative to the NOV1a and NOV1 d sequences. This mismatch is not predicted to alter the RTQ-PCR results. Results from RTQ-PCR runs are shown in Tables 16, 17, 18, and 19.

TABLE 12

Probe Name Ag4164

| | | | | | |
|---|---|---|---|---|---|
| | | | | Start | SEQ ID |
| Primers | Sequences | TM | Length | Position | NO: |

| | | | | | |
|---|---|---|---|---|---|
| Forward | 5′-GCACTACAAGTGGAAGCCTTAC-3′ | 58.1 | 22 | 822 | 108 |
| Probe | FAM-5′-CTCAAGTAGAAGCCGACTTATGCAAA-3′-TAMRA | 64 | 26 | 845 | 109 |
| Reverse | 5′-TCAAATCCTTCTGCGATACAGT-3′ | 58.9 | 22 | 875 | 110 |

TABLE 13
Probe Name Ag1313b

| | | | | Start | SEQ ID |
|---|---|---|---|---|---|
| Primers | Sequences | TM | Length | Position | NO: |
| Forward | 5′-CAGCTGCACGATTAATGAAGAT-3′ | 59.4 | 22 | 264 | 111 |
| Probe | TET-5′-AGGTCTTGGACTGGCCTTCACCATT-3′-TAMRA | 69 | 25 | 288 | 112 |
| Reverse | 5′-CCAAAGTTGTGTCCAGACTCAT-3′ | 59.1 | 22 | 317 | 113 |

TABLE 14
Probe Name Ag2197

| | | | | Start | SEQ ID |
|---|---|---|---|---|---|
| Primers | Sequences | TM | Length | Position | NO: |
| Forward | 5′-CCAAGGAAGACCTCTTCATCTT-3′ | 58.8 | 22 | 1022 | 114 |
| Probe | FAM-5′-TCTTGCTTACGGCATAAGCGCTCTCT-3′-TAMRA | 69 | 26 | 1060 | 115 |
| Reverse | 5-TTCATTTCTATGGGACCTCAGA-3′ | 58.7 | 22 | 1086 | 116 |

TABLE 15
Probe Name Ag708

| | | | | Start | SEQ ID |
|---|---|---|---|---|---|
| Primers | Sequences | TM | Length | Position | NO: |
| Forward | 5′-AAAGATGGGACTCGTCATGAC-3′ | 59 | 21 | 232 | 117 |
| Probe | TET-5′-CACGCCATCTTACTGACTGGTCTGGA-3′-TAMRA | 26 | 253 | 118 | |
| Reverse | 5′-GTGCAAATCCCAAAGTGTCA-3′ | 59.5 | 20 | 306 | 119 |

TABLE 16
Panel 1.2

| | Relative Expression(%) | |
|---|---|---|
| | 1.2tm888t_{—} | 1.2tm1047t_{—} |
| Tissue Name | ag708 | ag708 |
| Endothelial cells | 0.0 | 0.0 |
| Heart (fetal) | 1.2 | 0.0 |
| Pancreas | 22.5 | 29.5 |
| Pancreatic ca. CAPAN 2 | 0.0 | 0.0 |
| Adrenal Gland (new lot*) | 0.9 | 0.0 |
| Thyroid | 0.5 | 0.0 |
| Salivary gland | 0.4 | 0.0 |
| Pituitary gland | 1.3 | 0.0 |
| Brain (fetal) | 4.0 | 0.0 |
| Brain (whole) | 7.9 | 6.5 |
| Brain (amygdala) | 2.1 | 0.0 |
| Brain (cerebellum) | 16.7 | 23.3 |
| Brain (hippocampus) | 4.6 | 3.1 |
| Brain (thalamus) | 1.5 | 0.0 |
| Cerebral Cortex | 10.7 | 5.8 |
| Spinal cord | 1.1 | 0.0 |
| CNS ca. (glio/astro) U87-MG | 4.4 | 11.7 |
| CNS ca. (glio/astro) U-118-MG | 0.6 | 0.1 |
| CNS ca. (astro) SW1783 | 1.2 | 0.7 |
| CNS ca.* (neuro; met) SK-N-AS | 0.2 | 0.0 |
| CNS ca (astro) SF-539 | 1.6 | 1.1 |
| CNS ca. (astro) SNB-75 | 7.7 | 1.7 |
| CNS ca. (glio) SNB-19 | 9.5 | 4.3 |
| CNS ca. (glio) U251 | 100.0 | 100.0 |
| CNS ca. (glio) SF-295 | 2.4 | 0.0 |
| Heart | 4.9 | 0.0 |
| Skeletal Muscle (new lot*) | 3.1 | 0.0 |
| Bone marrow | 0.0 | 0.0 |
| Thymus | 0.2 | 0.0 |
| Spleen | 0.0 | 0.0 |
| Lymph node | 0.2 | 0.0 |
| Colorectal | 0.0 | 0.0 |
| Stomach | 1.2 | 0.0 |
| Small intestine | 1.4 | 0.0 |
| Colon ca. SW480 | 1.5 | 0.0 |
| Colon ca.* (SW480 met) SW620 | 0.0 | 0.0 |
| Colon ca. HT29 | 0.0 | 0.0 |
| Colon ca. HCT-116 | 0.7 | 0.0 |
| Colon ca. CaCo-2 | 0.0 | 0.0 |
| 83219 CC Well to Mod Diff (ODO3866) | 0.1 | 0.0 |
| Colon ca. HCC-2998 | 0.0 | 0.0 |
| Gastric ca.* (liver met) NCI-N87 | 4.1 | 0.1 |
| Bladder | 3.5 | 0.0 |
| Trachea | 0.3 | 0.0 |
| Kidney | 5.4 | 10.7 |
| Kidney (fetal) | 37.9 | 94.0 |
| Renal ca. 786-0 | 4.6 | 5.3 |
| Renal ca. A498 | 12.2 | 20.2 |
| Renal ca. RXF 393 | 3.3 | 4.2 |
| Renal ca. ACHN | 2.8 | 1.1 |
| Renal ca. UO-31 | 2.3 | 0.2 |
| Renal ca. TK-10 | 7.6 | 12.7 |
| Liver | 0.9 | 0.0 |
| Liver (fetal) | 0.2 | 0.0 |
| Liver ca. (hepatoblast) HepG2 | 0.0 | 0.0 |
| Lung | 1.1 | 0.0 |
| Lung (fetal) | 1.2 | 0.0 |
| Lung ca. (small cell) LX-1 | 0.0 | 0.0 |
| Lung ca. (small cell) NCI-H69 | 0.1 | 0.0 |
| Lung ca. (s.cell var.) SHP-77 | 0.0 | 0.0 |
| Lung ca. (large cell) NCI-H460 | 0.0 | 0.0 |
| Lung ca. (non-sm. cell) A549 | 0.5 | 0.0 |
| Lung ca. (non-s.cell) NCI-H23 | 0.0 | 0.0 |
| Lung ca (non-s.cell) HOP-62 | 4.9 | 1.1 |
| Lung ca. (non-s.cl) NCI-H522 | 0.0 | 0.0 |
| Lung ca. (squam.) SW 900 | 3.7 | 2.0 |
| Lung ca. (squam.) NCI-H596 | 0.0 | 0.0 |
| Mammary gland | 3.1 | 4.5 |
| Breast ca.* (pl. effusion) MCF-7 | 0.0 | 0.0 |
| Breast ca.* (pl.ef) MDA-MB-231 | 3.1 | 6.2 |
| Breast ca.* (pl. effusion) T47D | 0.8 | 0.0 |
| Breast ca. BT-549 | 0.7 | 0.0 |
| Breast ca. MDA-N | 10.7 | 14.6 |
| Ovary | 26.4 | 39.5 |
| Ovarian ca. OVCAR-3 | 0.4 | 0.0 |
| Ovarian ca. OVCAR-4 | 0.9 | 0.0 |
| Ovarian ca. OVCAR-5 | 6.0 | 1.7 |
| Ovarian ca. OVCAR-8 | 0.7 | 0.0 |
| Ovarian ca. IGROV-1 | 8.1 | 15.2 |
| Ovarian ca.* (ascites) SK-OV-3 | 2.9 | 0.0 |
| Uterus | 0.7 | 0.0 |
| Placenta | 7.1 | 0.0 |
| Prostate | 0.3 | 0.0 |
| Prostate ca.* (bone met) PC-3 | 1.5 | 0.0 |
| Testis | 3.3 | 0.0 |
| Melanoma Hs688(A).T | 0.0 | 0.0 |
| Melanoma* (met) Hs688(B).T | 0.0 | 0.0 |
| Melanoma UACC-62 | 0.0 | 0.0 |
| Melanoma M14 | 0.0 | 0.0 |
| Melanoma LOX IMVI | 1.5 | 0.0 |
| Melanoma* (met) SK-MEL-5 | 0.0 | 0.0 |

TABLE 17
Panel 1.3D

| | Relative | Relative | Relative |
|---|---|---|---|
| | Expression(%) | Expression(%) | Expression(%) |
| | 1.3dtm4180fam_{—} | 1.3dx4tm5365t_{—} | 1.3dtm3301t_{—} |
| Tissue Name | ag2197 | ag1313b_b1 | ag708 |
| Liver adenocarcinoma | 2.6 | 0.5 | 2.3 |
| Pancreas | 2.8 | 1.4 | 4.4 |
| Pancreatic ca. CAPAN 2 | 0.6 | 0.2 | 0.8 |
| Adrenal gland | 0.2 | 0.0 | 0.0 |
| Thyroid | 0.2 | 0.1 | 0.0 |
| Salivary gland | 0.0 | 0.2 | 0.2 |
| Pituitary gland | 0.0 | 0.0 | 0.0 |
| Brain (fetal) | 0.2 | 0.1 | 0.7 |
| Brain (whole) | 3.4 | 2.0 | 2.5 |
| Brain (amygdala) | 1.8 | 1.1 | 1.7 |
| Brain (cerebellum) | 7.4 | 8.0 | 11.2 |
| Brain (hippocampus) | 7.6 | 1.0 | 7.9 |
| Brain (substantia nigra) | 0.0 | 0.0 | 0.0 |
| Brain (thalamus) | 0.2 | 0.1 | 0.0 |
| Cerebral Cortex | 5.6 | 0.5 | 4.6 |
| Spinal cord | 0.2 | 0.2 | 0.8 |
| CNS ca. (glio/astro) U87-MG | 7.0 | 1.8 | 4.6 |
| CNS ca. (glio/astro) U-118-MG | 12.2 | 2.3 | 7.3 |
| CNS ca. (astro) SW1783 | 4.9 | 2.0 | 5.7 |
| CNS ca.* (neuro; met) SK-N-AS | 0.0 | 0.0 | 0.0 |
| CNS ca. (astro) SF-539 | 4.4 | 1.8 | 3.8 |
| CNS ca. (astro) SNB-75 | 33.0 | 13.8 | 36.1 |
| CNS ca. (glio) SNB-19 | 6.0 | 3.4 | 6.7 |
| CNS ca. (glio) U251 | 100.0 | 100.0 | 100.0 |
| CNS ca. (glio) SF-295 | 2.0 | 0.5 | 4.2 |
| Heart (fetal) | 1.2 | 0.1 | 1.3 |
| Heart | 0.0 | 0.0 | 0.6 |
| Fetal Skeletal | 14.8 | 0.3 | 16.3 |
| Skeletal muscle | 0.1 | 0.2 | 0.0 |
| Bone marrow | 0.0 | 0.0 | 0.0 |
| Thymus | 0.3 | 0.0 | 0.5 |
| Spleen | 0.0 | 0.0 | 0.0 |
| Lymph node | 0.2 | 0.3 | 0.1 |
| Colorectal | 0.7 | 0.0 | 0.4 |
| Stomach | 0.7 | 1.2 | 0.7 |
| Small intestine | 1.2 | 0.6 | 2.6 |
| Colon ca. SW480 | 2.2 | 0.1 | 2.4 |
| Colon ca.* (SW480 met)SW620 | 0.0 | 0.0 | 0.0 |
| Colon ca. HT29 | 0.0 | 0.0 | 0.0 |
| Colon ca. HCT-116 | 0.6 | 0.0 | 0.6 |
| Colon ca. CaCo-2 | 0.0 | 0.0 | 0.0 |
| 83219 CC Well to Mod Diff (ODO3866) | 4.2 | 0.3 | 2.6 |
| Colon Ca. HCC-2998 | 0.0 | 0.0 | 0.0 |
| Gastric ca.* (liver met) NCI-N87 | 2.6 | 1.3 | 1.0 |
| Bladder | 0.7 | 0.2 | 0.7 |
| Trachea | 0.4 | 0.2 | 0.1 |
| Kidney | 1.9 | 1.3 | 2.8 |
| Kidney (fetal) | 12.6 | 3.0 | 18.3 |
| Renal ca. 786-0 | 10.7 | 2.1 | 15.1 |
| Renal ca. A498 | 47.3 | 9.7 | 34.4 |
| Renal ca. RXF 393 | 4.3 | 6.0 | 3.4 |
| Renal ca. ACHN | 2.6 | 0.8 | 2.5 |
| Renal ca. UO-31 | 10.7 | 3.0 | 13.5 |
| Renal ca. TK-10 | 7.4 | 1.6 | 9.3 |
| Liver | 0.0 | 0.0 | 0.3 |
| Liver (fetal) | 0.2 | 0.0 | 1.1 |
| Liver ca. (hepatoblast) HepG2 | 0.0 | 0.0 | 0.0 |
| Lung | 0.1 | 0.0 | 0.3 |
| Lung (fetal) | 0.2 | 0.0 | 0.4 |
| Lung ca. (small cell) LX-1 | 0.0 | 0.0 | 0.0 |
| Lung ca. (small cell) NCI-H69 | 0.0 | 0.0 | 0.0 |
| Lung ca. (s.cell var.) SHP-77 | 0.0 | 0.0 | 0.0 |
| Lung ca. (large cell) NCI-H460 | 0.0 | 0.4 | 0.0 |
| Lung ca. (non-sm. cell) A549 | 0.8 | 0.3 | 0.4 |
| Lung ca. (non-s.cell) NCI-H23 | 0.0 | 0.0 | 0.0 |
| Lung ca (non-s.cell) HOP-62 | 4.7 | 1.7 | 4.1 |
| Lung ca. (non-s.cl) NCI-H522 | 0.0 | 0.0 | 0.0 |
| Lung ca. (squam.) SW 900 | 3.7 | 1.6 | 4.0 |
| Lung ca. (squam.) NCI-H596 | 0.0 | 0.0 | 0.0 |
| Mammary gland | 4.0 | 1.1 | 5.8 |
| Breast ca.* (pl. effusion) MCF-7 | 0.0 | 0.1 | 0.0 |
| Breast ca.* (pl.ef) MDA-MB-231 | 46.3 | 7.8 | 12.4 |
| Breast ca.* (pl. effusion) T47D | 0.0 | 0.0 | 0.0 |
| Breast ca. BT-549 | 6.7 | 2.7 | 2.6 |
| Breast ca. MDA-N | 8.3 | 1.2 | 11.1 |
| Ovary | 59.9 | 5.8 | 88.9 |
| Ovarian ca. OVCAR-3 | 0.0 | 2.6 | 0.0 |
| Ovarian ca. OVCAR-4 | 0.0 | 0.0 | 0.0 |
| Ovarian ca. OVCAR-5 | 6.8 | 1.3 | 2.5 |
| Ovarian ca. OVCAR-8 | 1.8 | 0.1 | 1.1 |
| Ovarian ca. IGROV-1 | 4.3 | 0.9 | 4.2 |
| Ovarian ca.* (ascites) SK-OV-3 | 1.8 | 1.7 | 0.6 |
| Uterus | 0.3 | 0.4 | 1.2 |
| Placenta | 0.0 | 0.0 | 0.2 |
| Prostate | 0.1 | 0.1 | 0.1 |
| Prostate ca.* (bone met)PC-3 | 0.7 | 0.3 | 0.7 |
| Testis | 0.4 | 0.2 | 0.8 |
| Melanoma Hs688(A).T | 0.0 | 0.0 | 0.0 |
| Melanoma* (met) Hs688(B).T | 0.4 | 0.2 | 4.0 |
| Melanoma UACC-62 | 0.0 | 0.0 | 0.0 |
| Melanoma M14 | 0.0 | 0.1 | 0.0 |
| Melanoma LOX IMVI | 3.2 | 0.8 | 0.6 |
| Melanoma* (met) SK-MEL-5 | 0.0 | 0.0 | 0.0 |
| Adipose | 1.7 | 0.1 | 1.5 |

TABLE 18
Panel 2D

| | Relative | Relative | Relative |
|---|---|---|---|
| | Expression(%) | Expression(%) | Expression(%) |
| | 2dtm4181fam_{—} | 2Dtm2694t_{—} | 2dx4tm4810t_{—} |
| Tissue Name | ag2197 | ag708 | ag1313b_b2 |
| Normal Colon GENPAK 061003 | 100.0 | 28.3 | 18.4 |
| 83219 CC Well to Mod Diff (ODO3866) | 28.1 | 2.8 | 5.6 |
| 83220 CC NAT (ODO3866) | 0.3 | 0.5 | 3.7 |
| 83221 CC Gr.2 rectosigmoid (ODO3868) | 0.1 | 0.2 | 2.4 |
| 83222 CC NAT (ODO3868) | 0.0 | 0.5 | 0.2 |
| 83235 CC Mod Diff (ODO3920) | 0.0 | 0.0 | 0.2 |
| 83236 CC NAT (ODO3920) | 0.8 | 0.8 | 1.1 |
| 83237 CC Gr.2 ascend colon (ODO3921) | 0.5 | 1.6 | 3.8 |
| 83238 CC NAT (ODO3921) | 0.2 | 2.0 | 1.7 |
| 83241 CC from Partial Hepatectomy | 0.2 | 0.7 | 0.9 |
| (ODO4309) | | | |
| 83242 Liver NAT (ODO4309) | 2.0 | 0.0 | 0.2 |
| 87472 Colon mets to lung (OD04451-01) | 0.0 | 1.1 | 2.0 |
| 87473 Lung NAT (OD04451-02) | 4.8 | 0.0 | 0.0 |
| Normal Prostate Clontech A+ 6546-1 | 9.2 | 0.0 | 1.6 |
| 84140 Prostate Cancer (OD04410) | 1.0 | 0.2 | 0.3 |
| 84141 Prostate NAT (OD04410) | 2.2 | 0.0 | 0.0 |
| 87073 Prostate Cancer (OD04720-01) | 2.2 | 0.2 | 0.0 |
| 87074 Prostate NAT (OD04720-02) | 0.3 | 0.6 | 1.1 |
| Normal Lung GENPAK 061010 | 0.7 | 3.3 | 4.7 |
| 83239 Lung Met to Muscle (ODO4286) | 0.0 | 13.2 | 14.1 |
| 83240 Muscle NAT (ODO4286) | 0.0 | 1.2 | 0.4 |
| 84136 Lung Malignant Cancer (OD03126) | 0.5 | 8.2 | 3.7 |
| 84137 Lung NAT (OD03126) | 0.0 | 0.7 | 0.8 |
| 84871 Lung Cancer (OD04404) | 0.0 | 11.7 | 5.4 |
| 84872 Lung NAT (OD04404) | 0.0 | 4.1 | 3.9 |
| 84875 Lung Cancer (OD04565) | 0.2 | 4.8 | 8.9 |
| 84876 Lung NAT (OD04565) | 0.0 | 0.0 | 0.0 |
| 85950 Lung Cancer (OD04237-01) | 1.0 | 3.3 | 7.3 |
| 85970 Lung NAT (OD04237-02) | 0.8 | 1.4 | 0.5 |
| 83255 Ocular Mel Met to Liver (ODO4310) | 0.4 | 0.0 | 0.0 |
| 83256 Liver NAT (ODO4310) | 0.4 | 0.0 | 0.0 |
| 84139 Melanoma Mets to Lung (OD04321) | 0.9 | 48.3 | 43.1 |
| 84138 Lung NAT (OD04321) | 0.0 | 0.2 | 0.1 |
| Normal Kidney GENPAK 061008 | 0.4 | 100.0 | 100.0 |
| 83786 Kidney Ca, Nuclear grade 2 (OD04338) | 0.9 | 9.6 | 18.7 |
| 83787 Kidney NAT (OD04338) | 0.6 | 29.9 | 28.3 |
| 83788 Kidney Ca Nuclear grade 1/2 (OD04339) | 0.5 | 12.0 | 10.4 |
| 83789 Kidney NAT (OD04339) | 0.5 | 29.7 | 33.9 |
| 83790 Kidney Ca, Clear cell type (OD04340) | 0.2 | 3.0 | 3.4 |
| 83791 Kidney NAT (OD04340) | 0.1 | 38.2 | 34.8 |
| 83792 Kidney Ca, Nuclear grade 3 (OD04348) | 0.0 | 16.4 | 16.7 |
| 83793 Kidney NAT (OD04348) | 0.0 | 34.9 | 35.4 |
| 87474 Kidney Cancer (OD04622-01) | 1.1 | 0.0 | 0.4 |
| 87475 Kidney NAT (OD04622-03) | 0.7 | 5.0 | 4.6 |
| 85973 Kidney Cancer (OD04450-01) | 1.3 | 15.9 | 21.6 |
| 85974 Kidney NAT (OD04450-03) | 0.0 | 37.9 | 38.6 |
| Kidney Cancer Clontech 8120607 | 6.5 | 0.0 | 0.3 |
| Kidney NAT Clontech 8120608 | 1.9 | 7.9 | 8.0 |
| Kidney Cancer Clontech 8120613 | 4.2 | 2.2 | 2.3 |
| Kidney NAT Clontech 8120614 | 1.3 | 23.0 | 15.3 |
| Kidney Cancer Clontech 9010320 | 4.3 | 13.3 | 11.1 |
| Kidney NAT Clontech 9010321 | 0.3 | 30.4 | 30.6 |
| Normal Uterus GENPAK 061018 | 1.0 | 11.7 | 7.3 |
| Uterus Cancer GENPAK 064011 | 0.0 | 4.0 | 4.2 |
| Normal Thyroid Clontech A+ 6570-1 | 4.4 | 0.4 | 0.6 |
| Thyroid Cancer GENPAK 064010 | 2.9 | 0.0 | 0.0 |
| Thyroid Cancer INVITROGEN A302152 | 5.3 | 0.3 | 1.2 |
| Thyroid NAT INVITROGEN A302153 | 0.3 | 2.2 | 1.9 |
| Normal Breast GENPAK 061019 | 4.9 | 2.0 | 3.3 |
| 84877 Breast Cancer (OD04566) | 2.1 | 3.1 | 3.3 |
| 85975 Breast Cancer (OD04590-01) | 4.4 | 7.6 | 4.7 |
| 85976 Breast Cancer Mets (OD04590-03) | 13.2 | 4.3 | 6.3 |
| 87070 Breast Cancer Metastasis (OD04655-05) | 0.1 | 0.4 | 0.8 |
| GENPAK Breast Cancer 064006 | 5.7 | 5.4 | 4.1 |
| Breast Cancer Res. Gen. 1024 | 5.7 | 5.5 | 5.1 |
| Breast Cancer Clontech 9100266 | 0.0 | 4.0 | 3.3 |
| Breast NAT Clontech 9100265 | 0.0 | 3.7 | 3.5 |
| Breast Cancer INVITROGEN A209073 | 0.0 | 4.7 | 6.1 |
| Breast NAT INVITROGEN A2090734 | 0.9 | 4.1 | 4.9 |
| Normal Liver GENPAK 061009 | 0.0 | 0.0 | 0.0 |
| Liver Cancer GENPAK 064003 | 2.2 | 0.0 | 0.0 |
| Liver Cancer Research Genetics RNA 1025 | 0.4 | 0.0 | 0.0 |
| Liver Cancer Research Genetics RNA 1026 | 2.0 | 2.5 | 2.0 |
| Paired Liver Cancer Tissue Research Genetics | 1.4 | 0.0 | 0.0 |
| RNA 6004-T | | | |
| Paired Liver Tissue Research Genetics RNA | 0.0 | 0.0 | 0.4 |
| 6004-N | | | |
| Paired Liver Cancer Tissue Research Genetics | 1.0 | 3.2 | 2.0 |
| RNA 6005-T | | | |
| Paired Liver Tissue Research Genetics RNA | 0.0 | 0.0 | 0.0 |
| 6005-N | | | |
| Normal Bladder GENPAK 061001 | 0.4 | 6.4 | 6.2 |
| Bladder Cancer Research Genetics RNA 1023 | 0.2 | 1.2 | 2.2 |
| Bladder Cancer INVITROGEN A302173 | 0.0 | 6.1 | 6.9 |
| 87071 Bladder Cancer (OD04718-01) | 0.0 | 13.6 | 14.8 |
| 87072 Bladder Normal Adjacent (OD04718-03) | 0.0 | 8.7 | 9.6 |
| Normal Ovary Res. Gen. | 0.0 | 77.4 | 60.2 |
| Ovarian Cancer GENPAK 064008 | 0.0 | 32.8 | 32.1 |
| 87492 Ovary Cancer (OD04768-07) | 0.3 | 0.8 | 0.8 |
| 87493 Ovary NAT (OD04768-08) | 0.0 | 12.0 | 10.2 |
| Normal Stomach GENPAK 061017 | 0.0 | 2.9 | 2.3 |
| Gastric Cancer Clontech 9060358 | 0.5 | 1.1 | 1.1 |
| NAT Stomach Clontech 9060359 | 0.4 | 5.9 | 3.5 |
| Gastric Cancer Clontech 9060395 | 0.0 | 0.4 | 0.2 |
| NAT Stomach Clontech 9060394 | 0.3 | 1.8 | 1.1 |
| Gastric Cancer Clontech 9060397 | 0.1 | 9.3 | 5.7 |
| NAT Stomach Clontech 9060396 | 0.0 | 0.2 | 0.8 |
| Gastric Cancer GENPAK 064005 | 0.4 | 0.4 | 1.5 |

TABLE 19
Panel 4D

| | Relative | | Relative |
|---|---|---|---|
| | Expression(%) | | Expression(%) |
| | 4dtm4182fam_{—} | | 4dtm4182fam_{—} |
| Tissue Name | ag2197 | Tissue Name | ag2197 |
| 93768_Secondary Th1_anti- | 0.0 | 93100_HUVEC | 0.1 |
| CD28/anti-CD3 | | (Endothelial)_IL-1b | |
| 93769_Secondary Th2_anti- | 0.0 | 93779_HUVEC | 0.0 |
| CD28/anti-CD3 | | (Endothelial)_IFN gamma | |
| 93770_Secondary Tr1_anti- | 0.0 | 93102_HUVEC | 0.0 |
| CD28/anti-CD3 | | (Endothelial)_TNF alpha + IFN | |
| | | gamma | |
| 93573_Secondary Th1_resting | 0.0 | 93101_HUVEC | 0.0 |
| day 4-6 in IL-2 | | (Endothelial)_TNF alpha + IL4 | |
| 93572_Secondary Th2_resting | 0.0 | 93781_HUVEC | 0.0 |
| day 4-6 in IL-2 | | (Endothelial)_IL-11 | |
| 93571_Secondary Tr1_resting | 0.0 | 93583_Lung Microvascular | 0.0 |
| day 4-6 in IL-2 | | Endothelial Cells_none | |
| 93568_primary Th1_anti- | 0.0 | 93584_Lung Microvascular | 0.0 |
| CD28/anti-CD3 | | Endothelial Cells_TNFa | |
| | | (4 ng/ml) and IL1b (1 ng/ml) | |
| 93569_primary Th2_anti- | 0.0 | 92662_Microvascular Dermal | 0.0 |
| CD28/anti-CD3 | | endothelium_none | |
| 93570_primary Tr1_anti- | 0.0 | 92663_Microsvasular Dermal | 0.2 |
| CD28/anti-CD3 | | endothelium_TNFa (4 ng/ml) | |
| | | and IL1b (1 ng/ml) | |
| 93565_primary Th1_resting dy | 0.0 | 93773_Bronchial | 0.0 |
| 4-6 in IL-2 | | epithelium_TNFa (4 ng/ml) and | |
| | | IL1b (1 ng/ml)** | |
| 93566_primary Th2_resting dy | 0.0 | 93347_Small Airway | 0.4 |
| 4-6 in IL-2 | | Epithelium_none | |
| 93567_primary Tr1_resting dy | 0.0 | 93348_Small Airway | 0.0 |
| 4-6 in IL-2 | | Epithelium_TNFa (4 ng/ml) | |
| | | and IL1b (1 ng/ml) | |
| 93351_CD45RA CD4 | 0.0 | 92668_Coronery Artery | 0.3 |
| lymphocyte_anti-CD28/anti- | | SMC_resting | |
| CD3 | | | |
| 93352_CD45RO CD4 | 0.0 | 92669_Coronery Artery | 0.0 |
| lymphocyte_anti-CD28/anti- | | SMC_TNFa (4 ng/ml) and IL1b | |
| CD3 | | (1 ng/ml) | |
| 93251_CD8 Lymphocytes_{—} | 0.0 | 93107_astrocytes_resting | 100.0 |
| anti-CD28/anti-CD3 | | | |
| 93353_chronic CD8 | 0.0 | 93108_astrocytes_TNFa | 16.5 |
| Lymphocytes 2ry_resting | | (4 ng/ml) and IL1b (1 ng/ml) | |
| dy 4-6 in IL-2 | | | |
| 93574_chronic CD8 | 0.0 | 92666_KU-812 | 0.0 |
| Lymphocytes 2ry_activated | | (Basophil)_resting | |
| CD3/CD28 | | | |
| 93354_CD4_none | 0.0 | 92667_KU-812 | 0.0 |
| | | (Basophil)_PMA/ionoycin | |
| 93252_Secondary | 0.0 | 93579_CCD1106 | 0.0 |
| Th1/Th2/Tr1_anti-CD95 CH11 | | (Keratinocytes)_none | |
| 93103_LAK cells_resting | 0.0 | 93580_CCD1106 | 0.0 |
| | | (Keratinocytes)_TNFa and | |
| | | IFNg** | |
| 93788_LAK cells_IL-2 | 0.0 | 93791_Liver Cirrhosis | 1.3 |
| 93787_LAK cells_{—} | 0.0 | 93792_Lupus Kidney | 11.3 |
| IL-2 + IL-12 | | | |
| 93789_LAK cells_{—} | 0.0 | 93577_NCI-H292 | 8.4 |
| IL-2 + IFN gamma | | | |
| 93790_LAK cells_{—} | 0.0 | 93358_NCI-H292_IL-4 | 12.5 |
| IL-2 + IL-18 | | | |
| 93104_LAK | 0.8 | 93360 NCI-H292_IL-9 | 11.8 |
| cells_PMA/ionomycin and | | | |
| IL-18 | | | |
| 93578_NK Cells IL-2_resting | 0.0 | 93359_NCI-H292_IL-13 | 4.2 |
| 93109_Mixed Lymphocyte | 0.0 | 93357_NCI-H292_IFN gamma | 3.7 |
| Reaction_Two Way MLR | | | |
| 93110_Mixed Lymphocyte | 0.0 | 93777_HPAEC_- | 0.0 |
| Reaction_Two Way MLR | | | |
| 93111_Mixed Lymphocyte | 0.0 | 93778_HPAEC_IL-1 beta/TNA | 0.0 |
| Reaction_Two Way MLR | | alpha | |
| 93112_Mononuclear Cells | 0.0 | 93254_Normal Human Lung | 0.3 |
| (PBMCs)_resting | | Fibroblast_none | |
| 93113_Mononuclear Cells | 0.0 | 93253_Normal Human Lung | 0.0 |
| (PBMCs)_PWM | | Fibroblast_TNFa (4 ng/ml) and | |
| | | IL-1b (1 ng/ml) | |
| 93114_Mononuclear Cells | 0.1 | 93257_Normal Human Lung | 0.0 |
| (PBMCs)_PHA-L | | Fibroblast_IL-4 | |
| 93249_Ramos (B cell)_none | 0.0 | 93256_Normal Human Lung | 0.0 |
| | | Fibroblast_IL-9 | |
| 93250_Ramos (B | 0.0 | 93255_Normal Human Lung | 0.0 |
| cell)_ionomycin | | Fibroblast_IL-13 | |
| 93349_B lymphocytes_PWM | 0.0 | 93258_Normal Human Lung | 0.0 |
| | | Fibroblast_IFN gamma | |
| 93350_B lymphoytes_CD40L | 0.0 | 93106_Dermal Fibroblasts | 0.0 |
| and IL-4 | | CCD1070_resting | |
| 92665_EOL-1 | 0.0 | 93361_Dermal Fibroblasts | 0.0 |
| (Eosinophil)_dbcAMP | | CCD1070_TNF alpha 4 ng/ml | |
| differentiated | | | |
| 93248_EOL-1 | 0.0 | 93105_Dermal Fibroblasts | 0.0 |
| (Eosinophil)_dbcAMP/ | | CCD1070_IL-1 beta 1 ng/ml | |
| PMAionomycin | | | |
| 93356_Dendritic Cells_none | 0.0 | 93772_dermal fibroblast_IFN | 0.0 |
| | | gamma | |
| 93355_Dendritic Cells_LPS | 0.0 | 93771_dermal fibroblast_IL-4 | 0.0 |
| 100 ng/ml | | | |
| 93775_Dendritic Cells_anti- | 0.0 | 93260_IBD Colitis 2 | 0.1 |
| CD40 | | | |
| 93774_Monocytes_resting | 0.0 | 93261_IBD Crohns | 4.1 |
| 93776_Monocytes_{—} | 0.0 | 735010_Colon_normal | 1.2 |
| LPS 50 ng/ml | | | |
| 93581_Macrophages_resting | 0.3 | 735019_Lung_none | 12.2 |
| 93582_Macrophages_{—} | 0.0 | 64028-1_Thymus_none | 62.8 |
| LPS 100 ng/ml | | | |
| 93098_HUVEC | 0.0 | 64030-1_Kidney_none | 2.2 |
| (Endothelial)_none | | | |
| 93099_HUVEC | 0.0 | | |
| (Endothelial)_starved | | | |

Panel 1.2 Summary Ag708 Expression of the NOV1A gene was assessed in two independent experiments using the same probe/primer set. There appears to be poor concordance between runs for some tissues but there is good concordance for others; only those results that are in agreement will be discussed here. In both experiments, highest expression of the NOV1a gene in a sample derived from a glioblastoma cell line (CTs=24-26). Among normal tissues derived from the central nervous system, the NOV1a gene is also expressed at moderate levels in the cerebral cortex, cerebellum and hippocampus.

Among tissues with metabolic function, the NOV1a gene is expressed in the pancreas. Thus, this gene may be involved in the pathogenesis and/or treatment of diseases involving the pancreas, such as pancreatitis and diabetes. In addition, NOV1a gene expression is decreased in a pancreatic cancer cell line.

The NOV 1 a gene appears to be overexpressed in fetal kidney when compared to the adult kidney. This result suggests that the NOV1a gene could be used to distinguish between adult and fetal kidney tissue and that this gene may play an important role in kidney development, growth and survival. Furthermore, NOV 1 a gene expression is higher in normal ovary, mammary gland and lung when compared to the cancer cell lines obtained from these tissues suggesting that this can be used as a marker to differentiate malignant and normal tissue.

Panel 1.3D Summary Ag708/Ag1313b/Ag2197 Three experiments with three different probe and primer sets produced results that were in very good agreement. One run, designated 1.3dx4tm5365t, appears to have lower absolute expression, but produces the same expression profile as the other two experiments. Highest expression of the NOV1A gene in all three runs is seen in a sample derived from a CNS cancer cell line (CTs=27). Certain glioblastoma and astrocytoma cell lines also express this gene as well so it may have a role in different types of brain cancer. Among normal tissues derived from the central nervous system, the NOV1a gene is expressed in the amygdala, cerebellum, hippocampus and cerebral cortex.

Among tissues with metabolic function, the NOV1a gene is expressed in the pancreas and in adipose. Interestingly, this gene is also expressed at higher levels in fetal heart and skeletal tissue when compared to the adult tissues.

The NOV1a gene appears to be expressed at high levels in a sample from a renal cancer cell line and from a breast cancer cell line. In addition, the NOV1a gene is expressed in ovarian tissue, but not significantly in cell lines derived from ovarian cancer.

Panel 2D Summary Ag708/Az1313b In two runs using two different probe and primer sets, highest expression of the NOV 1A gene is seen in the normal ovary, colon and kidney. Furthermore, in all nine matched kidney pairs and in the matched tissue pair derived from the ovary, the NOV1A gene is expressed more highly in normal tissue than in the adjacent cancer samples. This result suggests that expression of the NOV1A gene could be used as a diagnostic marker for the presence of kidney and ovarian cancer. In addition, therapeutic upregulation of the gene activity of NOV1 A could be effective in the treatment of kidney and ovarian cancer. The NOV1 A gene is also expressed at higher levels in lung cancer samples, when compared to normal adjacent tissue in six out of seven matched tissue pairs. Thus, therapeutic inhibition of the NOV1a gene, through the application of antibodies or small molecule drugs, could be effective in the treatment of lung cancer.

Panel 4D Summary Ag2197 The NOV1a gene is expressed at a high level in astrocytes (CT 27.8) and its expression is down regulated upon treatment with TNF-a and IL-1b, suggesting that modulation of this protein could be beneficial in the treatment of CNS diseases-associated inflammation or neurodegeneration. The NOV1a gene is also expressed highly in the thymus (CT 28.4). More moderate expression of this gene is observed in the lung and in a muco-epidermoid cell line (H292). Thus, the protein encoded by the NOV1a gene could play an important role in the normal homeostasis of these tissues. Therapeutics designed with this protein could be important for maintaining or restoring normal function to these organs during inflammation.

Panel 4.1D Summary Ag4164 Expression of this gene is low/undetectable (CT values>35) among the samples on this panel (data not shown).

Panel CNS_neurodegeneration_v1.0 Summary Ag4164 Expression of this gene is low/undetectable (CT values >35) among the samples on this panel (data not shown).

NOV1b

The NOV1b sequence is a variant of gene NOV1a annotated above. However, the NOV1b sequence only matches a subset of the probe and primer sets discussed above and is discussed independently below in section B. Expression of gene NOV1b was assessed using the primer-probe sets Ag4164 and Ag2197 described in Tables 20 and 21. Please note that Ag4164 contains a single mismatch in the probe relative to the NOV1b sequence. This mismatch is not predicted to alter the RTQ-PCR results. Results from RTQ-PCR runs are shown in Tables 22, 23, and 24.

TABLE 20
Probe Name Ag4164

| | | | | Start | SEQ ID |
|---|---|---|---|---|---|
| Primers | Sequences | TM | Length | Position | NO: |
| Forward | 5′-GCACTACAAGTGGAAGCCTTAC-3′ | 58.1 | 22 | 822 | 120 |
| Probe | FAM-5′-CTCAAGTAGAAGCCGACTTATGCAAA-3′-TAMRA | 64 | 26 | 845 | 121 |
| Reverse | 5′-TCAAATCCTTCTGCGATACAGT-3′ | 58.9 | 22 | 875 | 122 |

TABLE 21
Probe Name Ag2197

| | | | | Start | SEQ ID |
|---|---|---|---|---|---|
| Primers | Sequences | TM | Length | Position | NO: |
| Forward | 5′-CCAAGGAAGACCTCTTCATCTT-3′ | 58.81 | 22 | 1022 | 123 |
| Probe | FAM-5′-TCTTGCTTACGGCATAAGCGCTCTCT-3′-TAMRA | 09 | 26 | 1060 | 124 |
| Reverse | 5′-TTCATTTCTATGGGACCTCAGA-3′ | 58.7 | 22 | 1086 | 125 |

TABLE 22
Panel 1.3D

| | Relative | | Relative |
|---|---|---|---|
| | Expression(%) | | Expression(%) |
| | 1.3dtm4180fam_{—} | | 1.3dtm4180fam_{—} |
| Tissue Name | ag2197 | Tissue Name | ag2197 |
| Liver adenocarcinoma | 2.6 | Kidney (fetal) | 12.6 |
| Pancreas | 2.8 | Renal ca. 786-0 | 10.7 |
| Pancreatic ca. CAPAN 2 | 0.6 | Renal ca. A498 | 47.3 |
| Adrenal gland | 0.2 | Renal ca. RXF 393 | 4.3 |
| Thyroid | 0.2 | Renal ca. ACHN | 2.6 |
| Salivary gland | 0.0 | Renal ca. UO-31 | 10.7 |
| Pituitary gland | 0.0 | Renal ca. TK-10 | 7.4 |
| Brain (fetal) | 0.2 | Liver | 0.0 |
| Brain (whole) | 3.4 | Liver (fetal) | 0.2 |
| Brain (amygdala) | 1.8 | Liver ca. (hepatoblast) HepG2 | 0.0 |
| Brain (cerebellum) | 7.4 | Lung | 0.1 |
| Brain (hippocampus) | 7.6 | Lung (fetal) | 0.2 |
| Brain (substantia nigra) | 0.0 | Lung ca. (small cell) LX-1 | 0.0 |
| Brain (thalamus) | 0.2 | Lung ca. (small cell) NCI-H69 | 0.0 |
| Cerebral Cortex | 5.6 | Lung ca. (s.cell var.) SHP-77 | 0.0 |
| Spinal cord | 0.2 | Lung ca. (large cell) NCI-H460 | 0.0 |
| CNS ca. (glio/astro) U87-MG | 7.0 | Lung ca. (non-sm. cell) A549 | 0.8 |
| CNS ca. (glio/astro) U-118-MG | 12.2 | Lung ca. (non-s.cell) NCI-H23 | 0.0 |
| CNS ca. (astro) SW1783 | 4.9 | Lung ca. (non-s.cell) HOP-62 | 4.7 |
| CNS ca.* (neuro; met) SK-N-AS | 0.0 | Lung ca. (non-s.cl) NCI-H522 | 0.0 |
| CNS ca. (astro) SF-539 | 4.4 | Lung ca. (squam.) SW 900 | 3.7 |
| CNS ca. (astro) SNB-75 | 33.0 | Lung ca. (squam.) NCI-H596 | 0.0 |
| CNS ca. (glio) SNB-19 | 6.0 | Mammary gland | 4.0 |
| CNS ca. (glio) U251 | 100.0 | Breast ca.* (pl. effusion) MCF-7 | 0.0 |
| CNS ca. (glio) SF-295 | 2.0 | Breast ca.* (pl. ef) MDA-MB- | 46.3 |
| | | 231 | |
| Heart (fetal) | 1.2 | Breast ca.* (pl. effusion) T47D | 0.0 |
| Heart | 0.0 | Breast ca. BT-549 | 6.7 |
| Fetal Skeletal | 14.8 | Breast ca. MDA-N | 8.3 |
| Skeletal muscle | 0.1 | Ovary | 59.9 |
| Bone marrow | 0.0 | Ovarian ca. OVCAR-3 | 0.0 |
| Thymus | 0.3 | Ovarian ca. OVCAR-4 | 0.0 |
| Spleen | 0.0 | Ovarian ca. OVCAR-5 | 6.8 |
| Lymph node | 0.2 | Ovarian ca. OVCAR-8 | 1.8 |
| Colorectal | 0.7 | Ovarian ca. IGROV-1 | 4.3 |
| Stomach | 0.7 | Ovarian ca.* (ascites) SK-OV-3 | 1.8 |
| Small intestine | 1.2 | Uterus | 0.3 |
| Colon ca. SW480 | 2.2 | Placenta | 0.0 |
| Colon ca.* (SW480 met) SW620 | 0.0 | Prostate | 0.1 |
| Colon ca. HT29 | 0.0 | Prostate ca.* (bone met) PC-3 | 0.7 |
| Colon ca. HCT-116 | 0.6 | Testis | 0.4 |
| Colon ca. CaCo-2 | 0.0 | Melanoma Hs688(A).T | 0.0 |
| 83219 CC Well to Mod Diff | 4.2 | Melanoma* (met) Hs688(B).T | 0.4 |
| (ODO3866) | | | |
| Colon ca. HCC-2998 | 0.0 | Melanoma UACC-62 | 0.0 |
| Gastric ca.* (liver met) NCI- | 2.6 | Melanoma M14 | 0.0 |
| N87 | | | |
| Bladder | 0.7 | Melanoma LOX IMVI | 3.2 |
| Trachea | 0.4 | Melanoma* (met) SK-MEL-5 | 0.0 |
| Kidney | 1.9 | Adipose | 1.7 |

TABLE 23
Panel 2D

| | Relative | | Relative |
|---|---|---|---|
| | Expression(%) | | Expression(%) |
| | 2dtm4181fam_{—} | | 2dtm4181fam_{—} |
| Tissue Name | ag2197 | Tissue Name | ag2197 |
| Normal Colon GENPAK | 100.0 | Kidney NAT Clontech 8120608 | 1.9 |
| 061003 | | | |
| 83219 CC Well to Mod Diff | 28.1 | Kidney Cancer Clontech | 4.2 |
| (ODO3866) | | 8120613 | |
| 83220 CC NAT (ODO3866) | 0.3 | Kidney NAT Clontech 8120614 | 1.3 |
| 83221 CC Gr.2 rectosigmoid | 0.1 | Kidney Cancer Clontech | 4.3 |
| (ODO3868) | | 9010320 | |
| 83222 CC NAT (ODO3868) | 0.0 | Kidney NAT Clontech 9010321 | 0.3 |
| 83235 CC Mod Diff | 0.0 | Normal Uterus GENPAK | 1.0 |
| (ODO3920) | | 061018 | |
| 83236 CC NAT (ODO3920) | 0.8 | Uterus Cancer GENPAK | 0.0 |
| | | 064011 | |
| 83237 CC Gr.2 ascend colon | 0.5 | Normal Thyroid Clontech | 4.4 |
| (ODO3921) | | A+ 6570-1 | |
| 83238 CC NAT (ODO3921) | 0.2 | Thyroid Cancer GENPAK | 2.9 |
| | | 064010 | |
| 83241 CC from Partial | 0.2 | Thyroid Cancer INVITROGEN | 5.3 |
| Hepatectomy (ODO4309) | | A302152 | |
| 83242 Liver NAT (ODO4309) | 2.0 | Thyroid NAT INVITROGEN | 0.3 |
| | | A302153 | |
| 87472 Colon mets to lung | 0.0 | Normal Breast GENPAK | 4.9 |
| (OD04451-01) | | 061019 | |
| 87473 Lung NAT (OD04451-02) | 4.8 | 84877 Breast Cancer | 2.1 |
| | | (OD04566) | |
| Normal Prostate Clontech A+ | 9.2 | 85975 Breast Cancer | 4.4 |
| 6546-1 | | (OD04590-01) | |
| 84140 Prostate Cancer | 1.0 | 85976 Breast Cancer Mets | 13.2 |
| (OD04410) | | (OD04590-03) | |
| 84141 Prostate NAT | 2.2 | 87070 Breast Cancer Metastasis | 0.1 |
| (OD04410) | | (OD04655-05) | |
| 87073 Prostate Cancer | 2.2 | GENPAK Breast Cancer | 5.7 |
| (OD04720-01) | | 064006 | |
| 87074 Prostate NAT | 0.3 | Breast Cancer Res. Gen. 1024 | 5.7 |
| (OD04720-02) | | | |
| Normal Lung GENPAK 061010 | 0.7 | Breast Cancer Clontech | 0.0 |
| | | 9100266 | |
| 83239 Lung Met to Muscle | 0.0 | Breast NAT Clontech 9100265 | 0.0 |
| (ODO4286) | | | |
| 83240 Muscle NAT | 0.0 | Breast Cancer INVITROGEN | 0.0 |
| (ODO4286) | | A209073 | |
| 84136 Lung Malignant Cancer | 0.5 | Breast NAT INVITROGEN | 0.9 |
| (OD03126) | | A2090734 | |
| 84137 Lung NAT (OD03126) | 0.0 | Normal Liver GENPAK | 0.0 |
| | | 061009 | |
| 84871 Lung Cancer (OD04404) | 0.0 | Liver Cancer GENPAK 064003 | 2.2 |
| 84872 Lung NAT (OD04404) | 0.0 | Liver Cancer Research Genetics | 0.4 |
| | | RNA 1025 | |
| 84875 Lung Cancer (OD04565) | 0.2 | Liver Cancer Research Genetics | 2.0 |
| | | RNA 1026 | |
| 84876 Lung NAT (OD04565) | 0.0 | Paired Liver Cancer Tissue | 1.4 |
| | | Research Genetics RNA 6004-T | |
| 85950 Lung Cancer | 1.0 | Paired Liver Tissue Research | 0.0 |
| (OD04237-01) | | Genetics RNA 6004-N | |
| 85970 Lung NAT (OD04237-02) | 0.8 | Paired Liver Cancer Tissue | 1.0 |
| | | Research Genetics RNA 6005-T | |
| 83255 Ocular Mel Met to Liver | 0.4 | Paired Liver Tissue Research | 0.0 |
| (ODO4310) | | Genetics RNA 6005-N | |
| 83256 Liver NAT (ODO4310) | 0.4 | Normal Bladder GENPAK | 0.4 |
| | | 061001 | |
| 84139 Melanoma Mets to Lung | 0.9 | Bladder Cancer Research | 0.2 |
| (OD04321) | | Genetics RNA 1023 | |
| 84138 Lung NAT (OD04321) | 0.0 | Bladder Cancer INVITROGEN | 0.0 |
| | | A302173 | |
| Normal Kidney GENPAK | 0.4 | 87071 Bladder Cancer | 0.0 |
| 061008 | | (OD04718-01) | |
| 83786 Kidney Ca, Nuclear | 0.9 | 87072 Bladder Normal | 0.0 |
| grade 2 (OD04338) | | Adjacent (OD04718-03) | |
| 83787 Kidney NAT (OD04338) | 0.6 | Normal Ovary Res. Gen. | 0.0 |
| 83788 Kidney Ca Nuclear grade | 0.5 | Ovarian Cancer GENPAK | 0.0 |
| 1/2 (OD04339) | | 064008 | |
| 83789 Kidney NAT (OD04339) | 0.5 | 87492 Ovary Cancer | 0.3 |
| | | (OD04768-07) | |
| 83790 Kidney Ca, Clear cell | 0.2 | 87493 Ovary NAT (OD04768-08) | 0.0 |
| type (OD04340) | | | |
| 83791 Kidney NAT (OD04340) | 0.1 | Normal Stomach GENPAK | 0.0 |
| | | 061017 | |
| 83792 Kidney Ca, Nuclear | 0.0 | Gastric Cancer Clontech | 0.5 |
| grade 3 (OD04348) | | 9060358 | |
| 83793 Kidney NAT (OD04348) | 0.0 | NAT Stomach Clontech | 0.4 |
| | | 9060359 | |
| 87474 Kidney Cancer | 1.1 | Gastric Cancer Clontech | 0.0 |
| (OD04622-01) | | 9060395 | |
| 87475 Kidney NAT | 0.7 | NAT Stomach Clontech | 0.3 |
| (OD04622-03) | | 9060394 | |
| 85973 Kidney Cancer | 1.3 | Gastric Cancer Clontech | 0.1 |
| (OD04450-01) | | 9060397 | |
| 85974 Kidney NAT | 0.0 | NAT Stomach Clontech | 0.0 |
| (OD04450-03) | | 9060396 | |
| Kidney Cancer Clontech | 6.5 | Gastric Cancer GENPAK | 0.4 |
| 8120607 | | 064005 | |

TABLE 24
Panel 4D

| | Relative | | Relative |
|---|---|---|---|
| | Expression(%) | | Expression(%) |
| | 4dtm4182fam_{—} | | 4dtm4182fam_{—} |
| Tissue Name | ag2197 | Tissue Name | ag2197 |
| 93768_Secondary Th1_anti- | 0.0 | 93100_HUVEC | 0.1 |
| CD28/anti-CD3 | | (Endothelial)_IL-1b | |
| 93769_Secondary Th2_anti- | 0.0 | 93799_HUVEC | 0.0 |
| CD28/anti-CD3 | | (Endothelial)_IFN gamma | |
| 93770_Secondary Tr1_anti- | 0.0 | 93102_HUVEC | 0.0 |
| CD28/anti-CD3 | | (Endothelial)_TNF alpha + IFN | |
| | | gamma | |
| 93573_Secondary Th1_resting | 0.0 | 93101_HUVEC | 0.0 |
| day 4-6 in IL-2 | | (Endothelial)_TNF alpha + IL4 | |
| 93572_Secondary Th2_resting | 0.0 | 93781_HUVEC | 0.0 |
| day 4-6 in IL-2 | | (Endothelial)_IL-11 | |
| 93571_Secondary Tr1_resting | 0.0 | 93583_Lung Microvascular | 0.0 |
| day 4-6 in IL-2 | | Endothelial Cells_none | |
| 93568_primary Th1_anti- | 0.0 | 93584_Lung Microvascular | 0.0 |
| CD28/anti-CD3 | | Endothelial Cells_TNFa (4 ng/ml) | |
| | | and IL1b (1 ng/ml) | |
| 93569_primary Th2_anti- | 0.0 | 92662_Microvascular Dermal | 0.0 |
| CD28/anti-CD3 | | endothelium_none | |
| 93570_primary Tr1_anti- | 0.0 | 92663_Microvascular Dermal | 0.2 |
| CD28/anti-CD3 | | endothelium_TNFa (4 ng/ml) | |
| | | and IL1b (1 ng/ml) | |
| 93565_primary Th1_resting dy | 0.0 | 93773_Bronchial | 0.0 |
| 4-6 in IL-2 | | epithelium_TNFa (4 ng/ml) and | |
| | | IL1b (1 ng/ml)** | |
| 93566_primary Th2_resting dy | 0.0 | 93347_Small Airway | 0.4 |
| 4-6 in IL-2 | | Epithelium_none | |
| 93567_primary Tr1_resting dy | 0.0 | 93348_Small Airway | 0.0 |
| 4-6 in IL-2 | | Epithelium_TNFa (4 ng/ml) | |
| | | and IL1b (1 ng/ml) | |
| 93351_CD45RA CD4 | 0.0 | 92668_Coronery Artery | 0.3 |
| lymphocyte_anti-CD28/anti- | | SMC_resting | |
| CD3 | | | |
| 93352_CD45RO CD4 | 0.0 | 92669_Coronery Artery | 0.0 |
| lymphocyte_anti-CD28/anti- | | SMC_TNFa (4 ng/ml) and IL1b | |
| CD3 | | (1 ng/ml) | |
| 93251_CD8 Lymphocytes_anti- | 0.0 | 93107_astrocytes_resting | 100.0 |
| CD28/anti-CD3 | | | |
| 93353_chronic CD8 | 0.0 | 93108_astrocytes_TNFa | 16.5 |
| Lymphocytes 2ry_resting dy 4-6 | | (4 ng/ml) and IL1b (1 ng/ml) | |
| in IL-2 | | | |
| 93574_chronic CD8 | 0.0 | 92666_KU-812 | 0.0 |
| Lymphocytes 2ry_activated | | (Basophil)_resting | |
| CD3/CD28 | | | |
| 93354_CD4_none | 0.0 | 92667_KU-812 | 0.0 |
| | | (Basophil)_PMA/ionoycin | |
| 93252_Secondary | 0.0 | 93579_CCD1106 | 0.0 |
| Th1/Th2/Tr1_anti-CD95 CH11 | | (Keratinocytes)_none | |
| 93103_LAK cells_resting | 0.0 | 93580_CCD1106 | 0.0 |
| | | (Keratinocytes)_TNFa and | |
| | | IFNg** | |
| 93788_LAK cells_IL-2 | 0.0 | 93791_Liver Cirrhosis | 1.3 |
| 93787_LAK cells_IL-2 + IL-12 | 0.0 | 93792_Lupus Kidney | 11.3 |
| 93789_LAK cells_IL-2 + IFN | 0.0 | 93577_NCI-H292 | 8.4 |
| gamma | | | |
| 93790_LAK cells_IL-2 + IL-18 | 0.0 | 93358_NCI-H292_IL-4 | 12.5 |
| 93104_LAK | 0.8 | 93360_NCI-H292_IL-9 | 11.8 |
| cells_PMA/ionomycin and | | | |
| IL-18 | | | |
| 93578_NK Cells IL-2_resting | 0.0 | 93359_NCI-H292_IL-13 | 4.2 |
| 93109_Mixed Lymphocyte | 0.0 | 93357_NCI-H292_IFN gamma | 3.7 |
| Reaction_Two Way MLR | | | |
| 93110_Mixed Lymphocyte | 0.0 | 93777_HPAEC_- | 0.0 |
| Reaction_Two Way MLR | | | |
| 93111_Mixed Lymphocyte | 0.0 | 93778_HPAEC_IL-1 beta/TNA | 0.0 |
| Reaction_Two Way MLR | | alpha | |
| 93112_Mononuclear Cells | 0.0 | 93254_Normal Human Lung | 0.3 |
| (PBMCs)_resting | | Fibroblast_none | |
| 93113_Mononuclear Cells | 0.0 | 93253_Normal Human Lung | 0.0 |
| (PBMCs)_PWM | | Fibroblast_TNFa (4 ng/ml) and | |
| | | IL-1b (1 ng/ml) | |
| 93114_Mononuclear Cells | 0.1 | 93257_Normal Human Lung | 0.0 |
| (PBMCs)_PHA-L | | Fibroblast_IL-4 | |
| 93249_Ramos (B cell)_none | 0.0 | 93256_Normal Human Lung | 0.0 |
| | | Fibroblast_IL-9 | |
| 93250_Ramos (B | 0.0 | 93255_Normal Human Lung | 0.0 |
| cell)_ionomycin | | Fibroblast_IL-13 | |
| 93349_B lymphocytes_PWM | 0.0 | 93258_Normal Human Lung | 0.0 |
| | | Fibroblast_IFN gamma | |
| 93350_B lymphoytes_CD40L | 0.0 | 93106_Dermal Fibroblasts | 0.0 |
| and IL-4 | | CCD1070_resting | |
| 92665_EOL-1 | 0.0 | 93361_Dermal Fibroblasts | 0.0 |
| (Eosinophil)_dbcAMP | | CCD1070_TNF alpha 4 ng/ml | |
| differentiated | | | |
| 93248_EOL-1 | 0.0 | 93105_Dermal Fibroblasts | 0.0 |
| (Eosinophil)_dbcAMP/ | | CCD1070_IL-1 beta 1 ng/ml | |
| PMAionomycin | | | |
| 93356_Dendritic Cells_none | 0.0 | 93772_dermal fibroblast_IFN | 0.0 |
| | | gamma | |
| 93355_Dendritic Cells_LPS | 0.0 | 93771_dermal fibroblasts_IL-4 | 0.0 |
| 100 ng/ml | | | |
| 93775_Dendritic Cells_anti- | 0.0 | 93260_IBD Colitis 2 | 0.1 |
| CD40 | | | |
| 93774_Monocytes_resting | 0.0 | 93261_IBD Crohns | 4.1 |
| 93776_Monocytes_LPS 50 ng/ml | 0.0 | 735010_Colon_normal | 1.2 |
| 93581_Macrophages_resting | 0.3 | 735019_Lung_none | 12.2 |
| 93582_Macrophages_LPS | 0.0 | 64028-1_Thymus_none | 62.8 |
| 100 ng/ml | | | |
| 93098_HUVEC | 0.0 | 64030-1_Kidney_none | 2.2 |
| (Endothelial)_none | | | |
| 93099_HUVEC | 0.0 | | |
| (Endothelial)_starved | | | |

Panel 1.3D Summary Ag2197 Highest expression of the NOV1a gene is seen in a sample derived from a CNS cancer cell line (CT=27). Certain glioblastoma and astrocytoma cell lines also express this gene as well so it may have a role in different types of brain cancer. Among normal tissues derived from the central nervous system, the NOV1a gene is expressed in the amygdala, cerebellum, hippocampus and cerebral cortex.

Among tissues with metabolic function, the NOV1a gene is expressed in the pancreas and in adipose. Interestingly, this gene is also expressed at higher levels in fetal heart and skeletal tissue when compared to the adult tissues.

The NOV1a gene appears to be expressed at high levels in a sample from a renal cancer cell line and from a breast cancer cell line. In addition, the NOV1a gene is expressed in ovarian tissue, but not significantly in cell lines derived from ovarian cancer.

Panel 2D Summary Ag2197 Expression of the NOV1b gene is highest in normal colon tissue (CT=25). The gene is also expressed at high levels in the uterus, but not in uterine cancer. Thus, expression of NOV1b could be used as a diagnostic marker for the presence of uterine cancer. Furthermore, therapeutic upregulation of the activity of the protein product could potentially be useful in the treatment of uterine cancer. The NOV1b gene also appears to be expressed at higher levels in liver cancers (two out of two matched tissue pairs) and kidney cancers (seven out nine matched tissue pairs) when compared to normal adjacent tissue. Thus, expression of the NOV1b gene could be used to diffentiate between kidney and liver cancers and other cancers or normal tissue. Furthermore, thereapeutic inhibition of the activity of the protein encoded by the NOV1b gene, through the use of antibodies or small molecule drugs, could be effective in treating kidney and liver cancers.

Panel 4D Summary Ag2197 The NOV1a gene is expressed at a high level in astrocytes (CT 27.8) and its expression is down regulated upon treatment with TNF-a and IL-1b, suggesting that modulation of this protein could be beneficial in the treatment of CNS diseases-associated inflammation or neurodegeneration. The NOV1a gene is also expressed highly in the thymus (CT 28.4). More moderate expression of this gene is observed in the lung and in a muco-epidermoid cell line (H292). Thus, the protein encoded by the NOV1 a gene could play an important role in the normal homeostasis of these tissues. Therapeutics designed with this protein could be important for maintaining or restoring normal function to these organs during inflammation.

Panel 4.1D Summary Ag4164 Expression of this gene is low/undetectable (CT values>35) among the samples on this panel (data not shown).

Panel CNS_neurodegeneration_v1.0 Summary Ag4164 Expression of this gene is low/undetectable (CT values >35) among the samples on this panel (data not shown).

NOV1c

Please note that the NOV1c sequence is a variant of gene NOV1a annotated in section A. However, the NOV1c sequence only matches a subset of the probe and primer sets discussed above and is discussed independently below in section C. Expression of gene NOV1c was assessed using the primer-probe sets Ag2197, Ag708 and Ag1313b described in Tables 25, 26, and 27. Results from RTQ-PCR runs are shown in Tables 28, 29, 30, and 31.

TABLE 25
Probe Name Ag2197

| | | | | Start | SEQ ID |
|---|---|---|---|---|---|
| Primers | Sequences | TM | Length | Position | NO: |
| Forward | 5′-CCAAGGAAGACCTCTTCATCTT-3′ | 58.8 | 22 | 1022 | 126 |
| Probe | FAM-5′-TCTTGCTTACGGCATAAGCGCTCTCT-3′-TAMRA | 69 | 26 | 1060 | 127 |
| Reverse | 5′-TTCATTTCTATGGGACCTCAGA-3 | 58.7 | 22 | 1086 | 128 |

TABLE 26
Probe Name Ag708

| | | | | Start | SEQ ID |
|---|---|---|---|---|---|
| Primers | Sequences | TM | Length | Position | NO: |
| Forward | 5′-AAAGATGGGACTCGTCATGAC-3′ | 59 | 21 | 232 | 129 |
| Probe | TET-5′-CACGCCATCTTACTGACTGGTCTGGA-3′-TAMRA | 69.5 | 26 | 253 | 130 |
| Reverse | 5′-GTGCAAATCCCAAAGTGTCA-3′ | 59.5 | 20 | 306 | 131 |

TABLE 27
Probe Name Ag1313b

| | | | | Start | SEQ ID |
|---|---|---|---|---|---|
| Primers | Sequences | TM | Length | Position | NO: |
| Forward | 5′-CAGCTGCACGATTAATGAAGAT-3′ | 59.4 | 22 | 264 | 132 |
| Probe | TET-5′-AGGTCTTGGACTGGCCTTCACCATT-3′-TAMRA | 69 | 25 | 288 | 133 |
| Reverse | 5′-CCAAAGTTGTGTCCAGACTCAT-3′ | 59.1 | 22 | 317 | 134 |

TABLE 28
Panel 1.2

| | Relative Expression(%) | |
|---|---|---|
| | 1.2tm888t_{—} | 1.2tm1047t_{—} |
| Tissue Name | ag708 | ag708 |
| Endothelial cells | 0.0 | 0.0 |
| Heart (fetal) | 1.2 | 0.0 |
| Pancreas | 22.5 | 29.5 |
| Pancreatic ca. CAPAN 2 | 0.0 | 0.0 |
| Adrenal Gland (new lot*) | 0.9 | 0.0 |
| Thyroid | 0.5 | 0.0 |
| Salivary gland | 0.4 | 0.0 |
| Pituitary gland | 1.3 | 0.0 |
| Brain (fetal) | 4.0 | 0.0 |
| Brain (whole) | 7.9 | 6.5 |
| Brain (amygdala) | 2.1 | 0.0 |
| Brain (cerebellum) | 16.7 | 23.3 |
| Brain (hippocampus) | 4.6 | 3.1 |
| Brain (thalamus) | 1.5 | 0.0 |
| Cerebral Cortex | 10.7 | 5.8 |
| Spinal cord | 1.1 | 0.0 |
| CNS ca. (glio/astro) U87-MG | 4.4 | 11.7 |
| CNS ca. (glio/astro) U-118-MG | 0.6 | 0.1 |
| CNS ca. (astro) SW1783 | 1.2 | 0.7 |
| CNS ca.* (neuro; met ) SK-N-AS | 0.2 | 0.0 |
| CNS ca. (astro) SF-539 | 1.6 | 1.1 |
| CNS ca. (astro) SNB-75 | 7.7 | 1.7 |
| CNS ca. (glio) SNB-19 | 9.5 | 4.3 |
| CNS ca. (glio) U251 | 100.0 | 100.0 |
| CNS ca. (glio) SF-295 | 2.4 | 0.0 |
| Heart | 4.9 | 0.0 |
| Skeletal Muscle (new lot*) | 3.1 | 0.0 |
| Bone marrow | 0.0 | 0.0 |
| Thymus | 0.2 | 0.0 |
| Spleen | 0.0 | 0.0 |
| Lymph node | 0.2 | 0.0 |
| Colorectal | 0.0 | 0.0 |
| Stomach | 1.2 | 0.0 |
| Small intestine | 1.4 | 0.0 |
| Colon ca. SW480 | 1.5 | 0.0 |
| Colon ca.* (SW480 met)SW620 | 0.0 | 0.0 |
| Colon ca. HT29 | 0.0 | 0.0 |
| Colon ca. HCT-116 | 0.7 | 0.0 |
| Colon ca. CaCo-2 | 0.0 | 0.0 |
| 83219 CC Well to Mod Diff (ODO3866) | 0.1 | 0.0 |
| Colon ca. HCC-2998 | 0.0 | 0.0 |
| Gastric ca.* (liver met) NCI-N87 | 4.1 | 0.1 |
| Bladder | 3.5 | 0.0 |
| Trachea | 0.3 | 0.0 |
| Kidney | 5.4 | 10.7 |
| Kidney (fetal) | 37.9 | 94.0 |
| Renal ca. 786-0 | 4.6 | 5.3 |
| Renal ca. A498 | 12.2 | 20.2 |
| Renal ca. RXF 393 | 3.3 | 4.2 |
| Renal ca. ACHN | 2.8 | 1.1 |
| Renal ca. UO-31 | 2.3 | 0.2 |
| Renal ca. TK-10 | 7.6 | 12.7 |
| Liver | 0.9 | 0.0 |
| Liver (fetal) | 0.2 | 0.0 |
| Liver ca. (hepatoblast) HepG2 | 0.0 | 0.0 |
| Lung | 1.1 | 0.0 |
| Lung (fetal) | 1.2 | 0.0 |
| Lung ca. (small cell) LX-1 | 0.0 | 0.0 |
| Lung ca. (small cell) NCI-H69 | 0.1 | 0.0 |
| Lung ca. (s.cell var.) SHP-77 | 0.0 | 0.0 |
| Lung ca. (large cell) NCI-H460 | 0.0 | 0.0 |
| Lung ca. (non-sm. cell) A549 | 0.5 | 0.0 |
| Lung ca. (non-s.cell) NCI-H23 | 0.0 | 0.0 |
| Lung ca (non-s.cell) HOP-62 | 4.9 | 1.1 |
| Lung ca. (non-s.cl) NCI-H522 | 0.0 | 0.0 |
| Lung ca. (squam.) SW 900 | 3.7 | 2.0 |
| Lung ca. (squam.) NCI-H596 | 0.0 | 0.0 |
| Mammary gland | 3.1 | 4.5 |
| Breast ca.* (pl. effusion) MCF-7 | 0.0 | 0.0 |
| Breast ca.* (pl.ef) MDA-MB-231 | 3.1 | 6.2 |
| Breast ca.* (pl. effusion) T47D | 0.8 | 0.0 |
| Breast ca. BT-549 | 0.7 | 0.0 |
| Breast ca. MDA-N | 10.7 | 14.6 |
| Ovary | 26.4 | 39.5 |
| Ovarian ca. OVCAR-3 | 0.4 | 0.0 |
| Ovarian ca. OVCAR-4 | 0.9 | 0.0 |
| Ovarian ca. OVCAR-5 | 6.0 | 1.7 |
| Ovarian ca. OVCAR-8 | 0.7 | 0.0 |
| Ovarian ca. IGROV-1 | 8.1 | 15.2 |
| Ovarian ca.* (ascites) SK-OV-3 | 2.9 | 0.0 |
| Uterus | 0.7 | 0.0 |
| Placenta | 7.1 | 0.0 |
| Prostate | 0.3 | 0.0 |
| Prostate ca.* (bone met) PC-3 | 1.5 | 0.0 |
| Testis | 3.3 | 0.0 |
| Melanoma Hs688(A).T | 0.0 | 0.0 |
| Melanoma* (met) Hs688(B).T | 0.0 | 0.0 |
| Melanoma UACC-62 | 0.0 | 0.0 |
| Melanoma M14 | 0.0 | 0.0 |
| Melanoma LOX IMVI | 1.5 | 0.0 |
| Melanoma* (met) SK-MEL-5 | 0.0 | 0.0 |

TABLE 29
Panel 1.3D

| | Relative | Relative | Relative |
|---|---|---|---|
| | Expression(%) | Expression(%) | Expression(%) |
| | 1.3dtm4180 | 1.3dx4tm5365 | 1.3dtm3301t_{—} |
| Tissue Name | fam_ag2197 | t_ag1313b_b1 | ag708 |
| Liver adenocarcinoma | 2.6 | 0.5 | 2.3 |
| Pancreas | 2.8 | 1.4 | 4.4 |
| Pancreatic ca. | 0.6 | 0.2 | 0.8 |
| CAPAN 2 | | | |
| Adrenal gland | 0.2 | 0.0 | 0.0 |
| Thyroid | 0.2 | 0.1 | 0.0 |
| Salivary gland | 0.0 | 0.2 | 0.2 |
| Pituitary gland | 0.0 | 0.0 | 0.0 |
| Brain (fetal) | 0.2 | 0.1 | 0.7 |
| Brain (whole) | 3.4 | 2.0 | 2.5 |
| Brain (amygdala) | 1.8 | 1.1 | 1.7 |
| Brain (cerebellum) | 7.4 | 8.0 | 11.2 |
| Brain (hippocampus) | 7.6 | 1.0 | 7.9 |
| Brain (substantia | 0.0 | 0.0 | 0.0 |
| nigra) | | | |
| Brain (thalamus) | 0.2 | 0.1 | 0.0 |
| Cerebral Cortex | 5.6 | 0.5 | 4.6 |
| Spinal cord | 0.2 | 0.2 | 0.8 |
| CNS ca. (glio/astro) | 7.0 | 1.8 | 4.6 |
| U87-MG | | | |
| CNS ca. (glio/astro) | 12.2 | 2.3 | 7.3 |
| U-118-MG | | | |
| CNS ca. (astro) | 4.9 | 2.0 | 5.7 |
| SW1783 | | | |
| CNS ca.* (neuro; met) | 0.0 | 0.0 | 0.0 |
| SK-N-AS | | | |
| CNS ca. (astro) | 4.4 | 1.8 | 3.8 |
| SF-539 | | | |
| CNS ca. (astro) | 33.0 | 13.8 | 36.1 |
| SNB-75 | | | |
| CNS ca. (glio) | 6.0 | 3.4 | 6.7 |
| SNB-19 | | | |
| CNS ca. (glio) U251 | 100.0 | 100.0 | 100.0 |
| CNS ca. (glio) SF-295 | 2.0 | 0.5 | 4.2 |
| Heart (fetal) | 1.2 | 0.1 | 1.3 |
| Heart | 0.0 | 0.0 | 0.6 |
| Fetal Skeletal | 14.8 | 0.3 | 16.3 |
| Skeletal muscle | 0.1 | 0.2 | 0.0 |
| Bone marrow | 0.0 | 0.0 | 0.0 |
| Thymus | 0.3 | 0.0 | 0.5 |
| Spleen | 0.0 | 0.0 | 0.0 |
| Lymph node | 0.2 | 0.3 | 0.1 |
| Colorectal | 0.7 | 0.0 | 0.4 |
| Stomach | 0.7 | 1.2 | 0.7 |
| Small intestine | 1.2 | 0.6 | 2.6 |
| Colon ca. SW480 | 2.2 | 0.1 | 2.4 |
| Colon ca.* (SW480 | 0.0 | 0.0 | 0.0 |
| met)SW620 | | | |
| Colon ca. HT29 | 0.0 | 0.0 | 0.0 |
| Colon ca. | 0.6 | 0.0 | 0.6 |
| HCT-116 | | | |
| Colon ca. CaCo-2 | 0.0 | 0.0 | 0.0 |
| 83219 CC Well to | 4.2 | 0.3 | 2.6 |
| Mod Diff (ODO3866) | | | |
| Colon ca. HCC-2998 | 0.0 | 0.0 | 0.0 |
| Gastric ca.* (liver met) | 2.6 | 1.3 | 1.0 |
| NCI-N87 | | | |
| Bladder | 0.7 | 0.2 | 0.7 |
| Trachea | 0.4 | 0.2 | 0.1 |
| Kidney | 1.9 | 1.3 | 2.8 |
| Kidney (fetal) | 12.6 | 3.0 | 18.3 |
| Renal ca. 786-0 | 10.7 | 2.1 | 15.1 |
| Renal ca. A498 | 47.3 | 9.7 | 34.4 |
| Renal ca. RXF 393 | 4.3 | 6.0 | 3.4 |
| Renal ca. ACHN | 2.6 | 0.8 | 2.5 |
| Renal ca. UO-31 | 10.7 | 3.0 | 13.5 |
| Renal ca. TK-10 | 7.4 | 1.6 | 9.3 |
| Liver | 0.0 | 0.0 | 0.3 |
| Liver (fetal) | 0.2 | 0.0 | 1.1 |
| Liver ca. (hepatoblast) | 0.0 | 0.0 | 0.0 |
| HepG2 | | | |
| Lung | 0.1 | 0.0 | 0.3 |
| Lung (fetal) | 0.2 | 0.0 | 0.4 |
| Lung ca. (small cell) | 0.0 | 0.0 | 0.0 |
| LX-1 | | | |
| Lung ca. (small cell) | 0.0 | 0.0 | 0.0 |
| NCI-H69 | | | |
| Lung ca. (s.cell var.) | 0.0 | 0.0 | 0.0 |
| SHP-77 | | | |
| Lung ca. (large cell) | 0.0 | 0.4 | 0.0 |
| NCI-H460 | | | |
| Lung ca. (non-sm. | 0.8 | 0.3 | 0.4 |
| cell) A549 | | | |
| Lung ca. (non-s.cell) | 0.0 | 0.0 | 0.0 |
| NCI-H23 | | | |
| Lung ca. (non-s.cell) | 4.7 | 1.7 | 4.1 |
| HOP-62 | | | |
| Lung ca. (non-s.cl) | 0.0 | 0.0 | 0.0 |
| NCI-H522 | | | |
| Lung ca. (squam.) | 3.7 | 1.6 | 4.0 |
| SW 900 | | | |
| Lung ca. (squam.) | 0.0 | 0.0 | 0.0 |
| NCI-H596 | | | |
| Mammary gland | 4.0 | 1.1 | 5.8 |
| Breast ca.* | 0.0 | 0.1 | 0.0 |
| (pl. effusion) MCF-7 | | | |
| Breast ca.* (pl.ef) | 46.3 | 7.8 | 12.4 |
| MDA-MB-231 | | | |
| Breast ca.* (pl. | 0.0 | 0.0 | 0.0 |
| effusion) T47D | | | |
| Breast ca. BT-549 | 6.7 | 2.7 | 2.6 |
| Breast ca. MDA-N | 8.3 | 1.2 | 11.1 |
| Ovary | 59.9 | 5.8 | 88.9 |
| Ovarian ca. OVCAR-3 | 0.0 | 2.6 | 0.0 |
| Ovarian ca. OVCAR-4 | 0.0 | 0.0 | 0.0 |
| Ovarian ca. OVCAR-5 | 6.8 | 1.3 | 2.5 |
| Ovarian ca. OVCAR-8 | 1.8 | 0.1 | 1.1 |
| Ovarian ca. IGROV-1 | 4.3 | 0.9 | 4.2 |
| Ovarian ca.* (ascites) | 1.8 | 1.7 | 0.6 |
| SK-OV-3 | | | |
| Uterus | 0.3 | 0.4 | 1.2 |
| Placenta | 0.0 | 0.0 | 0.2 |
| Prostate | 0.1 | 0.1 | 0.1 |
| Prostate ca.* (bone | 0.7 | 0.3 | 0.7 |
| met) PC-3 | | | |
| Testis | 0.4 | 0.2 | 0.8 |
| Melanoma | 0.0 | 0.0 | 0.0 |
| Hs688(A).T | | | |
| Melanoma* (met) | 0.4 | 0.2 | 4.0 |
| Hs688(B).T | | | |
| Melanoma UACC-62 | 0.0 | 0.0 | 0.0 |
| Melanoma M14 | 0.0 | 0.1 | 0.0 |
| Melanoma LOX IMVI | 3.2 | 0.8 | 0.6 |
| Melanoma* (met) | 0.0 | 0.0 | 0.0 |
| SK-MEL-5 | | | |
| Adipose | 1.7 | 0.1 | 1.5 |

TABLE 30
Panel 2D

| | Relative | Relative | Relative |
|---|---|---|---|
| | Expression (%) | Expression (%) | Expression (%) |
| | 2dtm4181fam_{—} | 2Dtm2694t_{—} | 2dx4tm4810t_{—} |
| Tissue Name | ag2197 | ag708 | ag1313b_b2 |
| Normal Colon GENPAK 061003 | 100.0 | 28.3 | 18.4 |
| 83219 CC Well to Mod Diff (ODO3866) | 28.1 | 2.8 | 5.6 |
| 83220 CC NAT (ODO3866) | 0.3 | 0.5 | 3.7 |
| 83221 CC Gr.2 rectosigmoid (ODO3868) | 0.1 | 0.2 | 2.4 |
| 83222 CC NAT (ODO3868) | 0.0 | 0.5 | 0.2 |
| 83235 CC Mod Diff (ODO3920) | 0.0 | 0.0 | 0.2 |
| 83236 CC NAT (ODO3920) | 0.8 | 0.8 | 1.1 |
| 83237 CC Gr.2 ascend colon (ODO3921) | 0.5 | 1.6 | 3.8 |
| 83238 CC NAT (ODO3921) | 0.2 | 2.0 | 1.7 |
| 83241 CC from Partial Hepatectomy | 0.2 | 0.7 | 0.9 |
| (ODO4309) | | | |
| 83242 Liver NAT (ODO4309) | 2.0 | 0.0 | 0.2 |
| 87472 Colon mets to lung (OD04451-01) | 0.0 | 1.1 | 2.0 |
| 87473 Lung NAT (OD04451-02) | 4.8 | 0.0 | 0.0 |
| Normal Prostate Clontech A + 6546-1 | 9.2 | 0.0 | 1.6 |
| 84140 Prostate Cancer (OD04410) | 1.0 | 0.2 | 0.3 |
| 84141 Prostate NAT (OD04410) | 2.2 | 0.0 | 0.0 |
| 87073 Prostate Cancer (OD04720-01) | 2.2 | 0.2 | 0.0 |
| 87074 Prostate NAT (OD04720-02) | 0.3 | 0.6 | 1.1 |
| Normal Lung GENPAK 061010 | 0.7 | 3.3 | 4.7 |
| 83239 Lung Met to Muscle (ODO4286) | 0.0 | 13.2 | 14.1 |
| 83240 Muscle NAT (ODO4286) | 0.0 | 1.2 | 0.4 |
| 84136 Lung Malignant Cancer (OD03126) | 0.5 | 8.2 | 3.7 |
| 84137 Lung NAT (OD03126) | 0.0 | 0.7 | 0.8 |
| 84871 Lung Cancer (OD04404) | 0.0 | 11.7 | 5.4 |
| 84872 Lung NAT (OD04404) | 0.0 | 4.1 | 3.9 |
| 84875 Lung Cancer (OD04565) | 0.2 | 4.8 | 8.9 |
| 84876 Lung NAT (OD04565) | 0.0 | 0.0 | 0.0 |
| 85950 Lung Cancer (OD04237-01) | 1.0 | 3.3 | 7.3 |
| 85970 Lung NAT (OD04237-02) | 0.8 | 1.4 | 0.5 |
| 83255 Ocular Mel Met to Liver (ODO4310) | 0.4 | 0.0 | 0.0 |
| 83256 Liver NAT (ODO4310) | 0.4 | 0.0 | 0.0 |
| 84139 Melanoma Mets to Lung (OD04321) | 0.9 | 48.3 | 43.1 |
| 84138 Lung NAT (OD04321) | 0.0 | 0.2 | 0.1 |
| Normal Kidney GENPAK 061008 | 0.4 | 100.0 | 100.0 |
| 83786 Kidney Ca, Nuclear grade 2 (OD04338) | 0.9 | 9.6 | 18.7 |
| 83787 Kidney NAT (OD04338) | 0.6 | 29.9 | 28.3 |
| 83788 Kidney Ca Nuclear grade 1/2 | 0.5 | 12.0 | 10.4 |
| (OD04339) | | | |
| 83789 Kidney NAT (OD04339) | 0.5 | 29.7 | 33.9 |
| 83790 Kidney Ca, Clear cell type (OD04340) | 0.2 | 3.0 | 3.4 |
| 83791 Kidney NAT (OD04340) | 0.1 | 38.2 | 34.8 |
| 83792 Kidney Ca, Nuclear grade 3 (OD04348) | 0.0 | 16.4 | 16.7 |
| 83793 Kidney NAT (OD04348) | 0.0 | 34.9 | 35.4 |
| 87474 Kidney Cancer (OD04622-01) | 1.1 | 0.0 | 0.4 |
| 87475 Kidney NAT (OD04622-03) | 0.7 | 5.0 | 4.6 |
| 85973 Kidney Cancer (OD04450-01) | 1.3 | 15.9 | 21.6 |
| 85974 Kidney NAT (OD04450-03) | 0.0 | 37.9 | 38.6 |
| Kidney Cancer Clontech 8120607 | 6.5 | 0.0 | 0.3 |
| Kidney NAT Clontech 8120608 | 1.9 | 7.9 | 8.0 |
| Kidney Cancer Clontech 8120613 | 4.2 | 2.2 | 2.3 |
| Kidney NAT Clontech 8120614 | 1.3 | 23.0 | 15.3 |
| Kidney Cancer Clontech 9010320 | 4.3 | 13.3 | 11.1 |
| Kidney NAT Clontech 9010321 | 0.3 | 30.4 | 30.6 |
| Normal Uterus GENPAK 061018 | 1.0 | 11.7 | 7.3 |
| Uterus Cancer GENPAK 064011 | 0.0 | 4.0 | 4.2 |
| Normal Thyroid Clontech A+ 6570-1 | 4.4 | 0.4 | 0.6 |
| Thyroid Cancer GENPAK 064010 | 2.9 | 0.0 | 0.0 |
| Thyroid Cancer INVITROGEN A302152 | 5.3 | 0.3 | 1.2 |
| Thyroid NAT INVITROGEN A302153 | 0.3 | 2.2 | 1.9 |
| Normal Breast GENPAK 061019 | 4.9 | 2.0 | 3.3 |
| 84877 Breast Cancer (OD04566) | 2.1 | 3.1 | 3.3 |
| 85975 Breast Cancer (OD04590-01) | 4.4 | 7.6 | 4.7 |
| 85976 Breast Cancer Mets (OD04590-03) | 13.2 | 4.3 | 6.3 |
| 87070 Breast Cancer Metastasis (OD04655-05) | 0.1 | 0.4 | 0.8 |
| GENPAK Breast Cancer 064006 | 5.7 | 5.4 | 4.1 |
| Breast Cancer Res. Gen. 1024 | 5.7 | 5.5 | 5.1 |
| Breast Cancer Clontech 9100266 | 0.0 | 4.0 | 3.3 |
| Breast NAT Clontech 9100265 | 0.0 | 3.7 | 3.5 |
| Breast Cancer INVITROGEN A209073 | 0.0 | 4.7 | 6.1 |
| Breast NAT INVITROGEN A2090734 | 0.9 | 4.1 | 4.9 |
| Normal Liver GENPAK 061009 | 0.0 | 0.0 | 0.0 |
| Liver Cancer GENPAK 064003 | 2.2 | 0.0 | 0.0 |
| Liver Cancer Research Genetics RNA 1025 | 0.4 | 0.0 | 0.0 |
| Liver Cancer Research Genetics RNA 1026 | 2.0 | 2.5 | 2.0 |
| Paired Liver Cancer Tissue Research Genetics | 1.4 | 0.0 | 0.0 |
| RNA 6004-T | | | |
| Paired Liver Tissue Research Genetics RNA | 0.0 | 0.0 | 0.4 |
| 6004-N | | | |
| Paired Liver Cancer Tissue Research Genetics | 1.0 | 3.2 | 2.0 |
| RNA 6005-T | | | |
| Paired Liver Tissue Research Genetics RNA | 0.0 | 0.0 | 0.0 |
| 6005-N | | | |
| Normal Bladder GENPAK 061001 | 0.4 | 6.4 | 6.2 |
| Bladder Cancer Research Genetics RNA 1023 | 0.2 | 1.2 | 2.2 |
| Bladder Cancer INVITROGEN A302173 | 0.0 | 6.1 | 6.9 |
| 87071 Bladder Cancer (OD04718-01) | 0.0 | 13.6 | 14.8 |
| 87072 Bladder Normal Adjacent (OD04718-03) | 0.0 | 8.7 | 9.6 |
| Normal Ovary Res. Gen. | 0.0 | 77.4 | 60.2 |
| Ovarian Cancer GENPAK 064008 | 0.0 | 32.8 | 32.1 |
| 87492 Ovary Cancer (OD04768-07) | 0.3 | 0.8 | 0.8 |
| 87493 Ovary NAT (OD04768-08) | 0.0 | 12.0 | 10.2 |
| Normal Stomach GENPAK 061017 | 0.0 | 2.9 | 2.3 |
| Gastric Cancer Clontech 9060358 | 0.5 | 1.1 | 1.1 |
| NAT Stomach Clontech 9060359 | 0.4 | 5.9 | 3.5 |
| Gastric Cancer Clontech 9060395 | 0.0 | 0.4 | 0.2 |
| NAT Stomach Clontech 9060394 | 0.3 | 1.8 | 1.1 |
| Gastric Cancer Clontech 9060397 | 0.1 | 9.3 | 5.7 |
| NAT Stomach Clontech 9060396 | 0.0 | 0.2 | 0.8 |
| Gastric Cancer GENPAK 064005 | 0.4 | 0.4 | 1.5 |

TABLE 31
Panel 4D

| | Relative | | Relative |
|---|---|---|---|
| | Expression (%) | | Expression (%) |
| | 4dtm4182fam_{—} | | 4dtm4182fam_{—} |
| Tissue Name | ag2197 | Tissue Name | ag2197 |
| 93768_Secondary Th1_anti- | 0.0 | 93100_HUVEC | 0.1 |
| CD28/anti-CD3 | | (Endothelial)_IL-1b | |
| 93769_Secondary Th2_anti- | 0.0 | 93799_HUVEC | 0.0 |
| CD28/anti-CD3 | | (Endothelial)_IFN gamma | |
| 93770_Secondary Tr1_anti- | 0.0 | 93102_HUVEC | 0.0 |
| CD28/anti-CD3 | | (Endothelial)_TNF alpha + IFN | |
| | | gamma | |
| 93573_Secondary Th1_resting | 0.0 | 93101_HUVEC | 0.0 |
| day 4-6 in IL-2 | | (Endothelial)_TNF alpha + IL4 | |
| 93572_Secondary Th2_resting | 0.0 | 93781_HUVEC | 0.0 |
| day 4-6 in IL-2 | | (Endothelial)_IL-11 | |
| 93571_Secondary Tr1_resting | 0.0 | 93583_Lung Microvascular | 0.0 |
| day 4-6 in IL-2 | | Endothelial Cells_none | |
| 93568_primary Th1_anti- | 0.0 | 93584_Lung Microvascular | 0.0 |
| CD28/anti-CD3 | | Endothelial Cells_TNFa (4 ng/ml) | |
| | | and IL1b (1 ng/ml) | |
| 93569_primary Th2_anti- | 0.0 | 92662_Microvascular Dermal | 0.0 |
| CD28/anti-CD3 | | endothelium_none | |
| 93570_primary Tr1_anti- | 0.0 | 92663_Microvascular Dermal | 0.2 |
| CD28/anti-CD3 | | endothelium_TNFa (4 ng/ml) | |
| | | and IL1b (1 ng/ml) | |
| 93565_primary Th1_resting dy | 0.0 | 93773_Bronchial | 0.0 |
| 4-6 in IL-2 | | epithelium_TNFa (4 ng/ml) and | |
| | | IL1b (1 ng/ml)** | |
| 93566_primary Th2_resting dy | 0.0 | 93347_Small Airway | 0.4 |
| 4-6 in IL-2 | | Epithelium_none | |
| 93567_primary Tr1_resting dy | 0.0 | 93348_Small Airway | 0.0 |
| 4-6 in IL-2 | | Epithelium_TNFa (4 ng/ml) | |
| | | and IL1b (1 ng/ml) | |
| 93351_CD45RA CD4 | 0.0 | 92668_Coronery Artery | 0.3 |
| lymphocyte_anti-CD28/anti- | | SMC_resting | |
| CD3 | | | |
| 93352_CD45RO CD4 | 0.0 | 92669_Coronery Artery | 0.0 |
| lymphocyte_anti-CD28/anti- | | SMC_TNFa (4 ng/ml) and IL1b | |
| CD3 | | (1 ng/ml) | |
| 93251_CD8 Lymphocytes_anti- | 0.0 | 93107_astrocytes_resting | 100.0 |
| CD28/anti-CD3 | | | |
| 93353_chronic CD8 | 0.0 | 93108_astrocytes_TNFa | 16.5 |
| | | (4 ng/ml) | |
| Lymphocytes 2ry_resting dy 4-6 | | and IL1b (1 ng/ml) | |
| in IL-2 | | | |
| 93574_chronic CD8 | 0.0 | 92666_KU-812 | 0.0 |
| Lymphocytes 2ry_activated | | (Basophil)_resting | |
| CD3/CD28 | | | |
| 93354_CD4_none | 0.0 | 92667_KU-812 | 0.0 |
| | | (Basophil)_PMA/ionoycin | |
| 93252_Secondary | 0.0 | 93579_CCD1106 | 0.0 |
| Th1/Th2/Tr1_anti-CD95 CH11 | | (Keratinocytes)_none | |
| 93103_LAK cells_resting | 0.0 | 93580_CCD1106 | 0.0 |
| | | (Keratinocytes)_TNFa and | |
| | | IFNg** | |
| 93788_LAK cells_IL-2 | 0.0 | 93791_Liver Cirrhosis | 1.3 |
| 93787_LAK cells_IL-2 + IL-12 | 0.0 | 93792_Lupus Kidney | 11.3 |
| 93789_LAK cells_IL-2 + IFN | 0.0 | 93577_NCI-H292 | 8.4 |
| gamma | | | |
| 93790_LAK cells_IL-2 + IL-18 | 0.0 | 93358_NCI-H292_IL-4 | 12.5 |
| 93104_LAK | 0.8 | 93360_NCI-H292_IL-9 | 11.8 |
| cells_PMA/ionomycin and IL- | | | |
| 18 | | | |
| 93578_NK Cells IL-2_resting | 0.0 | 93359_NCI-H292_IL-13 | 4.2 |
| 93109_Mixed Lymphocyte | 0.0 | 93357_NCI-H292_IFN gamma | 3.7 |
| Reaction_Two Way MLR | | | |
| 93110_Mixed Lymphocyte | 0.0 | 93777_HPAEC_- | 0.0 |
| Reaction_Two Way MLR | | | |
| 93111_Mixed Lymphocyte | 0.0 | 93778_HPAEC_IL-1 beta/TNA | 0.0 |
| Reaction_Two Way MLR | | alpha | |
| 93112_Mononuclear Cells | 0.0 | 93254_Normal Human Lung | 0.3 |
| (PBMCs)_resting | | Fibroblast_none | |
| 93113_Mononuclear Cells | 0.0 | 93253_Normal Human Lung | 0.0 |
| (PBMCs)_PWM | | Fibroblast_TNFa (4 ng/ml) and | |
| | | IL-1b (1 ng/ml) | |
| 93114_Mononuclear Cells | 0.1 | 93257_Normal Human Lung | 0.0 |
| (PBMCs)_PHA-L | | Fibroblast_IL-4 | |
| 93249_Ramos (B cell)_none | 0.0 | 93256_Normal Human Lung | 0.0 |
| | | Fibroblast_IL-9 | |
| 93250_Ramos (B | 0.0 | 93255_Normal Human Lung | 0.0 |
| cell)_ionomycin | | Fibroblast_IL-13 | |
| 93349_B lymphocytes_PWM | 0.0 | 93258_Normal Human Lung | 0.0 |
| | | Fibroblast_IFN gamma | |
| 93350_B lymphoytes_CD40L | 0.0 | 93106_Dermal Fibroblasts | 0.0 |
| and IL-4 | | CCD1070_resting | |
| 92665_EOL-1 | 0.0 | 93361_Dermal Fibroblasts | 0.0 |
| (Eosinophil)_dbcAMP | | CCD1070_TNF alpha 4 ng/ml | |
| differentiated | | | |
| 93248_EOL-1 | 0.0 | 93105_Dermal Fibroblasts | 0.0 |
| (Eosinophil)_dbcAMP/ | | CCD1070_IL-1 beta 1 ng/ml | |
| PMAionomycin | | | |
| 93356_Dendritic Cells_none | 0.0 | 93772_dermal fibroblast_IFN | 0.0 |
| | | gamma | |
| 93355_Dendritic Cells_LPS | 0.0 | 93771_dermal fibroblast_IL-4 | 0.0 |
| 100 ng/ml | | | |
| 93775_Dendritic Cells_anti- | 0.0 | 93260_IBD Colitis 2 | 0.1 |
| CD40 | | | |
| 93774_Monocytes_resting | 0.0 | 93261_IBD Crohns | 4.1 |
| 93776_Monocytes_LPS 50 ng/ml | 0.0 | 735010_Colon_normal | 1.2 |
| 93581_Macrophages_resting | 0.3 | 735019_Lung_none | 12.2 |
| 93582_Macrophages_LPS | 0.0 | 64028-1_Thymus_none | 62.8 |
| 100 ng/ml | | | |
| 93098_HUVEC | 0.0 | 64030-1_Kidney_none | 2.2 |
| (Endothelial)_none | | | |
| 93099_HUVEC | 0.0 | | |
| (Endothelial)_starved | | | |

Panel 1.2 Summary Ag708 Expression of the NOV1a gene was assessed in two independent experiments using the same probe/primer set. There appears to be poor concordance between runs for some tissues but there is good concordance for others; only those results that are in agreement will be discussed here. In both experiments, highest expression of the NOV11a gene in a sample derived from a glioblastoma cell line (CTs=24-26). Among normal tissues derived from the central nervous system, the NOV1a gene is also expressed at moderate levels in the cerebral cortex, cerebellum and hippocampus.

Among tissues with metabolic function, the NOV1a gene is expressed in the pancreas. Thus, this gene may be involved in the pathogenesis and/or treatment of diseases involving the pancreas, such as pancreatitis and diabetes. In addition, NOV1a gene expression is decreased in a pancreatic cancer cell line.

The NOV1a gene appears to be overexpressed in fetal kidney when compared to the adult kidney. This result suggests that the NOV1a gene could be used to distinguish between adult and fetal kidney tissue and that this gene may play an important role in kidney development, growth and survival. Furthermore, NOV1 a gene expression is higher in normal ovary, mammary gland and lung when compared to the cancer cell lines obtained from these tissues suggesting that this can be used as a marker to differentiate malignant and normal tissue.

Panel 1.3D Summary Ag708/Ag1313b/Ag2197 Three experiments with three different probe and primer sets produced results that were in very good agreement. One run, designated 1.3dx4tm5365t, appears to have lower absolute expression, but produces the same expression profile as the other two experiments. Highest expression of the NOV1a gene in all three runs is seen in a sample derived from a CNS cancer cell line (CTs=27). Certain glioblastoma and astrocytoma cell lines also express this gene as well so it may have a role in different types of brain cancer. Among normal tissues derived from the central nervous system, the NOV1a gene is expressed in the amygdala, cerebellum, hippocampus and cerebral cortex.

Among tissues with metabolic function, the NOV1a gene is expressed in the pancreas and in adipose. Interestingly, this gene is also expressed at higher levels in fetal heart and skeletal tissue when compared to the adult tissues.

The NOV1a gene appears to be expressed at high levels in a sample from a renal cancer cell line and from a breast cancer cell line. In addition, the NOV1a gene is expressed in ovarian tissue, but not significantly in cell lines derived from ovarian cancer.

Panel 2D Summary Ag708/AR1313b/Ag2197 In two runs using two different probe and primer sets, highest expression of the NOV1a gene is seen in the normal ovary, colon and kidney. Furthermore, in all nine matched kidney pairs and in the matched tissue pair derived from the ovary, the NOV1a gene is expressed more highly in normal tissue than in the adjacent cancer samples. This result suggests that expression of the NOV1a gene could be used as a diagnostic marker for the presence of kidney and ovarian cancer. In addition, therapeutic upregulation of the gene activity of NOV1a could be effective in the treatment of kidney and ovarian cancer. The NOV1a gene is also expressed at higher levels in lung cancer samples, when compared to normal adjacent tissue in six out of seven matched tissue pairs. Thus, therapeutic inhibition of the NOV 1 a gene, through the application of antibodies or small molecule drugs, could be effective in the treatment of lung cancer.

Panel 4D Summary Ag2197 The NOV1a gene is expressed at a high level in astrocytes (CT 27.8) and its expression is down regulated upon treatment with TNF-a and IL-1b, suggesting that modulation of this protein could be beneficial in the treatment of CNS diseases-associated inflammation or neurodegeneration. The NOV1a gene is also expressed highly in the thymus (CT 28.4). More moderate expression of this gene is observed in the lung and in a muco-epidermoid cell line (H292). Thus, the protein encoded by the NOV1a gene could play an important role in the normal homeostasis of these tissues. Therapeutics designed with this protein could be important for maintaining or restoring normal function to these organs during inflammation.

NOV3

Expression of gene NOV3 was assessed using the primer-probe set Ag1534 described in Table 32. Results from RTQ-PCR runs are shown in Tables 33, 34, 35, and 36.

TABLE 32
Probe Name Ag1534

| | | | | Start | SEQ ID |
|---|---|---|---|---|---|
| Primers | Sequences | TM | Length | Position | NO: |
| Forward | 5′-TTTCAAGACACCCTGTGATACC-3′ | 59 | 22 | 765 | 135 |
| Probe | FAM-5′-ACTTCGTGTCCTGAATGTTCCAGGCT-3′-TAMRA | 69.1 | 26 | 799 | 136 |
| Reverse | 5′-CAGAGGAATGAAGGCATAGATG-3′ | 58.8 | 22 | 825 | 137 |

TABLE 33
Panel 1.2

| | Relative | | Relative |
|---|---|---|---|
| | Expression(%) | | Expression(%) |
| | 1.2tm2210f_{—} | | 1.2tm2210f_{—} |
| Tissue Name | ag1534 | Tissue Name | ag1534 |
| Endothelial cells | 2.7 | Renal ca. 786-0 | 48.0 |
| Heart (fetal) | 2.1 | Renal ca. A498 | 5.4 |
| Pancreas | 0.5 | Renal ca. RXF 393 | 4.8 |
| Pancreatic ca. CAPAN 2 | 5.9 | Renal ca. ACHN | 40.6 |
| Adrenal Gland (new lot*) | 12.6 | Renal ca. UO-31 | 17.4 |
| Thyroid | 1.2 | Renal ca. TK-10 | 53.2 |
| Salivary gland | 46.3 | Liver | 5.4 |
| Pituitary gland | 0.6 | Liver (fetal) | 0.9 |
| Brain (fetal) | 3.1 | Liver ca. (hepatoblast) HepG2 | 0.3 |
| Brain (whole) | 0.2 | Lung | 1.2 |
| Brain (amygdala) | 4.5 | Lung (fetal) | 1.2 |
| Brain (cerebellum) | 0.8 | Lung ca. (small cell) LX-1 | 2.3 |
| Brain (hippocampus) | 18.6 | Lung ca. (small cell) NCI-H69 | 23.2 |
| Brain (thalamus) | 8.5 | Lung ca. (s.cell var.) SHP-77 | 0.6 |
| Cerebral Cortex | 29.9 | Lung ca. (large cell) NCI-H460 | 18.0 |
| Spinal cord | 4.8 | Lung ca. (non-sm. cell) A549 | 13.0 |
| CNS ca. (glio/astro) U87-MG | 14.3 | Lung ca. (non-s.cell) NCI-H23 | 42.6 |
| CNS ca. (glio/astro) U-118-MG | 6.6 | Lung ca. (non-s.cell) HOP-62 | 24.3 |
| CNS ca. (astro) SW1783 | 2.4 | Lung ca. (non-s.cl) NCI-H522 | 18.8 |
| CNS ca.* (neuro; met) SK-N- | 6.4 | Lung ca. (squam.) SW 900 | 90.8 |
| AS | | | |
| CNS ca. (astro) SF-539 | 2.6 | Lung ca. (squam.) NCI-H596 | 40.6 |
| CNS ca. (astro) SNB-75 | 6.0 | Mammary gland | 12.6 |
| CNS ca. (glio) SNB-19 | 11.0 | Breast ca.* (pl. effusion) MCF-7 | 17.8 |
| CNS ca. (glio) U251 | 5.4 | Breast ca.* (pl.ef) MDA-MB- | 1.5 |
| | | 231 | |
| CNS ca. (glio) SF-295 | 41.8 | Breast ca.* (pl. effusion) T47D | 26.6 |
| Heart | 32.1 | Breast ca. BT-549 | 2.4 |
| Skeletal Muscle (new lot*) | 2.7 | Breast ca. MDA-N | 0.2 |
| Bone marrow | 1.0 | Ovary | 3.7 |
| Thymus | 0.0 | Ovarian ca. OVCAR-3 | 10.3 |
| Spleen | 1.9 | Ovarian ca. OVCAR-4 | 6.8 |
| Lymph node | 1.6 | Ovarian ca. OVCAR-5 | 44.8 |
| Colorectal | 2.9 | Ovarian ca. OVCAR-8 | 21.3 |
| Stomach | 1.0 | Ovarian ca. IGROV-1 | 18.0 |
| Small intestine | 6.8 | Ovarian ca.* (ascites) SK-OV-3 | 22.5 |
| Colon ca. SW480 | 0.6 | Uterus | 6.2 |
| Colon ca.* (SW480 met)SW620 | 2.2 | Placenta | 1.4 |
| Colon ca. HT29 | 4.2 | Prostate | 50.3 |
| Colon ca. HCT-116 | 6.3 | Prostate ca.* (bone met)PC-3 | 47.0 |
| Colon ca. CaCo-2 | 2.1 | Testis | 1.1 |
| 83219 CC Well to Mod Diff | 0.1 | Melanoma Hs688(A).T | 11.3 |
| (ODO3866) | | | |
| Colon ca. HCC-2998 | 6.3 | Melanoma* (met) Hs688(B).T | 7.8 |
| Gastric ca.* (liver met) NCI- | 0.0 | Melanoma UACC-62 | 20.2 |
| N87 | | | |
| Bladder | 28.5 | Melanoma M14 | 16.7 |
| Trachea | 3.6 | Melanoma LOX IMVI | 1.3 |
| Kidney | 100.0 | Melanoma* (met) SK-MEL-5 | 33.2 |
| Kidney (fetal) | 65.5 | | |

TABLE 34
Panel 1.3D

| | Relative | | Relative |
|---|---|---|---|
| | Expression(%) | | Expression(%) |
| | 1.3Dtm2922f_{—} | | 1.3Dtm2922f_{—} |
| Tissue Name | ag1534 | Tissue Name | ag1534 |
| Liver adenocarcinoma | 6.2 | Kidney (fetal) | 36.9 |
| Pancreas | 0.0 | Renal ca. 786-0 | 98.6 |
| Pancreatic ca. CAPAN 2 | 14.8 | Renal ca. A498 | 1.5 |
| Adrenal gland | 3.5 | Renal ca. RXF 393 | 4.6 |
| Thyroid | 10.4 | Renal ca. ACHN | 28.3 |
| Salivary gland | 3.9 | Renal ca. UO-31 | 13.1 |
| Pituitary gland | 18.8 | Renal ca. TK-10 | 21.9 |
| Brain (fetal) | 35.8 | Liver | 1.8 |
| Brain (whole) | 13.9 | Liver (fetal) | 3.4 |
| Brain (amygdala) | 21.2 | Liver ca. (hepatoblast) HepG2 | 0.0 |
| Brain (cerebellum) | 5.2 | Lung | 10.4 |
| Brain (hippocampus) | 100.0 | Lung (fetal) | 47.3 |
| Brain (substantia nigra) | 1.7 | Lung ca. (small cell) LX-1 | 3.9 |
| Brain (thalamus) | 7.2 | Lung ca. (small cell) NCI-H69 | 29.9 |
| Cerebral Cortex | 20.9 | Lung ca. (s.cell var.) SHP-77 | 1.5 |
| Spinal cord | 5.4 | Lung ca. (large cell) NCI-H460 | 9.0 |
| CNS ca. (glio/astro) U87-MG | 5.1 | Lung ca. (non-sm. cell) A549 | 16.6 |
| CNS ca. (glio/astro) U-118-MG | 27.5 | Lung ca. (non-s.cell) NCI-H23 | 41.8 |
| CNS ca. (astro) SW1783 | 9.0 | Lung ca (non-s.cell) HOP-62 | 15.3 |
| CNS ca.* (neuro; met) SK-N- | 39.5 | Lung ca. (non-s.cl) NCI-H522 | 2.4 |
| AS | | | |
| CNS ca. (astro) SF-539 | 14.2 | Lung ca. (squam.) SW 900 | 65.1 |
| CNS ca. (astro) SNB-75 | 35.4 | Lung ca. (squam.) NCI-H596 | 9.0 |
| CNS ca. (glio) SNB-19 | 28.7 | Mammary gland | 5.7 |
| CNS ca. (glio) U251 | 12.5 | Breast ca.* (pl. effusion) MCF-7 | 14.8 |
| CNS ca. (glio) SF-295 | 26.8 | Breast ca.* (pl.ef) MDA-MB- | 4.9 |
| | | 231 | |
| Heart (fetal) | 0.0 | Breast ca.* (pl. effusion) T47D | 17.4 |
| Heart | 0.0 | Breast ca. BT-549 | 4.4 |
| Fetal Skeletal | 21.2 | Breast ca. MDA-N | 0.0 |
| Skeletal muscle | 0.0 | Ovary | 8.5 |
| Bone marrow | 0.0 | Ovarian ca. OVCAR-3 | 10.4 |
| Thymus | 0.0 | Ovarian ca. OVCAR-4 | 0.0 |
| Spleen | 7.2 | Ovarian ca. OVCAR-5 | 12.4 |
| Lymph node | 3.9 | Ovarian ca. OVCAR-8 | 17.4 |
| Colorectal | 8.4 | Ovarian ca. IGROV-1 | 10.9 |
| Stomach | 10.4 | Ovarian ca.* (ascites) SK-OV-3 | 27.7 |
| Small intestine | 3.7 | Uterus | 13.1 |
| Colon ca. SW480 | 2.8 | Placenta | 0.0 |
| Colon ca.* (SW480 met)SW620 | 3.1 | Prostate | 21.6 |
| Colon ca. HT29 | 5.5 | Prostate ca.* (bone met)PC-3 | 17.9 |
| Colon ca. HCT-116 | 1.3 | Testis | 36.9 |
| Colon ca. CaCo-2 | 1.7 | Melanoma Hs688(A).T | 27.0 |
| 83219 CC Well to Mod Diff | 5.6 | Melanoma* (met) Hs688(B).T | 37.6 |
| (ODO3866) | | | |
| Colon ca. HCC-2998 | 0.0 | Melanoma UACC-62 | 0.0 |
| Gastric ca.* (liver met) NCI- | 0.0 | Melanoma M14 | 8.0 |
| N87 | | | |
| Bladder | 8.8 | Melanoma LOX IMVI | 0.0 |
| Trachea | 40.3 | Melanoma* (met) SK-MEL-5 | 48.3 |
| Kidney | 14.6 | Adipose | 32.3 |

TABLE 35
Panel 2D

| | Relative |
|---|---|
| | Expression (%) |
| | 2Dtm2414f_{—} |
| Tissue Name | ag1534 |
| Normal Colon GENPAK 061003 | 20.3 |
| 83219 CC Well to Mod Diff (ODO3866) | 1.6 |
| 83220 CC NAT (ODO3866) | 1.7 |
| 83221 CC Gr.2 rectosigmoid (ODO3868) | 2.7 |
| 83222 CC NAT (ODO3868) | 2.9 |
| 83235 CC Mod Diff (ODO3920) | 16.0 |
| 83236 CC NAT (ODO3920) | 0.5 |
| 83237 CC Gr.2 ascend colon (ODO3921) | 11.0 |
| 83238 CC NAT (ODO3921) | 1.7 |
| 83241 CC from Partial Hepatectomy (ODO4309) | 4.5 |
| 83242 Liver NAT (ODO4309) | 0.0 |
| 87472 Colon mets to lung (OD04451-01) | 2.3 |
| 87473 Lung NAT (OD04451-02) | 16.4 |
| Normal Prostate Clontech A+ 6546-1 | 75.3 |
| 84140 Prostate Cancer (OD04410) | 29.9 |
| 84141 Prostate NAT (OD04410) | 24.1 |
| 87073 Prostate Cancer (OD04720-01) | 29.9 |
| 87074 Prostate NAT (OD04720-02) | 16.2 |
| Normal Lung GENPAK 061010 | 25.5 |
| 83239 Lung Met to Muscle (ODO4286) | 1.4 |
| 83240 Muscle NAT (ODO4286) | 1.8 |
| 84136 Lung Malignant Cancer (OD03126) | 14.1 |
| 84137 Lung NAT (OD03126) | 43.8 |
| 84871 Lung Cancer (OD04404) | 18.9 |
| 84872 Lung NAT (OD04404) | 33.7 |
| 84875 Lung Cancer (OD04565) | 7.2 |
| 84876 Lung NAT (OD04565) | 8.4 |
| 85950 Lung Cancer (OD04237-01) | 6.0 |
| 85970 Lung NAT (OD04237-02) | 6.6 |
| 83255 Ocular Mel Met to Liver (ODO4310) | 0.0 |
| 83256 Liver NAT (ODO4310) | 3.2 |
| 84139 Melanoma Mets to Lung (OD04321) | 8.0 |
| 84138 Lung NAT (OD04321) | 17.4 |
| Normal Kidney GENPAK 061008 | 62.0 |
| 83786 Kidney Ca, Nuclear grade 2 (OD04338) | 20.3 |
| 83787 Kidney NAT (OD04338) | 19.6 |
| 83788 Kidney Ca Nuclear grade 1/2 (OD04339) | 100.0 |
| 83789 Kidney NAT (OD04339) | 25.7 |
| 83790 Kidney Ca, Clear cell type (OD04340) | 7.9 |
| 83791 Kidney NAT (OD04340) | 24.7 |
| 83792 Kidney Ca, Nuclear grade 3 (OD04348) | 0.6 |
| 83793 Kidney NAT (OD04348) | 24.3 |
| 87474 Kidney Cancer (OD04622-01) | 4.1 |
| 87475 Kidney NAT (OD04622-03) | 0.9 |
| 85973 Kidney Cancer (OD04450-01) | 4.2 |
| 85974 Kidney NAT (OD04450-03) | 24.0 |
| Kidney Cancer Clontech 8120607 | 2.1 |
| Kidney NAT Clontech 8120608 | 0.0 |
| Kidney Cancer Clontech 8120613 | 0.8 |
| Kidney NAT Clontech 8120614 | 90.8 |
| Kidney Cancer Clontech 9010320 | 4.1 |
| Kidney NAT Clontech 9010321 | 4.2 |
| Normal Uterus GENPAK 061018 | 1.9 |
| Uterus Cancer GENPAK 064011 | 12.7 |
| Normal Thyroid Clontech A+ 6570-1 | 25.3 |
| Thyroid Cancer GENPAK 064010 | 2.4 |
| Thyroid Cancer INVITROGEN A302152 | 3.0 |
| Thyroid NAT INVITROGEN A302153 | 20.3 |
| Normal Breast GENPAK 061019 | 14.3 |
| 84877 Breast Cancer (OD04566) | 15.5 |
| 85975 Breast Cancer (OD04590-01) | 4.5 |
| 85976 Breast Cancer Mets (OD04590-03) | 5.0 |
| 87070 Breast Cancer Metastasis (OD04655-05) | 68.8 |
| GENPAK Breast Cancer 064006 | 11.8 |
| Breast Cancer Res. Gen. 1024 | 19.8 |
| Breast Cancer Clontech 9100266 | 0.3 |
| Breast NAT Clontech 9100265 | 0.0 |
| Breast Cancer INVITROGEN A209073 | 11.0 |
| Breast NAT INVITROGEN A2090734 | 9.9 |
| Normal Liver GENPAK 061009 | 0.8 |
| Liver Cancer GENPAK 064003 | 0.6 |
| Liver Cancer Research Genetics RNA 1025 | 1.5 |
| Liver Cancer Research Genetics RNA 1026 | 0.0 |
| Paired Liver Cancer Tissue Research Genetics RNA | 1.5 |
| 6004-T | |
| Paired Liver Tissue Research Genetics RNA 6004-N | 0.4 |
| Paired Liver Cancer Tissue Research Genetics RNA | 0.0 |
| 6005-T | |
| Paired Liver Tissue Research Genetics RNA 6005-N | 0.0 |
| Normal Bladder GENPAK 061001 | 6.0 |
| Bladder Cancer Research Genetics RNA 1023 | 3.4 |
| Bladder Cancer INVITROGEN A302173 | 13.0 |
| 87071 Bladder Cancer (OD04718-01) | 14.9 |
| 87072 Bladder Normal Adjacent (OD04718-03) | 9.9 |
| Normal Ovary Res. Gen. | 4.4 |
| Ovarian Cancer GENPAK 064008 | 37.1 |
| 87492 Ovary Cancer (OD04768-07) | 0.9 |
| 87493 Ovary NAT (OD04768-08) | 0.3 |
| Normal Stomach GENPAK 061017 | 5.5 |
| Gastric Cancer Clontech 9060358 | 0.0 |
| NAT Stomach Clontech 9060359 | 0.0 |
| Gastric Cancer Clontech 9060395 | 0.4 |
| NAT Stomach Clontech 9060394 | 0.0 |
| Gastric Cancer Clontech 9060397 | 0.0 |
| NAT Stomach Clontech 9060396 | 0.0 |
| Gastric Cancer GENPAK 064005 | 7.7 |

TABLE 36
Panel 3D

| | Relative | | Relative |
|---|---|---|---|
| | Expression(%) | | Expression(%) |
| | 3dtm4931f_{—} | | 3dtm4931f_{—} |
| Tissue Name | ag1534 | Tissue Name | ag1534 |
| 94905_Daoy_Medulloblastoma/ | 0.6 | 94954_Ca Ski_Cervical | 3.4 |
| Cerebellum_sscDNA | | epidermoid carcinoma | |
| | | (metastasis)_sscDNA | |
| 94906_TE671_Medulloblastom | 1.2 | 94955_ES-2_Ovarian clear cell | 2.3 |
| /Cerebellum_sscDNA | | carcinoma_sscDNA | |
| 94907_D283 | 100.0 | 94957_Ramos/6h stim_{—} | 0.0 |
| Med_Medulloblastoma/ | | Stimulated with | |
| Cerebellum_sscDNA | | PMA/ionomycin 6h_sscDNA | |
| 94908_PFSK-1_Primitive | 1.6 | 94958_Ramos/14h stim_{—} | 0.0 |
| Neuroectodermal/Cerebellum_{—} | | Stimulated with | |
| sscDNA | | PMA/ionomycin 14h_sscDNA | |
| 94909_XF-498_CNS_sscDNA | 5.1 | 94962_MEG-01_Chronic | 6.4 |
| | | myelogenous leukemia | |
| | | (megokaryoblast)_sscDNA | |
| 94910_SNB- | 2.6 | 94963_Raji_Burkitt's | 0.0 |
| 78_CNS/glioma_sscDNA | | lymphoma_sscDNA | |
| 94911_SF- | 1.1 | 94964_Daudi_Burkitt's | 1.2 |
| 268_CNS/glioblastoma_sscDNA | | lymphoma_sscDNA | |
| 94912_T98G_Glioblastoma_{—} | 4.1 | 94965_U266_B-cell | 0.0 |
| sscDNA | | plasmacytoma/myeloma_sscDNA | |
| 96776_SK-N- | 2.5 | 94968_CA46_Burkitt's | 0.0 |
| SH_Neuroblastoma | | lymphoma_sscDNA | |
| (metastasis)_sscDNA | | | |
| 94913_SF- | 2.1 | 94970_RL_non-Hodgkin's B- | 0.0 |
| 295_CNS/glioblastoma_sscDNA | | cell lymphoma_sscDNA | |
| 94914_Cerebellum_sscDNA | 6.3 | 94972_JM1_pre-B-cell | 0.7 |
| | | lymphoma/leukemia_sscDNA | |
| 96777_Cerebellum_sscDNA | 0.0 | 94973_Jurkat_T cell | 0.0 |
| | | leukemia_sscDNA | |
| 94916_NCI- | 6.7 | 94974_TF- | 3.5 |
| H292_Mucoepidermoid lung | | 1_Erythroleukemia_sscDNA | |
| carcinoma_sscDNA | | | |
| 94917_DMS-114_Small cell | 0.0 | 94975_HUT 78_T-cell | 0.6 |
| lung cancer_sscDNA | | lymphoma_sscDNA | |
| 94918_DMS-79_Small cell | 0.8 | 94977_U937_Histiocytic | 0.8 |
| lung | | lymphoma_sscDNA | |
| cancer/neuroendocrine_sscDNA | | | |
| 94919_NCI-H146_Small cell | 1.5 | 94980_KU-812_Myelogenous | 2.8 |
| lung | | leukemia_sscDNA | |
| cancer/neuroendocrine_sscDNA | | | |
| 94920_NCI-H526_Small cell | 2.5 | 94981_769-P_Clear cell renal | 9.0 |
| lung | | carcinoma_sscDNA | |
| cancer/neuroendocrine_sscDNA | | | |
| 94921_NCI-N417_Small cell | 1.7 | 94983_Caki-2_Clear cell renal | 1.1 |
| lung | | carcinoma_sscDNA | |
| cancer/neuroendocrine_sscDNA | | | |
| 94923_NCI-H82_Small cell | 18.6 | 94984_SW 839_Clear cell renal | 12.5 |
| lung | | carcinoma_sscDNA | |
| cancer/neuroendocrine_sscDNA | | | |
| 94924_NCI-H157_Squamous | 1.6 | 94986_G401_Wilms' | 0.0 |
| cell lung cancer | | tumor_sscDNA | |
| (metastasis)_sscDNA | | | |
| 94925_NCI-H1155_Large cell | 5.2 | 94987_Hs766T_Pancreatic | 2.0 |
| lung | | carcinoma (LN | |
| cancer/neuroendocrine_sscDNA | | metastasis)_sscDNA | |
| 94926_NCI-H1299_Large cell | 3.3 | 94988_CAPAN-1_Pancreatic | 0.0 |
| lung | | adenocarcinoma (liver | |
| cancer/neuroendocrine_sscDNA | | metastasis)_sscDNA | |
| 94927_NCI-H727_Lung | 0.0 | 94989_SU86.86_Pancreatic | 0.7 |
| carcinoid_sscDNA | | carcinoma (liver | |
| | | metastasis)_sscDNA | |
| 94928_NCI-UMC-11_Lung | 2.9 | 94990_BxPC-3_Pancreatic | 3.1 |
| carcinoid_sscDNA | | adenocarcinoma_sscDNA | |
| 94929_LX-1_Small cell lung | 0.0 | 94991_HPAC_Pancreatic | 0.4 |
| cancer_sscDNA | | adenocarcinoma_sscDNA | |
| 94930_Colo-205_Colon | 0.0 | 94992_MIA PaCa-2_Pancreatic | 0.7 |
| cancer_sscDNA | | carcinoma_sscDNA | |
| 94931_KM12_Colon | 0.7 | 94993_CFPAC-1_Pancreatic | 11.8 |
| cancer_sscDNA | | ductal | |
| | | adenocarcinoma_sscDNA | |
| 94932_KM20L2_Colon | 0.0 | 94994_PANC-1_Pancreatic | 2.3 |
| cancer_sscDNA | | epithelioid ductal | |
| | | carcinoma_sscDNA | |
| 94933_NCI-H716_Colon | 0.0 | 94996_T24_Bladder carcinma | 4.7 |
| cancer_sscDNA | | (transitional cell)_sscDNA | |
| 94935_SW-48_Colon | 0.0 | 94997_5637_Bladder | 3.1 |
| adenocarcinoma_sscDNA | | carcinoma_sscDNA | |
| 94936_SW1116_Colon | 0.4 | 94998_HT-1197_Bladder | 2.9 |
| adenocarcinoma_sscDNA | | carcinoma_sscDNA | |
| 94937_LS 174T_Colon | 8.7 | 94999_UM-UC-3_Bladder | 0.0 |
| adenocarcinoma_sscDNA | | carcinma (transitional | |
| | | cell)_sscDNA | |
| 94938_SW-948_Colon | 0.7 | 95000_A204_Rhabdomyosarco | 1.3 |
| adenocarcinoma_sscDNA | | ma_sscDNA | |
| 94939_SW-480_Colon | 4.8 | 95001_HT- | 4.2 |
| adenocarcinoma_sscDNA | | 1080_Fibrosarcoma_sscDNA | |
| 94940_NCI-SNU-5_Gastric | 1.3 | 95002_MG-63_Osteosarcoma | 1.4 |
| carcinoma_sscDNA | | (bone)_sscDNA | |
| 94941_KATO III_Gastric | 0.0 | 95003_SK-LMS- | 3.2 |
| carcinoma_sscDNA | | 1_Leiomyosarcoma | |
| | | (vulva)_sscDNA | |
| 94943_NCI-SNU-16_Gastric | 0.4 | 95004_SJRH30_Rhabdomyosar | 1.7 |
| carcinoma_sscDNA | | coma (met to bone | |
| | | marrow)_sscDNA | |
| 94944_NCI-SNU-1_Gastric | 0.3 | 95005_A431_Epidermoid | 4.8 |
| carcinoma_sscDNA | | carcinoma_sscDNA | |
| 94946_RF-1_Gastric | 9.2 | 95007_WM266- | 0.0 |
| adenocarcinoma_sscDNA | | 4_Melanoma_sscDNA | |
| 94947_RF-48_Gastric | 5.1 | 95010_DU 145_Prostate | 0.0 |
| adenocarcinoma_sscDNA | | carcinoma (brain | |
| | | metastasis)_sscDNA | |
| 96778_MKN-45_Gastric | 1.1 | 95012_MDA-MB-468_Breast | 2.8 |
| carcinoma_sscDNA | | adenocarcinoma_sscDNA | |
| 94949_NCI-N87_Gastric | 0.8 | 95013_SCC-4_Squamous cell | 0.0 |
| carcinoma_sscDNA | | carcinoma of tongue_sscDNA | |
| 94951_OVCAR-5_Ovarian | 0.0 | 95014_SCC-9_Squamous cell | 0.0 |
| carcinoma_sscDNA | | carcinoma of tongue_sscDNA | |
| 94952_RL95-2_Uterine | 6.4 | 95015_SCC-15_Squamous cell | 0.8 |
| carcinoma_sscDNA | | carcinoma of tongue_sscDNA | |
| 94953_HelaS3_Cervical | 0.2 | 95017_CAL 27_Squamous cell | 3.7 |
| adenocarcinoma_sscDNA | | carcinoma of tongue_sscDNA | |

Panel 1.2 Summary Ag1534 The NOV3 gene encodes a protein with homology to the ileal sodium/bile cotransporter. Highest expression of this gene is detected in the kidney (CT=28.1). The NOV3 gene appears to be expressed in clusters of cell lines derived from breast cancer, ovarian cancer, lung cancer, renal cancer and melanoma. Thus expression of this gene could be used to detect the presence of any of these cancers. Furthermore, therapeutic modulation of the expression of the NOV3 gene or the activity of its protein product may be beneficial in the treatment of breast cancer, ovarian cancer, lung cancer, renal cancer and melanoma.

Among tissues involved in metabolic function, the NOV3 gene is expressed in the thyroid, adrenal gland, heart, liver, and skeletal muscle. Thus, the protein encoded by the NOV3 gene could be involved in the pathogenesis and/or treatment of diseases that involve any of these tissues. Furthermore, the NOV3 gene is expressed at higher levels in adult heart tissue (CT=29.7) than in fetal heart tissue (CT=33.6). Therefore, expression of the NOV3 gene could also be used to differentiate between adult and fetal heart tissue.

The NOV3 gene is also widely expressed in tissues originating in the central nervous system. These tissues include the fetal brain, amygdala, hippocampus, thalamus, cerebral cortex and spinal cord. This transporter gene most likely plays a role in the uptake of nutrients. Blockade of this transporter may decrease the loss of neurons due to excitotoxicity during ischemic stroke.

Panel 1.3D Summary Ag1534 Highest expression of the NOV3 gene in Panel 1.3D is detected in the hippocampus (CT=31.2). This gene is also expressed in the amygdala and cerebral cortex. Please see Panel 1.2 summary for discussion of potential utility of this gene with respect to CNS function.

Among tissues with metabolic function, the NOV3 gene is expressed in the thyroid, pituitary gland, adipose, and fetal skeletal muscle. Interestingly, this gene is much more highly expressed in fetal skeletal muscle (CT=33.4) than in adult skeletal muscle (CT=40), suggesting that this gene could be used to distinguish the two. In addition, the increased NOV3 gene expression in fetal skeletal muscle when compared to adult suggests that the protein product may enhance muscular growth or development in the fetus and thus may also act in a regenerative capacity in the adult. Therefore, therapeutic modulation of the NOV3 gene could be useful in treatment of muscular related disease. More specifically, treatment of weak or dystrophic muscle with the protein encoded by this gene could restore muscle mass or function. This gene is also more highly expressed in fetal kidney, lung and brain when compared to the corresponding adult tissues.

Panel 2D Summary Ag1534 The NOV3 gene is most highly expressed in a kidney cancer (CT=31.1). In general, however, this gene is more commonly expressed at higher levels in normal tissues than the adjacent tumor tissues. Specifically, this gene is expressed at higher levels in normal adjacent tissues next to thyroid cancer as well as adjacent to some kidney and lung cancers. These data indicate that NOV3 gene expression might be used to distinguish normal tissue from malignant tissue and also that therapeutic modulation of this gene product might be of use in the treatment of these types of cancer.

Panel 3D Summary Ag 1534 Highest expression of the NOV3 gene is detected in a cell line derived from a medulloblastoma (CT=30.1). Additionally there is expression in a chronic myelogenous leukemia (megokaryoblast) cell line, a gastric adenocarcinoma cell line, a clear cell renal carcinoma cell line, a pancreatic ductal adenocarcinoma cell line and a small cell lung cancer cell line. Thus, the expression of this gene could be used to distinguish some cancer cell lines from others. In addition, these data indicate that the expression of the NOV3 gene might be associated with these forms of cancer and thus, therapeutic modulation of this gene might be of use in the treatment of cancer.

References:

1. Oelkers P, Kirby L C, Heubi J E, Dawson P A. Primary bile acid malabsorption caused by mutations in the ileal sodium-dependent bile acid transporter gene (SLC10A2). J Clin Invest 1997 Apr 15;99(8):1880-7.

Primary bile acid malabsorption (PBAM) is an idiopathic intestinal disorder associated with congenital diarrhea, steatorrhea, interruption of the enterohepatic circulation of bile acids, and reduced plasma cholesterol levels. The molecular basis of PBAM is unknown, and several conflicting mechanisms have been postulated. In this study, we cloned the human ileal Na+/bile acid cotransporter gene (SLC10A2) and employed single-stranded conformation polymorphism analysis to screen for PBAM-associated mutations. Four polymorphisms were identified and sequenced in a family with congenital PBAM. One allele encoded an A171 S missense mutation and a mutated donor splice site for exon 3. The other allele encoded two missense mutations at conserved amino acid positions, L243P and T262M. In transfected COS cells, the L243P, T262M, and double mutant (L243P/T262M) did not affect transporter protein expression or trafficking to the plasma membrane; however, transport of taurocholate and other bile acids was abolished. In contrast, the A171S mutation had no effect on taurocholate uptake. The dysfunctional mutations were not detected in 104 unaffected control subjects, whereas the A171 S was present in 28% of that population. These findings establish that SLC 10A2 mutations can cause PBAM and underscore the ileal Na+/bile acid cotransporter's role in intestinal reclamation of bile acids.

NOV4

Expression of gene NOV4 was assessed using the primer-probe sets Ag2432 and Ag1250 described in Tables 37 and 38. Results from RTQ-PCR runs are shown in Tables 39, 40, 41, and 42.

TABLE 37
Probe Name Ag2432

| | | | | Start | SEQ ID |
|---|---|---|---|---|---|
| Primers | Sequences | TM | Length | Position | NO: |
| Forward | 5′-GTAGGCAAAGGGACTCACTGT-3′ | 58.3 | 21 | 153 | 138 |
| Probe | FAM-5′-CAGAAATCAATAATCTTTGACTGCCG-3′-TAMRA | 64.1 | 26 | 189 | 139 |
| Reverse | 5′-GCACATTACGTGGCTGAGA-3′ | 58.4 | 19 | 216 | 140 |

TABLE 38
Probe Name Ag1250

| | | | | Start | |
|---|---|---|---|---|---|
| Primers | Sequences | TM | Length | Position | SEQ ID NO: |
| Forward | 5′-CGTGGTGAACTCTGCCTTATAT-3′ | 58.3 | 22 | 112 | 141 |
| Probe | TET-5′-CACAGAGCTGTCGTCTTTGACCGATT-3′-TAMRA | 68.7 | 26 | 149 | 142 |
| Reverse | 5′-AGTCCCTTTGCCTACCACAAT-3′ | 59.9 | 21 | 191 | 143 |

TABLE 39
Panel 1.2

| | Relative | | Relative |
|---|---|---|---|
| | Expression(%) | | Expression(%) |
| | 1.2tm1412t_{—} | | 1.2tm1412t_{—} |
| Tissue Name | ag1250 | Tissue Name | ag1250 |
| Endothelial cells | 24.8 | Renal ca. 786-0 | 8.4 |
| Heart (fetal) | 12.7 | Renal ca. A498 | 27.4 |
| Pancreas | 13.3 | Renal ca. RXF 393 | 4.1 |
| Pancreatic ca. CAPAN 2 | 10.4 | Renal ca. ACHN | 15.2 |
| Adrenal Gland (new lot*) | 23.8 | Renal ca. UO-31 | 11.0 |
| Thyroid | 10.0 | Renal ca. TK-10 | 27.5 |
| Salivary gland | 31.6 | Liver | 18.8 |
| Pituitary gland | 10.9 | Liver (fetal) | 13.4 |
| Brain (fetal) | 7.1 | Liver ca. (hepatoblast) HepG2 | 25.0 |
| Brain (whole) | 12.7 | Lung | 5.2 |
| Brain (amygdala) | 5.7 | Lung (fetal) | 6.7 |
| Brain (cerebellum) | 10.1 | Lung ca. (small cell) LX-1 | 38.4 |
| Brain (hippocampus) | 14.2 | Lung ca. (small cell) NCI-H69 | 12.8 |
| Brain (thalamus) | 9.1 | Lung ca. (s. cell var.) SHP-77 | 12.3 |
| Cerebral Cortex | 15.0 | Lung ca. (large cell) NCI-H460 | 32.1 |
| Spinal cord | 7.3 | Lung ca. (non-sm.cell) A549 | 25.9 |
| CNS ca. (glio/astro) U87-MG | 15.7 | Lung ca. (non-s.cell) NCI-H23 | 11.3 |
| CNS ca. (glio/astro) U-118-MG | 9.7 | Lung ca (non-s.cell) HOP-62 | 40.3 |
| CNS ca. (astro) SW1783 | 7.1 | Lung ca. (non-s.cl) NCI-H522 | 100.0 |
| CNS ca.* (neuro; met) SK-N- | 45.7 | Lung ca. (squam.) SW 900 | 18.4 |
| AS | | | |
| CNS ca. (astro) SF-539 | 5.0 | Lung ca. (squam.) NCI-H596 | 27.9 |
| CNS ca. (astro) SNB-75 | 3.7 | Mammary gland | 13.8 |
| CNS ca. (glio) SNB-19 | 12.3 | Breast ca.* (pl. effusion) MCF-7 | 23.3 |
| CNS ca. (glio) U251 | 6.4 | Breast ca.* (pl.ef) MDA-MB- | 13.6 |
| | | 231 | |
| CNS ca. (glio) SF-295 | 14.5 | Breast ca.* (pl. effusion) T47D | 21.3 |
| Heart | 39.8 | Breast ca. BT-549 | 14.1 |
| Skeletal Muscle (new lot*) | 74.2 | Breast ca. MDA-N | 0.0 |
| Bone marrow | 5.0 | Ovary | 11.7 |
| Thymus | 3.6 | Ovarian ca. OVCAR-3 | 29.1 |
| Spleen | 3.5 | Ovarian ca. OVCAR-4 | 19.2 |
| Lymph node | 6.0 | Ovarian ca. OVCAR-5 | 21.3 |
| Colorectal | 2.6 | Ovarian ca. OVCAR-8 | 13.5 |
| Stomach | 19.3 | Ovarian ca. IGROV-1 | 18.9 |
| Small intestine | 15.1 | Ovarian ca.* (ascites) SK-OV-3 | 43.8 |
| Colon ca. SW480 | 19.1 | Uterus | 6.0 |
| Colon ca.* (SW480 met)SW620 | 15.0 | Placenta | 12.0 |
| Colon ca. HT29 | 23.0 | Prostate | 18.2 |
| Colon ca. HCT-116 | 41.2 | Prostate ca.* (bone met)PC-3 | 75.8 |
| Colon ca. CaCo-2 | 50.0 | Testis | 8.7 |
| 83219 CC Well to Mod Diff | 4.6 | Melanoma Hs688(A).T | 6.5 |
| (ODO3866) | | | |
| Colon ca. HCC-2998 | 32.8 | Melanoma* (met) Hs688(B).T | 4.7 |
| Gastric ca.* (liver met) NCI- | 23.3 | Melanoma UACC-62 | 54.7 |
| N87 | | | |
| Bladder | 14.7 | Melanoma M14 | 19.6 |
| Trachea | 5.9 | Melanoma LOX IMVI | 31.0 |
| Kidney | 25.7 | Melanoma* (met) SK-MEL-5 | 43.8 |
| Kidney (fetal) | 23.2 | | |

TABLE 40
Panel 1.3D

| | Relative | | Relative |
|---|---|---|---|
| | Expression(%) | | Expression(%) |
| | 1.3dtm4246f_{—} | | 1.2dtm4246f_{—} |
| Tissue Name | ag2432 | Tissue Name | ag2432 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.5 | Liver | 0.0 |
| Brain (whole) | 0.0 | Liver (fetal) | 0.0 |
| Brain (amygdala) | 0.2 | Liver ca. (hepatoblast) HepG2 | 0.0 |
| Brain (cerebellum) | 0.4 | Lung | 0.0 |
| Brain (hippocampus) | 100.0 | Lung (fetal) | 0.0 |
| Brain (substantia nigra) | 0.0 | Lung ca. (small cell) LX-1 | 0.0 |
| Brain (thalamus) | 0.3 | Lung ca. (small cell) NCI-H69 | 0.0 |
| Cerebral Cortex | 0.2 | Lung ca. (s.cell var.) SHP-77 | 0.0 |
| Spinal cord | 0.0 | Lung ca. (large cell) NCI-H460 | 0.0 |
| CNS ca. (glio/astro) U87-MG | 0.0 | Lung ca. (non-sm. cell) A549 | 0.0 |
| CNS ca. (glio/astro) U-118-MG | 0.0 | Lung ca. (non-s.cell) NCI-H23 | 0.0 |
| CNS ca. (astro) SW1783 | 0.0 | Lung ca (non-s.cell) HOP-62 | 0.0 |
| CNS ca.* (neuro; met) SK-N- | 0.0 | Lung ca. (non-s.cl) NCI-H522 | 0.0 |
| AS | | | |
| CNS ca. (astro) SF-539 | 0.0 | Lung ca. (squam.) SW 900 | 0.0 |
| CNS ca. (astro) SNB-75 | 0.0 | Lung ca. (squam.) NCI-H596 | 0.0 |
| CNS ca. (glio) SNB-19 | 0.0 | Mammary gland | 0.0 |
| CNS ca. (glio) U251 | 0.0 | Breast ca.* (pl. effusion) MCF-7 | 0.0 |
| CNS ca. (glio) SF-295 | 0.0 | Breast ca.* (pl.ef) MDA-MB- | 0.0 |
| | | 231 | |
| Heart (fetal) | 0.0 | Breast ca.* (pl. effusion) T47D | 0.0 |
| Heart | 0.0 | Breast ca. BT-549 | 0.0 |
| Fetal Skeletal | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 0.0 | Ovarian ca.* (ascites) SK-OV-3 | 0.2 |
| Small intestine | 0.0 | Uterus | 0.0 |
| Colon ca. SW480 | 0.0 | Placenta | 0.0 |
| Colon ca.* (SW480 met)SW620 | 0.0 | Prostate | 0.0 |
| Colon ca. HT29 | 0.0 | Prostate ca.* (bone met)PC-3 | 0.0 |
| Colon ca. HCT-116 | 0.0 | Testis | 0.0 |
| Colon ca. CaCo-2 | 4.7 | Melanoma Hs688(A).T | 0.0 |
| 83219 CC Well to Mod Diff | 0.0 | Melanoma* (met) Hs688(B).T | 0.0 |
| (ODO3866) | | | |
| Colon ca. HCC-2998 | 0.0 | Melanoma UACC-62 | 0.0 |
| Gastric ca.* (liver met) NCI- | 0.0 | Melanoma M14 | 0.0 |
| N87 | | | |
| Bladder | 0.3 | Melanoma LOX IMVI | 0.0 |
| Trachea | 0.0 | Melanoma* (met) SK-MEL-5 | 0.0 |
| Kidney | 0.0 | Adipose | 0.0 |

TABLE 41
Panel 2D

| | Relative | | Relative |
|---|---|---|---|
| | Expression(%) | | Expression(%) |
| | 2dtm4247f_{—} | | 2dtm4247f_{—} |
| Tissue Name | ag2432 | Tissue Name | ag2432 |
| Normal Colon GENPAK | 100.0 | Kidney NAT Clontech 8120608 | 0.0 |
| 061003 | | | |
| 83219 CC Well to Mod Diff | 7.5 | Kidney Cancer Clontech | 0.0 |
| (ODO3866) | | 8120613 | |
| 83220 CC NAT (ODO3866) | 7.6 | Kidney NAT Clontech 8120614 | 0.0 |
| 83221 CC Gr.2 rectosigmoid | 0.0 | Kidney Cancer Clontech | 0.0 |
| (ODO3868) | | 9010320 | |
| 83222 CC NAT (ODO3868) | 15.5 | Kidney NAT Clontech 9010321 | 2.8 |
| 83235 CC Mod Diff | 36.1 | Normal Uterus GENPAK | 0.0 |
| (ODO3920) | | 061018 | |
| 83236 CC NAT (ODO3920) | 22.8 | Uterus Cancer GENPAK | 37.1 |
| | | 064011 | |
| 83237 CC Gr.2 ascend colon | 38.4 | Normal Thyroid Clontech A+ | 4.9 |
| (ODO3921) | | 6570-1 | |
| 83238 CC NAT (ODO3921) | 0.0 | Thyroid Cancer GENPAK | 0.0 |
| | | 064010 | |
| 83241 CC from Partial | 0.0 | Thyroid Cancer INVITROGEN | 9.4 |
| Hepatectomy (ODO4309) | | A302152 | |
| 83242 Liver NAT (ODO4309) | 0.0 | Thyroid NAT INVITROGEN | 18.9 |
| | | A302153 | |
| 87472 Colon mets to lung | 3.0 | Normal Breast GENPAK | 5.8 |
| (OD04451-01) | | 061019 | |
| 87473 Lung NAT (OD04451- | 6.6 | 84877 Breast Cancer | 6.9 |
| 02) | | (OD04566) | |
| Normal Prostate Clontech A+ | 6.3 | 85975 Breast Cancer | 0.0 |
| 6546-1 | | (OD04590-01) | |
| 84140 Prostate Cancer | 55.9 | 85976 Breast Cancer Mets | 6.7 |
| (OD04410) | | (OD04590-03) | |
| 84141 Prostate NAT | 4.9 | 87070 Breast Cancer Metastasis | 39.0 |
| (OD04410) | | (OD04655-05) | |
| 87073 Prostate Cancer | 65.1 | GENPAK Breast Cancer | 24.0 |
| (OD04720-01) | | 064006 | |
| 87074 Prostate NAT | 59.9 | Breast Cancer Res. Gen. 1024 | 12.7 |
| (OD04720-02) | | | |
| Normal Lung GENPAK 061010 | 37.4 | Breast Cancer Clontech | 5.3 |
| | | 9100266 | |
| 83239 Lung Met to Muscle | 3.7 | Breast NAT Clontech 9100265 | 6.1 |
| (ODO4286) | | | |
| 83240 Muscle NAT | 28.9 | Breast Cancer INVITROGEN | 6.4 |
| (ODO4286) | | A209073 | |
| 84136 Lung Malignant Cancer | 0.0 | Breast NAT INVITROGEN | 17.2 |
| (OD03126) | | A2090734 | |
| 84137 Lung NAT (OD03126) | 0.0 | Normal Liver GENPAK | 9.5 |
| | | 061009 | |
| 84871 Lung Cancer (OD04404) | 0.0 | Liver Cancer GENPAK 064003 | 15.2 |
| 84872 Lung NAT (OD04404) | 4.8 | Liver Cancer Research Genetics | 0.0 |
| | | RNA 1025 | |
| 84875 Lung Cancer (OD04565) | 0.0 | Liver Cancer Research Genetics | 0.0 |
| | | RNA 1026 | |
| 84876 Lung NAT (OD04565) | 9.7 | Paired Liver Cancer Tissue | 0.0 |
| | | Research Genetics RNA 6004-T | |
| 85950 Lung Cancer (OD04237- | 12.4 | Paired Liver Tissue Research | 0.0 |
| 01) | | Genetics RNA 6004-N | |
| 85970 Lung NAT (OD04237- | 3.9 | Paired Liver Cancer Tissue | 2.6 |
| 02) | | Research Genetics RNA 6005-T | |
| 83255 Ocular Mel Met to Liver | 4.8 | Paired Liver Tissue Research | 0.0 |
| (ODO4310) | | Genetics RNA 6005-N | |
| 83256 Liver NAT (ODO4310) | 2.5 | Normal Bladder GENPAK | 87.7 |
| | | 061001 | |
| 84139 Melanoma Mets to Lung | 0.0 | Bladder Cancer Research | 0.0 |
| (OD04321) | | Genetics RNA 1023 | |
| 84138 Lung NAT (OD04321) | 5.7 | Bladder Cancer INVITROGEN | 14.9 |
| | | A302173 | |
| Normal Kidney GENPAK | 23.0 | 87071 Bladder Cancer | 6.0 |
| 061008 | | (OD04718-01) | |
| 83786 Kidney Ca, Nuclear | 4.2 | 87072 Bladder Normal | 34.6 |
| grade 2 (OD04338) | | Adjacent (OD04718-03) | |
| 83787 Kidney NAT (OD04338) | 4.2 | Normal Ovary Res. Gen. | 0.0 |
| 83788 Kidney Ca Nuclear grade | 9.1 | Ovarian Cancer GENPAK | 7.4 |
| 1/2 (OD04339) | | 064008 | |
| 83789 Kidney NAT (OD04339) | 10.3 | 87492 Ovary Cancer | 0.0 |
| | | (OD04768-07) | |
| 83790 Kidney Ca, Clear cell | 3.0 | 87493 Ovary NAT (OD04768- | 3.8 |
| type (OD04340) | | 08) | |
| 83791 Kidney NAT (OD04340) | 4.6 | Normal Stomach GENPAK | 27.5 |
| | | 061017 | |
| 83792 Kidney Ca, Nuclear | 0.0 | Gastric Cancer Clontech | 0.0 |
| grade 3 (OD04348) | | 9060358 | |
| 83793 Kidney NAT (OD04348) | 6.9 | NAT Stomach Clontech | 3.8 |
| | | 9060359 | |
| 87474 Kidney Cancer | 0.0 | Gastric Cancer Clontech | 14.9 |
| (OD04622-01 | | 9060395 | |
| 87475 Kidney NAT (OD04622- | 0.0 | NAT Stomach Clontech | 27.5 |
| 03) | | 9060394 | |
| 85973 Kidney Cancer | 5.3 | Gastric Cancer Clontech | 0.0 |
| (OD04450-01) | | 9060397 | |
| 85974 Kidney NAT (OD04450- | 7.6 | NAT Stomach Clontech | 0.0 |
| 03) | | 9060396 | |
| Kidney Cancer Clontech | 0.0 | Gastric Cancer GENPAK | 38.2 |
| 8120607 | | 064005 | |

TABLE 42
Panel 4D

| | Relative | | Relative |
|---|---|---|---|
| | Expression | | Expression |
| | (%) | | (%) |
| | 4Dtm2109t_{—} | | 4Dtm2109t_{—} |
| Tissue Name | ag1250 | Tissue Name | ag1250 |
| 93768_Secondary Th1_anti- | 77.9 | 93100_HUVEC | 11.6 |
| CD28/anti-CD3 | | (Endothelial)_IL-1b | |
| 93769_Secondary Th2_anti- | 44.4 | 93779_HUVEC | 14.6 |
| CD28/anti-CD3 | | (Endothelial)_IFN gamma | |
| 93770_Secondary Tr1_anti- | 68.3 | 93102_HUVEC | 6.7 |
| CD28/anti-CD3 | | (Endothelial)_TNF alpha + IFN | |
| | | gamma | |
| 93573_Secondary Th1_resting | 5.9 | 93101_HUVEC | 13.4 |
| day 4-6 in IL-2 | | (Endothelial)_TNF alpha + IL4 | |
| 93572_Secondary Th2_resting | 12.4 | 93781_HUVEC | 3.9 |
| day 4-6 in IL-2 | | (Endothelial)_IL-11 | |
| 93571_Secondary Tr1_resting | 4.4 | 93583_Lung Microvascular | 17.1 |
| day 4-6 in IL-2 | | Endothelial Cells_none | |
| 93568_primary Th1_anti- | 92.0 | 93584_Lung Microvascular | 16.8 |
| CD28/anti-CD3 | | Endothelial Cells_TNFa (4 ng/ml) | |
| | | and IL1b (1 ng/ml) | |
| 93569_primary Th2_anti- | 85.3 | 92662_Microvascular Dermal | 23.3 |
| CD28/anti-CD3 | | endothelium_none | |
| 93570_primary Tr1_anti- | 61.1 | 92663_Microvascular Dermal | 18.9 |
| CD28/anti-CD3 | | endothelium_TNFa (4 ng/ml) | |
| | | and IL1b (1 ng/ml) | |
| 93565_primary Th1_resting dy | 12.3 | 93773_Bronchial | 24.3 |
| 4-6 in IL-2 | | epithelium_TNFa (4 ng/ml) and | |
| | | IL1b (1 ng/ml)** | |
| 93566_primary Th2_resting dy | 7.7 | 93347_Small Airway | 10.2 |
| 4-6 in IL-2 | | Epithelium_none | |
| 93567_primary Tr1_resting dy | 10.2 | 93348_Small Airway | 33.0 |
| 4-6 in IL-2 | | Epithelium_TNFa (4 ng/ml) | |
| | | and IL1b (1 ng/ml) | |
| 93351_CD45RA CD4 | 37.4 | 92668_Coronery Artery | 6.9 |
| lymphocyte_anti-CD28/anti- | | SMC_resting | |
| CD3 | | | |
| 93352_CD45RO CD4 | 28.5 | 92669_Coronery Artery | 7.2 |
| lymphocyte_anti-CD28/anti- | | SMC_TNFa (4 ng/ml) and IL1b | |
| CD3 | | (1 ng/ml) | |
| 93251_CD8 Lymphocytes_anti- | 42.9 | 93107_astrocytes_resting | 4.7 |
| CD28/anti-CD3 | | | |
| 93353_chronic CD8 | 47.0 | 93108_astrocytes_TNFa (4 ng/ml) | 8.8 |
| Lymphocytes 2ry_resting dy 4-6 | | and IL1b (1 ng/ml) | |
| in IL-2 | | | |
| 93574_chronic CD8 | 25.7 | 92666_KU-812 | 20.4 |
| Lymphocytes 2ry_activated | | (Basophil)_resting | |
| CD3/CD28 | | | |
| 93354_CD4_none | 3.2 | 92667_KU-812 | 31.2 |
| | | (Basophil)_PMA/ionoycin | |
| 93252_Secondary | 10.1 | 93579_CCD1106 | 19.2 |
| Th1/Th2/Tr1_anti-CD95 CH11 | | (Keratinocytes)_none | |
| 93103_LAK cells_resting | 14.9 | 93580_CCD1106 | 58.6 |
| | | (Keratinocytes)_TNFa and | |
| | | IFNg** | |
| 93788_LAK cells_IL-2 | 20.7 | 93791_Liver Cirrhosis | 6.3 |
| 93787_LAK cells_IL-2 + IL-12 | 29.3 | 93792_Lupus Kidney | 5.7 |
| 93789_LAK cells_IL-2 + IFN | 19.5 | 93577_NCI-H292 | 19.5 |
| gamma | | | |
| 93790_LAK cells_IL-2 + IL-18 | 14.0 | 93358_NCI-H292_IL-4 | 24.7 |
| 93104_LAK | 10.6 | 93360_NCI-H292_IL-9 | 30.1 |
| cells_PMA/ionomycin and IL- | | | |
| 18 | | | |
| 93578_NK Cells IL-2_resting | 13.3 | 93359_NCI-H292_IL-13 | 24.8 |
| 93109_Mixed Lymphocyte | 13.4 | 93357_NCI-H292_IFN gamma | 17.3 |
| Reaction_Two Way MLR | | | |
| 93110_Mixed Lymphocyte | 19.3 | 93777_HPAEC_- | 9.3 |
| Reaction_Two Way MLR | | | |
| 93111_Mixed Lymphocyte | 13.5 | 93778_HPAEC_IL-1 beta/TNA | 7.7 |
| Reaction_Two Way MLR | | alpha | |
| 93112_Mononuclear Cells | 4.9 | 93254_Normal Human Lung | 6.6 |
| (PBMCs)_resting | | Fibroblast_none | |
| 93113_Mononuclear Cells | 56.6 | 93253_Normal Human Lung | 4.2 |
| (PBMCs)_PWM | | Fibroblast_TNFa (4 ng/ml) and | |
| | | IL-1b (1 ng/ml) | |
| 93114_Mononuclear Cells | 34.6 | 93257_Normal Human Lung | 21.0 |
| (PBMCs)_PHA-L | | Fibroblast_IL-4 | |
| 93249_Ramos (B cell)_none | 63.3 | 93256_Normal Human Lung | 13.2 |
| | | Fibroblast_IL-9 | |
| 93250_Ramos (B | 73.7 | 93255_Normal Human Lung | 29.3 |
| cell)_ionomycin | | Fibroblast_IL-13 | |
| 93349_B lymphocytes_PWM | 57.4 | 93258_Normal Human Lung | 25.2 |
| | | Fibroblast_IFN gamma | |
| 93350_B lymphoytes_CD40L | 17.2 | 93106_Dermal Fibroblasts | 28.3 |
| and IL-4 | | CCD1070_resting | |
| 92665_EOL-1 | 10.2 | 93361_Dermal Fibroblasts | 25.5 |
| (Eosinophil)_dbcAMP | | CCD1070_RNF alpha 4 ng/ml | |
| differentiated | | | |
| 93248_EOL-1 | 5.2 | 93361_Dermal Fibroblasts | 24.8 |
| (Eosinophil)_dbcAMP/PMAionomycin | | CCD1070_IL-1 beta 1 ng/ml | |
| 93356_Dendritic Cells_none | 7.5 | 93772_dermal fibroblast_IFN | 10.7 |
| | | gamma | |
| 93355_Dendritic Cells_LPS | 6.1 | 93771_dermal fibroblast_IL-4 | 12.7 |
| 100 ng/ml | | | |
| 93775_Dendritic Cells_anti- | 9.7 | 93260_IBD Colitis 2 | 0.3 |
| CD40 | | | |
| 93774_Monocytes_resting | 4.8 | 99261_IBD Crohns | 0.9 |
| 93776_Monocytes_LPS 50 ng/ml | 3.5 | 735010_Colon_normal | 8.7 |
| 93581_Macrophages_resting | 23.2 | 735019_Lung_none | 15.8 |
| 93582_Macrophages_LPS 100 ng/ml | 13.1 | 64028-1_Thymus_none | 100.0 |
| 93098_HUVEC | 19.5 | 64030-1_Kidney_none | 11.5 |
| (Endothelial)_none | | | |
| 93099_HUVEC | 17.1 | | |
| (Endothelial)_starved | | | |

Panel 1.2 Summary Ag1250 The NOV4 gene is expressed at high levels in lung cancer cell lines. Overall, there is a predominant expression pattern that shows higher expression of this gene in cancer cell lines when compared to normal tissues. Specifically, the NOV4 gene is expressed at higher levels in samples derived from colon cancer, ovarian cancer, breast cancer, prostate cancer and melanoma cell lines. Thus, expression of the NOV4 gene could be used to distinguish cultured cell lines from normal tissues. In addition, these data indicate that the expression of this gene might be associated with these forms of cancer and thus, therapeutic modulation of the NOV4 gene product might be of use in the treatment of these cancers.

Panel 1.3D Summary A22432 Expression of the NOV4 gene is limited to the hippocampus (CT=27.6), where it is expressed at high levels. Therefore, expression of this gene could be used to distinguish hippocampus from other tissues.

Panel 2D Summary Ag2432 Significant but low expression of the NOV4 gene is detected in normal colon and bladder tissues. Therefore, expression of this gene could be used to distinguish colon and bladder from other tissues.

Panel 4D Summary Ag1250 The NOV4 gene encodes a protein with homology to prohibiting, which are proteins that have been shown to be involved various functions, including cell cycle regulation, apoptosis, assembly of mitochondrial respiratory chain enzymes, and aging (ref. 1). The NOV4 gene is expressed at moderate levels throughout the samples on this panel (CTs=30.8-33.6). Interestingly, however, this gene is expressed at highest levels in the thymus as well as in activated Th1 and Th2 T cells. Given this expression pattern, the NOV4 gene product may play an important role in the normal homeostasis of the thymus and might be associated with the activation process of T cells. Therefore, modulation of this protein by small molecule drugs might be important for controlling T cell activation and could have some benefit for treatment of diseases associated with hyperactive T cells, such as autoimmune disease, delayed type hypersentivity, and other T cell mediated diseases (such as asthma and psoriasis). In addition, the NOV4 gene is also expressed in activated B cells and in a Ramos B cell line. It has been suggested that increased prohibitin expression is associated with and may facilitate B-cell maturation (ref. 2). Thus, modulation of this protein by small molecule drugs might be important for controlling B differentiation and the generation of immunoglobulins by B cells and could therefore have some therapeutic benefit in the treatment of hypoglobulinemia.

Panel CNS_neurodegeneration_v1.0 Summary Ag2432 Expression of this gene is low/undetectable (CT values>35) among the samples on this panel (data not shown).

References:

1. Coates P J, Nenutil R, McGregor A, Picksley S M, Crouch D H, Hall P A, Wright E G. Mammalian prohibitin proteins respond to mitochondrial stress and decrease during cellular senescence. Exp Cell Res 2001 May 1;265(2):262-73

The two prohibitin proteins, Phb1p and Phb2p(BAP37), have been ascribed various functions, including cell cycle regulation, apoptosis, assembly of mitochondrial respiratory chain enzymes, and aging. We show that the mammalian prohibitins are present in the inner mitochondrial membrane and are always bound to each other, with no free protein detectable. They are coexpressed during development and in adult mammalian tissues, and expression levels are indicative of a role in mitochondrial metabolism, but are not compatible with roles in the regulation of cellular proliferation or apoptosis. High level expression of the proteins is consistently seen in primary human tumors, while cellular senescence of human and chick fibroblasts is accompanied by heterogeneous decreases in both proteins. The two proteins are induced by metabolic stress caused by an imbalance in the synthesis of mitochondrial- and nuclear-encoded mitochondrial proteins, but do not respond to oxidative stress, heat shock, or other cellular stresses. The gene promoter sequences contain binding sites for the Myc oncoprotein and overexpression of Myc induces expression of the prohibiting. The data support conserved roles for the prohibitins in regulating mitochondrial respiratory activity and in aging.

PMID: 11302691

2. Woodlock T J, Bethlendy G, Segel G B. Prohibitin expression is increased in phorbol ester-treated chronic leukemic B-lymphocytes. Blood Cells Mol Dis 2001 Jan-Feb;27(1):27-34

Chronic lymphocytic leukemia (CLL) is characterized by the gradual accumulation of immature B-lymphocytes. CLL B-lymphocytes mature to a plasmacytoid phenotype when treated in vitro with phorbol esters. CLL B-cell apparent maturation is associated with altered expression of specific plasma membrane and mitochondrial proteins including heightened expression of a 30-kDa heat shock protein 60 (hsp60) analog. During our efforts to further characterize this hsp60 analog by mass spectrometry, we detected the mitochondrial protein prohibitin in phorbol-ester-matured CLL B-lymphocytes. Prohibitin modulates cell proliferation and inhibits cell cycle traverse in several systems, although few data are available for lymphocytes. A twofold increase in prohibitin concentration wasobserved in phorbol-ester-matured compared to resting CLL B-cells as determined by quantitative Western immunoblot analysis. A similar increase in prohibitin was observed in phorbol-ester-treated normal human B-lymphocyte populations. An antisense oligonucleotide complementary to the 5′ coding region of the prohibitin gene blunted the increase in prohibitin protein in phorbol-ester-treated CLL B-cells by 42%. These data suggest that increased prohibitin expression is associated with and may facilitate B-cell maturation.

PMID: 11162143

NOV5

Expression of gene NOV5 was assessed using the primer-probe sets Ag3086 and Ag3797 described in Tables 43 and 44. Results from RTQ-PCR runs are shown in Tables 45, 46, 47, 48, 49, and 50.

TABLE 45
Probe Name Ag3086

| | | | | Start | SEQ ID |
|---|---|---|---|---|---|
| Primers | Sequences | TM | Length | Position | NO: |
| Forward | 5′-GGACCCCATTCGACTACTGT-3′ | 20 | 20 | 1309 | 144 |
| Probe | FAM-5′-CTGATGACCAGCCGCCATCAATC-3′-TAMRA | 23 | 23 | 1345 | 145 |
| Reverse | 5′-TTCTCAAACTGCACCTGGTC-3′ | 20 | 20 | 1399 | 146 |

TABLE 46
Probe Name Ag3797

| | | | | Start | SEQ ID |
|---|---|---|---|---|---|
| Primers | Sequences | TM | Length | Position | NO: |
| Forward | 5′-TCTCGACGACAACTATTGCC-3′ | 58.7 | 20 | 627 | 147 |
| Probe | FAM-5′-ATGGTGCTACACTACGGATCCGCAG-3′-TAMRA | 69.2 | 25 | 672 | 148 |
| Reverse | 5′-GTCACAGAATTCTCGCTCGA-3′ | 59.1 | 20 | 698 | 149 |

TABLE 47
Panel 1.3D

| | Relative | | Relative |
|---|---|---|---|
| | Expression(%) | | Expression(%) |
| | 1.3dx4tm5430 | | 1.3dx4tm5430 |
| Tissue Name | f_ag3086_a1 | Tissue Name | f_ag3086_a1 |
| Liver adenocarcinoma | 0.7 | Kidney (fetal) | 31.1 |
| Pancreas | 17.9 | Renal ca. 786-0 | 0.2 |
| Pancreatic ca. CAPAN 2 | 0.6 | Renal ca. A498 | 0.5 |
| Adrenal gland | 2.7 | Renal ca. RXF 393 | 0.7 |
| Thyroid | 3.3 | Renal ca. ACHN | 0.8 |
| Salivary gland | 1.2 | Renal ca. UO-31 | 0.4 |
| Pituitary gland | 3.6 | Renal ca. TK-10 | 0.2 |
| Brain (fetal) | 3.2 | Liver | 94.2 |
| Brain (whole) | 3.4 | Liver (fetal) | 100.0 |
| Brain (amygdala) | 2.1 | Liver ca. (hepatoblast) HepG2 | 58.4 |
| Brain (cerebellum) | 1.5 | Lung | 2.8 |
| Brain (hippocampus) | 3.0 | Lung (fetal) | 12.9 |
| Brain (substantia nigra) | 1.7 | Lung ca. (small cell) LX-1 | 1.3 |
| Brain (thalamus) | 3.0 | Lung ca. (small cell) NCI-H69 | 0.2 |
| Cerebral Cortex | 0.9 | Lung ca. (s.cell var.) SHP-77 | 1.2 |
| Spinal cord | 2.9 | Lung ca. (large cell)NCI-H460 | 1.4 |
| CNS ca. (glio/astro) U87-MG | 0.7 | Lung ca. (non-sm. cell) A549 | 0.2 |
| CNS ca. (glio/astro) U-118-MG | 0.9 | Lung ca. (non-s.cell) NCI-H23 | 0.9 |
| CNS ca. (astro) SW1783 | 0.4 | Lung ca (non-s.cell) HOP-62 | 0.5 |
| CNS ca.* (neuro; met) SK-N- | 0.7 | Lung ca. (non-s.cl) NCI-H522 | 0.6 |
| AS | | | |
| CNS ca. (astro) SF-539 | 0.5 | Lung ca. (squam.) SW 900 | 0.4 |
| CNS ca. (astro) SNB-75 | 1.2 | Lung ca. (squam.) NCI-H596 | 0.5 |
| CNS ca. (glio) SNB-19 | 1.6 | Mammary gland | 3.1 |
| CNS ca. (glio) U251 | 2.4 | Breast ca.* (pl. effusion) MCF-7 | 0.7 |
| CNS ca. (glio) SF-295 | 0.7 | Breast ca.* (pl.ef) MDA-MB- | 0.7 |
| | | 231 | |
| Heart (fetal) | 0.6 | Breast ca.* (pl. effusion) T47D | 2.7 |
| Heart | 0.5 | Breast ca. BT-549 | 0.7 |
| Fetal Skeletal | 0.2 | Breast ca. MDA-N | 0.0 |
| Skeletal muscle | 1.4 | Ovary | 0.4 |
| Bone marrow | 2.0 | Ovarian ca. OVCAR-3 | 0.6 |
| Thymus | 1.2 | Ovarian ca. OVCAR-4 | 0.4 |
| Spleen | 4.0 | Ovarian ca. OVCAR-5 | 0.4 |
| Lymph node | 3.1 | Ovarian ca. OVCAR-8 | 0.7 |
| Colorectal | 1.6 | Ovarian ca. IGROV-1 | 0.7 |
| Stomach | 10.3 | Ovarian ca.* (ascites) SK-OV-3 | 0.0 |
| Small intestine | 29.6 | Uterus | 3.2 |
| Colon ca. SW480 | 0.7 | Placenta | 4.6 |
| Colon ca.* (SW480 met)SW620 | 0.2 | Prostate | 2.1 |
| Colon ca. HT29 | 0.2 | Prostate ca.* (bone met)PC-3 | 0.9 |
| Colon ca. HCT-116 | 1.2 | Testis | 12.4 |
| Colon ca. CaCo-2 | 2.4 | Melanoma Hs688(A).T | 0.1 |
| 83219 CC Well to Mod Diff | 2.1 | Melanoma* (met) Hs688(B).T | 0.2 |
| (ODO3866) | | | |
| Colon ca. HCC-2998 | 1.4 | Melanoma UACC-62 | 0.4 |
| Gastric ca.* (liver met) NCI- | 1.8 | Melanoma M14 | 0.8 |
| N87 | | | |
| Bladder | 4.5 | Melanoma LOX IMVI | 0.0 |
| Trachea | 2.6 | Melanoma* (met) SK-MEL-5 | 0.4 |
| Kidney | 26.1 | Adipose | 2.2 |

TABLE 48
General Screening Panel_v1.4

| | Relative | | Relative |
|---|---|---|---|
| | Expression(%) | | Expression(%) |
| | 1.4x4tm7355f_{—} | | 1.4x4tm7355f_{—} |
| Tissue Name | ag3797_a1 | Tissue Name | ag3797_a1 |
| D6005-01_Human adipose | 1.4 | Renal ca._TK-10 | 28.9 |
| 112193_Metastatic melanoma | 0.4 | Bladder | 8.4 |
| 112192_Metastatic melanoma | 0.5 | Gastric ca.(liver met)_NCI-N87 | 2.7 |
| 95280_Epidermis (metastatic | 0.3 | 112197_Stomach | 1.4 |
| melanoma) | | | |
| 95279_Epidermis (metastatic | 0.3 | 94938_Colon Adenocarcinoma | 1.0 |
| melanoma) | | | |
| Melanoma (met)_SK-MEL-5 | 0.5 | Colon ca._SW480 | 3.9 |
| 112196_Tongue (oncology) | 0.8 | Colon ca.(SW480 met)_SW620 | 1.2 |
| 113461_Testis Pool | 2.0 | Colon ca._HT29 | 0.2 |
| Prostate ca.(bone met)_PC-3 | 1.5 | Colon ca._HCT-116 | 4.3 |
| 113455_Prostate Pool | 1.8 | Colon ca._CaCo-2 | 11.5 |
| 103396_Placenta | 1.7 | 83219_CC Well to Mod Diff | 2.8 |
| | | (ODO3866) | |
| 113463_Uterus Pool | 0.5 | 94936_Colon Adenocarcinoma | 2.9 |
| Ovarian carcinoma_OVCAR-3 | 1.0 | 94930_Colon | 0.5 |
| Ovarian | 0.8 | 94935_Colon Adenocarcinoma | 0.2 |
| carcinoma(ascites)_SK-OV-3 | | | |
| 95297_Adenocarcinoma | 0.3 | 113468_Colon Pool | 1.6 |
| (ovary) | | | |
| Ovarian carcinoma_OVCAR-5 | 6.4 | 113457_Small Intestine Pool | 2.0 |
| Ovarian carcinoma_IGROV-1 | 4.6 | 113460_Stomach Pool | 1.9 |
| Ovarian carcinoma_OVCAR-8 | 2.9 | 113467_Bone Marrow Pool | 0.4 |
| 103368_Ovary | 1.9 | 103371_Fetal Heart | 0.8 |
| MCF7_breast | 2.3 | 113451_Heart Pool | 0.7 |
| carcinoma(pleural effusion) | | | |
| Breast ca. (pleural | 2.2 | 113466_Lymph Node Pool | 1.7 |
| effusion)_MDA-MB-231 | | | |
| 112189_ductal cell | 3.0 | 103372_Fetal Skeletal Muscle | 0.7 |
| carcinoma(breast) | | | |
| Breast ca. (pleural | 18.5 | 113456_Skeletal Muscle Pool | 1.1 |
| effusion)_T47D | | | |
| Breast carcinoma_MDA-N | 0.7 | 113459_Spleen Pool | 2.5 |
| 113452_Breast Pool | 1.5 | 113462_Thymus Pool | 2.4 |
| 103398_Trachea | 1.2 | CNS ca. (glio/astro)_U87-MG | 2.7 |
| 112354_lung | 0.4 | CNS ca. (glio/astro)_U-118- | 3.0 |
| | | MG | |
| 103374_Fetal Lung | 2.3 | CNS ca. (neuro;met)_SK-N-AS | 2.1 |
| 94921_Small cell carcinoma of | 0.2 | 95264_Brain astrocytoma | 0.6 |
| the lung | | | |
| Lung ca.(small cell)_LX-1 | 3.3 | CNS ca. (astro)_SNB-75 | 1.8 |
| 94919_Small cell carcinoma of | 0.5 | CNS ca. (glio)_SNB-19 | 4.1 |
| the lung | | | |
| Lung ca.(s.cell var.)_SHP-77 | 2.4 | CNS ca. (glio)_SF-295 | 2.1 |
| 95268_Lung (Large cell | 0.6 | 113447_Brain (Amygdala) Pool | 0.9 |
| carcinoma) | | | |
| 94920_Small cell carcinoma of | 0.6 | 103382_Brain (cerebellum) | 1.9 |
| the lung | | | |
| Lung ca.(non-s.cell)_NCI-H23 | 3.7 | 64019-1_brain(fetal) | 2.8 |
| Lung ca.(large cell)_NCI-H460 | 0.9 | 113448_Brain (Hippocampus) | 1.0 |
| | | Pool | |
| Lung ca.(non-s.cell)_HOP-62 | 1.2 | 113464_Cerebral Cortex Pool | 0.7 |
| Lung ca.(non-s.cl)_NCI-H522 | 1.7 | 113449_Brain (Substantia | 0.9 |
| | | nigra) Pool | |
| 103392_Liver | 26.6 | 113450_Brain (Thalamus) Pool | 1.0 |
| 103393_Fetal Liver | 45.5 | 103384_Brain (whole) | 1.6 |
| Liver ca.(hepatoblast)_HepG2 | 100.0 | 113458_Spinal Cord Pool | 1.7 |
| 113465_Kidney Pool | 1.7 | 103375_Adrenal Gland | 3.1 |
| 103373_Fetal Kidney | 11.1 | 113454_Pituitary gland Pool | 1.7 |
| Renal ca._786-0 | 1.0 | 103397_Salivary Gland | 1.0 |
| 112188_renal cell carcinoma | 0.3 | 103369_Thyroid (female) | 2.8 |
| Renal ca._ACHN | 1.4 | Pancreatic ca._CAPAN2 | 0.8 |
| 112190_Renal cell carcinoma | 1.8 | 113453_Pancreas Pool | 7.5 |

TABLE 49
Panel 2.2

| | Relative | | Relative |
|---|---|---|---|
| | Expression(%) | | Expression(%) |
| | 2.2x4tm6408f_{—} | | 2.2x4tm6408f_{—} |
| Tissue Name | ag3086_a1 | Tissue Name | ag3086_a1 |
| Normal Colon GENPAK | 1.4 | 83793 Kidney NAT (OD04348) | 40.9 |
| 061003 | | | |
| 97759 Colon cancer (OD06064) | 0.1 | 98938 Kidney malignant cancer | 0.4 |
| | | (OD06204B) | |
| 97760 Colon cancer NAT | 0.0 | 98939 Kidney normal adjacent | 5.1 |
| (OD06064) | | tissue (OD06204E) | |
| 97778 Colon cancer (OD06159) | 0.0 | 85973 Kidney Cancer | 5.7 |
| | | (OD04450-01) | |
| 97779 Colon cancer NAT | 1.4 | 85974 Kidney NAT (OD04450- | 15.0 |
| (OD06159) | 1.4 | 03) | |
| 98861 Colon cancer (OD06297- | 0.0 | Kidney Cancer Clontech | 0.2 |
| 04) | | 8120613 | |
| 98862 Colon cancer NAT | 1.1 | Kidney NAT Clontech 8120614 | 5.9 |
| (OD06297-015) | | | |
| 83237 CC Gr.2 ascend colon | 0.4 | Kidney Cancer Clontech | 0.4 |
| (ODO3921) | | 9010320 | |
| 83238 CC NAT (ODO3921) | 0.2 | Kidney NAT Clontech 9010321 | 1.5 |
| 97766 Colon cancer metastasis | 0.0 | Kidney Cancer Clontech | 0.1 |
| (OD06104) | | 8120607 | |
| 97767 Lung NAT (OD06104) | 0.6 | Kidney NAT Clontech 8120608 | 3.5 |
| 87472 Colon mets to lung | 1.3 | Normal Uterus GENPAK | 0.2 |
| (OD04451-01) | | 061018 | |
| 87473 Lung NAT (OD04451- | 0.4 | Uterus Cancer GENPAK | 0.1 |
| 02) | | 064011 | |
| Normal Prostate Clontech A+ | 0.6 | Normal Thyroid Clontech A+ | 0.5 |
| 6546-1 (8090438) | | 6570-1 (7080817) | |
| 84140 Prostate Cancer | 0.2 | Thyroid Cancer GENPAK | 0.3 |
| (OD04410) | | 064010 | |
| 84141 Prostate NAT | 0.5 | Thyroid Cancer INVITROGEN | 2.1 |
| (OD04410) | | A302152 | |
| Normal Ovary Res. Gen. | 0.4 | Thyroid NAT INVITROGEN | 0.4 |
| | | A302153 | |
| 98863 Ovarian cancer | 0.2 | Normal Breast GENPAK | 0.7 |
| (OD06283-03) | | 061019 | |
| 98865 Ovarian cancer | 1.2 | 84877 Breast Cancer | 2.3 |
| NAT/fallopian tube (OD06283- | | (OD04566) | |
| 07) | | | |
| Ovarian Cancer GENPAK | 1.5 | Breast Cancer Res. Gen. 1024 | 1.9 |
| 064008 | | | |
| 97773 Ovarian cancer | 0.9 | 85975 Breast Cancer | 5.1 |
| (OD06145) | | (OD04590-01) | |
| 97775 Ovarian cancer NAT | 1.8 | 85976 Breast Cancer Mets | 1.3 |
| (OD06145) | | (OD04590-03) | |
| 98853 Ovarian cancer | 0.6 | 87070 Breast Cancer Metastasis | 0.7 |
| (OD06455-03) | | (OD04655-05) | |
| 98854 Ovarian NAT | 0.2 | GENPAK Breast Cancer | 0.5 |
| (OD06455-07) Fallopian tube | | 064006 | |
| Normal Lung GENPAK 061010 | 0.8 | Breast Cancer Clontech | 0.2 |
| | | 9100266 | |
| 92337 Invasive poor diff. lung | 0.3 | Breast NAT Clontech 9100265 | 0.5 |
| adeno (ODO4945-01 | | | |
| 92338 Lung NAT (ODO4945- | 1.1 | Breast Cancer INVITROGEN | 0.2 |
| 03) | | A209073 | |
| 84136 Lung Malignant Cancer | 1.6 | Breast NAT INVITROGEN | 1.4 |
| (OD03126) | | A2090734 | |
| 84137 Lung NAT (OD03126) | 0.3 | 97763 Breast cancer | 0.7 |
| | | (OD06083) | |
| 90372 Lung Cancer | 0.5 | 97764 Breast cancer node | 0.2 |
| (OD05014A) | | metastasis (OD06083) | |
| 90373 Lung NAT (OD05014B) | 0.6 | Normal Liver GENPAK | 28.7 |
| | | 061009 | |
| 97761 Lung cancer (OD06081) | 0.5 | Liver Cancer Research Genetics | 7.5 |
| | | RNA 1026 | |
| 97762 Lung cancer NAT | 1.2 | Liver Cancer Research Genetics | 45.0 |
| (OD06081) | | RNA 1025 | |
| 85950 Lung Cancer (OD04237- | 0.3 | Paired Liver Cancer Tissue | 35.7 |
| 01) | | Research Genetics RNA 6004-T | |
| 85970 Lung NAT (OD04237- | 1.1 | Paired Liver Tissue Research | 5.1 |
| 02) | | Genetics RNA 6004-N | |
| 83255 Ocular Mel Met to Liver | 0.2 | Paired Liver Cancer Tissue | 14.7 |
| (ODO4310) | | Research Genetics RNA 6005-T | |
| 83256 Liver NAT (ODO4310) | 21.6 | Paired Liver Tissue Research | 65.0 |
| | | Genetics RNA 6005-N | |
| 84139 Melanoma Mets to Lung | 0.2 | Liver Cancer GENPAK 064003 | 100.0 |
| (OD04321) | | | |
| 84138 Lung NAT (OD04321) | 0.2 | Normal Bladder GENPAK | 2.8 |
| | | 061001 | |
| Normal Kidney GENPAK | 5.5 | Bladder Cancer Research | 0.2 |
| 061008 | | Genetics RNA 1023 | |
| 83786 Kidney Ca, Nuclear | 20.6 | Bladder Cancer INVITROGEN | 0.7 |
| grade 2 (OD04338) | | A302173 | |
| 83787 Kidney NAT (OD04338) | 4.5 | Normal Stomach GENPAK | 2.5 |
| | | 061017 | |
| 83788 Kidney Ca Nuclear grade | 6.5 | Gastric Cancer Clontech | 0.0 |
| 1/2 (OD04339) | | 9060397 | |
| 83789 Kidney NAT (OD04339) | 6.0 | NAT Stomach Clontech | 0.1 |
| | | 9060396 | |
| 83790 Kidney Ca, Clear cell | 0.4 | Gastric Cancer Clontech | 0.0 |
| type (OD04340) | | 9060395 | |
| 83791 Kidney NAT (OD04340) | 8.7 | NAT Stomach Clontech | 0.3 |
| | | 9060394 | |
| 83792 Kidney Ca, Nuclear | 0.3 | Gastric Cancer GENPAK | 2.8 |
| grade 3 (OD04348) | | 064005 | |

TABLE 50
Panel 4D

| | Relative | | Relative |
|---|---|---|---|
| | Expression(%) | | Expression(%) |
| | 4dx4tm5510f_{—} | | 4dx4tm5510f_{—} |
| Tissue Name | ag3086_a2 | Tissue Name | ag3086_a2 |
| 93768_Secondary Th1_anti- | 0.7 | 93100_HUVEC | 0.4 |
| CD28/anti-CD3 | | (Endothelial)_IL-1b | |
| 93769_Secondary Th2_anti- | 0.9 | 93799_HUVEC | 1.2 |
| CD28/anti-CD3 | | (Endothelial)_IFN gamma | |
| 93770_Secondary Tr1_anti- | 1.1 | 93102_HUVEC | 0.3 |
| CD28/anti-CD3 | | (Endothelial)_TNF alpha + IFN | |
| | | gamma | |
| 93573_Secondary Th1_resting | 2.8 | 93101_HUVEC | 0.2 |
| day 4-6 in IL-2 | | (Endothelial)_TNF alpha + IL4 | |
| 93572_Secondary Th2_resting | 1.4 | 93781_HUVEC | 0.8 |
| day 4-6 in IL-2 | | (Endothelial)_IL-11 | |
| 93571_Secondary Tr1_resting | 1.3 | 93583_Lung Microvascular | 1.0 |
| day 4-6 in IL-2 | | Endothelial Cells_none | |
| 93568_primary Th1_anti- | 0.7 | 93584_Lung Microvascular | 0.9 |
| CD28/anti-CD3 | | Endothelial Cells_TNFa (4 ng/ml) | |
| | | and IL1b (1 ng/ml) | |
| 93569_primary Th2_anti- | 0.9 | 92662_Microvascular Dermal | 1.6 |
| CD28/anti-CD3 | | endothelium_none | |
| 93570_primary Tr1_anti- | 1.1 | 92663_Microsvascular Dermal | 0.8 |
| CD28/anti-CD3 | | endothelium_TNFa (4 ng/ml) | |
| | | and IL1b (1 ng/ml) | |
| 93565_primary Th1_resting dy | 6.4 | 93773_Bronchial | 3.2 |
| 4-6 in IL-2 | | epithelium_TNFa (4 ng/ml) and | |
| | | IL1b (1 ng/ml)** | |
| 93566_primary Th2_resting dy | 3.1 | 93347_Small Airway | 1.8 |
| 4-6 in IL-2 | | Epithelium_none | |
| 93567_primary Tr1_resting dy | 2.0 | 93348_Small Airway | 3.3 |
| 4-6 in IL-2 | | Epithelium_TNFa (4 ng/ml) | |
| | | and IL1b (1 ng/ml) | |
| 93351_CD45RA CD4 | 0.4 | 92668_Coronery Artery | 1.0 |
| lymphocyte_anti-CD28/anti- | | SMC_resting | |
| CD3 | | | |
| 93352_CD45RO CD4 | 1.4 | 92669_Coronery Artery | 0.6 |
| lymphocyte_anti-CD28/anti- | | SMC_TNFa (4 ng/ml) and IL1b | |
| CD3 | | (1 ng/ml) | |
| 93251_CD8 Lymphocytes_anti- | 1.4 | 93107_astrocytes_resting | 3.2 |
| CD28/anti-CD3 | | | |
| 93353_chronic CD8 | 1.7 | 93108_astrocytes_TNFa (4 ng/ml) | 2.7 |
| Lymphocytes 2ry_resting dy 4-6 | | and IL1b (1 ng/ml) | |
| in IL-2 | | | |
| 93574_chronic CD8 | 0.6 | 92666_KU-812 | 1.5 |
| Lymphocytes 2ry_activated | | (Basophil)_resting | |
| CD3/CD28 | | | |
| 93354_CD4_none | 2.1 | 92667_KU-812 | 1.2 |
| | | (Basophil)_PMA/ionoycin | |
| 93252_Secondary | 4.2 | 93579_CCD1106 | 1.0 |
| Th1/Th2/Tr1_anti-CD95 CH11 | | (Keratinocytes)_none | |
| 93103_LAK cells_resting | 1.2 | 93580_CCD1106 | 3.6 |
| | | (Keratinocytes)_TNFa and | |
| | | IFNg** | |
| 93788_LAK cells_IL-2 | 3.7 | 93791_Liver Cirrhosis | 84.6 |
| 93787_LAK cells_IL-2 + IL-12 | 2.2 | 93792_Lupus Kidney | 33.9 |
| 93789_LAK cells_IL-2 + IFN | 3.0 | 93577_NCI-H292 | 8.6 |
| gamma | | | |
| 93790_LAK cells_IL-2 + IL-18 | 2.6 | 93358_NCI-H292_IL-4 | 7.1 |
| 93104_LAK | 1.0 | 93360_NCI-H292_IL-9 | 6.5 |
| cells_PMA/ionomycin and IL- | | | |
| 18 | | | |
| 93578_NK Cells IL-2_resting | 1.5 | 93359_NCI-H292_IL-13 | 2.8 |
| 93109_Mixed Lymphocyte | 2.2 | 93357_NCI-H292_IFN gamma | 3.1 |
| Reaction_Two Way MLR | | | |
| 93110_Mixed Lymphocyte | 1.2 | 93777_HPAEC_- | 1.7 |
| Reaction_Two Way MLR | | | |
| 93111_Mixed Lymphocyte | 1.3 | 93778_HPAEC_IL-1 beta/TNA | 1.4 |
| Reaction_Two Way MLR | | alpha | |
| 93112_Mononuclear Cells | 0.7 | 93254_Normal Human Lung | 4.3 |
| (PBMCs)_resting | | Fibroblast_none | |
| 93113_Mononuclear Cells | 0.8 | 93253_Normal Human Lung | 4.9 |
| (PBMCs)_PWM | | Fibroblast_TNFa (4 ng/ml) and | |
| | | IL-1b (1 ng/ml) | |
| 93114_Mononuclear Cells | 0.6 | 93257_Normal Human Lung | 2.2 |
| (PBMCs)_PHA-L | | Fibroblast_IL-4 | |
| 93249_Ramos (B cell)_none | 1.9 | 93256_Normal Human Lung | 1.2 |
| | | Fibroblast_IL-9 | |
| 93250_Ramos (B | 1.4 | 93255_Normal Human Lung | 1.6 |
| cell)_ionomycin | | Fibroblast_IL-13 | |
| 93349_B lymphocytes_PWM | 1.2 | 93258_Normal Human Lung | 1.9 |
| | | Fibroblast_IFN gamma | |
| 93350_B lymphoytes_CD40L | 2.2 | 93106_Dermal Fibroblasts | 3.3 |
| and IL-4 | | CCD1070_resting | |
| 92665_EOL-1 | 2.4 | 93361_Dermal Fibroblasts | 4.7 |
| (Eosinophil)_dbcAMP | | CCD1070_TNF alpha 4 ng/ml | |
| differentiated | | | |
| 93248_EOL-1 | 2.6 | 93105_Dermal Fibroblasts | 0.7 |
| (Eosinophil)_dbcAMP/PMAionomycin | | CCD1070_IL-1 beta 1 ng/ml | |
| 93356_Dendritic Cells_none | 2.2 | 93772_dermal fibroblast_IFN | 0.8 |
| | | gamma | |
| 93355_Dendritic Cells_LPS | 2.1 | 93771_dermal fibroblasts_IL-4 | 2.4 |
| 100 ng/ml | | | |
| 93775_Dendritic Cells_anti- | 1.8 | 93260_IBD Colitis 2 | 11.6 |
| CD40 | | | |
| 93774_Monocytes_resting | 1.5 | 93261_IBD Crohns | 14.2 |
| 93776_Monocytes_LPS 50 ng/ml | 0.6 | 735010_Colon_normal | 61.0 |
| 93581_Macrophages_resting | 1.7 | 735019_Lung_none | 3.6 |
| 93582_Macrophages_LPS 100 ng/ml | 1.1 | 64028-1_Thymus_none | 100.0 |
| 93098_HUVEC | 1.3 | 64030-1_Kidney_none | 5.7 |
| (Endothelial)_none | | | |
| 93099_HUVEC | 1.2 | | |
| (Endothelial)_starved | | | |

TABLE 51
Panel 4.1D

| | Relative Expression(%) | |
|---|---|---|
| | 4.1dx4tm5986 | 4.1dtm6034f_{—} |
| Tissue Name | f_ag3797_a1 | ag3797 |
| 93768_Secondary Th1_anti-CD28/anti-CD3 | 8.9 | 1.3 |
| 93769_Secondary Th2_anti-CD28/anti-CD3 | 2.6 | 1.3 |
| 93770_Secondary Tr1_anti-CD28/anti-CD3 | 3.1 | 0.9 |
| 93573_Secondary Th1_resting day 4-6 in IL-2 | 1.5 | 1.6 |
| 93572_Secondary Th2_resting day 4-6 in IL-2 | 3.4 | 0.7 |
| 93571_Secondary Tr1_resting day 4-6 in IL-2 | 3.4 | 0.9 |
| 93568_primary Th1_anti-CD28/anti-CD3 | 3.2 | 0.3 |
| 93569_primary Th2_anti-CD28/anti-CD3 | 2.0 | 1.1 |
| 93570_primary Tr1_anti-CD28/anti-CD3 | 2.7 | 1.0 |
| 93565_primary Th1_resting dy 4-6 in IL-2 | 3.2 | 0.4 |
| 93566_primary Th2_resting dy 4-6 in IL-2 | 4.5 | 0.0 |
| 93567_primary Tr1_resting dy 4-6 in IL-2 | 2.3 | 0.7 |
| 93351_CD45RA CD4 lymphocyte_anti-CD28/anti-CD3 | 2.0 | 0.7 |
| 93352_CD45RO CD4 lymphocyte_anti-CD28/anti-CD3 | 1.8 | 2.0 |
| 93251_CD8 Lymphocytes_anti-CD28/anti-CD3 | 5.6 | 0.9 |
| 93353_chronic CD8 Lymphocytes 2ry_resting dy 4-6 in IL-2 | 4.6 | 1.1 |
| 93574_chronic CD8 Lymphocytes 2ry_activated CD3/CD28 | 1.8 | 0.2 |
| 93354_CD4_none | 7.2 | 1.3 |
| 93252_Secondary Th1/Th2/Tr1_anti-CD95 CH11 | 6.6 | 1.1 |
| 93103_LAK cells_resting | 7.6 | 0.4 |
| 93788_LAK cells_IL-2 | 4.8 | 0.3 |
| 93787_LAK cells_IL-2 + IL-12 | 5.7 | 0.8 |
| 93789_LAK cells_IL-2 + IFN gamma | 5.5 | 0.2 |
| 93790_LAK cells_IL-2 + IL-18 | 1.6 | 0.4 |
| 93104_LAK cells_PMA/ionomycin and IL-18 | 2.7 | 1.3 |
| 93578_NK Cells IL-2_resting | 5.6 | 2.0 |
| 93109_Mixed Lymphocyte Reaction_Two Way MLR | 4.9 | 2.0 |
| 93110_Mixed Lymphocyte Reaction_Two Way MLR | 0.5 | 0.8 |
| 93111_Mixed Lymphocyte Reaction_Two Way MLR | 6.0 | 0.2 |
| 93112_Mononuclear Cells (PBMCs)_resting | 1.3 | 0.4 |
| 93113_Mononuclear Cells (PBMCs)_PWM | 7.9 | 0.5 |
| 93114_Mononuclear Cells (PBMCs)_PHA-L | 5.1 | 0.5 |
| 93249_Ramos (B cell)_none | 5.7 | 0.6 |
| 93250_Ramos (B cell)_ionomycin | 4.4 | 0.3 |
| 93349_B lymphocytes_PWM | 1.1 | 0.2 |
| 93350_B lymphoytes_CD40L and IL-4 | 4.3 | 0.6 |
| 92665_EOL-1 (Eosinophil)_dbcAMP differentiated | 8.4 | 3.5 |
| 93248_EOL-1 (Eosinophil)_dbcAMP/PMAionomycin | 7.2 | 5.1 |
| 93356_Dendritic Cells_none | 3.4 | 1.0 |
| 93355_Dendritic Cells_LPS 100 ng/ml | 5.5 | 0.5 |
| 93775_Dendritic Cells_anti-CD40 | 2.6 | 0.3 |
| 93774_Monocytes_resting | 1.1 | 0.9 |
| 93776_Monocytes_LPS 50 ng/ml | 2.6 | 0.3 |
| 93581_Macrophages_resting | 5.2 | 0.2 |
| 93582_Macrophages_LPS 100 ng/ml | 1.4 | 0.3 |
| 93098_HUVEC (Endothelial)_none | 1.2 | 0.2 |
| 93099_HUVEC (Endothelial)_starved | 3.4 | 0.2 |
| 93100_HUVEC (Endothelial)_IL-1b | 2.4 | 0.1 |
| 93779_HUVEC (Endothelial)_IFN gamma | 2.6 | 1.2 |
| 93102_HUVEC (Endothelial)_TNF alpha + IFN gamma | 0.0 | 0.3 |
| 93101_HUVEC (Endothelial)_TNF alpha + IL4 | 1.6 | 0.4 |
| 93781_HUVEC (Endothelial)_IL-11 | 2.2 | 0.4 |
| 93583_Lung Microvascular Endothelial Cells_none | 1.8 | 0.9 |
| 93584_Lung Microvascular Endothelial Cells_TNFa (4 ng/ml) | 2.2 | 0.3 |
| and IL1b (1 ng/ml) | | |
| 92662_Microvascular Dermal endothelium_none | 1.4 | 0.5 |
| 92663_Microsvasular Dermal endothelium_TNFa (4 ng/ml) and | 2.2 | 0.3 |
| IL1b (1 ng/ml) | | |
| 93773_Bronchial epithelium_TNFa (4 ng/ml) and IL1b (1 ng/ml)** | 17.8 | 0.5 |
| 93347_Small Airway Epithelium_none | 1.5 | 0.2 |
| 93348_Small Airway Epithelium_TNFa (4 ng/ml) and IL1b (1 ng/ml) | 3.0 | 0.6 |
| 92668_Coronery Artery SMC_resting | 1.1 | 0.6 |
| 92669_Coronery Artery SMC_TNFa (4 ng/ml) and IL1b (1 ng/ml) | 1.9 | 0.8 |
| 93107_astrocytes_resting | 2.5 | 1.5 |
| 93108_astrocytes_TNFa (4 ng/ml) and IL1b (1 ng/ml) | 0.5 | 1.2 |
| 92666_KU-812 (Basophil)_resting | 4.3 | 0.8 |
| 92667_KU-812 (Basophil)_PMA/ionoycin | 3.0 | 0.6 |
| 93579_CCD1106 (Keratinocytes)_none | 1.6 | 0.9 |
| 93580_CCD1106 (Keratinocytes)_TNFa and IFNg** | 2.4 | 0.0 |
| 93791_Liver Cirrhosis | 76.6 | 9.8 |
| 93577_NCI-H292 | 5.4 | 4.1 |
| 93358_NCI-H292_IL-4 | 10.3 | 0.6 |
| 93360_NCI-H292_IL-9 | 16.5 | 1.3 |
| 93359_NCI-H292_IL-13 | 8.5 | 3.6 |
| 93357_NCI-H292_IFN gamma | 5.8 | 3.4 |
| 93777_HPAEC_- | 1.2 | 1.0 |
| 93778_HPAEC_IL-1 beta/TNA alpha | 1.5 | 0.3 |
| 93254_Normal Human Lung Fibroblast_none | 2.5 | 0.8 |
| 93253_Normal Human Lung Fibroblast_TNFa (4 ng/ml) and IL- | 5.7 | 0.9 |
| 1b (1 ng/ml) | | |
| 93257_Normal Human Lung Fibroblast_IL-4 | 2.9 | 0.4 |
| 93256_Normal Human Lung Fibroblast_IL-9 | 2.5 | 0.4 |
| 93255_Normal Human Lung Fibroblast_IL-13 | 2.7 | 1.9 |
| 93258_Normal Human Lung Fibroblast_IFN gamma | 0.0 | 2.2 |
| 93106_Dermal Fibroblasts CCD1070_resting | 6.1 | 3.4 |
| 93361_Dermal Fibroblasts CCD1070_TNF alpha 4 ng/ml | 2.1 | 3.8 |
| 93105_Dermal Fibroblasts CCD1070_IL-1 beta 1 ng/ml | 4.0 | 1.5 |
| 93772_dermal fibroblast_IFN gamma | 1.6 | 0.9 |
| 93771_dermal fibroblast_IL-4 | 1.4 | 1.3 |
| 93892_Dermal fibroblasts_none | 2.3 | 1.5 |
| 99202_Neutrophils_TNFa + LPS | 0.0 | 1.7 |
| 99203_Neutrophils_none | 0.8 | 0.6 |
| 735010_Colon_normal | 21.7 | 6.7 |
| 735019_Lung_none | 3.7 | 10.6 |
| 64028-1_Thymus_none | 11.7 | 27.0 |
| 64030-1_Kidney_none | 100.0 | 100.0 |

TABLE 52
Panel CNS_Neurodegeneration_v1.0

| | Relative | | Relative |
|---|---|---|---|
| | Expression(%) | | Expression(%) |
| | tm7142f_{—} | | tm7142f_{—} |
| Tissue Name | ag3797_b2 | Tissue Name | ag3797_b2 |
| AD 1 Hippo | 53.6 | Control (Path) 3 Temporal Ctx | 12.5 |
| AD 2 Hippo | 69.7 | Control (Path) 4 Temporal Ctx | 62.2 |
| AD 3 Hippo | 25.6 | AD 1 Occipital Ctx | 48.0 |
| AD 4 Hippo | 33.9 | AD 2 Occipital Ctx (Missing) | 0.0 |
| AD 5 Hippo | 91.6 | AD 3 Occipital Ctx | 13.0 |
| AD 6 Hippo | 39.7 | AD 4 Occipital Ctx | 40.7 |
| Control 2 Hippo | 38.4 | AD 5 Occipital Ctx | 51.9 |
| Control 4 Hippo | 59.4 | AD 6 Occipital Ctx | 28.4 |
| Control (Path) 3 Hippo | 7.8 | Control 1 Occipital Ctx | 2.6 |
| AD 1 Temporal Ctx | 41.0 | Control 2 Occipital Ctx | 100.0 |
| AD 2 Temporal Ctx | 70.4 | Control 3 Occipital Ctx | 33.1 |
| AD 3 Temporal Ctx | 21.3 | Control 4 Occipital Ctx | 18.6 |
| AD 4 Temporal Ctx | 46.7 | Control (Path) 1 Occipital Ctx | 82.7 |
| AD 5 Inf Temporal Ctx | 92.1 | Control (Path) 2 Occipital Ctx | 18.7 |
| AD 5 Sup Temporal Ctx | 74.8 | Control (Path) 3 Occipital Ctx | 3.4 |
| AD 6 Inf Temporal Ctx | 44.5 | Control (Path) 4 Occipital Ctx | 49.0 |
| AD 6 Sup Temporal Ctx | 57.9 | Control 1 Parietal | 19.8 |
| Control 1 Temporal Ctx | 22.8 | Control 2 Parietal | 60.6 |
| Control 2 Temporal Ctx | 45.7 | Control 3 Parietal | 31.0 |
| Control 3 Temporal Ctx | 13.8 | Control (Path) 1 Parietal | 57.3 |
| Control 3 Temporal Ctx | 51.4 | Control (Path) 2 Parietal | 31.0 |
| Control (Path) 1 Temporal Ctx | 62.8 | Control (Path) 3 Parietal | 5.7 |
| Control (Path) 2 Temporal Ctx | 41.4 | Control (Path) 4 Parietal | 52.1 |

Panel 1.3D Summary Ag3086 The NOV5gene is highly expressed in both fetal and adult liver tissue (CTs=26) and liver cancer cell lines (CT=27). The gene is also expressed at moderate to low levels in most of the other tissues in the panel. Thus, since the NOV5gene appears to be highly expressed in liver tissue, it could therefore be used to distinguish liver derived tissue from other tissues. The NOV5gene product may also be a potential therapeutic treatment of liver disease.

Among tissues involved in the central nervous system, the NOV5gene is moderately expressed in the fetal and adult brain, including the adult thalamus, substantia nigra, hippocampus, amygdala and is also expressed at low but significant levels in the cerebellum and cerebral cortex. This expression profile suggests that the NOV5gene has functional significance in the CNS. The close homologue to the NOV5 gene product, hepatocyte growth factor, has numerous therapeutic applications in the CNS, including prevention of neuronal death in animal models of stroke and ischemia. Hepatocyte growth factor has mitogenic activity, crossing the blood brain barrier when disrupted, and thus has potential application as a protein therapeutic to treat brain pathologies when administered directly to the cortico spinal fluid or systemically when the blood brain barrier is disrupted. Hepatocyte growth factor-like protein is a neurotrophic factor useful in the prevention of motoneuron atrophy upon axotomy. Therefore, the protein encoded by the NOV5gene may be useful as a therapeutic agent in treating stroke and neurodegenerative diseases including Alzheimer's disease, Parkinson's disease, and Huntington's disease. The potential role of the NOV5gene or its protein product in brain plasticity and regeneration affords utility in treating brain damage and aging related disorders, such as memory impairment that has hippocampal dysfunction as its primary focus.

General_Screening_Panel_{—}1.4 Ag3797 The expression of the NOV5gene in panel 1.4 appears to be highest in a sample derived from a liver cancer cell line (HepG2) (CT=25.3). In addition there is substantial expression of this gene associated with other liver derived material (adult liver CT=27.2; fetal liver CT=26.5). Thus, the expression of the NOV5gene could be used to distinguish liver derived specimens from other samples. In addition, therapeutic modulation of this gene might be of benefit in the treatment of liver related disorders, such as cirrhosis.

Panel 2.2 Summary Ag3086 The expression of the NOV5gene appears to be highest in a sample derived from a liver cancer specimen (CT=26) and is also significant in a number of samples derived from liver tissue. This result is consistent with what is seen in Panels 1.4 and 2D. In addition there appears to be substantial expression of this gene associated with normal kidney tissue (CT=27.2) when compared to adjacent kidney cancer specimens. Thus, this gene could be used to distinguish liver tissue from non-liver tissue as well as distinguish normal kidney tissue when compared to adjacent kidney cancer. Moreover, therapeutic modulation of the expression of the NOV5 gene or function of its product might be of benefit in the treatment of kidney cancer.

Panel 4D Summary Ag3086 The NOV5gene is highly expressed in the thymus (CT=24), colon (CT=28.4), and IBD Colitis 2 (CT=27.2) and is expressed at lower levels in mature T cells. The NOV5gene encodes a putative hepatocyte like growth factor homologue. There are reports that hepatocyte growth factor (HGF) is expressed in the thymus and colon. In the thymus, HGF may promote T cell production and in the colon, overexpression of HGF has been shown to leads to IBD like disease in mice. Therapies designed with the protein encoded for by the NOV5gene could be important in the regulation of T cell development and immune function and be useful in organ transplantation. In addition, blocking the function of the NOV5 gene product could help in the treatment of IBD colitis.

Panel 4.1D Summary A,3797 Results from two experiments using the same probe and primer set are in very good agreement. In both experiments, highest expression of the NOV5gene is detected in kidney (CT=29, 27.4). Moderate expression is also detected in liver cirrhosis (CT=29.4, 30.7). Moderate to low expression of the gene is detected in many of the tissues in this panel. Thus, expression of the NOV5gene could be used to distinguish those tissues from other tissues.

Panel CNS_Neurodegeneration_v1.0 Summary Ag3797 Highest expression of the NOV5 gene is detected in the occipital cortex of a control patient (CT=31.3). Moderate to low expression is detected throughought the tissue samples in this panel. Please see panel 1.3 for a discussion of potential utility of this gene with regards to the CNS.

References:

1. Korhonen L, Sjoholm U, Takei N, Kern M A, Schirmacher P, Castren E, Lindholm D. (2000) Eur J. Neurosci. 12:3453-61.

Hepatocyte growth factor-scatter factor (HGF) is expressed in different parts of the nervous system, and has been shown to exhibit neurotrophic activity. Here we show that c-Met, the receptor for HGF, is expressed in developing rat hippocampus, with the highest levels during the first postnatal weeks. To study the function of HGF, hippocampal neurons were prepared from embryonic rats and treated with different HGF concentrations. In these cultures, HGF increased the number of neurons expressing the 28-kDa calcium-binding protein (calbindin D) in a dose-dependent manner. The effect of HGF was larger than that observed with either brain-derived neurotrophic factor (BDNF) or neurotrophin-3 (NT-3), and cotreatment of the cultures with HGF and the neurotrophins was additive with respect to calbindin D neurons. Besides affecting the number of neurons, HGF significantly increased the degree of sprouting of calbindin D-positive neurons, suggesting an influence on neuronal maturation. BDNF and NT-3 stimulated neurite outgrowth of calbindin D neurons to a much smaller degree. In contrast to calbindin D neurons, HGF did not significantly increase the number of neurons immunoreactive with the neurotransmitter gamma-aminobutyric acid (GABA) in the hippocampal cultures. Immunohistochemical studies showed that c-Met-, calbindin D- and HGF-immunoreactive cells are all present in the dentate gyrus and partly colocalize within neurons. These results show that HGF acts on calbindin D-containing hippocampal neurons and increases their neurite outgrowth, suggesting that HGF plays an important role for the maturation and function of these neurons in the hippocampus.

PMID: 11029614

2. Powell E M, Mars W M, Levitt P. (2001) Neuron 30:79-89.

Cortical interneurons arise from the proliferative zone of the ventral telencephalon, the ganglionic eminence, and migrate into the developing neocortex. The spatial patterns of migratory interneurons reflect the complementary expression of hepatocyte growth factor/scatter factor (HGF/SF) and its receptor, MET, in the forebrain. Scatter assays on forebrain explants demonstrate regionally specific motogenic activity due to HGF/SF. In addition, exogenous ligand disrupts normal cell migration. Mice lacking the urokinase-type plasminogen activator receptor (u-PAR), a key component of HGF/SF activation, exhibit deficient scatter activity in the forebrain, abnormal interneuron migration from the ganglionic eminence, and reduced interneurons in the frontal and parietal cortex. The data suggest that HGF/SF motogenic activity, which is essential for normal development of other organ systems, is a conserved mechanism that regulates trans-telencephalic migration of interneurons.

PMID: 11343646

3. Stella M C, Vercelli A, Repici M, Follenzi A, Comoglio P M. (2001) Mol Biol Cell 12:1341-52.

Macrophage stimulating protein (MSP), also known as hepatocyte growth factor-like, is a soluble cytokine that belongs to the family of the plasminogen-related growth factors (PRGFs). PRGFs are alpha/beta heterodimers that bind to transmembrane tyrosine kinase receptors. MSP was originally isolated as a chemotactic factor for peritoneal macrophages. Through binding to its receptor, encoded by the RON gene, it stimulates dissociation of epithelia and works as an inflammatory mediator by repressing the production of nitric oxide (NO). Here, we identify a novel role for MSP in the central nervous system. As a paradigm to analyze this function we chose the hypoglossal system of adult mice. We demonstrate in vivo that either administration of exogenous MSP or transplantation of MSP-producing cells at the proximal stump of the resected nerve is sufficient to prevent motoneuron atrophy upon axotomy. We also show that the MSP gene is expressed in the tongue, the target of the hypoglossal nerve, and that MSP induces biosynthesis of Ron receptor in the motoneuron somata. Finally, we show that MSP suppresses NO production in the injured hypoglossal nuclei. Together, these data suggest that MSP is a novel neurotrophic factor for cranial motoneurons and, by regulating the production of NO, may have a role in brain plasticity and regeneration.

PMID: 11359926

4. Kern M A, Bamborschke S, Nekic M, Schubert D, Rydin C, Lindholm D, Schirmacher P. (2001) Cytokine 14:170-6.

Hepatocyte growth factor (HGF) and its specific receptor, MET, are expressed in the developing and adult mammalian brain. Recent studies have shown a neurotrophic activity of HGF in the nervous system. The present study focused on HGF concentrations in the cerebrospinal fluid (CSF) and serum in normal persons and in different central nervous system (CNS) diseases considering blood-CSF barrier (BCB) function. Concentrations of HGF were analyzed using an enzyme-linked immunosorbent assay (ELISA). HGF was present in normal human CSF (346+/−126 pg/ml) representing approximately half of the HGF serum concentrations. The CSF HGF levels were not significantly changed in chronic CNS disease and in aseptic meningitis (419+/−71 pg/ml), but significantly increased in patients with bacterial meningitis (6101+/−5200 pg/ml). The HGF levels in CSF were not influenced by increased serum concentrations in patients with normal or mildly affected BCB function. The results show that HGF is present in normal CSF and does not appear to cross the CSF barrier significantly unless it is severely disrupted. So far, strong increases of HGF concentration in CSF are only present in acute bacterial meningitis. Copyright 2001 Academic Press.

PMID: 11396995

5. Hayashi K, Morishita R, Nakagami H, Yoshimura S, Hara A, Matsumoto K, Nakamura T, Ogihara T, Kaneda Y, Sakai N. (2001) Gene Ther 8:1167-73.

To develop a novel strategy to prevent delayed neuronal death (DND) following transient occlusion of arteries, the gene of hepatocyte growth factor (HGF), a novel neurotrophic factor, was transfected into the subarachnoid space of gerbils after transient forebrain ischemia. Importantly, transfection of HGF gene into the subarachnoid space prevented DND, accompanied by a significant increase in HGF in the cerebrospinal fluid. Prevention of DND by HGF is due to the inhibition of apoptosis through the blockade of bax translocation from the cytoplasm to the nucleus. HGF gene transfer into the subarachnoid space may provide a new therapeutic strategy for cerebrovascular disease.

PMID: 11509947

6. Tamura S, Sugawara T, Tokoro Y, Taniguchi H, Fukao K, Nakauchi H, Takahama Y. (1998) Scand J. Immunol. 47:296-301.

The c-Met oncoprotein is a cell-surface receptor for hepatocyte growth factor (HGF). Signals through HGF and c-Met have been appreciated for their crucial roles in the development of many cell types, including liver cells. The present study examined whether c-Met is expressed in the thymus and whether c-Met/HGF signals can regulate T-cell development in the thymus. We have found that mRNA transcripts encoding c-Met are expressed in mouse thymus. The c-Met transcripts were expressed at higher levels in fetal and neonatal thymus than in adult thymus, and were mostly expressed by lymphoid cells rather than by stromal cells. Interestingly, the addition of HGF to fetal thymus organ cultures increased the generation of mature T cells expressing high levels of T-cell antigen receptors. These results indicate that c-Met is expressed in the thymus during early ontogeny, and that c-Met/HGF signals can promote T-cell development.

PMID: 9600310

7. Takayama H, Takagi H, Larochelle W J, Kapur R P, Merlino G. (2001) Lab Invest. 81:297-305.

Hepatocyte growth factor/scatter factor (HGF/SF) can stimulate growth of gastrointestinal epithelial cells in vitro; however, the physiological role of HGF/SF in the digestive tract is poorly understood. To elucidate this in vivo function, mice were analyzed in which an HGF/SF transgene was overexpressed throughout the digestive tract. Nearly a third of all HGF/SF transgenic mice in this study (28 of 87) died by 6 months of age as a result of sporadic intestinal obstruction of unknown etiology. Enteric ganglia were not overtly affected, indicating that the pathogenesis of this intestinal lesion was different from that operating in Hirschsprung's disease. Transgenic mice also exhibited a rectal inflammatory bowel disease (IBD) with a high incidence of anorectal prolapse. Expression of interleukin-2 was decreased in the transgenic colon, indicating that HGF/SF may influence regulation of the local intestinal immune system within the colon. These results suggest that HGF/SF plays an important role in the development of gastrointestinal paresis and chronic intestinal inflammation. HGF/SF transgenic mice may represent a useful model for the study of molecular mechanisms associated with a subset of IBD and intestinal pseudo-obstruction. Moreover, our data identify previously unappreciated side effects that may be encountered when using HGF/SF as a therapeutic agent.

PMID: 11310823

NOV6

Expression of gene NOV6 was assessed using the primer-probe set Ag2439 described in Table 53. Results from RTQ-PCR runs are shown in Tables 54, 55, 56, and 57.

TABLE 53
Probe Name Ag2439

| | | | | Start | SEQ ID |
|---|---|---|---|---|---|
| Primers | Sequences | TM | Length | Position | NO: |
| Forward | 5′-TATCATCACTTGTGATGGCAAA-3′ | 59 | 22 | 189 | 150 |
| Probe | FAM-5′-AAAACCGAGAGCACTTTGAAAACACA-3′-TAMRA | 66.1 | 26 | 223 | 151 |
| Reverse | 5′-AAACTTCTCTCCCAGGGTACAA-3′ | 59.1 | 22 | 255 | 152 |

TABLE 54
Panel 1.3D

| | Relative | | Relative |
|---|---|---|---|
| | Expression(%) | | Expression(%) |
| | 1.3dtm3781t_{—} | | 1.3dtm3781t_{—} |
| Tissue Name | ag2439 | Tissue Name | ag2439 |
| Liver adenocarcinoma | 11.3 | Kidney (fetal) | 7.6 |
| Pancreas | 3.7 | Renal ca. 786-0 | 4.9 |
| Pancreatic ca. CAPAN 2 | 4.9 | Renal ca. A498 | 10.4 |
| Adrenal gland | 5.3 | Renal ca. RXF 393 | 4.1 |
| Thyroid | 7.4 | Renal ca. ACHN | 2.2 |
| Salivary gland | 5.3 | Renal ca. UO-31 | 0.6 |
| Pituitary gland | 7.6 | Renal ca. TK-10 | 5.4 |
| Brain (fetal) | 23.5 | Liver | 4.2 |
| Brain (whole) | 6.2 | Liver (fetal) | 12.9 |
| Brain (amygdala) | 12.9 | Liver ca. (hepatoblast) HepG2 | 11.9 |
| Brain (cerebellum) | 8.5 | Lung | 35.1 |
| Brain (hippocampus) | 100.0 | Lung (fetal) | 12.3 |
| Brain (substantia nigra) | 4.9 | Lung ca. (small cell) LX-1 | 8.8 |
| Brain (thalamus) | 9.2 | Lung ca. (small cell) NCI-H69 | 5.6 |
| Cerebral Cortex | 12.9 | Lung ca. (s.cell var.) SHP-77 | 21.8 |
| Spinal cord | 5.1 | Lung ca. (large cell)NCI-H460 | 5.8 |
| CNS ca. (glio/astro) U87-MG | 11.8 | Lung ca. (non-sm. cell) A549 | 14.9 |
| CNS ca. (glio/astro) U-118-MG | 15.5 | Lung ca. (non-s.cell) NCI-H23 | 11.9 |
| CNS ca. (astro) SW1783 | 3.7 | Lung ca (non-s.cell) HOP-62 | 4.6 |
| CNS ca.* (neuro; met) SK-N- | 46.7 | Lung ca. (non-s.cl) NCI-H522 | 2.1 |
| AS | | | |
| CNS ca. (astro) SF-539 | 8.8 | Lung ca. (squam.) SW 900 | 3.1 |
| CNS ca. (astro) SNB-75 | 4.2 | Lung ca. (squam.) NCI-H596 | 3.1 |
| CNS ca. (glio) SNB-19 | 9.9 | Mammary gland | 3.9 |
| CNS ca. (glio) U251 | 5.7 | Breast ca.* (pl. effusion) MCF-7 | 8.6 |
| CNS ca. (glio) SF-295 | 6.2 | Breast ca.* (pl.ef) MDA-MB- | 23.3 |
| | | 231 | |
| Heart (fetal) | 6.1 | Breast ca.* (pl. effusion) T47D | 3.0 |
| Heart | 22.2 | Breast ca. BT-549 | 31.2 |
| Fetal Skeletal | 40.1 | Breast ca. MDA-N | 7.6 |
| Skeletal muscle | 2.5 | Ovary | 3.5 |
| Bone marrow | 11.3 | Ovarian ca. OVCAR-3 | 5.0 |
| Thymus | 6.7 | Ovarian ca. OVCAR-4 | 0.3 |
| Spleen | 12.2 | Ovarian ca. OVCAR-5 | 8.9 |
| Lymph node | 14.1 | Ovarian ca. OVCAR-8 | 9.3 |
| Colorectal | 22.8 | Ovarian ca. IGROV-1 | 5.4 |
| Stomach | 10.4 | Ovarian ca.* (ascites) SK-OV-3 | 12.6 |
| Small intestine | 21.2 | Uterus | 9.3 |
| Colon ca. SW480 | 2.1 | Placenta | 17.7 |
| Colon ca.* (SW480 met)SW620 | 9.3 | Prostate | 5.1 |
| Colon ca. HT29 | 8.4 | Prostate ca.* (bone met)PC-3 | 3.7 |
| Colon ca. HCT-116 | 6.0 | Testis | 5.1 |
| Colon ca. CaCo-2 | 16.7 | Melanoma Hs688(A).T | 1.5 |
| 83219 CC Well to Mod Diff | 13.5 | Melanoma* (met) Hs688(B).T | 1.0 |
| (ODO3866) | | | |
| Colon ca. HCC-2998 | 32.1 | Melanoma UACC-62 | 0.7 |
| Gastric ca.* (liver met) NCI- | 34.2 | Melanoma M14 | 4.6 |
| N87 | | | |
| Bladder | 14.0 | Melanoma LOX IMVI | 4.4 |
| Trachea | 24.8 | Melanoma* (met) SK-MEL-5 | 45.4 |
| Kidney | 1.7 | Adipose | 11.7 |

TABLE 55
Panel 2D

| | Relative | | Relative |
|---|---|---|---|
| | Expression(%) | | Expression(%) |
| | 2dtm3782t_{—} | | 2dtm3782t_{—} |
| Tissue Name | ag2439 | Tissue Name | ag2439 |
| Normal Colon GENPAK | 100.0 | Kidney NAT Clontech 8120608 | 1.2 |
| 061003 | | | |
| 83219 CC Well to Mod Diff | 17.2 | Kidney Cancer Clontech | 2.8 |
| (ODO3866) | | 8120613 | |
| 83220 CC NAT (ODO3866) | 13.8 | Kidney NAT Clontech 8120614 | 1.6 |
| 83221 CC Gr.2 rectosigmoid | 16.6 | Kidney Cancer Clontech | 7.7 |
| (ODO3868) | | 9010320 | |
| 83222 CC NAT (ODO3868) | 3.7 | Kidney NAT Clontech 9010321 | 3.3 |
| 83235 CC Mod Diff | 31.2 | Normal Uterus GENPAK | 5.2 |
| (ODO3920) | | 061018 | |
| 83236 CC NAT (ODO3920) | 17.2 | Uterus Cancer GENPAK | 22.1 |
| | | 064011 | |
| 83237 CC Gr.2 ascend colon | 74.2 | Normal Thyroid Clontech A+ | 10.5 |
| (ODO3921) | | 6570-1 | |
| 83238 CC NAT (ODO3921) | 15.3 | Thyroid Cancer GENPAK | 17.3 |
| | | 064010 | |
| 83241 CC from Partial | 32.5 | Thyroid Cancer INVITROGEN | 14.5 |
| Hepatectomy (ODO4309) | | A302152 | |
| 83242 Liver NAT (ODO4309) | 8.2 | Thyroid NAT INVITROGEN | 16.2 |
| | | A302153 | |
| 87472 Colon mets to lung | 5.3 | Normal Breast GENPAK | 16.7 |
| (OD04451-01) | | 061019 | |
| 87473 Lung NAT (OD04451- | 11.1 | 84877 Breast Cancer | 7.3 |
| 02) | | (OD04566) | |
| Normal Prostate Clontech A+ | 11.6 | 85975 Breast Cancer | 22.2 |
| 6546-1 | | (OD04590-01) | |
| 84140 Prostate Cancer | 21.0 | 85976 Breast Cancer Mets | 24.8 |
| (OD04410) | | (OD04590-03) | |
| 84141 Prostate NAT | 21.3 | 87070 Breast Cancer Metastasis | 15.3 |
| (OD04410) | | (OD04655-05) | |
| 87073 Prostate Cancer | 24.1 | GENPAK Breast Cancer | 18.0 |
| (OD04720-01) | | 064006 | |
| 87074 Prostate NAT | 22.5 | Breast Cancer Res. Gen. 1024 | 17.7 |
| (OD04720-02) | | | |
| Normal Lung GENPAK 061010 | 84.1 | Breast Cancer Clontech | 13.3 |
| | | 9100266 | |
| 83239 Lung Met to Muscle | 11.3 | Breast NAT Clontech 9100265 | 6.9 |
| (ODO4286) | | | |
| 83240 Muscle NAT | 9.4 | Breast Cancer INVITROGEN | 16.3 |
| (ODO4286) | | A209073 | |
| 84136 Lung Malignant Cancer | 19.9 | Breast NAT INVITROGEN | 11.0 |
| (OD03126) | | A2090734 | |
| 84137 Lung NAT (OD03126) | 36.9 | Normal Liver GENPAK | 7.1 |
| | | 061009 | |
| 84871 Lung Cancer (OD04404) | 28.7 | Liver Cancer GENPAK 064003 | 8.0 |
| 84872 Lung NAT (OD04404) | 11.7 | Liver Cancer Research Genetics | 5.1 |
| | | RNA 1025 | |
| 84875 Lung Cancer (OD04565) | 37.4 | Liver Cancer Research Genetics | 1.3 |
| | | RNA 1026 | |
| 84876 Lung NAT (OD04565) | 13.3 | Paired Liver Cancer Tissue | 5.4 |
| | | Research Genetics RNA 6004-T | |
| 85950 Lung Cancer (OD04237- | 25.9 | Paired Liver Tissue Research | 11.9 |
| 01) | | Genetics RNA 6004-N | |
| 85970 Lung NAT (OD04237- | 21.3 | Paired Liver Cancer Tissue | 1.6 |
| 02) | | Research Genetics RNA 6005-T | |
| 83255 Ocular Mel Met to Liver | 9.5 | Paired Liver Tissue Research | 0.5 |
| (ODO4310) | | Genetics RNA 6005-N | |
| 83256 Liver NAT (ODO4310) | 6.8 | Normal Bladder GENPAK | 39.8 |
| | | 061001 | |
| 84139 Melanoma Mets to Lung | 11.2 | Bladder Cancer Research | 8.9 |
| (OD04321) | | Genetics RNA 1023 | |
| 84138 Lung NAT (OD04321) | 30.4 | Bladder Cancer INVITROGEN | 93.3 |
| | | A302173 | |
| Normal Kidney GENPAK | 23.2 | 87071 Bladder Cancer | 22.5 |
| 061008 | | (OD04718-01) | |
| 83786 Kidney Ca, Nuclear | 14.2 | 87072 Bladder Normal | 37.1 |
| grade 2 (OD04338) | | Adjacent (OD04718-03) | |
| 83787 Kidney NAT (OD04338) | 9.7 | Normal Ovary Res. Gen. | 2.0 |
| 83788 Kidney Ca Nuclear grade | 26.2 | Ovarian Cancer GENPAK | 21.3 |
| 1/2 (OD04339) | | 064008 | |
| 83789 Kidney NAT (OD04339) | 11.3 | 87492 Ovary Cancer | 33.4 |
| | | (OD04768-07) | |
| 83790 Kidney Ca, Clear cell | 24.5 | 87493 Ovary NAT (OD04768- | 4.9 |
| type (OD04340) | | 08) | |
| 83791 Kidney NAT (OD04340) | 17.2 | Normal Stomach GENPAK | 23.7 |
| | | 061017 | |
| 83792 Kidney Ca, Nuclear | 6.7 | Gastric Cancer Clontech | 5.6 |
| grade 3 (OD04348) | | 9060358 | |
| 83793 Kidney NAT (OD04348) | 9.2 | NAT Stomach Clontech | 18.9 |
| | | 9060359 | |
| 87474 Kidney Cancer | 7.7 | Gastric Cancer Clontech | 34.6 |
| (OD04622-01) | | 9060395 | |
| 87475 Kidney NAT (OD04622- | 0.9 | NAT Stomach Clontech | 27.4 |
| 03) | | 9060394 | |
| 85973 Kidney Cancer | 10.1 | Gastric Cancer Clontech | 64.2 |
| (OD04450-01) | | 9060397 | |
| 85974 Kidney NAT (OD04450- | 7.2 | NAT Stomach Clontech | 5.3 |
| 03) | | 9060396 | |
| Kidney Cancer Clontech | 1.2 | Gastric Cancer GENPAK | 48.0 |
| 8120607 | | 064005 | |

TABLE 56
Panel 4D

| | Relative | | Relative |
|---|---|---|---|
| | Expression | | Expression |
| | (%) | | (%) |
| | 4dtm3783t_{—} | | 4dtm3783t_{—} |
| Tissue Name | ag2439 | Tissue Name | ag2439 |
| 93768_Secondary Th1_anti- | 51.8 | 93100_HUVEC | 5.9 |
| CD28/anti-CD3 | | (Endothelial)_IL-1b | |
| 93769_Secondary Th2_anti- | 39.0 | 93779_HUVEC | 6.0 |
| CD28/anti-CD3 | | (Endothelial)_IFN gamma | |
| 93770_Secondary Tr1_anti- | 54.0 | 93102_HUVEC | 7.2 |
| CD28/anti-CD3 | | (Endothelial)_TNF alpha + IFN | |
| | | gamma | |
| 93573_Secondary Th1_resting | 4.2 | 93101_HUVEC | 18.9 |
| day 4-6 in IL-2 | | (Endothelial)_TNF alpha + IL4 | |
| 93572_Secondary Th2_resting | 5.7 | 93781_HUVEC | 6.2 |
| day 4-6 in IL-2 | | (Endothelial)_IL-11 | |
| 93571_Secondary Tr1_resting | 7.4 | 93583_Lung Microvascular | 21.8 |
| day 4-6 in IL-2 | | Endothelial Cells_none | |
| 93568_primary Th1_anti- | 82.4 | 93584_Lung Microvascular | 21.2 |
| CD28/anti-CD3 | | Endothelial Cells_TNFa (4 ng/ml) | |
| | | and IL1b (1 ng/ml) | |
| 93569_primary Th2_anti- | 52.1 | 92662_Microvascular Dermal | 33.7 |
| CD28/anti-CD3 | | endothelium_none | |
| 93570_primary Tr1_anti- | 76.8 | 92663_Microvascular Dermal | 15.1 |
| CD28/anti-CD3 | | endothelium_TNFa (4 ng/ml) | |
| | | and IL1b (1 ng/ml) | |
| 93565_primary Th1_resting dy | 36.3 | 93773_Bronchial | 1.0 |
| 4-6 in IL-2 | | epithelium_TNFa (4 ng/ml) and | |
| | | IL1b (1 ng/ml)** | |
| 93566_primary Th2_resting dy | 16.8 | 93347_Small Airway | 4.1 |
| 4-6 in IL-2 | | Epithelium_none | |
| 93567_primary Tr1_resting dy | 9.1 | 93348_Small Airway | 34.2 |
| 4-6 in IL-2 | | Epithelium_TNFa (4 ng/ml) | |
| | | and IL1b (1 ng/ml) | |
| 93351_CD45RA CD4 | 18.0 | 92668_Coronery Artery | 4.9 |
| lymphocyte_anti-CD28/anti- | | SMC_resting | |
| CD3 | | | |
| 93352_CD45RO CD4 | 37.1 | 92669_Coronery Artery | 0.6 |
| lymphocyte_anti-CD28/anti- | | SMC_TNFa (4 ng/ml) and IL1b | |
| CD3 | | (1 ng/ml) | |
| 93251_CD8 Lymphocytes_anti- | 40.6 | 93107_astrocytes_resting | 5.0 |
| CD28/anti-CD3 | | | |
| 93353_chronic CD8 | 41.2 | 93108_astrocytes_TNFa (4 ng/ml) | 6.6 |
| Lymphocytes 2ry_resting dy 4-6 | | and IL1b (1 ng/ml) | |
| in IL-2 | | | |
| 93574_chronic CD8 | 23.5 | 92666_KU-812 | 17.9 |
| Lymphocytes 2ry_activated | | (Basophil)_resting | |
| CD3/CD28 | | | |
| 93354_CD4_none | 4.8 | 92667_KU-812 | 43.5 |
| | | (Basophil)_PMA/ionoycin | |
| 93252_Secondary | 8.5 | 93579_CCD1106 | 10.2 |
| Th1/Th2/Tr1_anti-CD95 CH11 | | (Keratinocytes)_none | |
| 93103_LAK cells_resting | 32.8 | 93580_CCD1106 | 0.7 |
| | | (Keratinocytes)_TNFa and | |
| | | IFNg** | |
| 93788_LAK cells_IL-2 | 29.1 | 93791_Liver Cirrhosis | 2.6 |
| 93787_LAK cells_IL-2 + IL-12 | 26.2 | 93792_Lupus Kidney | 1.7 |
| 93789_LAK cells_IL-2 + IFN | 25.3 | 93577_NCI-H292 | 10.0 |
| gamma | | | |
| 93790_LAK cells_IL-2 + IL-18 | 28.5 | 93358_NCI-H292_IL-4 | 18.0 |
| 93104_LAK | 12.8 | 93360_NCI-H292_IL-9 | 15.6 |
| cells_PMA/ionomycin and IL- | | | |
| 18 | | | |
| 93578_NK Cells IL-2_resting | 10.5 | 93359_NCI-H292_IL-13 | 5.8 |
| 93109_Mixed Lymphocyte | 21.5 | 93357_NCI-H292_IFN gamma | 7.1 |
| Reaction_Two Way MLR | | | |
| 93110_Mixed Lymphocyte | 14.8 | 93777_HPAEC_- | 12.2 |
| Reaction_Two Way MLR | | | |
| 93111_Mixed Lymphocyte | 14.4 | 93778_HPAEC_IL-1 beta/TNA | 13.8 |
| Reaction_Two Way MLR | | alpha | |
| 93112_Mononuclear Cells | 3.9 | 93254_Normal Human Lung | 4.8 |
| (PBMCs)_resting | | Fibroblast_none | |
| 93113_Mononuclear Cells | 75.8 | 93253_Normal Human Lung | 2.3 |
| (PBMCs)_PWM | | Fibroblast_TNFa (4 ng/ml) and | |
| | | IL-1b (1 ng/ml) | |
| 93114_Mononuclear Cells | 36.3 | 93257_Normal Human Lung | 12.5 |
| (PBMCs)_PHA-L | | Fibroblast_IL-4 | |
| 93249_Ramos (B cell)_none | 44.1 | 93256_Normal Human Lung | 10.2 |
| | | Fibroblast_IL-9 | |
| 93250_Ramos (B | 100.0 | 93255_Normal Human Lung | 7.6 |
| cell)_ionomycin | | Fibroblast_IL-13 | |
| 93349_B lymphocytes_PWM | 93.3 | 93258_Normal Human Lung | 10.9 |
| | | Fibroblast_IFN gamma | |
| 93350_B lymphoytes_CD40L | 22.4 | 93106_Dermal Fibroblasts | 12.7 |
| and IL-4 | | CCD1070_resting | |
| 92665_EOL-1 | 6.1 | 93361_Dermal Fibroblasts | 32.3 |
| (Eosinophil)_dbcAMP | | CCD1070_TNF alpha 4 ng/ml | |
| differentiated | | | |
| 93248_EOL-1 | 16.2 | 93105_Dermal Fibroblasts | 4.9 |
| (Eosinophil)_dbcAMP/PMAionomycin | | CCD1070_IL-1 beta 1 ng/ml | |
| 93356_Dendritic Cells_none | 16.2 | 93772_dermal fibroblast_IFN | 6.3 |
| | | gamma | |
| 93355_Dendritic Cells_LPS | 30.8 | 93771_dermal fibroblast_IL-4 | 9.8 |
| 100 ng/ml | | | |
| 93775_Dendritic Cells_anti- | 39.0 | 93260_IBD Colitis 2 | 1.5 |
| CD40 | | | |
| 93774_Monocytes_resting | 4.9 | 93261_IBD Crohns | 1.2 |
| 93776_Monocytes_LPS 50 ng/ml | 6.8 | 735010_Colon_normal | 11.0 |
| 93581_Macrophages_resting | 41.5 | 735019_Lung_none | 17.9 |
| 93582_Macrophages_LPS 100 ng/ml | 7.1 | 64028-1_Thymus_none | 11.4 |
| 93098_HUVEC | 12.2 | 64030-1_Kidney_none | 41.5 |
| (Endothelial)_none | | | |
| 93099_HUVEC | 20.0 | | |
| (Endothelial)_starved | | | |

TABLE 57
Panel CNS_neurodegeneration_v1.0

| | Relative | | Relative |
|---|---|---|---|
| | Expression(%) | | Expression(%) |
| | tm6902t_ | | tm6902t_ |
| Tissue Name | ag2439_a2s1 | Tissue Name | ag2439_a2s1 |
| 106655_4951 Hippo | 1.7 | 106677_4624 BA21 | 13.8 |
| 106657_4986 Hippo | 5.4 | 106681_4640 BA21 | 2.5 |
| 106652_4933 Hippo | 10.3 | 106654_4951 BA17 | 20.2 |
| 106649_4901 Hippo | 2.4 | cns_water | 0.0 |
| 110138_3087 hippo | 100.0 | 106651_4933 BA17 | 14.8 |
| 110121_3027 Hippo | 0.0 | 106648_4901 BA17 | 2.2 |
| 106670_4971 Hippo | 2.4 | 110140_3087 occ ctx | 4.3 |
| 106666_4867 Hippo | 0.9 | 110123_3027 Occ Ctx | 0.0 |
| 106680_4624 Hippo | 0.0 | 106659_4595 BA17 | 23.7 |
| 106653_4951 BA21 | 32.6 | 106668_4971 BA17 | 5.2 |
| 106656_4986 BA21 | 1.7 | 106662_4737 BA17 | 31.4 |
| 106650_4933 BA21 | 26.2 | 106665_4867 BA17 | 11.1 |
| 106647_4901 BA21 | 24.3 | 106675_3975 BA17 | 56.4 |
| 110136_3087 inf temp ctx | 1.6 | 106672_3954 BA17 | 6.8 |
| 110137_3087 sup temp ctx | 8.1 | 106678_4624 BA17 | 9.0 |
| 110118_3027 Inf Temp Ctx | 1.5 | 106682_4640 BA17 | 29.9 |
| 110119_3027 Sup Temp Ctx | 0.0 | 106660_4595 BA7 | 14.7 |
| 106658_4595 BA21 | 14.7 | 113670_106669 pool | 53.3 |
| 106667_4971 BA21 | 10.4 | 106663_4737 BA7 | 1.8 |
| 106661_4737 BA21 | 31.6 | 106676_3975 BA7 | 39.3 |
| 106664_4867 BA21 | 1.5 | 106673_3954 BA7 | 0.5 |
| 106674_3975 BA21 | 37.3 | 106679_4624 BA7 | 18.2 |
| 106671_3954 BA21 | 58.2 | 106683_4640 BA7 | 93.7 |

Panel 1.3D Summary Ag2439 The NOV6 gene is widely expressed across the samples in this panel, with highest expression detected in the hippocampus (CT=28.1). This gene is also expressed at lower levels in the other tissues originating from the central nervous system, including the amygdala, cerebellum, cerebral cortex, substantia nigra, thalamus, and spinal cord. Thus, NOV6 gene expression may be used to distinguish hippocampus from other tissues. Please see CNS_neurodegeneration_panel_v1.0 summary for a discussion of the potential utility of this gene in CNS disorders.

Among tissues involved in metabolic function, the NOV6 gene is also expressed in thyroid, adrenal gland, pituitary gland, pancreas, heart (adult and fetal), liver (adult and fetal), and adipose. Interestingly, this gene is more highly expressed in fetal skeletal muscle (CT=29.4) than in adult skeletal muscle (CT=33.4). This observation suggests that the NOV6 protein product may enhance muscular growth or development in the fetus and thus may also act in a regenerative capacity in the adult. Therefore, therapeutic modulation of the NOV6 gene could be useful in treatment of muscular related disease. More specifically, treatment of weak or dystrophic muscle with the protein encoded by this gene could restore muscle mass or function.

The NOV6 gene is also expressed at higher levels in gastric, colon, melanoma, lung, and colon cancer cell lines than in normal tissues. Therefore, this gene may be used as a marker for gastric cancer, colon cancer, melanoma, lung, and colon cancer cell lines. In addition, therapeutic modulation of the NOV6 gene product might be of use in the treatment of these cancers.

Panel 2D Summary Ag2439 The NOV6 gene is most highly expressed in a sample derived from normal colon (CT=26). However, in general this gene appears to be more highly expressed in cancers than in normal tissues. Specifically, NOV6 gene expression is slightly higher in lung cancer (squamous cell type), gastric cancer, ovarian cancer, a kidney cancer sample and a sample of breast cancer relative to the normal controls. Thus, the expression of this gene could be used to distinguish malignant colon, lung, stomach, ovary and some breast and kidney tissue from normal tissue from these organs. In addition, therapeutic modulation of the NOV6 gene product might be of use in the treatment of these cancers.

Panel 4D Summary Ag2439 The NOV6 gene is most highly expressed in ionomycin-treated Ramos B cells (CT=26). This gene is also expressed at moderate levels in T cells, monocytes, dendritic cells, endothelial cells, smooth muscle cells, and airway epithelial cells both under resting and cytokine-stimulated conditions. Therefore, this gene may be useful as a marker for these resting and activated cells.

Panel CNS_neurodegeneration_v1.0 Summary Ag2439 The NOV6 gene encodes a protein with homology to fatty acid binding protein and is expressed across the brain, although expression appears to be the highest in the hippocampus (Panel 1.3D). This gene does not appear to be differentially expressed in Alzheimer's disease based on the results from panel CNS_Neurodegeneration_V1.0, although the transcript is detected at low levels in many of the brain samples. Fatty acid binding protein expression is increased in development during axon growth and during the response to injury, probably for the transport of fatty acids for use as membrane components (ref. 1). Therefore, upregulation of the NOV6 gene or its protein product may be beneficial during neurite outgrowth and synaptogenesis in response to neuronal death or injury (Parkinson's disease, Alzheimer's disease, Huntington's disease, spinocerebellar ataxia, stroke, and head/spinal cord trauma).

References:

1. Liu Y, Molina C A, Welcher A A, Longo L D, De Leon M. Expression of DA11, a neuronal-injury-induced fatty acid binding protein, coincides with axon growth and neuronal differentiation during central nervous system development. J Neurosci Res 1997 Jun 15;48(6):551-62.

DA11 is the first fatty acid binding protein (FABP) for which gene expression has been shown to be upregulated following neuronal injury in the adult peripheral nervous system. To understand better the potential regulatory role(s) of this unique FABP in axonal growth and neuronal differentiation, we undertook a temporal and spatial study of DA11 gene expression in the developing rat central nervous system (CNS). Transient upregulation of DA11 mRNA and protein levels in CNS tissues were quantified by Northern blot hybridization and Western immunoblot analyses at different developmental ages. Homogenates of embryonic and neonatal cerebral cortex, cerebellum, brain stem, and hippocampal tissues contained 100-fold more DA11 mRNA and protein than corresponding adult tissues. Significant increase in DA11 mRNA was observed as early as embryonic day (E) 14 in cerebral cortex and cerebellum and E19 in brain stem and hippocampus. Postnatal levels of DA11 remained elevated through postnatal day (P) 10 in cerebral cortex, P14 in brain stem and hippocampus, and P20 in cerebellum. Localization of DA11-like immunoreactivity to specific CNS tissues, cell types, and intracellular compartments at P9 revealed a spatial pattern of neuronal expression different than that reported for other FABPs. DA 10 protein was detected in the nucleus, cytoplasm, axons, and dendrites of differentiating neurons in cerebral cortex, hippocampus, cerebellum, brain stem, spinal cord, and olfactory bulb. The strong association of DAII gene expression with development throughout the CNS suggests that this unique FABP plays an important role in axonal growth and neuronal differentiation in many different neuronal populations.

PMID: 9210525

NOV9

Expression of gene NOV9 was assessed using the primer-probe set Ag2771 described in Table 58. Results from RTQ-PCR runs are shown in Tables 59, 60, 61, and 62.

TABLE 58
Probe Name Ag2771

| | | | | Start | |
|---|---|---|---|---|---|
| Primers | Sequences | TM | Length | Position | SEQ ID NO: |
| Forward | 5′-TGAACAGAACTATGCGAAACAA-3′ | 58.5 | 22 | 223 | 153 |
| Probe | FAM-5′-TCTGGTTAAGAAGTACTGCCCCAAACG-3′-TAMRA | 68 | 27 | 253 | 154 |
| Reverse | 5′-GGCTCTTCATCTTTGGATGAA-3′ | 59.3 | 21 | 280 | 155 |

TABLE 59
Panel 1.3D

| | Relative | | Relative |
|---|---|---|---|
| | Expression(%) | | Expression(%) |
| | 1.3Dx4tm4869 | | 1.3Dx4tm4869 |
| Tissue Name | f_ag2771_a1 | Tissue Name | f_ag2771_a1 |
| Liver adenocarcinoma | 12.7 | Kidney (fetal) | 32.3 |
| Pancreas | 3.2 | Renal ca. 786-0 | 22.1 |
| Pancreatic ca. CAPAN 2 | 4.7 | Renal ca. A498 | 13.0 |
| Adrenal gland | 2.1 | Renal ca. RXF 393 | 13.1 |
| Thyroid | 14.5 | Renal ca. ACHN | 4.0 |
| Salivary gland | 10.5 | Renal ca. UO-31 | 15.9 |
| Pituitary gland | 2.5 | Renal ca. TK-10 | 22.2 |
| Brain (fetal) | 16.8 | Liver | 1.2 |
| Brain (whole) | 5.0 | Liver (fetal) | 3.7 |
| Brain (amygdala) | 5.0 | Liver ca. (hepatoblast) HepG2 | 20.9 |
| Brain (cerebellum) | 8.1 | Lung | 9.9 |
| Brain (hippocampus) | 7.7 | Lung (fetal) | 23.0 |
| Brain (substantia nigra) | 2.1 | Lung ca. (small cell) LX-1 | 20.5 |
| Brain (thalamus) | 5.5 | Lung ca. (small cell) NCI-H69 | 14.0 |
| Cerebral Cortex | 28.3 | Lung ca. (s.cell var.) SHP-77 | 38.9 |
| Spinal cord | 17.3 | Lung ca. (large cell) NCI-H460 | 4.6 |
| CNS ca. (glio/astro) U87-MG | 2.2 | Lung ca. (non-sm. cell) A549 | 9.5 |
| CNS ca. (glio/astro) U-118-MG | 5.1 | Lung ca. (non-s.cell) NCI-H23 | 20.1 |
| CNS ca. (astro) SW1783 | 21.6 | Lung ca. (non-s.cell) HOP-62 | 10.3 |
| CNS ca.* (neuro; met) SK-N- | 26.8 | Lung ca. (non-s.cl) NCI-H522 | 36.6 |
| AS | | | |
| CNS ca. (astro) SF-539 | 12.7 | Lung ca. (squam.) SW 900 | 21.3 |
| CNS ca. (astro) SNB-75 | 12.3 | Lung ca. (squam.) NCI-H596 | 11.8 |
| CNS ca. (glio) SNB-19 | 34.0 | Mammary gland | 21.5 |
| CNS ca. (glio) U251 | 20.2 | Breast ca.* (pl. effusion) MCF-7 | 25.3 |
| CNS ca. (glio) SF-295 | 12.4 | Breast ca.* (pl. ef) MDA-MB- | 10.5 |
| | | 231 | |
| Heart (fetal) | 6.2 | Breast ca.* (pl. effusion) T47D | 13.3 |
| Heart | 14.1 | Breast ca. BT-549 | 8.0 |
| Fetal Skeletal | 23.1 | Breast ca. MDA-N | 13.2 |
| Skeletal muscle | 20.0 | Ovary | 15.2 |
| Bone marrow | 0.7 | Ovarian ca. OVCAR-3 | 27.2 |
| Thymus | 10.1 | Ovarian ca. OVCAR-4 | 3.2 |
| Spleen | 1.5 | Ovarian ca. OVCAR-5 | 9.8 |
| Lymph node | 1.9 | Ovarian ca. OVCAR-8 | 25.5 |
| Colorectal | 1.6 | Ovarian ca. IGROV-1 | 12.1 |
| Stomach | 4.2 | Ovarian ca.* (ascites) SK-OV-3 | 10.6 |
| Small intestine | 5.2 | Uterus | 2.6 |
| Colon ca. SW480 | 10.7 | Placenta | 7.9 |
| Colon ca.* (SW480 met) SW620 | 31.7 | Prostate | 6.2 |
| Colon ca. HT29 | 41.9 | Prostate ca.* (bone met) PC-3 | 13.2 |
| Colon ca. HCT-116 | 13.9 | Testis | 11.8 |
| Colon ca. CaCo-2 | 25.6 | Melanoma Hs688(A).T | 5.2 |
| 83219 CC Well to Mod Diff | 51.7 | Melanoma* (met) Hs688(B).T | 6.1 |
| (ODO3866) | | | |
| Colon ca. HCC-2998 | 11.9 | Melanoma UACC-62 | 1.0 |
| Gastric ca.* (liver met) NCI- | 44.6 | Melanoma M14 | 2.3 |
| N87 | | | |
| Bladder | 100.0 | Melanoma LOX IMVI | 1.1 |
| Trachea | 23.9 | Melanoma* (met) SK-MEL-5 | 13.3 |
| Kidney | 34.9 | Adipose | 10.5 |

TABLE 60
Panel 2D

| | Relative | | Relative |
|---|---|---|---|
| | Expression(%) | | Expression(%) |
| | 2dx4tm4680f_ | | 2dx4tm4680f_ |
| Tissue Name | ag2771_a2 | Tissue Name | ag2771_a2 |
| Normal Colon GENPAK | 52.3 | Kidney NAT Clontech 8120608 | 1.9 |
| 061003 | | | |
| 83219 CC Well to Mod Diff | 5.0 | Kidney Cancer Clontech | 5.4 |
| (ODO3866) | | 8120613 | |
| 83220 CC NAT (ODO3866) | 5.0 | Kidney NAT Clontech 8120614 | 2.4 |
| 83221 CC Gr.2 rectosigmoid | 2.8 | Kidney Cancer Clontech | 2.4 |
| (ODO3868) | | 9010320 | |
| 83222 CC NAT (ODO3868) | 1.4 | Kidney NAT Clontech 9010321 | 5.2 |
| 83235 CC Mod Diff | 17.6 | Normal Uterus GENPAK | 2.5 |
| (ODO3920) | | 061018 | |
| 83236 CC NAT (ODO3920) | 5.7 | Uterus Cancer GENPAK | 17.1 |
| | | 064011 | |
| 83237 CC Gr.2 ascend colon | 19.3 | Normal Thyroid Clontech A+ | 12.9 |
| (ODO3921) | | 6570-1 | |
| 83238 CC NAT (ODO3921) | 7.3 | Thyroid Cancer GENPAK | 16.9 |
| | | 064010 | |
| 83241 CC from Partial | 18.0 | Thyroid Cancer INVITROGEN | 11.4 |
| Hepatectomy (ODO4309) | | A302152 | |
| 83242 Liver NAT (ODO4309) | 5.6 | Thyroid NAT INVITROGEN | 17.6 |
| | | A302153 | |
| 87472 Colon mets to lung | 4.7 | Normal Breast GENPAK | 27.2 |
| (OD04451-01) | | 061019 | |
| 87473 Lung NAT (OD04451- | 8.4 | 84877 Breast Cancer | 26.0 |
| 02) | | (OD04566) | |
| Normal Prostate Clontech A+ | 39.8 | 85975 Breast Cancer | 52.2 |
| 6546-1 | | (OD04590-01) | |
| 84140 Prostate Cancer | 22.5 | 85976 Breast Cancer Mets | 23.4 |
| (OD04410) | | (OD04590-03) | |
| 84141 Prostate NAT | 32.5 | 87070 Breast Cancer Metastasis | 22.5 |
| (OD04410) | | (OD04655-05) | |
| 87073 Prostate Cancer | 18.4 | GENPAK Breast Cancer | 19.3 |
| (OD04720-01) | | 064006 | |
| 87074 Prostate NAT | 19.5 | Breast Cancer Res. Gen. 1024 | 20.9 |
| (OD04720-02) | | | |
| Normal Lung GENPAK 061010 | 23.5 | Breast Cancer Clontech | 11.1 |
| | | 9100266 | |
| 83239 Lung Met to Muscle | 1.4 | Breast NAT Clontech 9100265 | 6.9 |
| (ODO4286) | | | |
| 83240 Muscle NAT | 5.2 | Breast Cancer INVITROGEN | 23.8 |
| (ODO4286) | | A209073 | |
| 84136 Lung Malignant Cancer | 17.4 | Breast NAT INVITROGEN | 17.2 |
| (OD03126) | | A2090734 | |
| 84137 Lung NAT (OD03126) | 25.0 | Normal Liver GENPAK | 4.1 |
| | | 061009 | |
| 84871 Lung Cancer (OD04404) | 9.2 | Liver Cancer GENPAK 064003 | 3.4 |
| 84872 Lung NAT (OD04404) | 7.9 | Liver Cancer Research Genetics | 1.4 |
| | | RNA 1025 | |
| 84875 Lung Cancer (OD04565) | 3.2 | Liver Cancer Research Genetics | 2.1 |
| | | RNA 1026 | |
| 84876 Lung NAT (OD04565) | 5.2 | Paired Liver Cancer Tissue | 4.1 |
| | | Research Genetics RNA 6004-T | |
| 85950 Lung Cancer (OD04237- | 15.9 | Paired Liver Tissue Research | 3.0 |
| 01) | | Genetics RNA 6004-N | |
| 85970 Lung NAT (OD04237- | 8.5 | Paired Liver Cancer Tissue | 2.2 |
| 02) | | Research Genetics RNA 6005-T | |
| 83255 Ocular Mel Met to Liver | 5.2 | Paired Liver Tissue Research | 0.6 |
| (ODO4310) | | Genetics RNA 6005-N | |
| 83256 Liver NAT (ODO4310) | 3.8 | Normal Bladder GENPAK | 37.1 |
| | | 061001 | |
| 84139 Melanoma Mets to Lung | 7.7 | Bladder Cancer Research | 2.4 |
| (OD04321) | | Genetics RNA 1023 | |
| 84138 Lung NAT (OD04321) | 21.5 | Bladder Cancer INVITROGEN | 14.4 |
| | | A302173 | |
| Normal Kidney GENPAK | 42.4 | 87071 Bladder Cancer | 11.7 |
| 061008 | | (OD04718-01) | |
| 83786 Kidney Ca, Nuclear | 26.6 | 87072 Bladder Normal | 4.8 |
| grade 2 (OD04338) | | Adjacent (OD04718-03) | |
| 83787 Kidney NAT (OD04338) | 11.5 | Normal Ovary Res. Gen. | 1.5 |
| 83788 Kidney Ca Nuclear grade | 24.5 | Ovarian Cancer GENPAK | 22.4 |
| 1/2 (OD04339) | | 064008 | |
| 83789 Kidney NAT (OD04339) | 29.5 | 87492 Ovary Cancer | 48.4 |
| | | (OD04768-07) | |
| 83790 Kidney Ca, Clear cell | 43.4 | 87493 Ovary NAT (OD04768- | 1.7 |
| type (OD04340) | | 08) | |
| 83791 Kidney NAT (OD04340) | 26.5 | Normal Stomach GENPAK | 12.0 |
| | | 061017 | |
| 83792 Kidney Ca, Nuclear | 1.4 | Gastric Cancer Clontech | 0.7 |
| grade 3 (OD04348) | | 9060358 | |
| 83793 Kidney NAT (OD04348) | 18.8 | NAT Stomach Clontech | 5.3 |
| | | 9060359 | |
| 87474 Kidney Cancer | 2.6 | Gastric Cancer Clontech | 5.6 |
| (OD04622-01) | | 9060395 | |
| 87475 Kidney NAT (OD04622- | 3.1 | NAT Stomach Clontech | 6.2 |
| 03) | | 9060394 | |
| 85973 Kidney Cancer | 100.0 | Gastric Cancer Clontech | 11.7 |
| (OD04450-01) | | 9060397 | |
| 85974 Kidney NAT (OD04450- | 34.1 | NAT Stomach Clontech | 0.7 |
| 03) | | 9060396 | |
| Kidney Cancer Clontech | 1.9 | Gastric Cancer GENPAK | 22.9 |
| 8120607 | | 064005 | |

TABLE 61
Panel 4D

| | Relative | | Relative |
|---|---|---|---|
| | Expression(%) | | Expression(%) |
| | 4dx4tm4548f_{—} | | 4dx4tm4548f_{—} |
| Tissue Name | ag2771_b2 | Tissue Name | ag2771_b2 |
| 93768_Secondary Th1_anti- | 0.1 | 93100_HUVEC | 54.2 |
| CD28/anti-CD3 | | (Endothelial)_IL-1b | |
| 93769_Secondary Th2_anti- | 0.3 | 93779_HUVEC | 50.4 |
| CD28/anti-CD3 | | (Endothelial)_IFN gamma | |
| 93770_Secondary Tr1_anti- | 0.0 | 93102_HUVEC | 15.6 |
| CD28/anti-CD3 | | (Endothelial)_TNF alpha + IFN | |
| | | gamma | |
| 93573_Secondary Th1_resting | 0.0 | 93101_HUVEC | 25.7 |
| day 4-6 in IL-2 | | (Endothelial)_TNF alpha + IL4 | |
| 93572_Secondary Th2_resting | 0.3 | 93781_HUVEC | 23.3 |
| day 4-6 in IL-2 | | (Endothelial)_IL-11 | |
| 93571_Secondary Tr1_resting | 0.0 | 93583_Lung Microvascular | 71.5 |
| day 4-6 in IL-2 | | Endothelial Cells_none | |
| 93568_primary Th1_anti- | 0.0 | 93584_Lung Microvascular | 15.2 |
| CD28/anti-CD3 | | Endothelial Cells_TNFa (4 ng/ml) | |
| | | and IL1b (1 ng/ml) | |
| 93569_primary Th2_anti- | 0.2 | 92662_Microvascular Dermal | 75.3 |
| CD28/anti-CD3 | | endothelium_none | |
| 93570_primary Tr1_anti- | 0.6 | 92663_Microvascular Dermal | 44.2 |
| CD28/anti-CD3 | | endothelium_TNFa (4 ng/ml) | |
| | | and IL1b (1 ng/ml) | |
| 93565_primary Th1_resting dy | 1.6 | 93773_Bronchial | 21.1 |
| 4-6 in IL-2 | | epithelium_TNFa (4 ng/ml) and | |
| | | IL1b (1 ng/ml)** | |
| 93566_primary Th2_resting dy | 1.4 | 93347_Small Airway | 14.4 |
| 4-6 in IL-2 | | Epithelium_none | |
| 93567_primary Tr1_resting dy | 0.5 | 93348_Small Airway | 5.3 |
| 4-6 in IL-2 | | Epithelium_TNFa (4 ng/ml) | |
| | | and IL1b (1 ng/ml) | |
| 93351_CD45RA CD4 | 0.8 | 92668_Coronery Artery | 47.2 |
| lymphocyte_anti-CD28/anti- | | SMC_resting | |
| CD3 | | | |
| 93352_CD45RO CD4 | 1.0 | 92669_Coronery Artery | 5.0 |
| lymphocyte_anti-CD28/anti- | | SMC_TNFa (4 ng/ml) and IL1b | |
| CD3 | | (1 ng/ml) | |
| 93251_CD8 Lymphocytes_anti- | 0.5 | 93107_astrocytes_resting | 46.9 |
| CD28/anti-CD3 | | | |
| 93353_chronic CD8 | 2.1 | 93108_astrocytes_TNFa (4 ng/ml) | 32.0 |
| Lymphocytes 2ry_resting dy 4-6 | | and IL1b (1 ng/ml) | |
| in IL-2 | | | |
| 93574_chronic CD8 | 0.0 | 92666_KU-812 | 3.2 |
| Lymphocytes 2ry_activated | | (Basophil)_resting | |
| CD3/CD28 | | | |
| 93354_CD4_none | 0.8 | 92667_KU-812 | 11.8 |
| | | (Basophil)_PMA/ionoycin | |
| 93252_Secondary | 0.2 | 93579_CCD1106 | 15.5 |
| Th1/Th2/Tr1_anti-CD95 CH11 | | (Keratinocytes)_none | |
| 93103_LAK cells_resting | 0.4 | 93580_CCD1106 | 11.6 |
| | | (Keratinocytes)_TNFa and | |
| | | IFNg** | |
| 93788_LAK cells_IL-2 | 0.5 | 93791_Liver Cirrhosis | 5.7 |
| 93787_LAK cells_IL-2 + IL-12 | 0.8 | 93792_Lupus Kidney | 13.5 |
| 93789_LAK cells_IL-2 + IFN | 1.5 | 93577_NCI-H292 | 55.3 |
| gamma | | | |
| 93790_LAK cells_IL-2 + IL-18 | 1.0 | 93358_NCI-H292_IL-4 | 57.1 |
| 93104_LAK | 1.7 | 93360_NCI-H292_IL-9 | 92.5 |
| cells_PMA/ionomycin and IL- | | | |
| 18 | | | |
| 93578_NK Cells IL-2_resting | 0.1 | 93359_NCI-H292_IL-13 | 37.0 |
| 93109_Mixed Lymphocyte | 1.3 | 93357_NCI-H292_IFN gamma | 84.6 |
| Reaction_Two Way MLR | | | |
| 93110_Mixed Lymphocyte | 0.7 | 93777_HPAEC_- | 31.5 |
| Reaction_Two Way MLR | | | |
| 93111_Mixed Lymphocyte | 0.2 | 93778_HPAEC_IL-1 beta/TNA | 15.7 |
| Reaction_Two Way MLR | | alpha | |
| 93112_Mononuclear Cells | 1.1 | 93254_Normal Human Lung | 3.4 |
| (PBMCs)_resting | | Fibroblast_none | |
| 93113_Mononuclear Cells | 14.1 | 93253_Normal Human Lung | 2.6 |
| (PBMCs)_PWM | | Fibroblast_TNFa (4 ng/ml) and | |
| | | IL-1b (1 ng/ml) | |
| 93114_Mononuclear Cells | 12.2 | 93257_Normal Human Lung | 4.9 |
| (PBMCs)_PHA-L | | Fibroblast_IL-4 | |
| 93249_Ramos (B cell)_none | 0.0 | 93256_Normal Human Lung | 3.8 |
| | | Fibroblast_IL-9 | |
| 93250_Ramos (B | 0.1 | 93255_Normal Human Lung | 3.2 |
| cell)_ionomycin | | Fibroblast_IL-13 | |
| 93349_B lymphocytes_PWM | 25.6 | 93258_Normal Human Lung | 6.2 |
| | | Fibroblast_IFN gamma | |
| 93350_B lymphoytes_CD40L | 3.1 | 93106_Dermal Fibroblasts | 6.8 |
| and IL-4 | | CCD1070_resting | |
| 92665_EOL-1 | 0.1 | 93361_Dermal Fibroblasts | 5.3 |
| (Eosinophil)_dbcAMP | | CCD1070_TNF alpha 4 ng/ml | |
| differentiated | | | |
| 93248_EOL-1 | 0.1 | 93105_Dermal Fibroblasts | 3.1 |
| (Eosinophil)_dbcAMP/PMAionomycin | | CCD1070_IL-1 beta 1 ng/ml | |
| 93356_Dendritic Cells_none | 3.2 | 93772_dermal fibroblast_IFN | 2.2 |
| | | gamma | |
| 93355_Dendritic Cells_LPS | 2.2 | 93771_dermal fibroblast_IL-4 | 6.1 |
| 100 ng/ml | | | |
| 93775_Dendritic Cells_anti- | 1.6 | 93260_IBD Colitis 2 | 1.1 |
| CD40 | | | |
| 93774_Monocytes_resting | 0.7 | 93261_IBD Crohns | 6.2 |
| 93776_Monocytes_LPS 50 ng/ml | 1.0 | 735010_Colon_normal | 29.5 |
| 93581_Macrophages_resting | 5.2 | 735019_Lung_none | 20.0 |
| 93582_Macrophages_LPS 100 ng/ml | 1.1 | 64028-1_Thymus_none | 60.1 |
| 93098_HUVEC | 50.9 | 64030-1_Kidney_none | 16.6 |
| (Endothelial)_none | | | |
| 93099_HUVEC | 100.0 | | |
| (Endothelial)_starved | | | |

TABLE 62
Panel CNS_neurodegeneration_v1.0

| | Relative | | Relative |
|---|---|---|---|
| | Expression(%) | | Expression(%) |
| | tm7061f_{—} | | tm7061f_{—} |
| Tissue Name | ag2771_b1s2 | Tissue Name | ag2771_b1s2 |
| 106655_4951 Hippo | 15.5 | 106677_4624 BA21 | 7.7 |
| 106657_4986 Hippo | 35.1 | 106681_4640 BA21 | 50.4 |
| 106652_4933 Hippo | 11.5 | 106654_4951 BA17 | 20.3 |
| 106649_4901 Hippo | 16.4 | cns_water | 0.0 |
| 110138_3087 hippo | 72.0 | 106651_4933 BA17 | 8.5 |
| 110121_3027 Hippo | 100.0 | 106648_4901 BA17 | 27.5 |
| 106670_4971 Hippo | 33.6 | 110123_3027 Occ Ctx | 27.8 |
| 106666_4867 Hippo | 22.9 | 110140_3087 occ ctx | 38.3 |
| 106680_4624 Hippo | 15.0 | 106659_4595 BA17 | 4.5 |
| 106653_4951 BA21 | 27.1 | 106668_4971 BA17 | 29.0 |
| 106656_4986 BA21 | 43.7 | 106662_4737 BA17 | 17.4 |
| 106650_4933 BA21 | 10.0 | 106665_4867 BA17 | 11.4 |
| 106647_4901 BA21 | 21.3 | 106675_3975 BA17 | 83.9 |
| 110136_3087 inf temp ctx | 89.6 | 106672_3954 BA17 | 16.4 |
| 110137_3087 sup temp ctx | 63.8 | 106678_4624 BA17 | 5.3 |
| 110118_3027 Inf Temp Ctx | 77.7 | 106682_4640 BA17 | 17.5 |
| 110119_3027 Sup Temp Ctx | 80.4 | 106660_4595 BA7 | 9.5 |
| 106658_4595 BA21 | 10.5 | 113670_106669 pool | 62.9 |
| 106667_4971 BA21 | 31.8 | 106663_4737 BA7 | 11.5 |
| 106661_4737 BA21 | 20.8 | 106676_3975 BA7 | 53.6 |
| 106664_4867 BA21 | 13.2 | 106673_3954 BA7 | 28.4 |
| 106674_3975 BA21 | 72.4 | 106679_4624 BA7 | 7.7 |
| 106671_3954 BA21 | 48.4 | 106683_4640 BA7 | 35.3 |

Panel 1.3D Summary Ag2771 Expression of the NOV9 gene is highest in normal bladder (CT=27.2). This gene is more highly expressed in colon cancer cell lines relative to normal colon as well as in some lung cancer cell lines relative to normal lung. Thus, expression of this gene could be used to distinguish between colon or lung cancer cell lines and other cell lines. Furthermore, therapeutic inhibition of the NOV9 gene or its protein product, throught the use of antibodies, small molecule or protein drugs, may be effective in the treatment of colon and lung cancers.

The NOV9 gene is expressed at moderate levels throughout CNS, with expression detected in fetal brain, amygdala, cerebellum, hippocampus, substantia nigra, thalamus, cerebral cortex and spinal cord (CTs=29-33). See CNS_neurodegeneration_panel_v1.0 summary for potential utility of this gene in CNS disorders.

Among tissues with metabolic function, this gene shows low expression in pancreas, adrenal gland, pituitary gland, and liver (adult and fetal) with higher expression in thyroid, heart (adult and fetal), skeletal muscle (adult and fetal), and adipose. Therefore, the NOV9 gene product may play a role in the pathogenesis and/or treatment of metabolic diseases in any or all of these tissues, including obesity and diabetes.

Panel 2D Summary Ag2771 Highest expression of the NOV9 gene is found in a kidney cancer sample (CT=23). However, this gene is rather ubiquitously expressed at moderate levels in all the tissue samples on this panel. Interestingly, the level of NOV9 gene expression appears to be lower in liver and lung tissues when compared to other organs. In addition, this gene appears to be overexpressed in ovarian cancers as well as in several colon cancers relative to the normal controls. The expression patterns suggest that this gene is required for the survival and proliferation of the majority of cell types.

Panel 4D Summary Ag2771 The NOV9 gene encodes protein with homology to CDC-42-interacting protein 4 and is highly expressed in resting HUVEC endothelial cells (CT=24-25), lung microvascular endothelium, bronchial epithelium, small airway epithelium, coronary artery smooth muscle cells, as well as in mucoepidermoid cells (NC1-H292). Basal expression of this gene in these cells appears to be decreased by various treatments with proinflammatory cytokines, such as IL-1beta, IL-4, IL-11, IFN-gamma, and TNF-alpha. CDC-42-interacting protein 4 is a Cdc42 effector protein involved in cytoskeletal organization (ref. 1-2). Since cytokine-activated cells express lower levels of the NOV9 gene, increasing the activity of this gene product may reduce the pro-inflammatory effects of these cytokines. Therefore, the NOV9 gene product may be a useful target for agonistic small molecule therapeutics that increase activity of the protein, and such small molecule drugs may reduce the severity of symptoms of asthma and inflammatory bowel disease.

Panel CNS_neurodegeneration_v1.0 Summary Ag2771 CDC42 has been implicated as a neuronal death effector in Alzheimer's disease and as playing an essential role in cerebellar granule neuron survival (refs. 4-6). Cdc42-interacting protein 4, which is homologous to the NOV9 gene, has been identified as a substrate of CDC42 (ref. 2). Therefore, drugs that inhibit NOV9 gene product activity may be effective in blocking processes downstream of CDC42, such as neuronal death in Alzheimer's disease. Since CDC42 can mediate both desirable (cerebellar neuronal survival) and undesirable (Alzheimer's disease) processes, it is likely that specifically targeting distinct downstream substrates may enable the effective targeting of distinct processes without affecting other CDC42-mediated processes. The NOV9 gene is expressed in the human brain in all regions examined, including the cerebral cortex, hippocampus, amygdala, cerebellum, substantia nigra, spinal cord and thalamus (see Panel 1.3D). Additionally, this gene is expressed more highly in the hippocampus of some patients with Alzheimer's disease than in normal control brains, indicating a possible pathological role in neurodegenerative brain disease. Therefore, targeting the NOV9 gene product may have utility in selective targeting of undesirable CDC42-mediated processes, such as Alzheimer's disease.

References:

1. Linder S, Hufner K, Wintergerst U, Aepfelbacher M. Microtubule-dependent formation of podosomal adhesion structures in primary human macrophages. J Cell Sci 2000 Dec;113 Pt 23:4165-76.

Podosomes are unique actin-rich adhesion structures of monocyte-derived cells such as macrophages and osteoclasts. They clearly differ from other substratum-contacting organelles like focal adhesions in morphological and functional regards. Formation of podosomes has been shown to be dependent on the small GTPase CDC42Hs and its effector Wiskott-Aldrich syndrome protein (WASp). In this study, we investigated the functional relation between podosomes and the microtubule system in primary human macrophages. We demonstrate that, in contrast to focal adhesions, assembly of podosomes in macrophages and their monocytic precursors is dependent on an intact microtubule system. In contrast, experiments using Wiskott-Aldrich syndrome (WAS) macrophages indicate that the microtubule system is not reciprocally dependent on podosomes. A potential linker between podosomes and microtubules may be WASp itself, considering that microinjection of the WASp polyproline domain prevents podosome reassembly. This polyproline domain is thought to link WASp to microtubules via CDC42 interacting protein 4 (CIP4). Consistently, macrophages microinjected with CIP4 constructs deficient in either the microtubule- or the WASp-binding domain also fail to reassemble podosomes. In sum, our findings show that microtubules are essential for podosome formation in primary human macrophages and that WASp and CIP4 may be involved in this phenomenon.

PMID: 11069762

2. Tian L, Nelson D L, Stewart D M. Cdc42-interacting protein 4 mediates binding of the Wiskott-Aldrich syndrome protein to microtubules. J Biol Chem 2000 Mar 17;275(11):7854-61

The Wiskott-Aldrich syndrome is an inherited X-linked immunodeficiency characterized by thrombocytopenia, eczema, and a tendency toward lymphoid malignancy. Lymphocytes from affected individuals have cytoskeletal abnormalities, and monocytes show impaired motility. The Wiskott-Aldrich syndrome protein (WASP) is a multi-domain protein involved in cytoskeletal organization. In a two-hybrid screen, we identified the protein Cdc42-interacting protein 4 (CIP4) as a WASP interactor. CIP4, like WASP, is a Cdc42 effector protein involved in cytoskeletal organization. We found that the WASP-CIP4 interaction is mediated by the binding of the Src homology 3 domain of CIP4 to the proline-rich segment of WASP. Cdc42 was not required for this interaction. Co-expression of CIP4 and green fluorescent protein-WASP in COS-7 cells led to the association of WASP with microtubules. In vitro experiments showed that CIP4 binds to microtubules via its NH(2) terminus. The region of CIP4 responsible for binding to active Cdc42 was localized to amino acids 383-417, and the mutation I398S abrogated binding. Deletion of the Cdc42-binding domain of CIP4 did not affect the colocalization of WASP with microtubules in vivo. We conclude that CIP4 can mediate the association of WASP with microtubules. This may facilitate transport of WASP to sites of substrate adhesion in hematopoietic cells.

PMID: 10713100

3. Aspenstrom P.A Cdc42 target protein with homology to the non-kinase domain of FER has a potential role in regulating the actin cytoskeleton. Curr Biol 1997 Jul 1;7(7):479-87

BACKGROUND: Members of the Rho family of small GTPases have been shown to have a diverse role in cell signalling events. They were originally identified as proteins that, by regulating the assembly of the actin cytoskeleton, are important determinants of cell morphology, and have recently been shown to be involved in transcriptional activation by the JNK/SAPK signalling pathway. In order to understand the mechanisms underlying the effects of Rho GTPases on these processes, the yeast two-hybrid system has been used to identify proteins that bind to an activated mutant of Cdc42, a Rho-family member. RESULTS: A cDNA encoding a previously unidentified Cdc42 target protein, CIP4, which is 545 amino-acids long and contains an SH3 domain at its carboxyl terminus, was cloned from a human B-cell library. The amino terminus of CIP4 bears resemblance to the non-kinase domain of the FER and Fes/Fps family of tyrosine kinases. In addition, similarities to a number of proteins with roles in regulating the actin cytoskeleton were noticed. CIP4 binds to activated Cdc42 in vitro and in vivo and overexpression of CIP4 in Swiss 3T3 fibroblasts reduces the amount of stress fibres in these cells. Moreover, coexpression of activated Cdc42 and CIP4 leads to clustering of CIP4 to a large number of foci at the dorsal side of the cells. CONCLUSIONS: CIP4 is a downstream target of activated GTP-bound Cdc42, and is similar in sequence to proteins involved in signalling and cytoskeletal control. Together, these findings suggest that CIP4 may act as a link between Cdc42 signalling and regulation of the actin cytoskeleton.

PMID: 9210375

4. Mota M, Reeder M, Chernoff J, Bazenet C E. Evidence for a role of mixed lineage kinases in neuronal apoptosis. JNeurosci 2001 Jul 15;21(14):4949-57

Superior cervical ganglion (SCG) sympathetic neurons die by apoptosis when deprived of nerve growth factor (NGF). It has been shown previously that the induction of apoptosis in these neurons at NGF withdrawal requires both the activity of the small GTP-binding protein Cdc42 and the activation of the c-Jun N-terminal kinase (JNK) pathway. The mixed lineage kinase 3 (MLK3) belongs to a family of mitogen-activated protein (MAP) kinase kinase kinases. MLK3 contains a Cdc42/Rac interactive-binding (CRIB) domain and activates both the JNK and the p38 MAP kinase pathways. In this study the role of MLK3 in the induction of apoptosis in sympathetic neurons has been investigated. Overexpression of an active MLK3 induces activation of the JNK pathway and apoptosis in SCG neurons. In addition, overexpression of kinase dead mutants of MLK3 blocks apoptosis as well as c-Jun phosphorylation induced by NGF deprivation. More importantly, MLK3 activity seems to increase by 5 hr after NGF withdrawal in both differentiated PC 12 cells and SCG neurons. We also show that MLK3 lies downstream of Cdc42 in the neuronal death pathway. Regulation of MLK3 in neurons seems to be dependent on MLK3 activity and possibly on an additional cellular component, but not on its binding to Cdc42. These results suggest that MLK3, or a closely related kinase, is a physiological element of NGF withdrawal-induced activation of the Cdc42-c-Jun pathway and neuronal death. MLK3 therefore could be an interesting therapeutic target in a number of neurodegenerative diseases involving neuronal apoptosis.

PMID: 11438570

5. Linseman D A, Laessig T, Meintzer N K, McClure M, Barth H, Aktories K, Heidenreich K A. An essential role for Rac/Cdc42 GTPases in cerebellar granule neuron survival. J Biol Chem 2001 Aug 16; [epub ahead of print]

Rho family GTPases are critical molecular switches that regulate the actin cytoskeleton and cell function. In the current study, we investigated the involvement of Rho GTPases in regulating neuronal survival using primary cerebellar granule neurons. C. difficile toxin B, a specific inhibitor of Rho, Rac and Cdc42, induced apoptosis of granule neurons characterized by c-Jun phosphorylation, caspase-3 activation and nuclear condensation. Serum and depolarization-dependent survival signals could not compensate for the loss of GTPase function. Unlike trophic factor withdrawal, toxin B did not affect the anti-apoptotic kinase Akt or its target glycogen synthase kinase-3beta. The pro-apoptotic effects of toxin B were mimicked by C. sordellii lethal toxin, a selective inhibitor of Rac/Cdc42. Although Rac/Cdc42 GTPase inhibition led to F-actin disruption, direct cytoskeletal disassembly with C. botulinum C2 toxin was insufficient to induce c-Jun phosphorylation or apoptosis. Granule neurons expressed high basal JNK and low p38 MAPK activities that were unaffected by toxin B. However, pyridylimidazole inhibitors of JNK/p38 attenuated c-Jun phosphorylation. Moreover, both pyridylimidazoles and adenoviral dominant-negative c-Jun attenuated apoptosis, suggesting that JNK/c-Jun signaling was required for cell death. The results indicate that Rac/Cdc42 GTPases, in addition to trophic factors, are critical for survival of cerebellar granule neurons.

PMID: 11509562

6. Zhu X, Raina A K, Boux H, Simmons Z L, Takeda A, Smith M A. Activation of oncogenic pathways in degenerating neurons in Alzheimer disease. Int J Dev Neurosci 2000 Jul-Aug;18(4-5):433-7

A number of recent findings have highlighted the similarities between neurogenesis during development and neurodegeneration during Alzheimer disease. In fact, neuronal populations that are known to degenerate in Alzheimer disease exhibit phenotypic changes characteristic of cells re-entering the cell division cycle. In this study, we extended these findings by investigating components of the cell cycle, known to trigger progression through GI through activation of signal transduction cascades. Specifically, we found that proteins implicated in G1 transition, namely Cdc42/Rac, are upregulated in select neuronal populations in cases of Alzheimer disease in comparison to age-matched controls. Importantly, Cdc42/Rac shows considerable overlap with early cytoskeletal abnormalities suggesting that these changes are an extremely proximal event in the pathogenesis of the disease. Given the functional role of Cdc42/Rac in various cellular processes known to be perturbed in Alzheimer disease, namely cytoskeletal organization, oxidative balance, and oncogenic signaling, it is likely that increased neuronal Cdc42/Rac is highly significant in relation to the pathogenic process and contributes to neuronal degeneration. In fact, these findings suggest that Alzheimer disease is an oncogenic process.

PMID: 10817927

NOV10a

Expression of gene NOV10a was assessed using the primer-probe set Ag1674 described in Table 63. Results from RTQ-PCR runs are shown in Tables 64, 65, and 66.

TABLE 63
Probe Name Ag1674

| | | | | Start | SEQ ID |
|---|---|---|---|---|---|
| Primers | Sequences | TM | Length | Position | NO: |
| Forward | 5′-CTCACTCACCACAAGGGAGTAA-3′ | 59.3 | 22 | 519 | 156 |
| Probe | FAM-5′-TGACATCAAACTCAACAGTTCCCAGGA-3′-TAMRA | 68.8 | 27 | 548 | 157 |
| Reverse | 5′-GTCTAGGAGAGAGCTGAGCAAA-3′ | 58.1 | 22 | 576 | 158 |

TABLE 64
Panel CNS_1

| | Relative Expression(%) | |
|---|---|---|
| | cns1x4tm6180f_{—} | cns1tm6571f_{—} |
| Tissue Name | ag1674_b2 | ag1674 |
| 102633_BA4 Control | 1.3 | 3.6 |
| 102641_BA4 Control2 | 0.8 | 3.7 |
| 102625_BA4 Alzheimer's2 | 2.4 | 0.7 |
| 102649_BA4 Parkinson's | 15.3 | 12.4 |
| 102656_BA4 Parkinson's2 | 30.0 | 23.0 |
| 102664_BA4 Huntington's | 4.7 | 8.2 |
| 102671_BA4 Huntington's2 | 5.7 | 1.0 |
| 102603_BA4 PSP | 3.3 | 3.9 |
| 102610_BA4 PSP2 | 0.9 | 4.2 |
| 102588_BA4 Depression | 5.2 | 4.2 |
| 102596_BA4 Depression2 | 1.6 | 4.0 |
| 102634_BA7 Control | 3.7 | 3.7 |
| 102642_BA7 Control2 | 0.0 | 3.7 |
| 102626_BA7 Alzheimer's2 | 0.0 | 0.8 |
| 102650_BA7 Parkinson's | 1.5 | 15.2 |
| 102657_BA7 Parkinson's2 | 21.1 | 21.3 |
| 102665_BA7 Huntington's | 7.9 | 3.3 |
| 102672_BA7 Huntington's2 | 14.7 | 10.3 |
| 102604_BA7 PSP | 11.5 | 7.4 |
| 102611_BA7 PSP2 | 2.2 | 2.4 |
| 102589_BA7 Depression | 3.0 | 2.5 |
| 102632_BA9 Control | 0.0 | 2.5 |
| 102640_BA9 Control2 | 8.7 | 6.8 |
| 102617_BA9 Alzheimer's | 2.4 | 0.8 |
| 102624_BA9 Alzheimer's2 | 3.6 | 5.4 |
| 102648_BA9 Parkinson's | 3.6 | 8.4 |
| 102655_BA9 Parkinson's2 | 21.1 | 33.0 |
| 102663_BA9 Huntington's | 15.7 | 12.5 |
| 102670_BA9 Huntington's2 | 6.7 | 8.4 |
| 102602_BA9 PSP | 5.0 | 5.5 |
| 102609_BA9 PSP2 | 0.7 | 0.0 |
| 102587_BA9 Depression | 1.0 | 2.9 |
| 102595_BA9 Depression2 | 3.8 | 4.8 |
| 102635_BA17 Control | 2.8 | 7.7 |
| 102643_BA17 Control2 | 0.7 | 3.1 |
| 102627_BA17 Alzheimer's2 | 1.3 | 2.7 |
| 102651_BA17 Parkinson's | 11.8 | 6.9 |
| 102658_BA17 Parkinson's2 | 33.4 | 27.5 |
| 102666_BA17 Huntington's | 4.1 | 5.1 |
| 102673_BA17 Huntington's2 | 5.7 | 2.0 |
| 102590_BA17 Depression | 5.1 | 5.1 |
| 102597_BA17 Depression2 | 2.4 | 9.3 |
| 102605_BA17 PSP | 5.0 | 14.7 |
| 102612_BA17 PSP2 | 2.8 | 0.8 |
| 102637_Sub Nigra Control | 12.3 | 15.9 |
| 102645_Sub Nigra Control | 3.1 | 9.7 |
| 102629_Sub Nigra | 3.6 | 1.5 |
| Alzheimer's2 | | |
| 102660_Sub Nigra | 100.0 | 95.9 |
| Parkinson's2 | | |
| 102667_Sub Nigra | 16.4 | 6.4 |
| Huntington's | | |
| 102674_Sub Nigra | 15.3 | 16.7 |
| Huntington's2 | | |
| 102614_Sub Nigra PSP2 | 7.2 | 3.7 |
| 102592_Sub Nigra | 2.4 | 1.6 |
| Depression | | |
| 102599_Sub Nigra | 5.9 | 3.7 |
| Depression2 | | |
| 102636_Glob Palladus | 3.7 | 6.4 |
| Control | | |
| 102644_Glob Palladus | 0.9 | 1.1 |
| Control2 | | |
| 102620_Glob Palladus | 3.5 | 2.7 |
| Alzheimer's | | |
| 102628_Glob Palladus | 0.0 | 2.8 |
| Alzheimer's2 | | |
| 102652_Glob Palladus | 26.1 | 27.2 |
| Parkinson's | | |
| 102659_Glob Palladus | 21.7 | 38.2 |
| Parkinson's2 | | |
| 102606_Glob Palladus | 7.4 | 4.8 |
| PSP | | |
| 102613_Glob Palladus | 0.3 | 0.9 |
| PSP2 | | |
| 102591_Glob Palladus | 3.9 | 3.7 |
| Depression | | |
| 102638_Temp Pole | 0.7 | 1.9 |
| Control | | |
| 102646_Temp Pole | 2.6 | 4.2 |
| Control2 | | |
| 102622_Temp Pole | 0.5 | 3.9 |
| Alzheimer's | | |
| 102630_Temp Pole | 1.8 | 2.1 |
| Alzheimer's2 | | |
| 102653_Temp Pole | 6.2 | 11.3 |
| Parkinson's | | |
| 102661_Temp Pole | 16.5 | 23.7 |
| Parkinson's2 | | |
| 102668_Temp Pole | 1.0 | 4.0 |
| Huntington's | | |
| 102607_Temp Pole PSP | 1.5 | 4.0 |
| 102615_Temp Pole PSP2 | 1.4 | 0.9 |
| 102600_Temp Pole | 2.3 | 1.7 |
| Depression2 | | |
| 102639_Cing Gyr | 9.0 | 23.5 |
| Control | | |
| 102647_Cing Gyr | 6.2 | 3.8 |
| Control2 | | |
| 102623_Cing Gyr | 4.0 | 6.5 |
| Alzheimer's | | |
| 102631_Cing Gyr | 1.5 | 1.7 |
| 102654_Cing Gyr | 0.0 | 15.8 |
| Parkinson's | | |
| 102662_Cing Gyr | 46.1 | 100.0 |
| Parkinson's2 | | |
| 102669_Cing Gyr | 14.0 | 10.9 |
| Huntington's | | |
| 102676_Cing Gyr | 14.9 | 13.6 |
| Huntington's2 | | |
| 102608_Cing Gyr | 16.1 | 32.8 |
| PSP | | |
| 102616_Cing Gyr | 1.8 | 0.8 |
| PSP2 | | |
| 102594_Cing Gyr | 3.2 | 4.9 |
| Depression | | |
| 102601_Cing Gyr | 6.6 | 11.0 |
| Depression2 | | |

TABLE 65
Panel CNS_1.1

| | Relative Expression(%) | |
|---|---|---|
| | | cns_1.1tm |
| | cns_1.1tm6733 | 6734f_{—} |
| Tissue Name | f_ag1674_a2 | ag1674_a2 |
| 102601_Cing Gyr Depression2 | 3.0 | 2.4 |
| 102594_Cing Gyr Depression | 1.6 | 0.4 |
| 102616_Cing Gyr PSP2 | 1.2 | 0.6 |
| 102608_Cing Gyr PSP | 20.8 | 8.2 |
| 102676_Cing Gyr Huntington's2 | 19.7 | 5.5 |
| 102669_Cing Gyr Huntington's | 9.6 | 6.6 |
| 102662_Cing Gyr Parkinson's2 | 45.3 | 100.0 |
| 102654_Cing Gyr Parkinson's | 18.7 | 4.6 |
| 102631_Cing Gyr Alzheimer's2 | 0.0 | 0.4 |
| 102623_Cing Gyr Alzheimer's | 4.3 | 0.8 |
| 102647_Cing Gyr Control2 | 2.8 | 1.2 |
| 102639_Cing Gyr Control | 13.3 | 11.5 |
| 102600_Temp Pole Depression2 | 2.3 | 0.4 |
| 102615_Temp Pole PSP2 | 0.0 | 0.0 |
| 102607_Temp Pole PSP | 1.4 | 0.4 |
| 102668_Temp Pole Huntington's | 6.2 | 2.3 |
| 102661_Temp Pole Parkinson's2 | 10.0 | 10.0 |
| 102653_Temp Pole Parkinson's | 6.0 | 0.3 |
| 102630_Temp Pole Alzheimer's2 | 1.3 | 0.0 |
| 102622_Temp Pole Alzheimer's | 0.7 | 0.0 |
| 102646_Temp Pole Control2 | 3.3 | 2.3 |
| 102638_Temp Pole Control | 4.1 | 0.0 |
| 102591_Glob Palladus Depression | 1.4 | 0.8 |
| 102613_Glob Palladus PSP2 | 0.7 | 0.3 |
| 102606_Glob Palladus PSP | 4.4 | 2.9 |
| 102659_Glob Palladus Parkinson's2 | 16.3 | 10.5 |
| 102652_Glob Palladus Parkinson's | 16.6 | 7.0 |
| 102628_Glob Palladus Alzheimer's2 | 2.0 | 1.2 |
| 102620_Glob Palladus Alzheimer's | 0.7 | 56.0 |
| 102644_Glob Palladus Control2 | 0.8 | 0.4 |
| 102636_Glob Palladus Control | 6.3 | 2.3 |
| 102599_Sub Nigra Depression2 | 3.2 | 2.2 |
| 102592_Sub Nigra Depression | 0.7 | 0.5 |
| 102614_Sub Nigra PSP2 | 4.5 | 1.4 |
| 102674_Sub Nigra Huntington's2 | 20.9 | 7.8 |
| 102667_Sub Nigra Huntington's | 6.0 | 7.4 |
| 102660_Sub Nigra Parkinson's2 | 100.0 | 52.5 |
| 102629_Sub Nigra Alzheimer's2 | 0.7 | 0.7 |
| 102645_Sub Nigra Control2 | 2.1 | 1.0 |
| 102637_Sub Nigra Control | 13.2 | 4.5 |
| 102597_BA17 Depression2 | 3.3 | 1.3 |
| 102590_BA17 Depression | 1.4 | 1.0 |
| 102612_BA17 PSP2 | 0.2 | 0.3 |
| 102605_BA17 PSP | 3.6 | 1.1 |
| 102673_BA17 Huntington's2 | 4.6 | 1.2 |
| 102666_BA17 Huntington's | 4.8 | 2.8 |
| 102658_BA17 Parkinson's2 | 35.7 | 13.0 |
| 102651_BA17 Parkinson's | 7.3 | 5.6 |
| 102627_BA17 Alzheimer's2 | 2.8 | 0.6 |
| 102643_BA17 Control2 | 1.9 | 1.1 |
| 102635_BA17 Control | 0.0 | 3.6 |
| 102595_BA9 Depression2 | 0.7 | 0.2 |
| 102587_BA9 Depression | 1.5 | 0.4 |
| 102609_BA9 PSP2 | 0.7 | 0.7 |
| 102602_BA9 PSP | 2.7 | 0.6 |
| 102670_BA9 Huntington's2 | 7.6 | 3.1 |
| 102663_BA9 Huntington's | 6.2 | 6.0 |
| 102655_BA9 Parkinson's2 | 26.7 | 10.8 |
| 102648_BA9 Parkinson's | 6.3 | 1.3 |
| 102624_BA9 Alzheimer's2 | 1.3 | 0.4 |
| 102617_BA9 Alzheimer's | 0.7 | 0.8 |
| 102640_BA9 Control2 | 4.7 | 2.8 |
| 102632_BA9 Control | 0.0 | 1.9 |
| 102589_BA7 Depression | 2.0 | 1.4 |
| 102611_BA7 PSP2 | 2.2 | 0.7 |
| 102604_BA7 PSP | 7.4 | 5.3 |
| 102672_BA7 Huntington's2 | 8.4 | 4.3 |
| 102665_BA7 Huntington's | 11.0 | 3.8 |
| 102657_BA7 Parkinson's2 | 19.8 | 6.9 |
| 102650_BA7 Parkinson's | 3.1 | 2.9 |
| 102626_BA7 Alzheimer's2 | 0.7 | 1.7 |
| 102642_BA7 Control2 | 1.3 | 0.6 |
| 102634_BA7 Control | 3.4 | 3.1 |
| 102596_BA4 Depression2 | 2.6 | 0.6 |
| 102588_BA4 Depression | 0.8 | 2.1 |
| 102610_BA4 PSP2 | 1.7 | 0.0 |
| 102603_BA4 PSP | 2.8 | 1.0 |
| 102671_BA4 Huntington's2 | 4.6 | 1.0 |
| 102664_BA4 Huntington's | 5.5 | 1.7 |
| 102656_BA4 Parkinson's2 | 22.1 | 12.1 |
| 102649_BA4 Parkinson's | 10.1 | 5.6 |
| 102625_BA4 Alzheimer's2 | 0.8 | 0.4 |
| 102641_BA4 Control2 | 2.6 | 1.3 |
| 102633_BA4 Control | 3.3 | 1.4 |

TABLE 66
Panel CNS_neurodegeneration_v1.0

| | Relative | | Relative |
|---|---|---|---|
| | Expression(%) | | Expression(%) |
| | tm7056f_{—} | | tm7056f_{—} |
| Tissue Name | ag1674_a2_s1 | Tissue Name | ag1674_a2_s1 |
| 106655_4951 Hippo | 20.8 | 106677_4624 BA21 | 2.9 |
| 106657_4986 Hippo | 10.4 | 106681_4640 BA21 | 13.7 |
| 106652_4933 Hippo | 3.6 | 106654_4951 BA17 | 15.2 |
| 106649_4901 Hippo | 7.7 | cns_water | 0.0 |
| 110138_3087 hippo | 45.2 | 106651_4933 BA17 | 2.3 |
| 110121_3027 Hippo | 19.1 | 106648_4901 BA17 | 9.6 |
| 106670_4971 Hippo | 12.9 | 110140_3087 occ ctx | 7.7 |
| 106666_4867 Hippo | 17.3 | 110123_3027 Occ Ctx | 4.3 |
| 106680_4624 Hippo | 2.2 | 106659_4595 BA17 | 2.3 |
| 106653_4951 BA21 | 21.6 | 106668_4971 BA17 | 16.3 |
| 106656_4986 BA21 | 9.1 | 106662_4737 BA17 | 8.5 |
| 106650_4933 BA21 | 3.4 | 106665_4867 BA17 | 10.9 |
| 106647_4901 BA21 | 19.7 | 106675_3975 BA17 | 22.0 |
| 110136_3087 inf temp ctx | 100.0 | 106672_3954 BA17 | 2.4 |
| 110137_3087 sup temp ctx | 51.9 | 106678_4624 BA17 | 0.4 |
| 110118_3027 Inf Temp Ctx | 18.9 | 106682_4640 BA17 | 6.9 |
| 110119_3027 SupTemp Ctx | 18.1 | 106660_4595 BA7 | 4.6 |
| 106658_4595 BA21 | 3.7 | 113670_106669 pool | 46.1 |
| 106667_4971 BA21 | 11.4 | 106663_4737 BA7 | 12.3 |
| 106661_4737 BA21 | 9.5 | 106676_3975 BA7 | 14.3 |
| 106664_4867 BA21 | 10.4 | 106673_3954 BA7 | 6.9 |
| 106674_3975 BA21 | 10.9 | 106679_4624 BA7 | 1.9 |
| 106671_3954 BA21 | 10.3 | 106683_4640 BA7 | 13.9 |

Panel CNS_{—}1/CNS_{—}1.1/Panel CNS_neurodegeneration_v1.0 Summary Ag1674

The NOV10a gene encodes a protein with homology to hepsin/plasma transmembrane serine proteases. This gene is more highly expressed in the substantia nigra, globus palladus, BA17, BA4, BA9 and cingulate gyrus region of a Parkinson's disease brain than in the control brains. In addition, expression of this gene is 5-fold higher in the substantia nigra and cingulate gyrus of a Parkinson's disease brain than in similar regions from the brains of patients with other neurodegenerative diseases, such as Alzheimer's or Huntington's disease; this observation suggests that overexpression of the NOV10a gene in these regions is particular to Parkinson's disease. The substantia nigra is particularly vulnerable in Parkinson's disease, indicating that the NOV10a gene product may be a component of the biological dysregulation that contributes to Parkinson's disease pathogenesis. In particular, the NOV10a gene may be involved in the neuronal death that occurs in the substantia nigra in Parkinson's disease.

The NOV 10a gene is also more highly expressed in the superior/inferior temporal cortex, globus palladus, and hippocampus of an Alzheimer's brain when compared to the control brains. This observation suggests that this gene may play a role the neurodegeneration of other diseases in addition to Parkinson's disease, such as Alzheimer's disease. Based on the proposed role of transmembrane proteases such as beta secretase in neurodegenerative disorders, agents that influence the activity of the NOV10a gene product may be useful in treating these disorders, especially Parkinson's disease in which this gene appears to be dramatically upregulated.

### Example 3

### SNP Analysis of NOVX Clones

SeqCalling™ Technology: cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. The cDNA thus derived was then sequenced using CuraGen's proprietary SeqCalling technology. Sequence traces were evaluated manually and edited for corrections if appropriate. cDNA sequences from all samples were assembled with themselves and with public ESTs using bioinformatics programs to generate CuraGen's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations.

Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of novel SNP Confirmation: SNPs are confirmed employing a validated method know as Pyrosequencing (Pyrosequencing, Westborough, Mass.). Detailed protocols for Pyrosequencing can be found in: Alderborn et al. Determination of Single Nucleotide Polymorphisms by Real-time Pyrophosphate DNA Sequencing. (2000). Genome Research. 10, Issue 8, August. 1249-1265. In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PPi) that is released from each dNTP upon DNA chain elongation. Following Klenow polymerase-mediated base incorporation, PPi is released and used as a substrate, together with adenosine 5′-phosphosulfate (APS), for ATP sulfuirylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels. The DNA and protein sequences for the novel single nucleotide polymorphic variants are reported. Variants are reported individually but any combination of all or a select subset of variants are also included. In addition, the positions of the variant bases and the variant amino acid residues are underlined.

### Results

Variants are reported individually but any combination of all or a select subset of variants are also included as contemplated NOVX embodiments of the invention.

NOV1a SNP data:

NOV1a has two SNP variants, whose variant positions for its nucleotide and amino acid sequences is numbered according to SEQ ID NOs: 1 and 2, respectively. The nucleotide sequence of the NOV1a variant differs as shown in Table 67.

TABLE 67
cSNP and Coding Variants for NOV1a

| NT Position | Wild Type | | Amino Acid | Amino Acid |
|---|---|---|---|---|
| of cSNP | NT | Variant NT | position | Change |
| 472 | A | G | 155 | R->G |
| 481 | G | C | 158 | A->P |
| 1121 | T | C | 373 | V->A |
| 1516 | T | C | | No change |
| 1566 | T | C | 553 | C->R |

NOV4 SNP Data:

NOV4 has four SNP variants, whose variant positions for its nucleotide and amino acid sequences is numbered according to SEQ ID NOs: 13 and 14, respectively. The nucleotide sequence of the NOV4 variant differs as shown in Table 68.

TABLE 68
cSNP and Coding Variants for NOV7

| NT Position | Wild Type | | Amino Acid | Amino Acid |
|---|---|---|---|---|
| of cSNP | NT | Variant NT | position | Change |
| 347 | T | A | 114 | H->Q |

NOV7 SNP Data:

NOV7 has one SNP variant, whose variant position for its nucleotide and amino acid sequences is numbered according to SEQ ID NOs: 19 and 20, respectively. The nucleotide sequence of the NOV7 variant differs as shown in Table 69.

TABLE 69
cSNP and Coding Variants for NOV8

| NT Position | Wild Type | | Amino Acid | Amino Acid |
|---|---|---|---|---|
| of cSNP | NT | Variant NT | position | Change |
| 50 | T | C | 17 | S->P |
| 73 | G | A | 24 | W->Stop |
| 111 | G | A | 35 | V->M |
| 120 | C | T | 40 | A->V |
| 160 | C | T | | No change |
| 177 | G | A | 59 | G->D |
| 238 | A | G | | No change |
| 250 | G | A | | No change |
| 278 | A | G | 93 | M->V |
| 285 | G | A | 96 | A->T |
| 297 | A | G | 122 | K->E |
| 746 | A | — | | Frameshift |

SNP at nucleotide 746 has a putative allele frequency of 0.250.

### OTHER EMBODIMENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 27, and 29;
(b) a variant of a mature form of an amino acid sequence selected from the group consisting of SEQ ID NOS:2, 4, 6, 8, 10, 12, 14, 16, 18, 20, 22, 24, 27, and 29, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting SEQ ID NOS:2, 4, 6, 8, 10, 12, 14, 16, 18, 20, 22, 24, 27, and 29; and
(d) a variant of an amino acid sequence selected from the group consisting of SEQ ID NOS:2, 4, 6, 8, 10, 12, 14, 16, 18, 20, 22, 24, 27, and 29, wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting SEQ ID NOS:2, 4, 6, 8, 10, 12, 14, 16, 18, 20, 22, 24, 27, and 29.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS:1, 3, 5, 7, 9, 11, 13, 15, 17, 19, 21, 23, 25, and 28.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 27, and 29;
(b) a variant of a mature form of an amino acid sequence selected from the group consisting of SEQ ID NOS:2, 4, 6, 8, 10, 12, 14, 16, 18, 20, 22, 24, 27, and 29, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 20, 22, 24, 27, and 29;
(d) a variant of an amino acid sequence selected from the group consisting SEQ ID NOS:2, 4, 6, 8, 10, 12, 14, 16, 18, 20, 22, 24, 27, and 29, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 4, 6, 8, 10, 12, 14, 16, 18, 20, 22, 24, 27, and 29, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1, 3, 5, 7, 9, 11, 13, 15, 17, 19, 21, 23, 25, and 28.

9. The nucleic acid molecule of claim 5, wherein said nucleic acid molecule comprises a nucleotide sequence selected from the group consisting of:
(a) a nucleotide sequence selected from the group consisting of SEQ ID NOS: 1, 3, 5, 7, 9, 11, 13, 15, 17, 19, 21, 23, 25, and 28;
(b) a nucleotide sequence differing by one or more nucleotides from a nucleotide sequence selected from the group consisting of SEQ ID NOS:1, 3, 5, 7, 9, 11, 13, 15, 17, 19, 21, 23, 25, and 28, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting SEQ ID NOS:1, 3, 5, 7, 9, 11, 13, 15, 17, 19, 21, 23, 25, and 28, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

27. The method of claim 26 wherein the disorder is selected from the group consisting of cardiomyopathy and atherosclerosis.

28. The method of claim 26 wherein the disorder is related to cell signal processing and metabolic pathway modulation.

29. The method of claim 26, wherein said subject is a human.

30. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

31. The method of claim 30 wherein the disorder is selected from the group consisting of cardiomyopathy and atherosclerosis.

32. The method of claim 30 wherein the disorder is related to cell signal processing and metabolic pathway modulation.

33. The method of claim 30, wherein said subject is a human.

34. A method of treating or preventing a NOVX-associated disorder, said method comprising administering to a subject in which such treatment or prevention is desired the antibody of claim 15 in an amount sufficient to treat or prevent said NOVX-associated disorder in said subject.

35. The method of claim 34 wherein the disorder is diabetes.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to a cancer.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to a cancer.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 4, 6, 8, 10, 12, 14, 16, 18, 20, 22, 24, 27, and 29, or a biologically active fragment thereof.

49. A method of treating a pathological state in a mammal, the method comprising administering to the mammal the antibody of claim 15 in an amount sufficient to alleviate the pathological state.
